(19) United States
(12) Patent Application Publication
(10) Pub. No.: US 2024/0409908 A1
(43) Pub. Date: Dec. 12, 2024

(54) NOVEL CRISPR-ASSOCIATED TRANSPOSON SYSTEMS AND COMPONENTS

(71) Applicant: Arbor Biotechnologies, Inc., Cambridge, MA (US)

(72) Inventors: David A. Scott, Cambridge, MA (US); David R. Cheng, Cambridge, MA (US); Winston X. Yan, Cambridge, MA (US)

(73) Assignee: Arbor Biotechnologies, Inc., Cambridge, MA (US)

(21) Appl. No.: 18/742,602

(22) Filed: Jun. 13, 2024

Related U.S. Application Data
(63) Continuation of application No. 16/761,206, filed on May 1, 2020, now Pat. No. 12,054,754.
(60) Provisional application No. 62/587,381, filed on Nov. 16, 2017.
(60) Provisional application No. 62/580,880, filed on Nov. 2, 2017.

(51) Int. Cl. C12N 9/22; C12N 9/12; C12N 15/10; C12N 15/11; C12N 15/113; C12N 15/63; C12N 15/70; C12N 15/85

(52) U.S. Cl. CPC C12N 9/22; C12N 9/1241; C12N 15/102; C12N 15/1058; C12N 15/11; C12N 15/113; C12N 15/63; C12N 15/70; C12N 15/85; C12N 2310/20; C12N 2800/80

(57) ABSTRACT

The disclosure describes novel systems, methods, and compositions for the manipulation of nucleic acids in a targeted fashion. The disclosure describes non-naturally occurring, engineered CRISPR systems, components, and methods for targeted modification of DNA, RNA, and protein substrates. Each system includes one or more protein components and one or more nucleic acid components that together target DNA, RNA, or protein substrates.

### CROSS REFERENCE TO RELATED APPLICATIONS

This application claims the benefit of priority of U.S. Application No. 62/580,880, filed on Nov. 2, 2017; and U.S. Application No. 62/587,381, filed on Nov. 16, 2017. The content of each of the foregoing applications is hereby incorporated by reference in its entirety.

### FIELD OF THE INVENTION

The present disclosure relates to novel CRISPR systems and components, systems for detecting CRISPR systems, and methods and compositions for use of the CRISPR systems in, for example, nucleic acid targeting and manipulation.

### BACKGROUND

Recent application of advances in genome sequencing technologies and analysis have yielded significant insights into the genetic underpinning of biological activities in many diverse areas of nature, ranging from prokaryotic biosynthetic pathways to human pathologies. To fully understand and evaluate the vast quantities of information produced by genetic sequencing technologies, equivalent increases in the scale, efficacy, and ease of technologies for genome and epigenome manipulation are needed. These novel genome and epigenome engineering technologies will accelerate the development of novel applications in numerous areas, including biotechnology, agriculture, and human therapeutics.

Clustered Regularly Interspaced Short Palindromic Repeats (CRISPR) and the CRISPR-associated (Cas) genes, collectively known as the CRISPR-Cas or CRISPR/Cas systems, are currently understood to provide immunity to bacteria and archaea against phage infection. The CRISPR-Cas systems of prokaryotic adaptive immunity are an extremely diverse group of proteins effectors, non-coding elements, as well as loci architectures, some examples of which have been engineered and adapted to produce important biotechnologies.

The components of the system involved in host defense include one or more effector proteins capable of modifying DNA or RNA and an RNA guide element that is responsible to targeting these protein activities to a specific sequence on the phage DNA or RNA. The RNA guide is composed of a CRISPR RNA (crRNA) and may require an additional trans-activating RNA (tracrRNA) to enable targeted nucleic acid manipulation by the effector protein(s). The crRNA consists of a direct repeat responsible for protein binding to the crRNA and a spacer sequence that is complementary to the desired nucleic acid target sequence. CRISPR systems can be reprogrammed to target alternative DNA or RNA targets by modifying the spacer sequence of the crRNA.

CRISPR-Cas systems can be broadly classified into two classes: Class 1 systems are composed of multiple effector proteins that together form a complex around a crRNA, and Class 2 systems consist of a single effector protein that complexes with the erRNA to target DNA or RNA substrates. The single-subunit effector composition of the Class 2 systems provides a simpler component set for engineering and application translation, and have thus far been an important source of programmable effectors. Thus, the discovery, engineering, and optimization of novel Class 2 systems may lead to widespread and powerful programmable technologies for genome engineering and beyond.

Citation or identification of any document in this application is not an admission that such document is available as prior art to the present invention.

### SUMMARY

The present disclosure provides methods for computational identification of new CRISPR-Cas systems from genomic databases, together with the development of the natural loci into an engineered system, and experimental validation and application translation.

In one aspect, provided herein is an engineered, non-naturally occurring Clustered Interspaced Short Palindromic Repeat (CRISPR)-Cas system of CLUST.004377 including a Guide consisting of a direct repeat sequence and a spacer sequence capable of hybridizing to a target nucleic acid, wherein the Guide is CRISPR RNA (crRNA) or DNA, and any one of the following: a CRISPR-associated protein containing charged N-terminal residues that are capable of binding to the Guide, either as a monomer or multimer, and of targeting the target nucleic acid sequence complementary to the Guide spacer; a CRISPR-associated protein containing charged N-terminal residues and an HTH domain-containing protein that together are capable of binding to the Guide and of targeting the target nucleic acid sequence complementary to the spacer sequence; a CRISPR-associated protein containing charged N-terminal residues and a transposase module that together are capable of binding to the Guide and of targeting the target nucleic acid sequence complementary to the spacer sequence; a CRISPR-associated protein containing charged N-terminal residues, an HTH domain-containing protein, and a transposase module that together are capable of binding to the Guide and of targeting the target nucleic acid sequence complementary to the spacer sequence; and a payload nucleic acid flanked by transposon end sequences.

In some embodiments, the target nucleic acid is a DNA or an RNA. In some embodiments, the target nucleic acid is double-stranded DNA.

In some embodiments, the targeting of the target nucleic acid by the protein and the Guide results in a modification in the target nucleic acid.

In some embodiments, the modification in the target nucleic acid is a double-stranded cleavage event. In some embodiments, the modification in the target nucleic acid is a single-stranded cleavage event.

In some embodiments, the system further comprises a donor template nucleic acid. In some embodiments, the donor template nucleic acid is a DNA. In some embodiments, the target nucleic acid is a double stranded DNA and the targeting of the double stranded DNA results in scarless DNA insertion.

In some embodiments, the modification results in cell toxicity.

In some embodiments, the systems described herein are located within a cell. In some embodiments, the cell comprises a prokaryotic or eukaryotic cell.

In yet another aspect, the disclosure provides methods of targeting and editing a target nucleic acid, wherein the methods include contacting the target nucleic acid with one or more of the systems described herein.

In another aspect, the disclosure provides methods of targeting the insertion of a payload nucleic acid at a site of the target nucleic acid, wherein the methods include contacting the target nucleic acid with one or more of the systems described herein.

In another aspect, the disclosure provides methods of targeting the excision of a payload nucleic acid from a site at a target nucleic acid, wherein the methods include contacting the target nucleic acid with one or more systems described herein.

In some embodiments, the transposase module comprises Mu-transposase. TniQ, and/or TniB.

In some embodiments, the CRISPR-associated protein comprises an amino acid sequence having at least 85%, 90%, 95%, 96%, 97%, 98%, 99%, or 100% similarity to an amino acid sequence provided in any one of Tables 2-6.

In some embodiments, the crRNA or Guide comprises a nucleic acid sequence having at least 85%, 90%, 95%, 96%, 97%, 98%, 99%, or 100% similarity to a nucleic acid sequence provided in Table 7.

In some embodiments, the payload nucleic acid is flanked by transposon end sequences comprising a nucleic acid sequence having at least 85%. 90%, 95%, 96%, 97%, 98%, 99%, or 100% similarity to a nucleic acid sequence provided in Table 8.

In some embodiments, the payload nucleic acid is flanked by transposon end sequences comprising a nucleic acid sequence having at least 85%, 90%, 95%, 96%, 97%, 98%, 99%, or 100% similarity to a nucleic acid sequence contained in Table 9.

In some embodiments, the CRISPR-associated protein comprises at least one nuclear localization signal.

In some embodiments, the CRISPR-associated protein comprises at least one nuclear export signal.

In some embodiments, at least one component of the system is encoded by a codon-optimized nucleic acid for expression in a cell.

In some embodiments, the codon-optimized nucleic acid is present within at least one vector.

In some embodiments, the at least one vector comprise one or more regulatory elements operably-linked to a nucleic acid encoding the component of the system.

In some embodiments, the one or more regulatory elements comprise at least one promoter.

In some embodiments, the at least one promoter comprises an inducible promoter or a constitutive promoter.

In some embodiments, the at least one vector comprises a plurality of vectors. In some embodiments, the at least one vector comprises a viral vector. In some embodiments, the viral vector is selected from the group consisting of a retroviral vector, a lentiviral vector, an adenoviral vector, an adeno-associated vector, and a herpes simplex vector.

In some embodiments, the system is present in a delivery system.

In some embodiments, the delivery system comprises a delivery vehicle selected from the group consisting of a liposome, an exosome, a microvesicle, and a gene-gun.

In yet another aspect, the disclosure provides a cell comprising the system described herein.

In some embodiments, the cell is a eukaryotic cell.

In some embodiments, the eukaryotic cell is a mammalian cell or a plant cell.

In some embodiments, the mammalian cell is a human cell.

In some embodiments, the cell is a prokaryotic cell.

In some embodiments, the method results in an insertion or substitution of DNA to correct a native locus.

In some embodiments, the method results in a targeted insertion of a DNA payload into a specific genomic target site.

In some embodiments, the method results in a targeted deletion of DNA to correct a native locus.

The term “CRISPR-associated transposon” as used herein refers to a mobile genetic element having terminal transposon ends on both sides, which is acted upon by a CRISPR system described herein. In some embodiments, the CRISPR-associated transposon includes a gene encoding a CRISPR-associated transposase that is capable of facilitating the mobility (e.g., excision or deletion) of the CRISPR-associated transposon from a first site in a nucleic acid to a second site in a nucleic acid.

The term “CRISPR-associated transposase” as used herein refers to a protein including one or more transposase domains that is encoded by a gene that in nature is present in a CRISPR-associated transposon. In some embodiments, the CRISPR-associated transposase is capable of facilitating the mobility of a CRISPR-associated transposon from a first site in a nucleic acid to a second site in a nucleic acid. In some embodiments, the CRISPR-associated transposase has integration activity. In some embodiments, the CRISPR-associated transposase has excision activity. In some embodiments, the CRISPR-associated transposase specifically targets a CRISPR-associated transposon for mobilization via an RNA guide.

The term “Guide” for a CRISPR-associated transposase system refers to either an RNA or DNA sequence that includes one or more direct repeat and spacer sequences, and that is capable of hybridizing to a target nucleic acid and to the proteins and/or nucleic acid of the CRISPR-associated transposon complex.

The term “cleavage event,” as used herein, refers to a DNA break in a target nucleic acid created by a nuclease of a CRISPR system described herein. In some embodiments, the cleavage event is a double-stranded DNA break. In some embodiments, the cleavage event is a single-stranded DNA break.

The term “CRISPR system” as used herein refers to nucleic acids and/or proteins involved in the expression of, or directing the activity of, CRISPR-effectors, including sequences encoding CRISPR effectors, RNA guides, and other sequences and transcripts from a CRISPR locus.

The term “CRISPR array” as used herein refers to the nucleic acid (e.g., DNA) segment that includes CRISPR repeats and spacers, starting with the first nucleotide of the first CRISPR repeat and ending with the last nucleotide of the last (terminal) CRISPR repeat. Typically, each spacer in a CRISPR array is located between two repeats. The term “CRISPR repeat,” or “CRISPR direct repeat,” or “direct repeat.” as used herein, refers to multiple short direct repeating sequences, which show very little or no sequence variation within a CRISPR array. The term “CRISPR RNA” or “crRNA” as used herein refers to an RNA molecule comprising a guide sequence used by a CRISPR effector to specifically target a nucleic acid sequence. Typically crRNAs contains a sequence that mediates target recognition and a sequence that forms a duplex with a tracrRNA. The crRNA: tracrRNA duplex binds to a CRISPR effector. The term “donor template nucleic acid,” as used herein refers to a nucleic acid molecule that can be used by one or more cellular proteins to alter the structure of a target nucleic acid after a CRISPR enzyme described herein has altered a target nucleic acid. In some embodiments, the donor template nucleic acid is a double-stranded nucleic acid. In some embodiments, the donor template nucleic acid is a single-stranded nucleic acid. In some embodiments, the donor template nucleic acid is linear. In some embodiments, the donor template nucleic acid is circular (e.g., a plasmid). In some embodiments, the donor template nucleic acid is an exogenous nucleic acid molecule. In some embodiments, the donor template nucleic acid is an endogenous nucleic acid molecule (e.g., a chromosome).

The term “CRISPR effector,” “effector,” “CRISPR-associated protein,” or “CRISPR enzyme” as used herein refers to a protein that carries out an enzymatic acitivity or that binds to a target site on a nucleic acid specified by an RNA guide. In some embodiments, a CRISPR effector has endonuclease activity, nickase activity, exonuclease activity, transposase activity, and/or excision activity.

The term “guide RNA” or “gRNA” as used herein refers to an RNA molecule capable of directing a CRISPR effector having nuclease activity to target and cleave a specified target nucleic acid.

The term “RNA guide” as used herein refers to any RNA molecule that facilitates the targeting of a protein described herein to a target nucleic acid. Exemplary “RNA guides” include, but are not limited to, tracrRNAs, and crRNAs.

The term “mobile genetic element” as used herein refers to a nucleic acid capable of being specifically recognized and mobilized from a nucleic acid. In some embodiments, the mobile genetic element comprises nucleic acid sequences at flanking terminal ends that are specifically recognized by a CRISPR-associated transposase.

The term “origin of replication,” as used herein, refers to a nucleic acid sequence in a replicating nucleic acid molecule (e.g., a plasmid or a chromosome) at which replication is initiated.

As used herein, the term “target nucleic acid” refers to a specific nucleic acid sequence that is to be modified by a CRISPR system described herein. In some embodiments, the target nucleic acid comprises a gene. In some embodiments, the target nucleic acid comprises a non-coding region (e.g., a promoter). In some embodiments, the target nucleic acid is single-stranded. In some embodiments, the target nucleic acid is double-stranded.

The terms “trans-activating crRNA” or “tracrRNA” as used herein refer to an RNA including a sequence that forms a structure required for a CRISPR effector to bind to a specified target nucleic acid.

A “transcriptionally-active site” as used herein refers to a site in a nucleic acid sequence comprising promoter regions at which transcription is initiated and actively occurring.

The term “collateral RNAse activity,” as used herein in reference to a CRISPR enzyme, refers to non-specific RNAse activity of a CRISPR enzyme after the enzyme has modified a specifically-targeted nucleic acid.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF FIGURE DESCRIPTION

FIGS. 1A-B shows the conserved effectors and CRISPR array elements by bacterial genome accession and species for representative CLUST.004377 loci.

FIGS. 2A-D show the left (A, labeled TER-L) and right (B, labeled TER-R) transposon ends by bacterial genome accession and species for representative CLUST.004377 loci.

FIG. 3 shows a phylogenetic tree of CLUST.004377 effector A proteins.

FIG. 4 shows an alignment of CLUST.004377 effector A proteins by genome accession and species, highlighting sequence location of conserved residues, with color denoting nucleotide polarity (Yellow: Non-polar side chain, Green: Polar side chain, Blue: Basic, Red: Acidic).

FIG. 5 shows PFAM domains identified within CLUST.004377 effector A proteins.

FIG. 6 shows a phylogenetic tree of CLUST.004377 effector B proteins.

FIG. 7 shows an alignment of CLUST.004377 effector B proteins by genome accession and species, highlighting sequence location of conserved residues, with color denoting nucleotide polarity (Yellow: Non-polar side chain, Green: Polar side chain, Blue: Basic, Red: Acidic).

FIG. 8 shows PFAM domains identified within CLUST.004377 effector B proteins.

FIG. 9 shows a phylogenetic tree of CLUST.004377 effector C1 proteins.

FIG. 10 shows an alignment of CLUST.004377 effector C1 proteins by genome accession and species, highlighting sequence location of conserved residues, with color denoting nucleotide polarity (Yellow: Non-polar side chain, Green: Polar side chain, Blue: Basic, Red: Acidic).

FIG. 11 shows PFAM domains identified within CLUST.004377 effector C1 proteins.

FIG. 12 shows a phylogenetic tree of CLUST.004377 effector C2 proteins.

FIG. 13 shows an alignment of CLUST.004377 effector C2 proteins by genome accession and species, highlighting sequence location of conserved residues, with color denoting nucleotide polarity (Yellow: Non-polar side chain, Green: Polar side chain, Blue: Basic, Red: Acidic).

FIG. 14 shows PFAM domains identified within CLUST.004377 effector C2 proteins.

FIG. 15 shows a phylogenetic tree of CLUST.004377 effector C3 proteins.

FIG. 16 shows an alignment of CLUST.004377 effector C3 proteins by genome accession and species, highlighting sequence location of conserved residues, with color denoting nucleotide polarity (Yellow: Non-polar side chain, Green: Polar side chain, Blue: Basic, Red: Acidic).

FIG. 17 shows PFAM domains identified within CLUST.004377 effector C3 proteins.

FIG. 18 shows a schematic of natural and engineered components for the CRISPR transposition system of CLUST.004377.

FIG. 19 shows a schematic of transposon excision in the engineered system of CLUST.004377.

FIG. 20 shows a schematic of RNA guided transposon insertion in the engineered system of CLUST.004377.

FIG. 21 shows a schematic CRISPR negative selection screening

FIGS. 22 and 23 show depletion distributions for direct repeats and spacers targeting pACYC and E. coli essential genes. To quantify depletion, a fold-depletion ratio was calculated as R_{treated}/Rᵢₙₚᵤₜ for each direct repeat and spacer. The normalized input read count is computed as:
Rᵢₙₚᵤₜ= #reads containing DR+spacer/total reads without expressing the CLUST.004377 system and RNA guide. The treated read count is computed as
R_{treated}=(1+ #reads containing DR+spacer)/total #reads with expression of the CLUST.004377 system and RNA guide. A strongly depleted target has a fold depletion greater than 3, which is marked by the red lines.

FIGS. 24A and 24B show the target site mapping of depleted RNA guides targeting pACYC (A) and E. coli essential genes (B).

FIGS. 25A-C shows a weblogo of the sequences flanking the left (A) and right (B) sides of depleted targets for the KV757663 CLUST.004377 CRISPR-Cas system.

### DETAILED DESCRIPTION

The disclosure relates to the use of computational methods and algorithms to predict new CRISPR-Cas systems and identify their components.

In one embodiment, the disclosure includes new computational methods for identifying novel CRISPR loci by:
detecting all potential CRISPR arrays in prokaryotic data sources (contig, scaffold, or complete genome);
identifying all predicted protein coding genes in close proximity to a CRISPR array (e.g. 10 kb);
forming protein clusters (putative protein families) around identified genes, using, for example, mmseqs2;
selecting clusters of proteins of unknown function, and identifying homologs in the wider prokaryotic set of proteins using, e.g., BLAST or UBLAST;
identifying clusters of proteins with a large percentage of homologs co-occurring with CRISPR arrays; and
predicting the functional domains of the proteins in the identified cluster, e.g., by using hmmsearch on each member of the cluster individually, or by, for example, using a profile hidden Markov model (HMM) constructed from the multiple alignment.

External databases of functional domains include, for example. Pfam and Uniprot. Multiple alignment can be done using, e.g., mafft.

In another aspect, the disclosure relates to defining the minimal elements of novel CRISPR systems by:
identifying conserved elements (both coding genes and non-coding) in the loci surrounding each cluster (in one aspect, this can be done manually by inspection on a case by case analysis);
identifying specific conserved non-coding elements that may be terminal repeats required for transposon activity;
identifying the RNA guide associated with each protein (identify conserved direct repeat structures and then attach a non-natural spacer sequence to the direct repeat. The effect of the non-natural spacer is to induce the effector to target a novel DNA or RNA substrate);
identifying the minimal RNA or DNA target (identify RNA and DNA targeting a by testing targeting activity of the effector(s) in combination with crRNAs containing multiple different engineered spacer sequences targeting a high diversity of DNA and RNA substrates);
identifying the minimal system necessary to achieve activity; and
testing all conserved elements together and then systematically removing different proteins while preserving activity.

The broad natural diversity of CRISPR-Cas defense systems contains a wide range of activity mechanisms and functional elements that can be harnessed for programmable biotechnologies. In a natural system, these mechanisms and parameters enable efficient defense against foreign DNA and viruses while providing self vs. non-self discrimination to avoid self-targeting. In an engineered system, the same mechanisms and parameters also provide a diverse toolbox of molecular technologies and define the boundaries of the targeting space. For instance. systems Cas9 and Cas13a have canonical DNA and RNA endonuclease activity and their targeting spaces are defined by the protospacer adjacent motif (PAM) on targeted DNA and protospacer flanking sites (PFS) on targeted RNA, respectively.

The methods described herein can be used to discover additional mechanisms and parameters within single subunit Class 2 effector systems that can be more effectively harnessed for programmable biotechnologies.

### Pooled-Screening

To efficiently validate the activity of the engineered novel CRISPR-Cas systems and simultaneously evaluate in an unbiased manner different activity mechanisms and functional parameters, we used a new pooled-screening approach in E. coli. First, from the computational identification of the conserved protein and noncoding elements of the novel CRISPR-Cas system, DNA synthesis and molecular cloning was used to assemble the separate components into a single artificial expression vector, which in one embodiment is based on a pET-28a+backbone. In a second embodiment, the effectors and noncoding elements are transcribed on a single mRNA transcript, and different ribosomal binding sites are used to translate individual effectors.

Second, the natural erRNA and targeting spacers were replaced with a library of unprocessed crRNAs containing non-natural spacers targeting the essential genes of the host E. coli, or a second plasmid bearing antibiotic resistance, pACYC184. This crRNA library was cloned into the vector backbone containing the protein effectors and noncoding elements (e.g. pET-28a+), and then subsequently transformed the library into E. coli along with the pACYC184 plasmid target. Consequently, each resulting E. coli cell contains no more than one targeting spacer.

Third, the E. coli were grown under antibiotic selection. In one embodiment, triple antibiotic selection is used: kanamycin for ensuring successful transformation of the pET-28a+vector containing the engineered CRISPR-Cas effector system, and chloramphenicol and tetracycline for ensuring successful co-transformation of the pACYC184 target vector. Since pACYC184 normally confers resistance to chloramphenicol and tetracycline, under antibiotic selection, positive activity of the novel CRISPR-Cas system targeting the plasmid will eliminate cells that actively express the effectors, noncoding elements, and specific active elements of the crRNA library. Examining the population of surviving cells at a later time point compared to an earlier time point results in a depleted signal compared to the inactive crRNAs.

Since the pACYC184 plasmid contains a diverse set of features and sequences that may affect the activity of a CRISPR-Cas system, mapping the active crRNAs from the pooled screen onto pACYC184 provides patterns of activity that can be suggestive of different activity mechanisms and functional parameters in a broad, hypothesis-agnostic manner. In this way, the features required for reconstituting the novel CRISPR-Cas system in a heterologous prokaryotic species can be more comprehensively tested and studied.

The key advantages of the in vivo pooled-screen described herein include:

(1) Versatility—Plasmid design allows multiple effectors and/or noncoding elements to be expressed; library cloning strategy enables both transcriptional directions of the computationally predicted erRNA to be expressed;

(2) Comprehensive tests of activity mechanisms & functional parameters—Evaluates diverse interference mechanisms, including DNA or RNA cleavage; DNA excision and/or insertion via transposition; examines co-occurrence of features such as transcription, plasmid DNA replication; and flanking sequences for erRNA library can be used to reliably determine PAMs with complexity equivalence of 4N's;

(3) Sensitivity-by using as targets the low copy number of pACYC184 and the single copy of the E. coli genome, this screen design enables high sensitivity for CRISPR-Cas activity since even modest interference rates can result in loss of cell viability through loss of antibiotic resistance or essential gene targeting; and

(4) Efficiency-Optimized molecular biology steps to enable greater speed and throughput, as RNA-sequencing and protein expression samples can be directly harvested from the surviving cells in the screen.

The novel CRISPR-Cas families described herein were evaluated using this in vivo pooled-screen to evaluate their operational elements, mechanisms and parameters, as well as their ability to be active and reprogrammed in an engineered system outside of their natural cellular environment.

### CRISPR Enzymes Associated with Mobile Genetic Elements

This disclosure provides mobile genetic elements (e.g., CRISPR-associated transposons) associated with CRISPR systems described herein. These mobile genetic elements can be genetically-altered to delete and/or add one or more components (e.g., a gene encoding a therapeutic product), thereby resulting in mobile genetic elements that can be readily inserted into or removed from a target nucleic acid. In some embodiments, the CRISPR systems encoded by these mobile genetic elements include one or more effector proteins, referred to hercin as “CRISPR-associated transposases,” which facilitate the movement of the mobile genetic elements from a first site in a nucleic acid to a second site in a nucleic acid. As described in further detail below, in some embodiments, the activity (e.g., excision activity or integration activity) of the CRISPR-associated transposases can be directed to a particular site on a target nucleic acid using a RNA guide that is complementary to a nucleic acid sequence on the target nucleic acid. In some embodiments, the RNA guides are engineered to specifically target the insertion, excision, and/or mobilization of the mobile genetic element to any site in a target nucleic acid of interest.

In some embodiments, the disclosure provides CRISPR-associated transposons that include one or more genes encoding a CRISPR-associated transposase and an RNA guide. In some embodiments, the CRISPR-associated transposons include a payload nucleic acid. In some embodiments, the payload nucleic acid includes a gene of interest. In some embodiments, the gene of interest is operably linked to a promoter (e.g., a constitutive promoter or an inducible promoter). In some embodiments, the gene of interest encodes a therapeutic protein.

This disclosure further provides a class of CRISPR effectors, referred to hercin as CRISPR-associated transposases, capable of facilitating the movement of a mobile genetic element (e.g., a CRISPR-associated transposon), wherein the targeting of the mobile genetic element is facilitated by RNA guides. Mobile genetic elements in CLUST.004377, CLUST.009467, and CLUST.009925 comprise CRISPR-associated transposons, including genes encoding CRISPR-associated transposases having a rve integrase domain (also referred to herein as rve integrase domain-containing effectors).

### RNA-Guided DNA Insertion

In some embodiments, a CRISPR system described herein mediates the insertion of a nucleotide payload into a target nucleic acid sequence. This activity is facilitated by one or more CRISPR-associated transposases present in the CRISPR system. In some embodiments, the CRISPR-associated transposase comprises one or more of a rve integrase domain a TniQ domain, a TniB domain, or a TnpB domain. In some embodiments, the site of the target nucleic acid where the nucleotide payload is to be inserted is specified by a guide RNA.

### RNA-Guided DNA Excision

In some embodiments, a CRISPR system described herein mediates the excision of a nucleotide payload from a target nucleic acid sequence. This activity is facilitated by one or more CRISPR-associated transposases present in the CRISPR system. In some embodiments, the CRISPR-associated transposase comprises one or more of a rve domain a TniQ domain, a TniB domain, or a TnpB domain. In some embodiments, the site of the target nucleic acid where the nucleotide payload is to be excised is specified by a guide RNA.

### RNA-Guided DNA Mobilization

In some embodiments, a CRISPR system described herein mediates the excision of a nucleotide payload from a first site in a target nucleic acid, mobilization of the nucleotide payload, and insertion of the nucleotide payload at a second site in a target nucleic acid. This activity is facilitated by one or more CRISPR-associated transposases present in the CRISPR system. In some embodiments, the CRISPR-associated transposase comprises one or more of a rve domain a TniQ domain, a TniB domain, or a TnpB domain. In some embodiments, the first site of the target nucleic acid where the nucleotide payload is to be excised is specified by a guide RNA. In some embodiments, the second site in the target nucleic acid where the nucleotide payload is to be inserted is specified by a guide RNA.

### Transposase Activity

In some embodiments, a a CRISPR effector described herein (e.g., a CRISPR-associated transposase) comprises transposase activity. DNA transposition is one of the mechanisms by which genome rearrangement and horizontal gene transfer occurs in prokaryotic and eukaryotic cells. Transposons are DNA sequences that are capable of being moved within a genome. During DNA transposition a transposase recognizes transposon-specific sequences that flank an intervening DNA sequence. The transposase recognizes the transposon, excises the transposon from one location in a nucleic acid sequence, and inserts it (including the inverted repeat sequences with the intervening sequence) into another location. In some instances, the end sequences of the transposon comprise short inverted terminal repeats comprising duplications of a short segment of the sequence flanking the insertion sites that are characteristic for each transposon.

The mechanisms involved in transposition mediated by several transposon systems including Tc1, Tol2, Minos, Himar1, Hsmar1, Mos1, Frog Prince, Piggyback, and Sleeping Beauty have been characterized (see, e.g., Chitilian et al. (2014) Stem Cells 32:204-15; Ivics et al. (1997) Cell 91:501-10; Miskey et al. (2003) Nucleic Acids Res. 31:6873-81; Urschitz et al. (2013) Mob. Genet. Elements 3: e25167; Jursch et al. (2013) Mob. DNA 4:15; Pflieger et al. (2014) J. Biol. Chem. 289:100-11); and Hou et al. (2015) Cancer Biol. Ther. 16(1): 8-16). For example, the Sleeping Beauty transposase (SBase) functions through a “cut-and-paste” process (see, e.g., Yant et al. (2004) Mol. Cell. Biol. 4:9239-47). In some embodiments, a CRISPR-associated transposase described herein comprise a transposase domain or a transposase-like domain. For example, in some embodiments, the CRISPR-associated transposase comprises a Mu-transposase domain or module. In some embodiments, the CRISPR-associated transposase comprises a TniQ transposase domain. In some embodiments, the CRISPR-associated transposase comprises a TniB transposase domain. In some embodiments, the transposase domain is a OrfB_IS605 domain.

In some embodiments, the transposase domain of a CRISPR-associated transposase described herein is capable of one or more of the following activities: (a) excising a nucleic acid fragment from its situs on in a genome (i.e., excision activity); (b) mediating the integration of a nucleic acid fragment into a situs in a genome (i.e., integration activity); and/or (c) specifically recognizing a transposon element (e.g., a specific inverted repeat sequence). In some embodiments, a transposase domain of a CRISPR-associated transposase described herein is modified to eliminate one or more activities. In some embodiments, a transposase domain of a CRISPR-associated transposase described herein may be mutated such that the transposase domain comprises excision activity, but does not comprise integration activity. In some embodiments, a transposase domain of a CRISPR-associated transposase described herein is mutated such that a transpose domain comprises integration activity, but does not comprise excision activity. In some embodiments, a transposase domain of a CRISPR-associated transposase described herein is mutated such that the transposase domain comprises integration activity and excision activity, but lacks the ability to recognize a specific nucleic acid sequence. In some embodiments, a transposase domain of a CRISPR-associated transposase described herein is mutated such that the transposase domain comprises integration activity, but lacks excision activity and the ability to recognize a specific nucleic acid sequence. In some embodiments, a transposase domain of a CRISPR-associated transposase described herein is mutated such that the transposase domain comprises excision activity, but lacks integration activity and the ability to recognize a specific nucleic acid sequence.

In some embodiments, the CRISPR-associated transposases described herein can be used to facilitate the random insertion of a nucleic acid sequence into the genome of a cell. In some embodiments, the CRISPR-associated transposases described herein may be used to facilitate the targeted insertion or excision of a nucleic acid sequence into or out of the genome of a cell. In some embodiments, the nucleic acid sequence that is inserted into the cell is flanked by inverted repeat sequences that are specifically recognized by a transposase domain of the CRISPR-associated transposase. In some embodiments, the intervening nucleic acid sequence between these inverted repeat sequences is at least 10 bp, at least 20 bp, at least 30 bp, at least 40 bp, at least 50 bp, at least 60 bp, at least 70 bp, at least 80 bp, at least 90 bp, at least 100 bp, at least 150 bp, at least 200 bp, at least 250 bp, at least 300 bp, at least 400 bp, at least 500 bp, at least 600 bp, at least 700 bp, at least 800 bp, at least 900 bp, at least 1000 bp, at least 1,500 bp, at least 2,000 bp, at least 3,000 bp, at least 4,000 bp, at least 5,000 bp in length, at least 6,000 bp in length, at least 7,000 bp in length, at least 8,000 bp in length, at least 9,000 bp in length, or at least 10,000 bp in length,. In some embodiments, the intervening nucleic acid sequence between these inverted repeat sequences is less than 10 bp, less than 20 bp, less than 30 bp, less than 40 bp, less than 50 bp, less than 60 bp, less than 70 bp, less than 80 bp, less than 90 bp, less than 100 bp, less than 150 bp, less than 200 bp, less than 250 bp, less than 300 bp, less than 400 bp, less than 500 bp, less than 600 bp, less than 700 bp, less than 800 bp, less than 900 bp, less than 1000 bp, less than 1,500 bp, less than 2,000 bp, less than 3,000 bp, less than 4,000 bp, less than 5,000 bp in length, or less than 10,000 bp in length.

### Effectors Comprising Helix-Turn-Helix Domains

In some embodiments, effectors within the systems described herein may contain a helix-turn-helix (HTH) domain. HTH domains typically include or consist of two α-helices forming an internal angle of approximately 120 degrees that are connected by a short strand or turn of amino acid residues, and are present in a multitude of prokaryotic and eukaryotic DNA-binding proteins, such as transcription factors (see, e.g., Aravind et al. (2005) FEMS Microbiol. Rev. 29(2): 231-62). Beyond transcriptional regulation, HTH domains are involved in a wide range of functions including DNA repair and replication. RNA metabolism and protein-protein interactions. In some embodiments, an effector described herein comprises DNA-binding activity. In some embodiments, an effector described herein comprises RNA-binding activity. In some embodiments, an effector described herein comprises a HTH domain that mediates a protein-protein interaction.

### CRISPR Enzyme Modifications

### Modulating Insertion/Excision/Transposition Activity of CRISPR Enzymes

The activity of CRISPR-associated transposases may be altered in ways to change the relative efficiencies of insertion and excision. Altering these activities enable greater control in the different modes of transposase directed genome editing; for instance, CRISPR-associated transposases with predominantly insertion activity can be used for locus-specific insertion of transgenes to enable applications including, but not limited to, therapeutics (e.g., fetal hemoglobin expression for treatment of thalassemia), genetic engineering (e.g., trait stacking in plant genome engineering), or engineered cells (e.g., introducing control circuits or custom chimeric antigen receptors for CAR-T cell engineering). Alternatively, CRISPR-associated transposases with predominantly excision activity can also be used in applications, such as restoring the genome of engineered cells through excising inserted transcription factors. These CRISPR-associated transposases can be modified to have diminished excision or insertion activity, e.g., excision or insertion inactivation of at least 40%, at least 50%, at least 60%, at least 70%, at least 80%, at least 90%, at least 95%, at least 97%, or 100% as compared with the wild type CRISPR-associated transposases. Modulation of the insertion or excision activity can be done with several methods known in the art, such as introducing mutations into the catalytic core of the transposase. In some embodiments, catalytic residues for the transposase activities are identified, and these amino acid residues can be substituted by different amino acid residues (e.g., glycine or alanine) to diminish the transposase activity. An example in which modifications to the catalytic core of a transposase yielded differential effects on excision versus insertion is with the generation of an excision competent/integration defective variant of the piggyBac transposase (Exc+/Int−PB) Li, Xianghong et al. “piggyBac transposase tools for genome engineering,” Proc. Nat'l. Acad. Sci., 1073.10 (2013): 2279-2287. In other embodiments, the variants to modulate excision/insertion activity can be combined with other CRISPR-associated transposase modifications, such as a mutations that relax or make more stringent the targeting space or PAM constraints. Furthermore, these mutations are not restricted to the protein effectors of the transposon, but may be found on the noncoding elements such as the transposase ends. Together, these may yield a set of mutations that enable the tuning of the enzymatic activities of the CRISPR-associated transposase.

### Generation of Fusion Proteins

Additionally, CRISPR-associated transposases, whether in its native functional form or with mutations to modulation its activity, can provide a foundation from which fusion proteins with additional functional proteins can be created. The inactivated CRISPR enzymes can comprise or be associated with one or more functional domains (e.g., via fusion protein, linker peptides, “GS” linkers, etc.). These functional domains can have various activities, e.g., methylase activity, demethylase activity, transcription activation activity, transcription repression activity, transcription release factor activity, histone modification activity, RNA cleavage activity, DNA cleavage activity, nucleic acid binding. In some embodiments, the functional domains are Krüppel associated box (KRAB), VP64, VP16, Fok1, P65, HSF1, MyoD1, and biotin-APEX.

The positioning of the one or more functional domains on the CRISPR transposase is one that allows for correct spatial orientation for the functional domain to affect the target with the attributed functional effect. For example, if the functional domain is a transcription activator (e.g., VP16, VP64, or p65), the transcription activator is placed in a spatial orientation that allows it to affect the transcription of the target. Likewise, a transcription repressor is positioned to affect the transcription of the target, and a nuclease (e.g., Fok1) is positioned to cleave or partially cleave the target. In some embodiments, the functional domain is positioned at the N-terminus of the CRISPR enzyme. In some embodiments, the functional domain is positioned at the C-terminus of the CRISPR enzyme. In some embodiments, the CRISPR enzyme is modified to comprise a first functional domain at the N-terminus and a second functional domain at the C-terminus.

The addition of functional domains to the CRISPR-associated transposase or onto other effector proteins in the complex may provide an ability for the transposase system to modify the the physical DNA (e.g., methylation, etc.) or its regulation (e.g., transcriptional or repression) in situ.

### Split Enzymes

The present disclosure also provides a split version of the CRISPR enzymes described herein. The split version of the CRISPR enzymes may be advantageous for delivery. In some embodiments, the CRISPR enzymes are split to two parts of the enzymes, which together substantially comprises a functioning CRISPR enzyme.

The split can be done in a way that the catalytic domain(s) are unaffected. The CRISPR enzymes may function as a nuclease or may be inactivated enzymes, which are essentially RNA-binding proteins with very little or no catalytic activity (e.g., due to mutation(s) in its catalytic domains).

In some embodiments, the nuclease lobe and a-helical lobe are expressed as separate polypeptides. Although the lobes do not interact on their own, the guide RNA recruits them into a ternary complex that recapitulates the activity of full-length CRISPR enzymes and catalyzes site-specific DNA cleavage. The use of a modified guide RNA abrogates split-enzyme activity by preventing dimerization, allowing for the development of an inducible dimerization system. The split enzyme is described, e.g., in Wright, Addison V., et al. “Rational design of a split-Cas9 enzyme complex.” Proc. Nat'l. Acad. Sci., 112.10 (2015): 2984-2989, which is incorporated herein by reference in its entirety.

In some embodiments, the split enzyme can be fused to a dimerization partner, e.g., by employing rapamycin sensitive dimerization domains. This allows the generation of a chemically inducible CRISPR enzyme for temporal control of CRISPR enzyme activity. The CRISPR enzymes can thus be rendered chemically inducible by being split into two fragments and rapamycin-sensitive dimerization domains can be used for controlled reassembly of the CRISPR enzymes.

The split point is typically designed in silico and cloned into the constructs. During this process, mutations can be introduced to the split enzyme and non-functional domains can be removed. In some embodiments, the two parts or fragments of the split CRISPR enzyme (i.e., the N-terminal and C-terminal fragments), can form a full CRISPR enzyme, comprising, e.g., at least 70%, at least 80%, at least 90%, at least 95%, or at least 99% of the sequence of the wild-type CRISPR enzyme.

### Self-Activating or Inactivating Enzymes

The CRISPR enzymes described herein can be designed to be self-activating or self-inactivating. In some embodiments, the CRISPR enzymes are self-inactivating. For example, the target sequence can be introduced into the CRISPR enzyme coding constructs. Thus, the CRISPR enzymes can modify the target sequence, as well as the construct encoding the enzyme thereby self-inactivating their expression. Methods of constructing a self-inactivating CRISPR system is described, e.g., in Epstein, Benjamin E., and David V. Schaffer. “Engineering a Self-Inactivating CRISPR System for AAV Vectors,” Mol. Ther., 24 (2016): S50, which is incorporated herein by reference in its entirety.

In some other embodiments, an additional guide RNA, expressed under the control of a weak promoter (e.g., 7SK promoter), can target the nucleic acid sequence encoding the CRISPR enzyme to prevent and/or block its expression (e.g., by preventing the transcription and/or translation of the nucleic acid). The transfection of cells with vectors expressing the CRISPR enzyme, guide RNAs, and guide RNAs that target the nucleic acid encoding the CRISPR enzyme can lead to efficient disruption of the nucleic acid encoding the CRISPR enzyme and decrease the levels of CRISPR enzyme, thereby limiting the genome editing activity.

In some embodiments, the genome editing activity of the CRISPR enzymes can be modulated through endogenous RNA signatures (e.g., miRNA) in mammalian cells. The CRISPR enzyme switch can be made by using a miRNA-complementary sequence in the 5′-UTR of mRNA encoding the CRISPR enzyme. The switches selectively and efficiently respond to miRNA in the target cells. Thus, the switches can differentially control the genome editing by sensing endogenous miRNA activities within a heterogeneous cell population. Therefore, the switch systems can provide a framework for cell-type selective genome editing and cell engineering based on intracellular miRNA information (Hirosawa, Moe et al. “Cell-type-specific genome editing with a microRNA-responsive CRISPR-Cas9 switch,” Nucl. Acids Res., 2017 Jul. 27; 45(13): e118).

### Inducible CRISPR Enzymes

The CRISPR enzymes can be inducible, e.g., light inducible or chemically inducible. This mechanism allows for activation of the functional domain in the CRISPR enzymes. Light inducibility can be achieved by various methods known in the art, e.g., by designing a fusion complex wherein CRY2PHR/CIBN pairing is used in split CRISPR Enzymes (see, e.g., Konermann et al. “Optical control of mammalian endogenous transcription and epigenetic states,” Nature, 500.7463 (2013): 472). Chemical inducibility can be achieved, e.g., by designing a fusion complex wherein FKBP/FRB (FK506 binding protein/FKBP rapamycin binding domain) pairing is used in split CRISPR Enzymes. Rapamycin is required for forming the fusion complex, thereby activating the CRISPR enzymes (see, e.g., Zetsche, Volz, and Zhang, “A split-Cas9 architecture for inducible genome editing and transcription modulation,” Nature Biotech., 33.2 (2015): 139-142).

Furthermore, expression of the CRISPR enzymes can be modulated by inducible promoters, e.g., tetracycline or doxycycline controlled transcriptional activation (Tet-On and Tet-Off expression system), hormone inducible gene expression system (e.g., an ecdysone inducible gene expression system), and an arabinose-inducible gene expression system. When delivered as RNA, expression of the RNA targeting effector protein can be modulated via a riboswitch, which can sense a small molecule like tetracycline (see, e.g., Goldfless, Stephen J. et al. “Direct and specific chemical control of eukaryotic translation with a synthetic RNA-protein interaction,” Nucl. Acids Res., 40.9 (2012): e64-e64).

Various embodiments of inducible CRISPR enzymes and inducible CRISPR systems are described, e.g., in US8871445, US20160208243, and WO2016205764, each of which is incorporated herein by reference in its entirety.

### Functional Mutations

Various mutations or modifications can be introduced into CRISPR enzymes as described herein to improve specificity and/or robustness. In some embodiments, the amino acid residues that recognize the Protospacer Adjacent Motif (PAM) are identified. The CRISPR enzymes described herein can be modified further to recognize different PAMs, e.g., by substituting the amino acid residues that recognize PAM with other amino acid residues.

In some embodiments, at least one Nuclear Localization Signal (NLS) is attached to the nucleic acid sequences encoding the CRISPR enzyme. In some embodiments, at least one Nuclear Export Signal (NES) is attached to the nucleic acid sequences encoding the CRISPR enzyme. In a preferred embodiment a C-terminal and/or N-terminal NLS or NES is attached for optimal expression and nuclear targeting in eukaryotic cells, e.g., human cells.

In some embodiments, the CRISPR enzymes described herein are mutated at one or more amino acid residues to alter one or more functional activities. For example, in some embodiments, the CRISPR enzyme is mutated at one or more amino acid residues to alter its helicase activity. In some embodiments, the CRISPR enzyme is mutated at one or more amino acid residues to alter its nuclease activity (e.g., endonuclease activity or exonuclease activity). In some embodiments, the CRISPR enzyme is mutated at one or more amino acid residues to alter its ability to functionally associate with a guide RNA. In some embodiments, the CRISPR enzyme is mutated at one or more amino acid residues to alter its ability to functionally associate with a target nucleic acid.

In some embodiments, the CRISPR enzymes described herein are capable of modifying a target nucleic acid molecule. In some embodiments, the CRISPR enzyme modifies both strands of the target nucleic acid molecule. However, in some embodiments, the CRISPR enzyme is mutated at one or more amino acid residues to alter its nucleic acid manipulation activity. For example, in some embodiments, the CRISPR enzyme may comprise one or more mutations which render the enzyme incapable of cleaving a target nucleic acid or inserting/excising a target sequence.

In some embodiments, a CRISPR enzyme described herein may be engineered to comprise a deletion in one or more amino acid residues to reduce the size of the enzyme while retaining one or more desired functional activities (e.g., nuclease activity and the ability to functionally interact with a guide RNA). The truncated CRISPR enzyme may be advantageously used in combination with delivery systems having load limitations.

In one aspect, the present disclosure provides nucleotide sequences that are at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the sequences described herein. In another aspect, the present disclosure also provides amino acid sequences that are at least 10%, 15%, 20%, 25%, 30%, 35%, 40%, 45%, 50%, 55%, 60%, 65%, 70%, 75%, 80%. 85%, 90%, 91%, 92%, 93%, 94%, 95%, 96%, 97%, 98%, or 99% identical to the sequences described herein.

In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are the same as the sequences described herein. In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80. 90, or 100 nucleotides, e.g., contiguous or non-contiguous nucleotides) that is different from the sequences described herein.

In some embodiments, the amino acid sequences have at least a portion (e.g., at least 1, 2, 3, 4. 5, 6, 7, 8. 9, 10, 11, 12, 13, 14, 15, 20, 30, 40, 50, 60, 70, 80, 90, or 100 amino acid residues, e.g., contiguous or non-contiguous amino acid residues) that is the same as the sequences described herein. In some embodiments, the amino acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from the sequences described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In general, the length of a reference sequence aligned for comparison purposes should be at least 80% of the length of the reference sequence, and in some embodiments is at least 90%, 95%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blosum 62 scoring matrix with a gap penalty of 12,a gap extend penalty of 4, and a frameshift gap penalty of 5.

### Guide RNA Modifications

### Spacer Lengths

The spacer length of guide RNAs can range from about 15 to 50 nucleotides. In some embodiments, the spacer length of a guide RNA is at least 16 nucleotides, at least 17 nucleotides, at least 18 nucleotides, at least 19 nucleotides, at least 20 nucleotides, at least 21 nucleotides, or at least 22 nucleotides. In some embodiments, the spacer length is from 15 to 17 nucleotides, from 17 to 20 nucleotides, from 20 to 24 nucleotides (e.g., 20, 21, 22, 23, or 24 nucleotides), from 23 to 25 nucleotides (e.g., 23, 24, or 25 nucleotides), from 24 to 27 nucleotides, from 27 to 30 nucleotides, from 30 to 45 nucleotides (e.g., 30, 31, 32, 33, 34, 35, 40, or 45 nucleotides), from 30 or 35 to 40 nucleotides, from 41 to 45 nucleotides, from 45 to 50 nucleotides, or longer. In some embodiments, the direct repeat length of the guide RNA is at least 16 nucleotides, or is from 16 to 20 nucleotides (e.g., 16, 17, 18, 19, or 20 nucleotides). In some embodiments, the direct repeat length of the guide RNA is 19 nucleotides.

The guide RNA sequences can be modified in a manner that allows for formation of the CRISPR complex and successful binding to the target, while at the same time not allowing for successful effector activity (i.e., without excision activity/without insertion activity/without nuclease activity). These modified guide sequences are referred to as “dead guides” or “dead guide sequences.” These dead guides or dead guide sequences may be catalytically inactive or conformationally inactive with regard to nuclease activity. Dead guide sequences are typically shorter than respective guide sequences that result in active modification. In some embodiments. dead guides are 5%, 10%, 20%, 30%, 40%, or 50%, shorter than respective guide RNAs that have nuclease activity. Dead guide sequences of guide RNAs can be from 13 to 15 nucleotides in length (e.g., 13, 14, or 15 nucleotides in length), from 15 to 19 nucleotides in length, or from 17 to 18 nucleotides in length (e.g., 17 nucleotides in length).

Thus, in one aspect, the disclosure provides non-naturally occurring or engineered CRISPR systems including a functional CRISPR enzyme as described herein, and a guide RNA (gRNA) wherein the gRNA comprises a dead guide sequence whereby the gRNA is capable of hybridizing to a target sequence such that the CRISPR system is directed to a genomic locus of interest in a cell without detectable nucleic acid modification activity.

A detailed description of dead guides is described, e.g., in WO 2016094872, which is incorporated herein by reference in its entirety.

### Inducible Guides

Guide RNAs can be generated as components of inducible systems. The inducible nature of the systems allows for spatiotemporal control of gene editing or gene expression. In some embodiments, the stimuli for the inducible systems include, e.g., electromagnetic radiation, sound energy, chemical energy, and/or thermal energy.

In some embodiments, the transcription of guide RNA can be modulated by inducible promoters, e.g., tetracycline or doxycycline controlled transcriptional activation (Tet-On and Tet-Off expression systems), hormone inducible gene expression systems (e.g., ccdysone inducible gene expression systems), and arabinose-inducible gene expression systems. Other examples of inducible systems include, e.g., small molecule two-hybrid transcription activations systems (FKBP, ABA, etc.), light inducible systems (Phytochrome, LOV domains, or cryptochrome), or Light Inducible Transcriptional Effector (LITE). These inducible systems are described, e.g., in WO 2016205764 and U.S. Pat. No. 8,795,965, both of which are incorporated herein by reference in the entirety.

### Chemical Modifications

Chemical modifications can be applied to the guide RNA's phosphate backbone, sugar, and/or base. Backbone modifications such as phosphorothioates modify the charge on the phosphate backbone and aid in the delivery and nuclease resistance of the oligonucleotide (see, e.g., Eckstein, “Phosphorothioates, essential components of therapeutic oligonucleotides,” Nucl. Acid Ther., 24 (2014), pp. 374-387); modifications of sugars, such as 2′—O-methyl (2′—OMe), 2′—F, and locked nucleic acid (LNA), enhance both base pairing and nuclease resistance (see, e.g., Allerson et al. “Fully 2 ‘-modified oligonucleotide duplexes with improved in vitro potency and stability compared to unmodified small interfering RNA,” J. Med. Chem., 48.4 (2005): 901-904). Chemically modified bases such as 2-thiouridine or N6-methyladenosine, among others, can allow for either stronger or weaker base pairing (see, e.g., Bramsen et al., “Development of therapeutic-grade small interfering RNAs by chemical engineering,” Front. Genet., 2012 Aug. 20; 3:154). Additionally, RNA is amenable to both 5′ and 3′ end conjugations with a variety of functional moicties including fluorescent dyes, polyethylene glycol, or proteins.

A wide variety of modifications can be applied to chemically synthesized guide RNA molecules. For example, modifying an oligonucleotide with a 2′—OMe to improve nuclease resistance can change the binding energy of Watson-Crick base pairing. Furthermore, a 2′—OMe modification can affect how the oligonucleotide interacts with transfection reagents, proteins or any other molecules in the cell. The effects of these modifications can be determined by empirical testing.

In some embodiments, the guide RNA includes one or more phosphorothioate modifications. In some embodiments, the guide RNA includes one or more locked nucleic acids for the purpose of enhancing base pairing and/or increasing nuclease resistance.

A summary of these chemical modifications can be found, e.g., in Kelley et al., “Versatility of chemically synthesized guide RNAs for CRISPR-Cas9 genome editing,” J. Biotechnol. 2016 Sep. 10; 233:74-83; WO 2016205764; and U.S. Pat. No. 8,795,965 B2; each which is incorporated by reference in its entirety.

### Sequence Modifications

The sequences and the lengths of the guide RNAs, tracrRNAs, and crRNAs described herein can be optimized. In some embodiments, the optimized length of guide RNA can be determined by identifying the processed form of tracrRNA and/or crRNA, or by empirical length studies for guide RNAs, tracrRNAs, crRNAs, and the tracrRNA tetraloops.

The guide RNAs can also include one or more aptamer sequences. Aptamers are oligonucleotide or peptide molecules that can bind to a specific target molecule. The aptamers can be specific to gene effectors, gene activators, or gene repressors. In some embodiments, the aptamers can be specific to a protein, which in turn is specific to and recruits/binds to specific gene effectors, gene activators, or gene repressors. The effectors, activators, or repressors can be present in the form of fusion proteins. In some embodiments, the guide RNA has two or more aptamer sequences that are specific to the same adaptor proteins. In some embodiments, the two or more aptamer sequences are specific to different adaptor proteins. The adaptor proteins can include, e.g., MS2, PP7, Qβ, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s, and PRR1. Accordingly, in some embodiments, the aptamer is selected from binding proteins specifically binding any one of the adaptor proteins as described herein. In some embodiments, the aptamer sequence is a MS2 loop. A detailed description of aptamers can be found, e.g., in Nowak et al., “Guide RNA engineering for versatile Cas9 functionality,” Nucl. Acid. Res., 2016 Nov. 16;44(20):9555-9564; and WO 2016205764, which are incorporated herein by reference in their entirety.

### Guide: Target Sequence Matching Requirements

In classic CRISPR systems, the degree of complementarity between a guide sequence and its corresponding target sequence can be about 50%, 60%, 75%, 80%, 85%, 90%, 95%, 97.5%, 99%, or 100%. In some embodiments, the degree of complementarity is 100%. The guide RNAs can be about 5, 10, 11, 12, 13, 14, 15, 16, 17, 18, 19, 20, 21, 22, 23, 24, 25, 26, 27, 28, 29, 30, 35, 40, 45. 50, 75, or more nucleotides in length.

To reduce off-target interactions, e.g., to reduce the guide interacting with a target sequence having low complementarity, mutations can be introduced to the CRISPR systems so that the CRISPR systems can distinguish between target and off-target sequences that have greater than 80%, 85%, 90%, or 95% complementarity. In some embodiments, the degree of complementarity is from 80% to 95%, e.g., about 83%, 84%, 85%, 86%, 87%, 88%, 89%, 90%, 91%, 92%, 93%, 94%, or 95% (for example, distinguishing between a target having 18 nucleotides from an off-target of 18 nucleotides having 1, 2, or 3 mismatches). Accordingly, in some embodiments, the degree of complementarity between a guide sequence and its corresponding target sequence is greater than 94.5%, 95%, 95.5%, 96%, 96.5%, 97%, 97.5%, 98%, 98.5%, 99%, 99.5%, or 99.9%. In some embodiments, the degree of complementarity is 100%.

It is known in the field that complete complementarity is not required, provided there is sufficient complementarity to be functional. For CRISPR nucleases, modulations of cleavage efficiency can be exploited by introduction of mismatches, e.g., one or more mismatches, such as 1 or 2 mismatches between spacer sequence and target sequence, including the position of the mismatch along the spacer/target. The more central (i.e., not at the 3′ or 5′ ends) a mismatch, e.g., a double mismatch, is located; the more cleavage efficiency is affected. Accordingly, by choosing mismatch positions along the spacer sequence, cleavage efficiency can be modulated. For example, if less than 100% cleavage of targets is desired (e.g., in a cell population), 1 or 2 mismatches between spacer and target sequence can be introduced in the spacer sequences.

### Methods of Using CRISPR Systems

The CRISPR-associated transposon systems described herein have a wide variety of utilities including modifying (e.g., deleting, inserting, translocating, inactivating, or activating) a target polynucleotide in a multiplicity of cell types. The CRISPR transposon systems have a broad spectrum of applications in, e.g., tracking and labeling of nucleic acids, drug screening, and treating various genetic disorders.

### High-Throughput Screening

The CRISPR systems described herein can be used for preparing next generation sequencing (NGS) libraries. For example, to create a cost-effective NGS library, the CRISPR systems can be used to disrupt the coding sequence of a target gene, and the CRISPR enzyme transfected clones can be screened simultaneously by next-generation sequencing (e.g., on the Illumina system). CRISPR-associated transposases may enable more efficient preparation due to the ability to directly insert barcodes and adaptor sequences on a transposase payload.

### Engineered Microorganisms

Microorganisms (e.g., E. coli, yeast, and microalgae) are widely used for synthetic biology. The development of synthetic biology has a wide utility, including various clinical applications. For example, the programmable CRISPR systems can be used to split proteins of toxic domains for targeted cell death, e.g., using cancer-linked RNA as target transcript. Further, pathways involving protein-protein interactions can be influenced in synthetic biological systems with e.g. fusion complexes with the appropriate effectors such as kinases or enzymes.

In some embodiments, guide RNA sequences that target phage sequences can be introduced into the microorganism. Thus, the disclosure also provides methods of vaccinating a microorganism (e.g., a production strain) against phage infection.

In some embodiments, the CRISPR systems provided herein can be used to engineer microorganisms, e.g., to improve yield or improve fermentation efficiency. For example, the CRISPR systems described herein can be used to engineer microorganisms, such as yeast, to generate biofuel or biopolymers from fermentable sugars, or to degrade plant-derived lignocellulose derived from agricultural waste as a source of fermentable sugars. More particularly, the methods described herein can be used to modify the expression of endogenous genes required for biofuel production and/or to modify endogenous genes, which may interfere with the biofuel synthesis. These methods of engineering microorganisms are described e.g., in Verwaal et al., “CRISPR/Cpf1 enables fast and simple genome editing of Saccharomyces cerevisiae,” Yeast, 2017 Scp 8. doi: 10.1002/yea.3278; and Hlavova et al., “Improving microalgae for biotechnology—from genetics to synthetic biology,” Biotechnol. Adv., 2015 Nov. 1; 33:1194-203, both of which are incorporated herein by reference in the entirety.

### Application in Plants

The CRISPR systems described herein have a wide variety of utility in plants. In some embodiments, the CRISPR systems can be used to engineer genomes of plants (e.g., improving production, making products with desired post-translational modifications, or introducing genes for producing industrial products). In some embodiments, the CRISPR systems can be used to introduce a desired trait to a plant (e.g., with or without heritable modifications to the genome), or regulate expression of endogenous genes in plant cells or whole plants.

In some embodiments, the CRISPR systems can be used to identify, edit, and/or silence genes encoding specific proteins, e.g., allergenic proteins (e.g., allergenic proteins in peanuts, soybeans, lentils, peas, green beans, and mung beans). A detailed description regarding how to identify, edit, and/or silence genes encoding proteins is described, e.g., in Nicolaou et al., “Molecular diagnosis of peanut and legume allergy,” Curr. Opin. Allergy Clin. Inununol., 2011 Jun.; 11(3):222-8, and WO 2016205764 A1; both of which are incorporated herein by reference in the entirety.

### Gene Drives

Gene drive is the phenomenon in which the inheritance of a particular gene or set of genes is favorably biased. The CRISPR systems described herein can be used to build gene drives. For example, the CRISPR systems can be designed to target and disrupt a particular allele of a gene, causing the cell to copy the second allele to fix the sequence. Because of the copying, the first allele will be converted to the second allele, increasing the chance of the second allele being transmitted to the offspring. A detailed method regarding how to use the CRISPR systems described herein to build gene drives is described, e.g., in Hammond et al., “A CRISPR-Cas9 gene drive system targeting female reproduction in the malaria mosquito vector Anopheles gambiae,” Nat. Biotechnol., 2016 January; 34(1):78-83, which is incorporated herein by reference in its entirety.

### Pooled-Screening

As described herein, pooled CRISPR screening is a powerful tool for identifying genes involved in biological mechanisms such as cell proliferation, drug resistance, and viral infection. Cells are transduced in bulk with a library of guide RNA (gRNA)-encoding vectors described herein, and the distribution of gRNAs is measured before and after applying a selective challenge. Pooled CRISPR screens work well for mechanisms that affect cell survival and proliferation, and they can be extended to measure the activity of individual genes (e.g., by using engineered reporter cell lines). CRISPR-associated transposases may enable more efficient disruption due to the potential for inserting larger sequences. Arrayed CRISPR screens, in which only one gene is targeted at a time, make it possible to use RNA-seq as the readout. In some embodiments, the CRISPR systems as described herein can be used in single-cell CRISPR screens. A detailed description regarding pooled CRISPR screenings can be found, e.g., in Datlinger et al., “Pooled CRISPR screening with single-cell transcriptome read-out,” Nat. Methods., 2017 March; 14(3):297-301, which is incorporated herein by reference in its entirety.

### Saturation Mutagenesis (Bashing)

The CRISPR systems described herein can be used for in situ saturating mutagenesis. In some embodiments, a pooled guide RNA library can be used to perform in situ saturating mutagenesis for particular genes or regulatory elements. In other embodiments, a pooled library of DNA inserts containing a saturating mutagenesis library can be inserted by the CRISPR-associated transposase. Such methods can reveal critical minimal features and discrete vulnerabilities of these genes or regulatory elements (e.g., enhancers). These methods are described, e.g., in Canver et al., “BCL11A enhancer dissection by Cas9-mediated in situ saturating mutagenesis,” Nature. 2015 Nov. 12; 527(7577):192-7, which is incorporated herein by reference in its entirety.

### RNA-Related Applications

The CRISPR systems described herein can have various RNA-related applications, e.g., modulating gene expression, inhibiting RNA expression, screening RNA or RNA products, determining functions of lincRNA or non-coding RNA, inducing cell dormancy, inducing cell cycle arrest, reducing cell growth and/or cell proliferation, inducing cell anergy, inducing cell apoptosis, inducing cell necrosis, inducing cell death, and/or inducing programmed cell death. A detailed description of these applications can be found, e.g., in WO 2016205764 A1, which is incorporated herein by reference in its entirety.

### Modulating Gene Expression

The CRISPR systems described herein can be used to modulate gene expression. The CRISPR systems can be used, together with suitable guide RNAs, to target gene expression, via control of RNA processing. The control of RNA processing can include, e.g., RNA processing reactions such as RNA splicing (e.g., alternative splicing), viral replication, and tRNA biosynthesis. The RNA targeting proteins in combination with suitable guide RNAs can also be used to control RNA activation (RNAa). RNA activation is a small RNA-guided and Argonaute (Ago)-dependent gene regulation phenomenon in which promoter-targeted short double-stranded RNAs (dsRNAs) induce target gene expression at the transcriptional/epigenetic level. RNAa leads to the promotion of gene expression, so control of gene expression may be achieved that way through disruption or reduction of RNAa. In some embodiments, the methods include the use of the RNA targeting CRISPR as substitutes for e.g., interfering ribonucleic acids (such as siRNAs, shRNAs, or dsRNAs). The methods of modulating gene expression are described, e.g., in WO 2016205764, which is incorporated herein by reference in its entirety.

### Controlling RNA Interference

Control over interfering RNAs or microRNAs (miRNA) can help reduce off-target effects by reducing the longevity of the interfering RNAs or miRNAs in vivo or in vitro. In some embodiments, the target RNAs can include interfering RNAs, i.e., RNAs involved in the RNA interference pathway, such as small hairpin RNAs (shRNAs), small interfering (siRNAs), etc. In some embodiments, the target RNAs include, e.g., miRNAs or double stranded RNAs (dsRNA).

In some embodiments, if the RNA targeting protein and suitable guide RNAs are selectively expressed (for example spatially or temporally under the control of a regulated promoter, for example a tissue-or cell cycle-specific promoter and/or enhancer), this can be used to protect the cells or systems (in vivo or in vitro) from RNA interference (RNAi) in those cells. This may be useful in neighboring tissues or cells where RNAi is not required or for the purposes of comparison of the cells or tissues where the effector proteins and suitable guide RNAs are and are not expressed (i.e., where the RNAi is not controlled and where it is, respectively). The RNA targeting proteins can be used to control or bind to molecules comprising or consisting of RNAs, such as ribozymes, ribosomes, or riboswitches. In some embodiments, the guide RNAs can recruit the RNA targeting proteins to these molecules so that the RNA targeting proteins are able to bind to them. These methods are described, e.g., in WO 2016205764 and WO 2017070605, both of which are incorporated herein by reference in the entirety.

### Therapeutic Applications

The CRISPR-associated transposon systems described herein can have diverse therapeutic applications. Without wishing to be limiting, one framework to organize the range of therapeutic applications enabled by CRISPR-associated transposon systems is determining whether the therapeutic genetic modification is a correction of a native locus, or locus-agnostic gene augmentation.

For therapeutic correction of a native locus, the new CRISPR systems can be used to correct mutations responsible for monogenic diseases (e.g., Duchenne Muscular Dystrophy, Cystic Fibrosis, etc.), or introduce beneficial mutations (e.g., Pesk9 for lowered cardiovascular disease risk. BCL11a for increasing fetal hemoglobin expression in treating hemoglobinapthies, CCR5 for HIV resistance, etc.) Using CRISPR-associated transposons may have key advantages. The first advantage is the potential to use a single therapeutic construct to correct a diverse set of genetic mutations, whether in a single patient or across the patient population. This is due to the fact that transposition enables the replacement of a large gene fragment, rather than the short-range corrections enabled by homology directed repair following DNA cleavage or base editing. Second, using CRISPR-associated transposons may enable therapeutic modifications in a broad range of post-mitotic cells and tissues, given that the enzymatic mechanism of action of the transposon is anticipated to be independent from DNA repair mechanisms such as homologous recombination or homology directed repair.

For locus-agnostic gene augmentation, the new CRISPR systems can be used to introduce gene fragments that provide a therapeutic benefit. This includes gene therapies to replace missing or defective native enzymes, including, but not limited to, RPE65 in Leber's Congenital Amaurosis, adenosine de-aminase in Severe Combined Immunodeficiency (SCID), and any number of defective enzymes causing diseases of inborn errors of metabolism. In addition to supplementing defective enzymes, the CRISPR-associated transposons may provide augment existing cellular properties, such as the introduction of custom chimeric antigen T-cell receptors for the production of cell therapies. The CRISPR-associated transposons may have the advantages of greater therapeutic durability when the transgene is incorporated genomically (vs. cpisomal expression in some recombinant viral vectors), and of greater control in transgene insertion, to ensure that the transposon is directed towards chromosomal locations that are at once “safe harbors” for genome editing but still transcriptionally active. This is differentiated from the possible deleterious effects of pseudo-random insertion of integrating viruses, as well as the promiscuous insertion using transposases such as Tn5, piggyBac, and Sleeping Beauty.

Altogether, a programmable transposon would enable a range of genome modifications that may prove highly valuable to therapeutic development, whether directly via therapeutic gene corrections, or indirectly by enabling the engineering of cells and disease models.

### Delivery

Through this disclosure and the knowledge in the art, the CRISPR systems described herein, or components thereof, nucleic acid molecules thereof, or nucleic acid molecules encoding or providing components thereof, can be delivered by various delivery systems such as vectors, e.g., plasmids, viral delivery vectors. The new CRISPR enzymes and/or any of the RNAs (e.g., guide RNAs) can be delivered using suitable vectors, e.g., plasmids or viral vectors, such as adeno-associated viruses (AAV), lentiviruses, adenoviruses, and other viral vectors, or combinations thereof. The proteins and one or more guide RNAs can be packaged into one or more vectors, e.g., plasmids or viral vectors.

In some embodiments, the vectors, e.g., plasmids or viral vectors, are delivered to the tissue of interest by, e.g., intramuscular injection, intravenous administration, transdermal administration, intranasal administration, oral administration, or mucosal administration. Such delivery may be either via a single dose, or multiple doses. One skilled in the art understands that the actual dosage to be delivered herein may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

In certain embodiments, the delivery is via adenoviruses, which can be at a single dose containing at least 1×10⁵ particles (also referred to as particle units, pu) of adenoviruses. In some embodiments, the dose preferably is at least about 1×10⁶ particles, at least about 1×10⁷ particles, at least about 1×10⁸ particles, and at least about 1×10⁹ particles of the adenoviruses. The delivery methods and the doses are described, e.g., in WO 2016205764 A1 and U.S. Pat. No. 8,454,972 B2, both of which are incorporated herein by reference in the entirety.

In some embodiments, the delivery is via plasmids. The dosage can be a sufficient number of plasmids to elicit a response. In some cases, suitable quantities of plasmid DNA in plasmid compositions can be from about 0.1 to about 2 mg. Plasmids will generally include (i) a promoter; (ii) a sequence encoding a nucleic acid-targeting CRISPR enzymes, operably linked to the promoter; (iii) a selectable marker; (iv) an origin of replication; and (v) a transcription terminator downstream of and operably linked to (ii). The plasmids can also encode the RNA components of a CRISPR complex, but one or more of these may instead be encoded on different vectors. The frequency of administration is within the ambit of the medical or veterinary practitioner (e.g., physician, veterinarian), or a person skilled in the art.

In another embodiment, the delivery is via liposomes or lipofectin formulations and the like, and can be prepared by methods known to those skilled in the art. Such methods are described, for example, in WO 2016205764 and U.S. Pat. Nos. 5.593,972; 5,589,466; and 5,580,859; each of which is incorporated herein by reference in its entirety.

In some embodiments, the delivery is via nanoparticles or exosomes. For example, exosomes have been shown to be particularly useful in delivery RNA.

Further means of introducing one or more components of the new CRISPR systems to the cell is by using cell penetrating peptides (CPP). In some embodiments, a cell penetrating peptide is linked to the CRISPR enzymes. In some embodiments, the CRISPR enzymes and/or guide RNAs are coupled to one or more CPPs to effectively transport them inside cells (e.g., plant protoplasts). In some embodiments, the CRISPR enzymes and/or guide RNA(s) are encoded by one or more circular or non-circular DNA molecules that are coupled to one or more CPPs for cell delivery.

CPPs are short peptides of fewer than 35 amino acids either derived from proteins or from chimeric sequences capable of transporting biomolecules across cell membrane in a receptor independent manner. CPPs can be cationic peptides, peptides having hydrophobic sequences, amphipathic peptides, peptides having proline-rich and anti-microbial sequences, and chimeric or bipartite peptides. Examples of CPPs include, e.g., Tat (which is a nuclear transcriptional activator protein required for viral replication by HIV type 1), penetratin, Kaposi fibroblast growth factor (FGF) signal peptide sequence, integrin β3 signal peptide sequence, polyarginine peptide Args sequence, Guanine rich-molecular transporters, and sweet arrow peptide. CPPs and methods of using them are described, e.g., in Hällbrink et al., “Prediction of cell-penetrating peptides,” Methods Mol. Biol., 2015;1324:39-58; Ramakrishna et al., “Gene disruption by cell-penetrating peptide-mediated delivery of Cas9 protein and guide RNA,” Genome Res., 2014 June;24(6):1020-7; and WO 2016205764 A1; each of which is incorporated herein by reference in its entirety.

Various delivery methods for the CRISPR systems described herein are also described, e.g., in U.S. Pat. No. 8,795,965, EP 3009511, WO 2016205764, and WO 2017070605; each of which is incorporated herein by reference in its entirety.

### EXAMPLES

The invention is further described in the following examples, which do not limit the scope of the invention described in the claims.

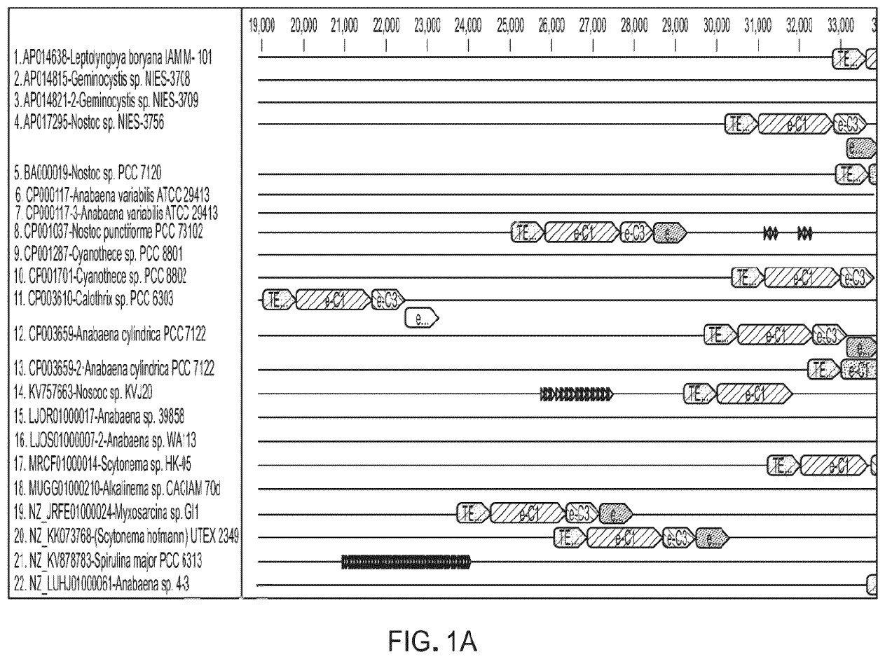

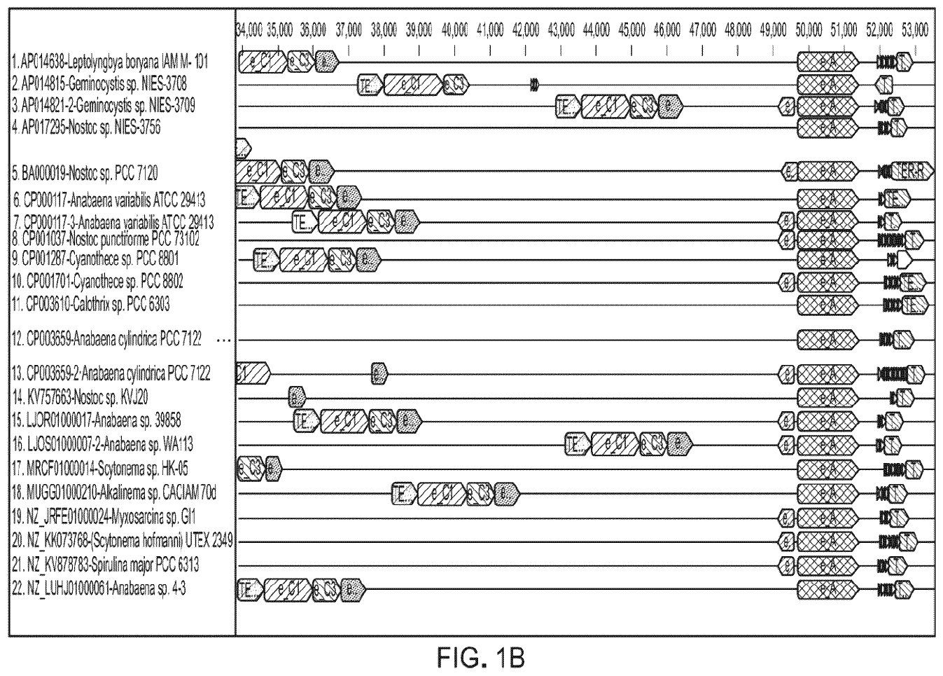

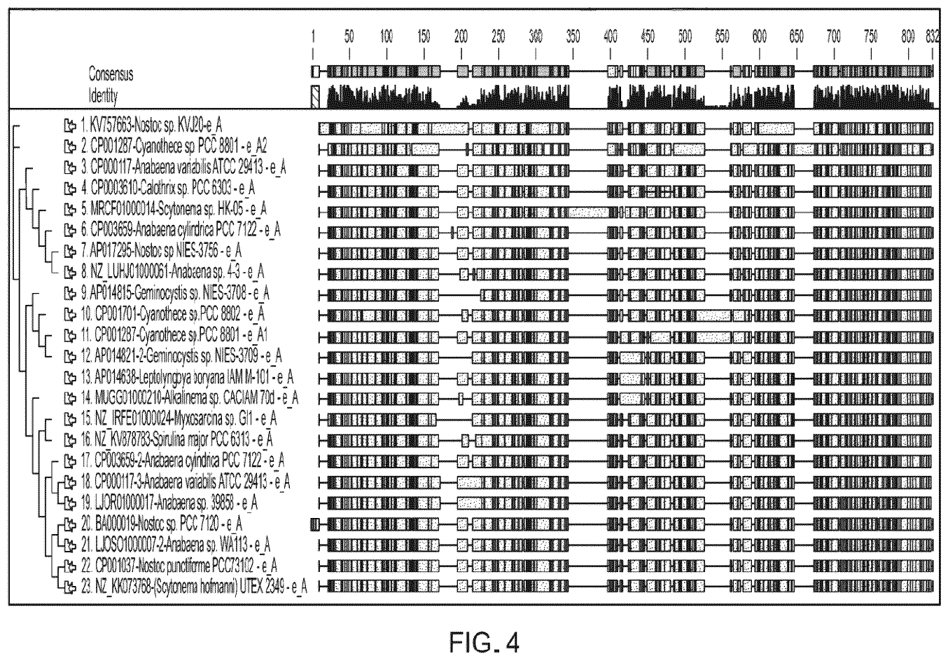

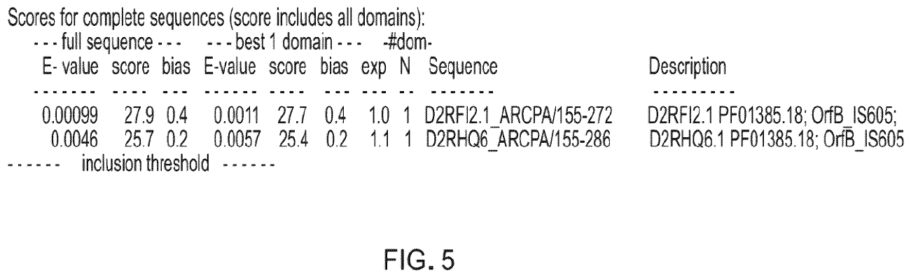

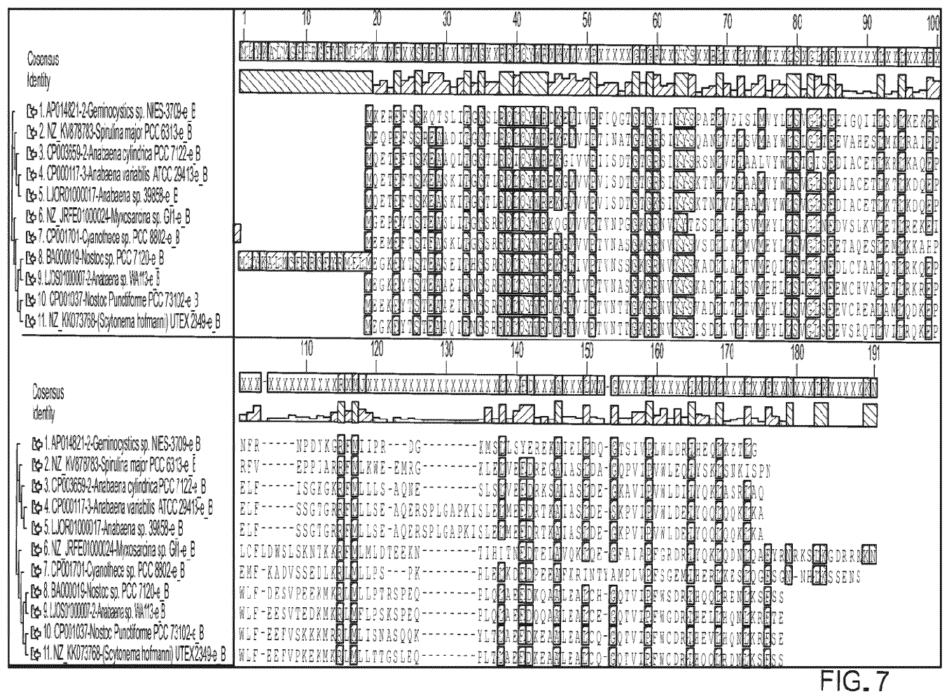

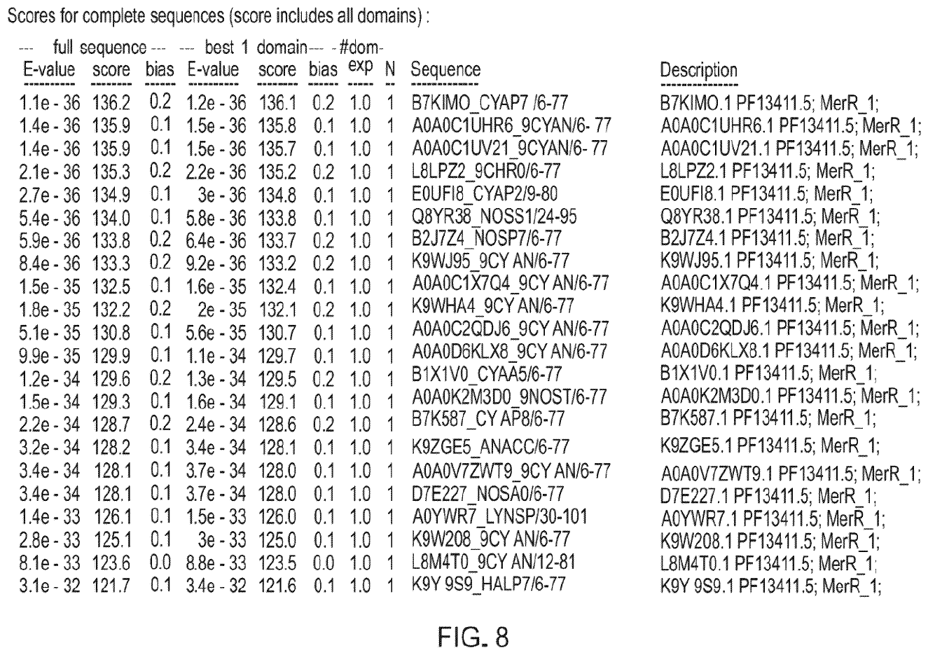

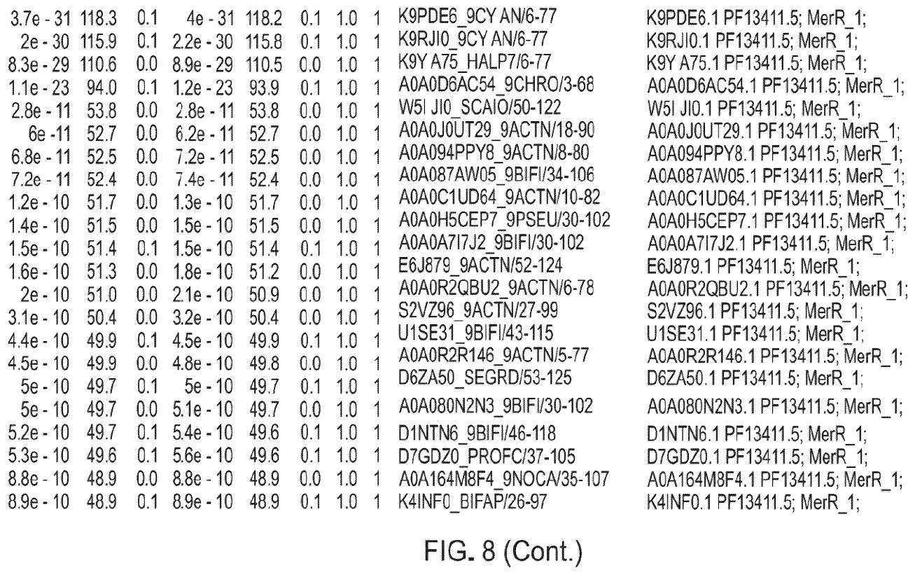

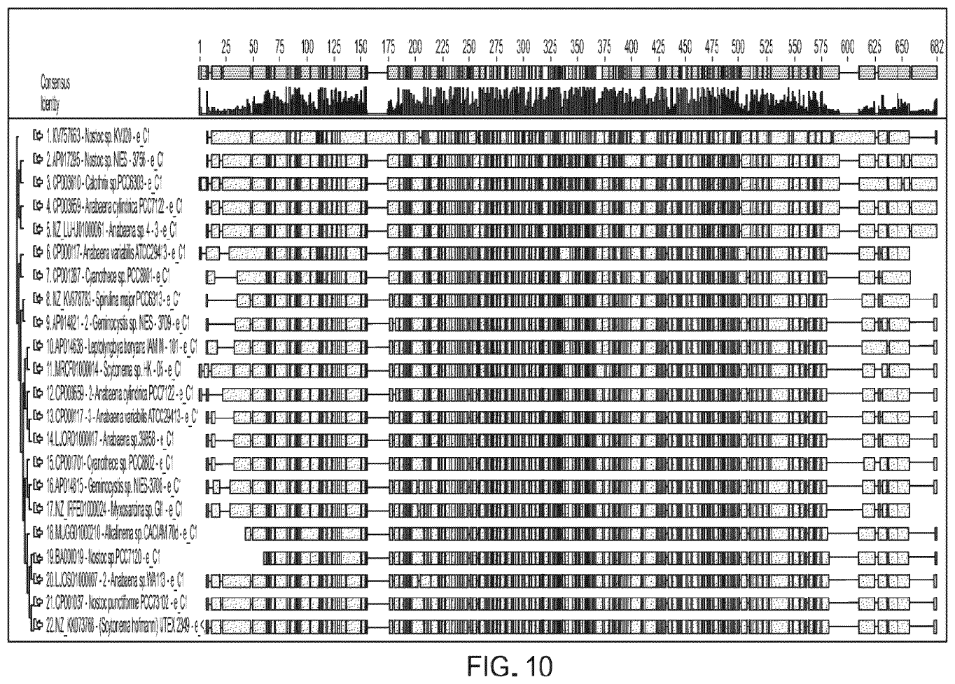

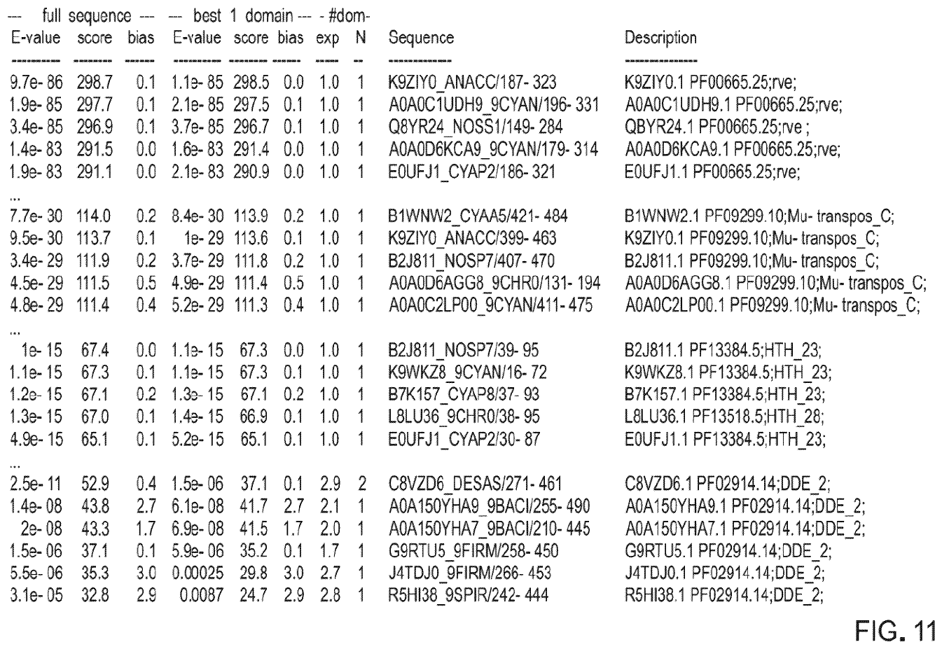

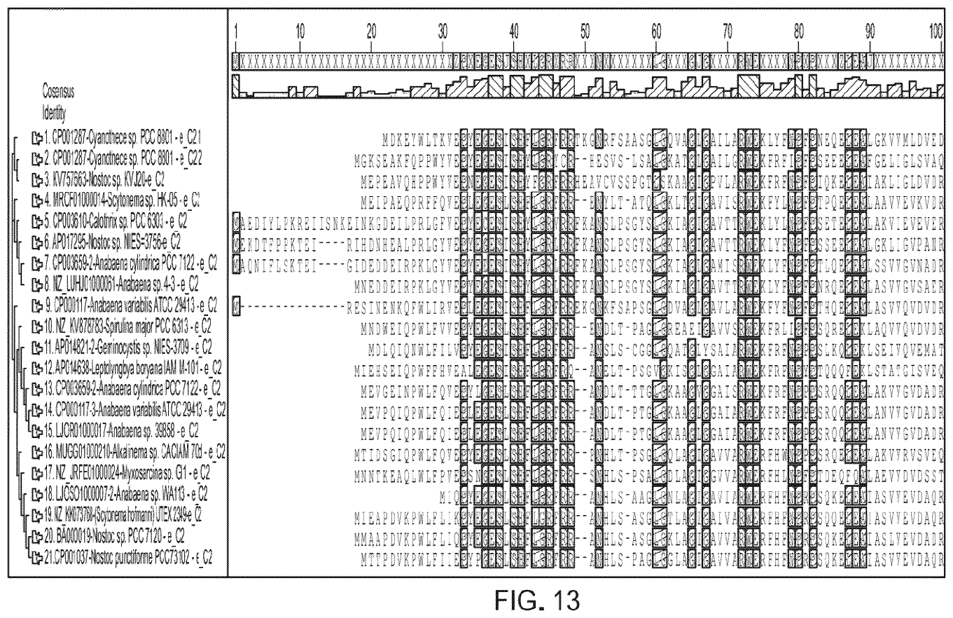

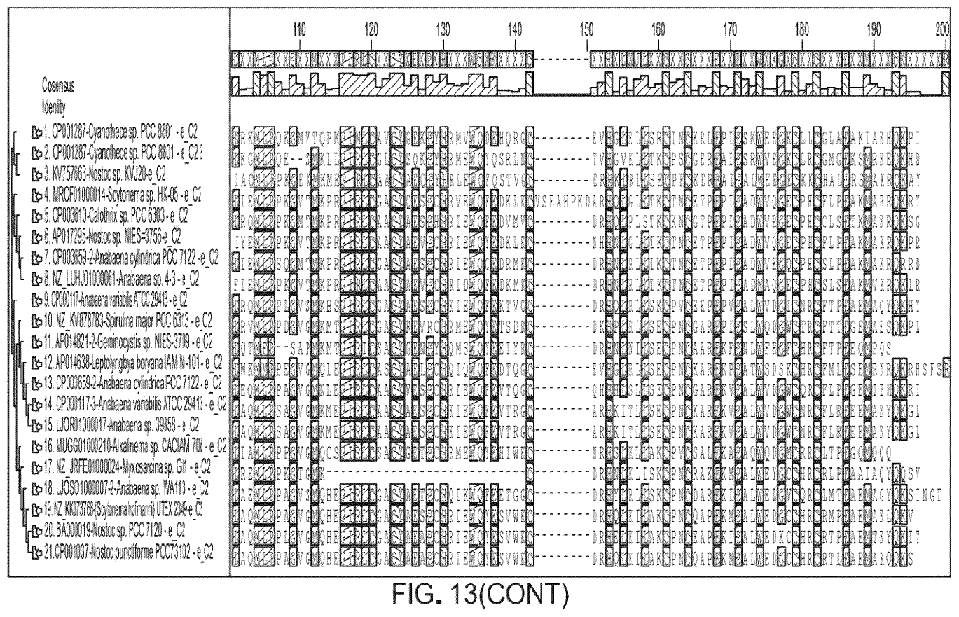

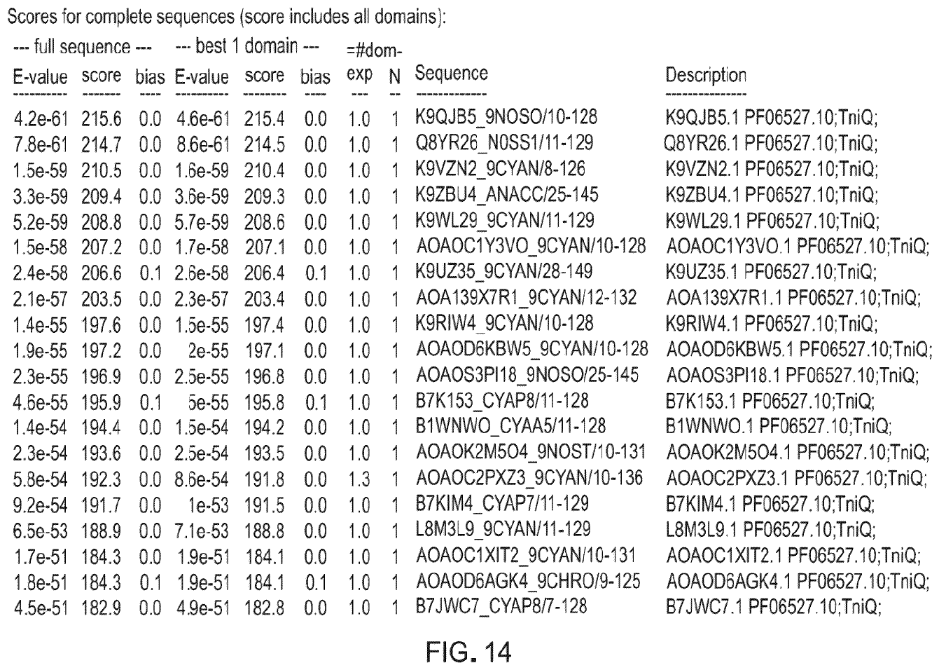

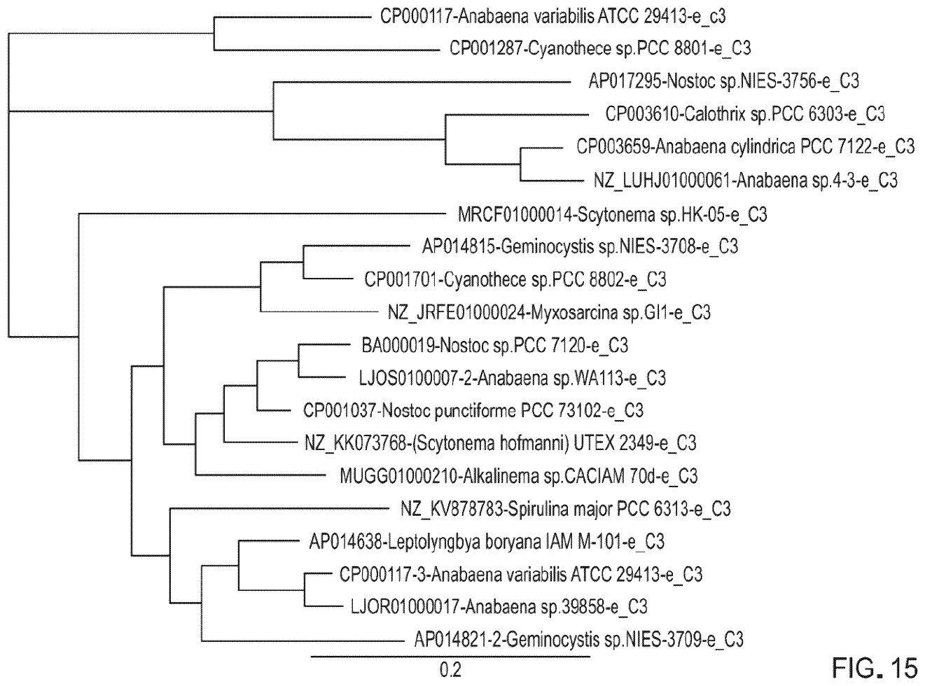

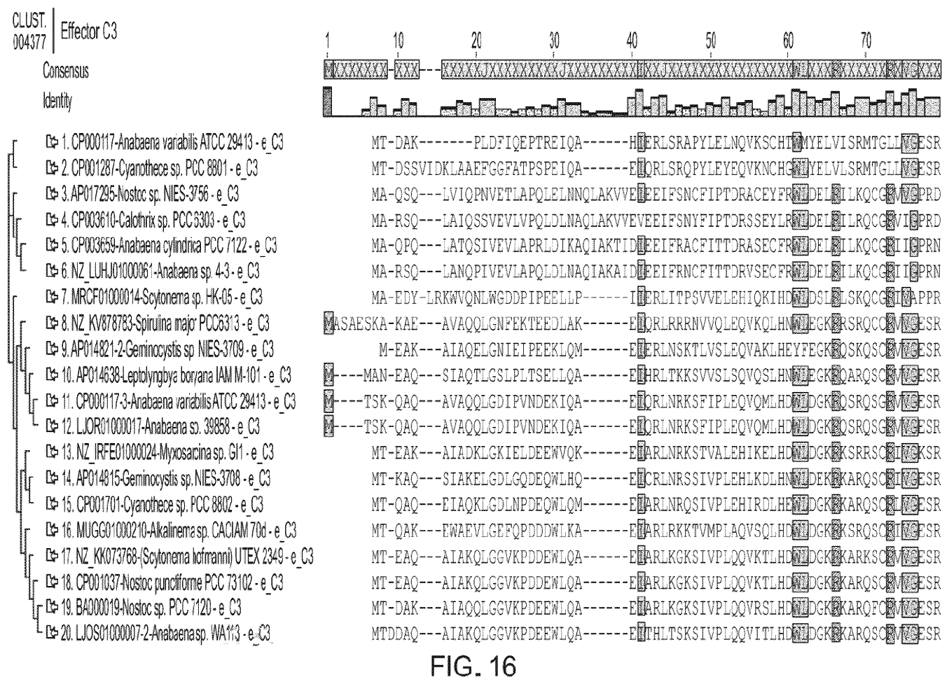

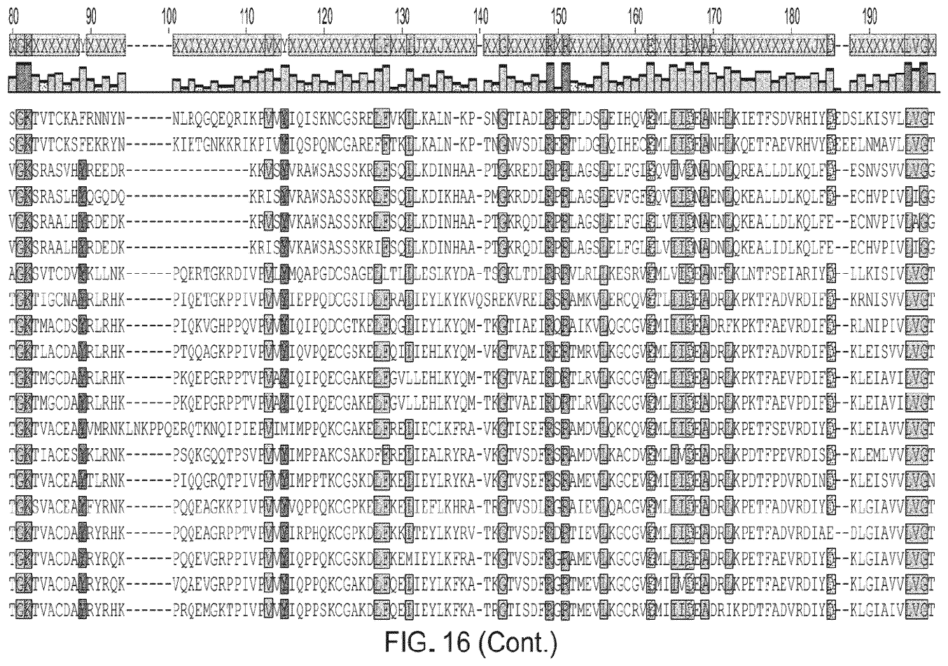

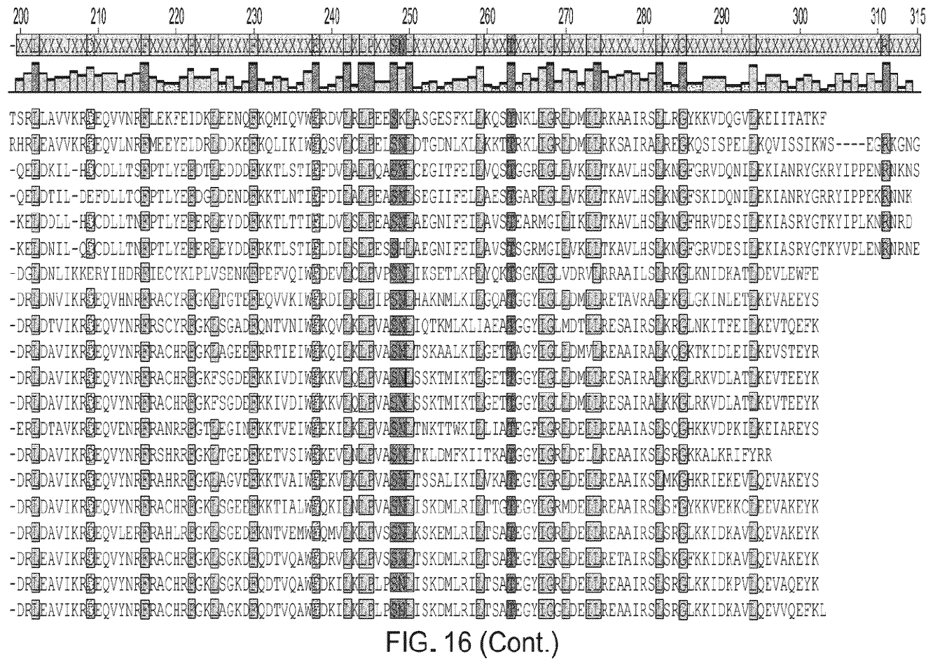

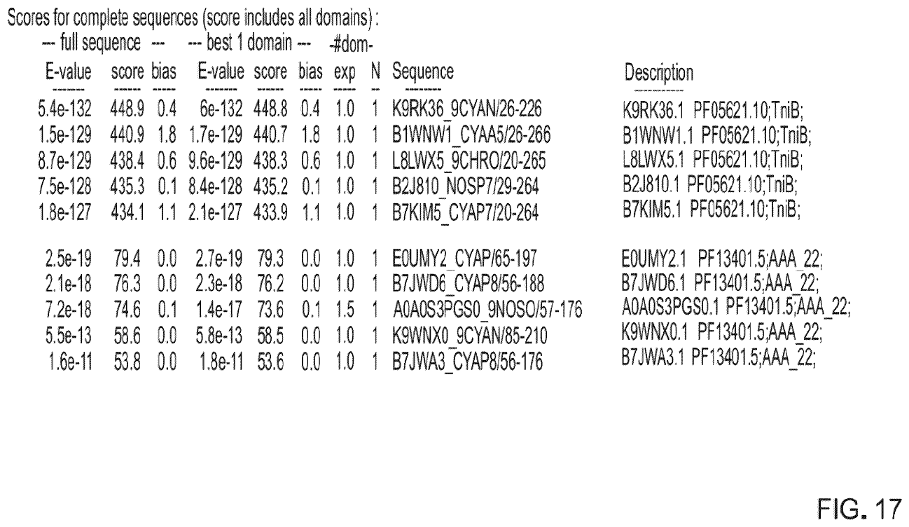

### Example 1—Identification of Minimal Components for a CLUST.004377 CRISPR System (FIGS. 1-17)

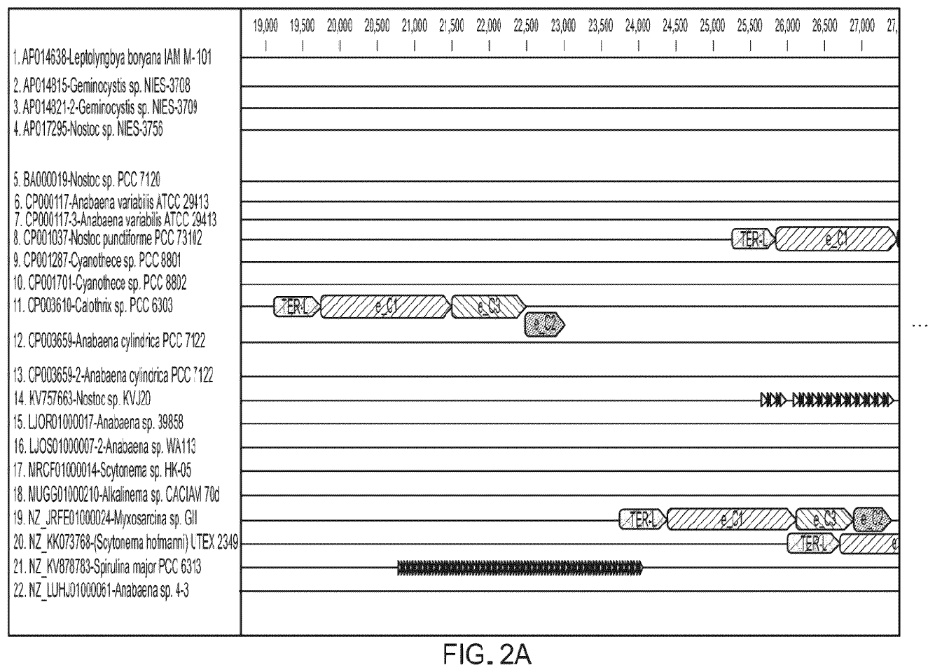

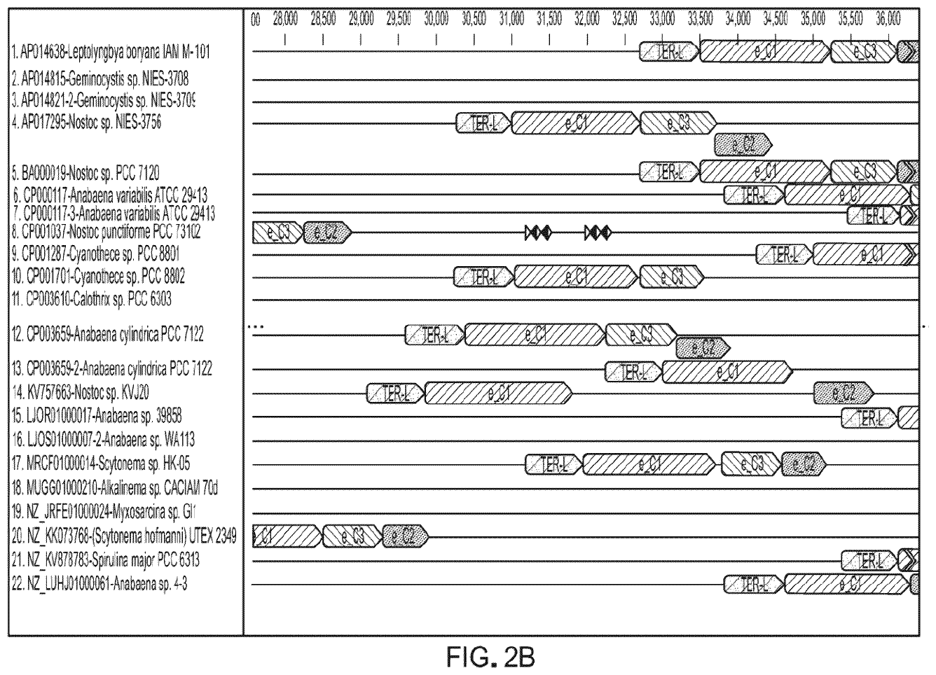

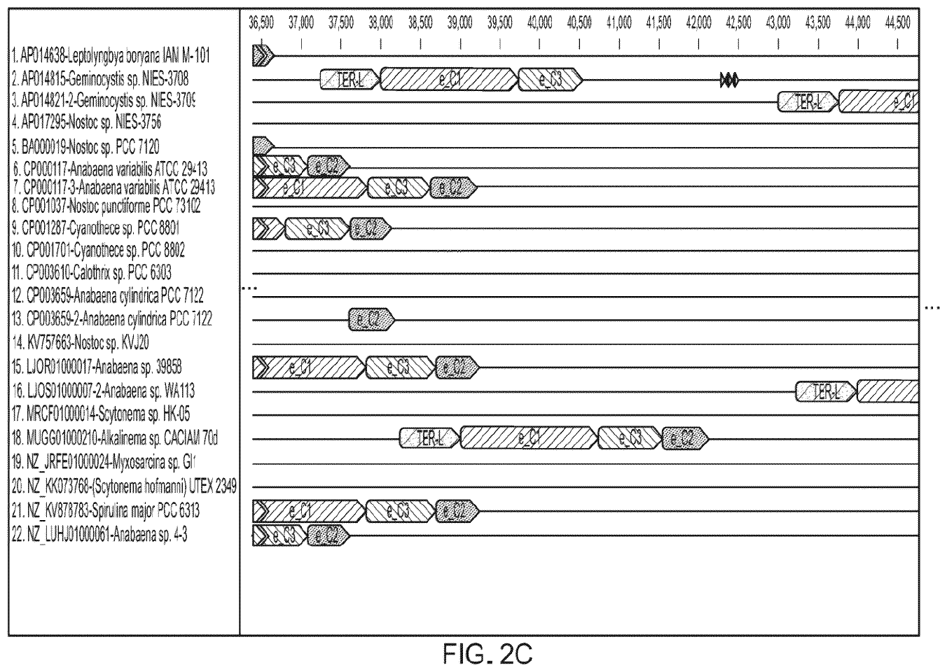

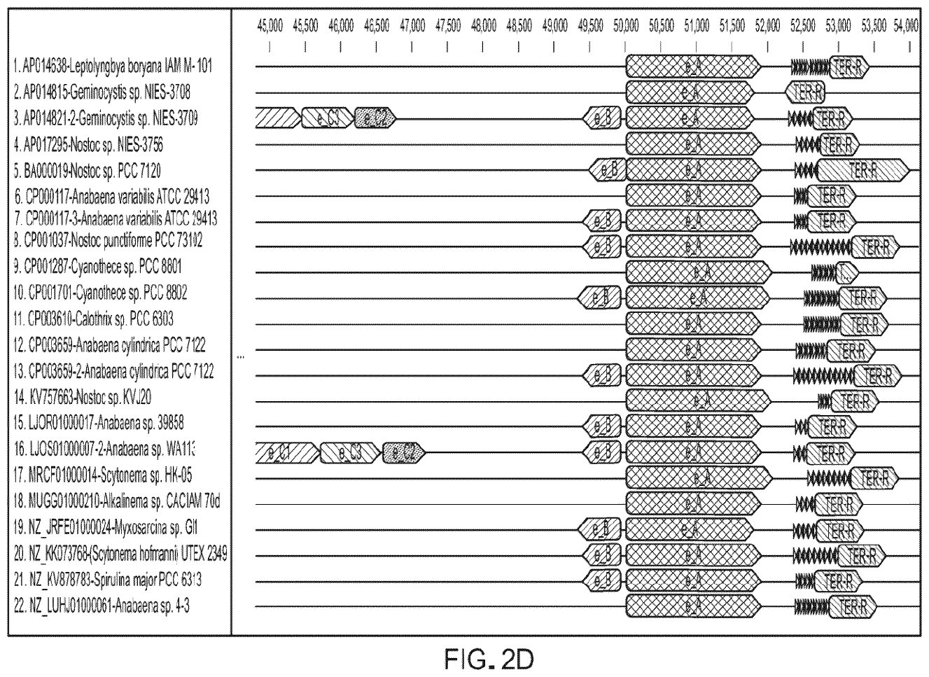

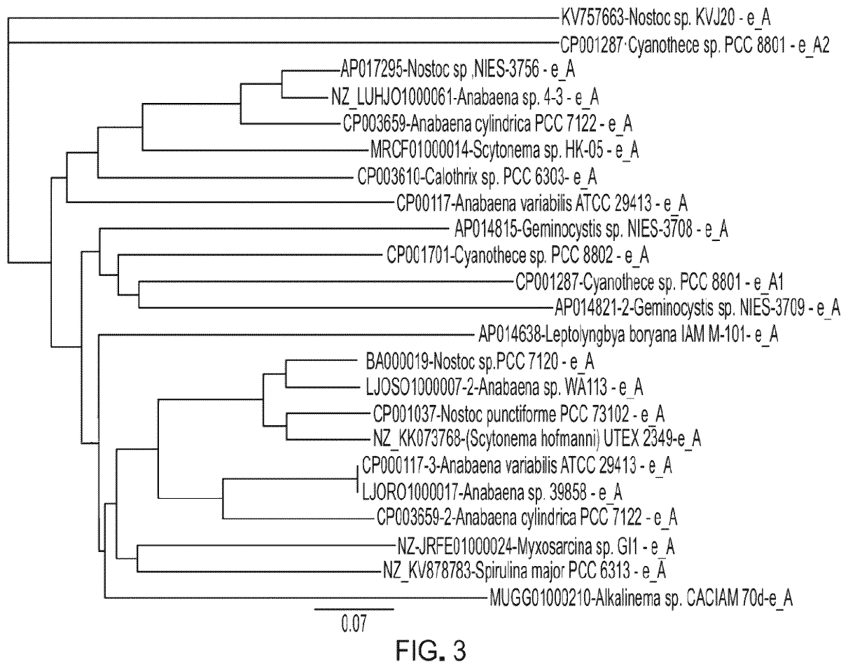

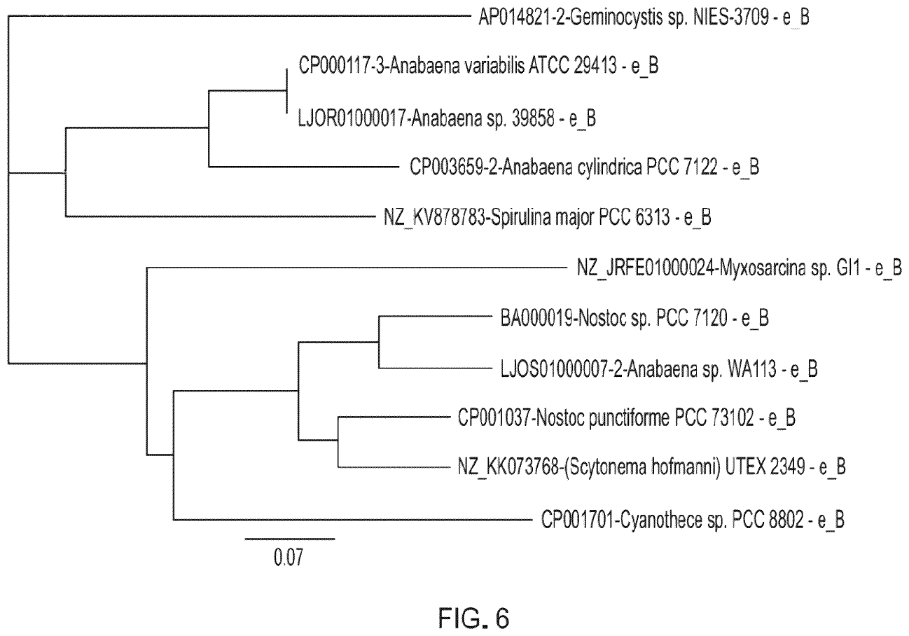

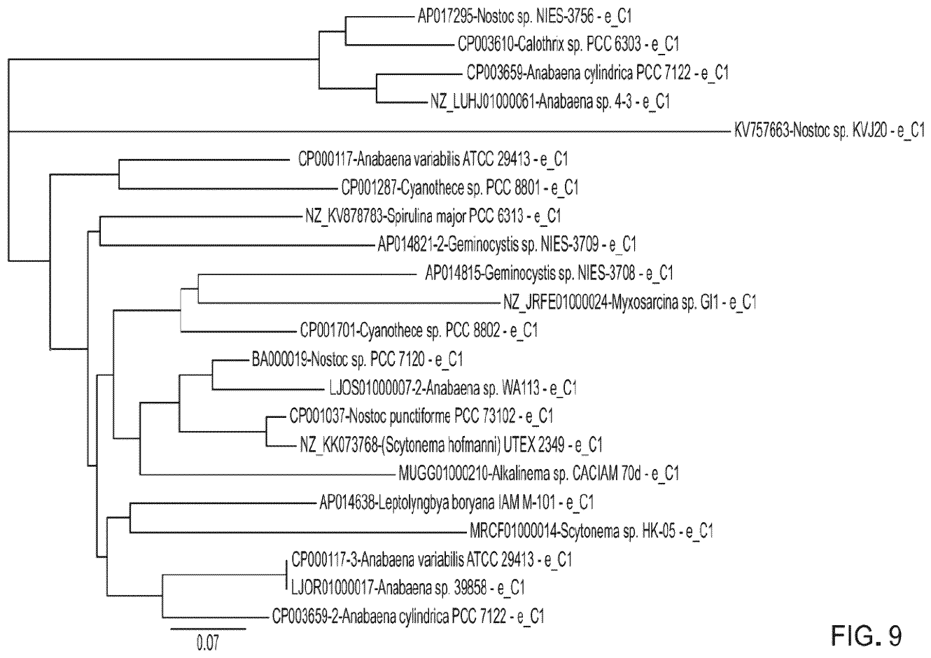

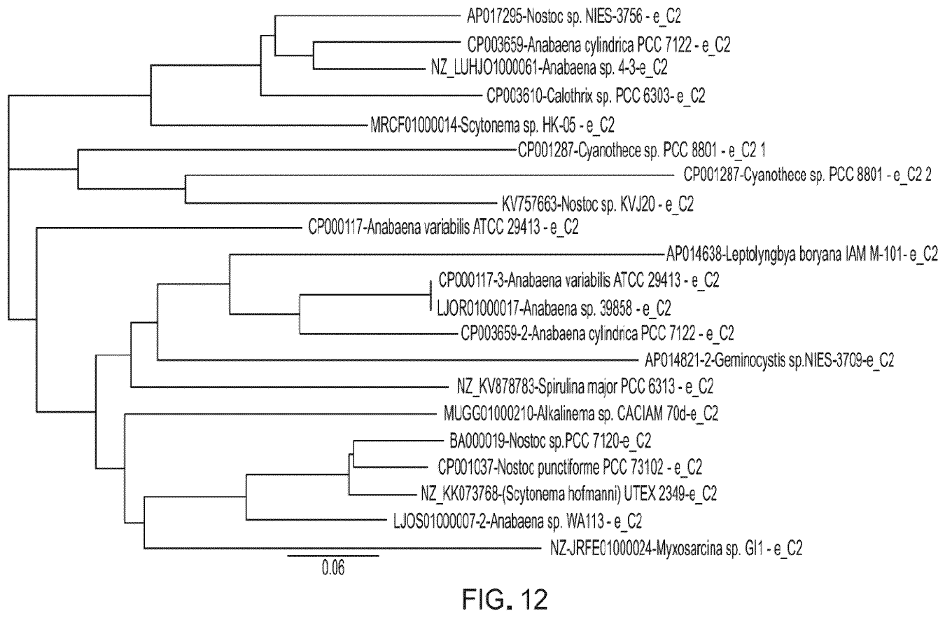

This protein family describes a mobile genetic element associated with CRISPR systems found in organisms including but not limited to Anabaena and Cyanothece (FIG. 1). The naturally occurring loci containing this effector are depicted in FIGS. 1, 2A-2B, indicative that Effector A (˜620 amino acids) has a high co-occurrence with Effectors C1, C2, and C3, as well as the effector protein Effector B (˜140 aa). CLUST.004377 effectors include the exemplary effectors detailed in TABLES 1-9.
.Effector A revealed an OrfB_IS605 domain, indicative that it is derived from the TnpB family of non-autonomous transposases (FIGS. 3-5).
A domain search for Effector B revealed a MerR_1 domain indicative of transcriptional regulation (FIGS. 6-8).
An HMM profile constructed from the multiple sequence alignment of Effector C1, and Pfam and Uniprot database searches revealed an rve domain indicative of integrase activity, as well as HTH, Mu-transposase, and DDE_2 domains, indicative of nucleic acid binding and transposition activity (FIGS. 9-11).
A domain search as described above for Effectors C2 and C3 revealed TniQ and TniB domains respectively, indicative of accessory functions to the transposition process (FIGS. 12-14, 15-17).

TABLE 1
Representative CLUST.004377 Effector Proteins

| Species | Effector A | Effector B | Effector C1 | Effector C2 |
|---|---|---|---|---|
| Alkalinema sp. CACIAM 70d | OUC12050.1 | N/A | OUC12067.1 | OUC12055.1 |
| (MUGG01000210) | | | | |
| Anabaena cylindrica PCC 7122 (CP003659) | AFZ58287.1 | AFZ58288.1 | AFZ58294.1 | AFZ58293.1 |
| Anabaena cylindrica PCC 7122 (CP003659) | AFZ56196.1 | N/A | AFZ56182.1 | AFZ56184.1 |
| Anabaena sp. 4-3 (NZ_LUHJ01000061) | WP_066425713.1 | N/A | WP_066425682.1 | WP_066425687.1 |
| Anabaena sp. WA113 (LIOS01000007) | OBQ23770.1 | OBQ23832.1 | OBQ23776.1 | OBQ23833.1 |
| Anabaena variabilis ATCC 29413 (CP000117) | WP_011318157.1 | N/A | ABA20966.1 | WP_011318169.1 |
| Anabaena variabilis ATCC 29413 (CP000117) | WP_011318983.1 | N/A | WP_011318973.1 | WP_011318975.1 |
| Anabaena variabilis ATCC 29413 (CP000117) | WP_011318008.1 | WP_011318007.1 | WP_011317997.1 | WP_011317999.1 |
| Aphanocapsa montana BDHKU210001 | WP_039730330.1 | N/A | WP_044151373.1 | WP_039728662.1 |
| (NZ_JTJD01000271) | | | | |
| Calothrix sp. PCC 6303 (CP003610) | AFZ00435.1 | N/A | AFZ00420.1 | AFZ00422.1 |
| Calothrix sp. PCC 7103 (NZ_KB217483) | WP_019497312.1 | N/A | WP_019497296.1 | WP_019497300.1 |
| Cyanothece sp. PCC 8801 (CP001287) | WP_012596241.1 | N/A | WP_012596233.1 | WP_012596235.1 |
| Cyanothece sp. PCC 8801 (CP001287) | WP_012596246.1 | N/A | WP_012596233.1 | WP_012596235.1 |
| Cyanothece sp. PCC 8802 (CP001701) | ACV01094.1 | ACV01093.1 | ACV01082.1 | N/A |
| Geminocystis sp. NIES-3708 (AP014815) | BAQ60380.1 | N/A | BAQ60391.1 | BAQ60389.1 |
| Geminocystis sp. NIES-3709 (AP014821) | BAQ63841.1 | BAQ63842.1 | BAQ63848.1 | BAQ63846.1 |
| Geminocystis sp. NIES-3709 (NZ_AP014821) | WP_066116114.1 | WP_066116116.1 | WP_066116124.1 | WP_066116120.1 |
| Leptolyngbya boryana dg5 (AP014642) | WP_017289534.1 | N/A | WP_017289545.1 | BAS55900.1 |
| Leptolyngbya boryana IAM M-101 | WP_017289534.1 | N/A | WP_017289545.1 | WP_026148796.1 |
| (NZ_AP014638) | | | | |
| Leptolyngbya boryana PCC 6306 | WP_017289534.1 | N/A | WP_017289545.1 | WP_026148796.1 |
| (NZ_KB731324) | | | | |
| Lyngbya confervoides BDU141951 | WP_039730330.1 | N/A | WP_044151373.1 | WP_039728662.1 |
| (NZ_JTHE01000274) | | | | |
| Myxosarcina sp. GI1 (NZ_RFE01000024) | WP_036484397.1 | WP_052055737.1 | WP_036484426.1 | WP_036484423.1 |
| Nostoc punctiforme PCC 73102 (CP001037) | WP_012411901.1 | WP_012411902.1 | WP_012411919.1 | WP_012411917.1 |
| Nostoc sp. KVJ20 (KV757663) | ODH02164.1 | N/A | N/A | N/A |
| Nostoc sp. NIES-3756 (AP017295) | BAT55382.1 | N/A | BAT55397.1 | BAT55395.1 |
| Nostoc sp. PCC 7120 (BA000019) | BAB75312.1 | WP_010997761.1 | BAB75329.1 | BAB75327.1 |
| Nostoc sp. PCC 7120 (NC_003272) | WP_044521835.1 | WP_010997761.1 | WP_044521841.1 | WP_044521839.1 |
| Nostoc sp. | WP_094343940.1 | N/A | WP_094343929.1 | WP_094343931.1 |
| ‘Peltigera membranacea cyanobiont’ | | | | |
| 232 (NOLK01000067) | | | | |
| Scytonema hofmanni UTEX 2349 | WP_029636312.1 | WP_029636314.1 | WP_084763316.1 | WP_029636334.1 |
| (NZ_KK073768) | | | | |
| Scytonema sp. HK-05 (NZ_MRCF01000014) | WP_073629970.1 | N/A | WP_073629994.1 | WP_073629990.1 |
| Spirulina major PCC 6313 (NZ_KV878783) | WP_072619878.1 | WP_072619877.1 | WP_072619869.1 | WP_072619870.1 |

| | | # | | | effector |
|---|---|---|---|---|---|
| Species | Effector C3 | spacers | cas1 | cas2 | size |
| Alkalinema sp. CACIAM 70d | OUC12056.1 | 3 | N | N | 629 |
| (MUGG01000210) | | | | | |
| Anabaena cylindrica PCC 7122 (CP003659) | N/A | 11 | N | N | 635 |
| Anabaena cylindrica PCC 7122 (CP003659) | AFZ56183.1 | 5 | N | N | 642 |
| Anabaena sp. 4-3 (NZ_LUHJ01000061) | WP_066425684.1 | 6 | N | N | 632 |
| Anabaena sp. WA113 (LIOS01000007) | OBQ23775.1 | 4 | N | N | 639 |
| Anabaena variabilis ATCC 29413 (CP000117) | WP_011318170.1 | 2 | N | N | 635 |
| Anabaena variabilis ATCC 29413 (CP000117) | WP_011318974.1 | 2 | N | N | 643 |
| Anabaena variabilis ATCC 29413 (CP000117) | WP_011317998.1 | 2 | N | N | 642 |
| Aphanocapsa montana BDHKU210001 | WP_044151374.1 | 2 | N | N | 641 |
| (NZ_JTJD01000271) | | | | | |
| Calothrix sp. PCC 6303 (CP003610) | AFZ00421.1 | 7 | N | N | 639 |
| Calothrix sp. PCC 7103 (NZ_KB217483) | WP_019497299.1 | 16 | Y | Y | 649 |
| Cyanothece sp. PCC 8801 (CP001287) | WP_012596234.1 | 7 | N | N | 682 |
| Cyanothece sp. PCC 8801 (CP001287) | WP_012596234.1 | 7 | N | N | 660 |
| Cyanothece sp. PCC 8802 (CP001701) | ACV01083.1 | 6 | N | N | 667 |
| Geminocystis sp. NIES-3708 (AP014815) | BAQ60390.1 | 2 | N | N | 608 |
| Geminocystis sp. NIES-3709 (AP014821) | BAQ63847.1 | 4 | N | N | 646 |
| Geminocystis sp. NIES-3709 (NZ_AP014821) | WP_066116122.1 | 4 | N | N | 616 |
| Leptolyngbya boryana dg5 (AP014642) | WP_026148797.1 | 7 | N | N | 636 |
| Leptolyngbya boryana IAM M-101 | WP_026148797.1 | 7 | N | N | 636 |
| (NZ_AP014638) | | | | | |
| Leptolyngbya boryana PCC 6306 | WP_026148797.1 | 9 | N | N | 636 |
| (NZ_KB731324) | | | | | |
| Lyngbya confervoides BDU141951 | WP_044151374.1 | 2 | N | N | 641 |
| (NZ_JTHE01000274) | | | | | |
| Myxosarcina sp. GI1 (NZ_RFE01000024) | WP_036484424.1 | 4 | N | N | 615 |
| Nostoc punctiforme PCC 73102 (CP001037) | WP_012411918.1 | 15 | N | N | 639 |
| Nostoc sp. KVJ20 (KV757663) | N/A | 2 | N | N | 679 |
| Nostoc sp. NIES-3756 (AP017295) | BAT55396.1 | 4 | N | N | 635 |
| Nostoc sp. PCC 7120 (BA000019) | WP_010997774.1 | 4 | N | N | 648 |
| Nostoc sp. PCC 7120 (NC_003272) | WP_010997774.1 | 4 | N | N | 639 |
| Nostoc sp. | WP_094343930.1 | 5 | N | N | 641 |
| ‘Peltigera membranacea cyanobiont’ | | | | | |
| 232 (NOLK01000067) | | | | | |
| Scytonema hofmanni UTEX 2349 | WP_029636336.1 | 28 | N | N | 639 |
| (NZ_KK073768) | | | | | |
| Scytonema sp. HK-05 (NZ_MRCF01000014) | WP_073629992.1 | 8 | N | N | 690 |
| Spirulina major PCC 6313 (NZ_KV878783) | WP_072622994.1 | 45 | N | N | 627 |

TABLE 2
Amino acid sequences of Representative CLUST.004377 Effector A Proteins

>OUC12050.1
[Alkalinema sp. CACIAM 70d]
MSQITIQCRLVAPPTARQHLWTLAAEKNTPLINTLIQEVANHEEFETWRLKGKHPTKIVSEICRRLRTEAPFSGQPARFYTSAE
KTVNYIFKSWFTLQSRLQRQLSRKQIWLSILKSDPELVELCGHPLETIQNKATQLLTQIEKTLADSNPEDPQSPCTNDLIRAQL
FKKYHGAKTPLIRCSIAYLIKNGGKLPKSPEIPHKFAQRRRKAEIQVQRLQDQLEGRLPKGRDLTGQAWLSTLITATSTVPKDN
HEQKQWNDRLLAKPCTTPFPILFETNEDLNWSKNHTGRICVRFNGLSEHTFQVYCDQRQLPWFQRFLEDQQTKHTSKNQHSSAL
FTLRSAHLAWQETHSNGAGWDNHYITLYCTVDVRLWTAEGTEEVHQEKAVDIAKQLTYLDQKEDLSETQAAFAQRLTSTLDRLN
KPFPRPHRHRAQQHSHIIVGLSVDWEAPLTLAIWNANTQEVLVYRSLRQLLGKDYPLFLQHRQEQQKQSHNRHKAQKRNKNCQF
GTSHLGEHIDRLLAKAVITIAQQYNAGSIAVPNLDNIRETLQATIDAKAEQKAPGCIEAQKRYTKQYKITIHRWSYGRLIDQII
SKAKQVGLGVEEAKQPLSGNVQEKAKLVAISAYNARLIVAF (SEQ ID NO: 1)
>OBQ23770.1
[Anabaena sp. WA113]
MSQITVQCRLIASESTRQQLWTLMAELNTPLINELLQQLSKHPDFEKWRKDGKFPSTVVSQLCQPLKTDPQFAGQPSRCYLSAI
HVVDYIYKSWLTIQKRLQQQLDGKIRWLEMLNSDAELVETSGYSLEAIRTKAAEILAMTTPESDTNVPLTKKRNTKKSKKSSAS
NPEPSLSHKLFNAYQETDDILSRSAISYLLKNGCKLNDKEEDTEKFAKRRRKVEIQIQRLTDKLTSRIPKGRDLTNSKWLETLF
TAITTVPEDNAEAKRWQDILSTRSSSLPFPLIFETNEDLKWSTNEKGRLCVHFNGLTDLTFEVYCDSRQLHWFKRFLEDQQTKR
KSKNQHSSGLFTLRNGRLAWQEGEGKGETWQIHRLTLSCCVDNRLWTAEGTEQVRQEKAEDITKFITKMKEKSDLSDTQQAFIQ
RKQSTLTRINNSFDRPCKPLYQGQSHILVGVSMGLEKPATVAVVDASANKVLTYRSIKQILGENYELLNRQRRQQRSSSHERHK
AQKSFSPNQFGTSELGQYIDRLLAKEIVAIAQTYKAGSIVLPKLGDMRENIQSEIQATAEIKCPGSVEIQQKYAKQYRINVHKW
SYGRLIQSIQSKAAQVGIVIEEGKQPVRDSPQDKAKELALSTYHLRLAKQS (SEQ ID NO: 2)
>WP_011318157.1
[Anabaena variabilis ATCC 29413]
MSVITIQCRLVAEEDILRQLWELMAEKNTPLINELLAQVGKHPEFETWLDKGRIPTELLKTLVNSFKTQERFADQPGRFYTSAI
ALVDYVYKSWFALQKRRKRQIEGKERWLTILKSDLQLEQESQCNLNVIRTKANEILTQFTPQSDQNKNPRKSKKAKKSAKLQKS
SLFQILLNTHEQTQETLTRCATAYLLKNNCQISERDEDPEEFNRNRRTKEIEIERLKDQLQSRIPKGRDLTGEEWLETLEIATV
NVPQNEKEAKAWQAALLRKTADVPFPVAYESNEDMTWLQNDKDRLFVRFNGLGKMTFEVYCDKRHLHYFKRFLEDQEIKRNSKN
QYSSSLFTLRSGRLAWLPGKQKGEPWKINQLHLYCTLDTRMWTAEGTQQVVNEKITKITETLTKAKQKDELNDKQQAFITRQQS
TLDRINNPFPRPSQPNYQGRPSILVGVSFGLEKPVTLAVVDVVKNEVLAYRSVKQLLGKNYNLLNRQRQQQQRLSHERHKAQKQ
NAPNSFGESELGQYVDRLLADEIVAIAKTYQAGSIVIPKLRDMREQISSEIQSRAEKKCPGYKEVQQKYAKEYRMSVHRWSYGR
LIESIKSQAAKAGIFTEIGTQPIRGSPQEKARDLAVFAYQERQAALI (SEQ ID NO: 3)
>WP_011318983.1
[Anabaena variabilis ATCC 29413]
MSVITIQCRLIASEATRSYLWQLMAQKNTPLINELIEQLGIHPEIEQWLKKGKLPDGVVKPLCDSLITQESFANQPKRFNKSAI
EVVEYIYKSWLALQKERQQTIDRKEHWLKMLKSDVELEQESKCTLDAIRSQATKILPKYLAQSEQNNNQTQSQNKKKSKKSKTK
NENSTLFDILFKAYDKAKNPLNRCTLAYLLKNNCQVSQKDEDPNQYALRRSKKEKEIERLKKQLQSRKPNGRDLTGREWQQTLI
MATSSVPESNDEANIWQKRLLKKDISLPFPIRFRTNEDLIWSKNEEGRICVSFSGEGLNDHIFETYCGNRQIHWFQRFLEDQNI
KNDNNDQHSSALFTLRSAILAWQENKQHKENSLPWNTRRLTLYCTLDTRLWTTDGTEKVKQEKVDEFTQQLANMEQKENLNQNQ
QNYVKRLQSTLNKLNNAYPRHNHDLYQGKPSILVGVSLGLEKPATLAIVDSSTNIVLAYRSIKQLLGDNYKLLNRQRQQQQRNS
HERHKAQKSNMPNKLSESDLGKYIDNLLAQATIALAKNYQAGSIVLPTMKNVRESIQSEIEARAVKRCPNYKEGQQQYAKQYRQ
SIHRWSYNRLMQFIQSQAVKANISIEQGPQPIRGSSQEKARDLAIAAYYLRQNKS (SEQ ID NO: 4)
>WP_011318008.1
[Anabaena variabilis ATCC 29413]
MSQITIQCRLVASEPSRHQLWKLMVDLNTPLINELLVQVAQHPEFETWRQKGKHPAKIVKELCEPLRTDPRFIGQPGRFYTSAI
ATVNYIYKSWFALMKRSQSQLEGKMRWWEMLKSDAELVEVSGVTLESLRTKAAEILSQFAPQPDTVEAQPAKGKKRKKTKKSDG
DCAERTLRERSISDYLFEAYRDTEEILTRCAINYLLKNGCKISNKEENAEKFAKRRRKLEIQIERLREKLEARIPKGRDLTDAK
WLETLLLATLNVPENEAEAKSWQDSLLKKSITVPFPVAYETNEDMTWFKNERGRICVKFSGLSEHTFQVYCDSRQLQWFQRFLE
DQQIKRNSKNQHSSSLFTLRSGRIAWQEGEGKSEPWKVNRLILYCSVDTRLWTAEGTNLVREEKAEEIAKAIAQTKAKGKLNDK
QQAHIKRKNSSLARINNLFPRPSKPLYKGQSHILVGVSLGLEKPTTLAVVDGSIGKVLTYRNIKQLLGDNYRLLNRQRQQKHTL
SHQRQVAQILASPNQLGESELGQYVDRLLAKEIVAITQTYKAGSIVLPKLGDMREQVQSEIQAKAEQKSDLIEVQQKYSKQYRV
SVHQWSYGRLIASIRSSAAKVGIVIEESKQPIRGSPQEKARELAIAAYNSRRRT (SEQ ID NO: 5)
>WP_039730330.1
[Aphanocapsa montana BDHKU210001]
MSKITIQCRLVASEATRQYLWHLMADIYTPFVNEILRQIREDDNFEQWRQSGKIPASVFEDYRKTLKTESRFQGMPGRWYYAGR
EEVKRIYKSWLALRRRLRNQLAGQNRWLEVLQSDETLMEVSGLDLSALQAEASQLLNILGSKNKTSKNRSKKAKGKPKGKSAKD
PTLYQALWELYRETEDIAKKCVIAYLLKHKCQVPDKPEDPKKFRHRRREAEIRAERLNEQLIKTRLPKGRDLTNEQWLQVLEIA
TRQVPKDEDEAAIWQSRLLTDAAKFPFPVAYETNEDLKWFLNGKGRLCVSFNGLSEHTFEVYCGQRQLYWFNRFLEDQQIKKEN
QGERSAGLFTLRSGRLVWKPYSSDASRSDPWMANQLTLQCSVDTRLWTAEGTEQVRQEKATSIAKVIAGTKAKGNLNQKQQDFI
TKREKTLELLHNPFPRPSKPLYQGKPSIIAAVSFGLEKPATLAIVDIVTDKAITYRSIRQLLGQNYKLFTKHRLKQQQCAHQRH
QNQVESAENRISEGGLGEHLDSLIAKAILETAAEYGASSIVLPELGNIREIIHAEIQAKAERKIPGLKEKQDEYAAKFRASVHR
WSYGRLAQKVTTKASLHGLETESTRQSLQGTPQEKARNLAISAYESRKVAQRA (SEQ ID NO: 6)
>WP_019497312.1
[Calothrix sp. PCC 7103]
MSIITIQCRLVANDRTLQHLWELMAEKNTPLISELLEQLGKHPDFETWLKNGKVPKDTIKILCDSLKTQSRFAGQPGRFYTSAI
SQVKEIYKSWLTLQKRRQRQIEGKQRWLGMLKSDVELQEESNCSLEKIRAKGTEILAEFVSKFTKDTTKKSKTKIKSTKKSNKK
TKKDTEESNSTLFQALCDIYDKTEDTLSKCAIIYLLKNNCQVIDTEENPDTFLKRKRAKEIEIKRLQDQIVGRIPKGRDLTDKK
WLDTIKLASSQVPQDENEAKSWQNQLLKTSSSVPYSVDYETNTDIKWVKHNNGSIFVNFNGLGEHQFEVYCDSRQLPYFQRFCE
DMQIWHNDEEKYSSALFMLRSARLVWLEKKGRGKPWNVNYLYLHCSLDTSLWTAEGTEQIRINKINETDEAIAKAKTKDKQELT
ENQLAYLQRQQSTRNKLNNSFPGRPSKPIYKGNSHILVGVSLGLEKPVTVAAVDVVSNKVLAYRSVKQLLGQNYKLLNRQRQQQ
KHLAQKRHESQKKQAPNQFGESELGLYVDRLLAKSIINFAKTYQASSIALPKLRDMREIIQSEIQAKAESKIPGYKEGQEKYAK
EYRMSVHRWSYGRLIGNIQAQAAQAGILIETSSGQIRGSPQEQAKHLAISAYIERQTILNK (SEQ ID NO: 7)
>WP_012596241.1
[Cyanothece sp. PCC 8801]
MTLKTLECRLYAPSDTLRYLWLLMAEKNTPLINEIINHLSEHPDFDQWFKAKQIPKSAISDICNDLKSQENYQNQPGRFYSSAI
SLTHYMFKSWFAVHKQLQRRIEGKRRWLNLLKSDQELEQNCGQSLEIIIQKAEEILKLMDSEKSQSSSKPKKPKKPKKKKKSSS
EETITLFDRLFKAYNQGNDSLESYALAYLLKNNGQIPEDDEDLDKFALRKRKKEIEIERLQQQLENRIPLGRDLTGELWQEMLT
IVNESIPQDENEASAWQAKLLKKSHNIPYPVAYETNTDLKWSKDSRGHLLVTFNGLVESLKKLNLNPEFEIRCDRRHLPWFQRF
CKDQEIKANNDQHSSALFVLRSARLIWREGQGKEDPWKIHQLYLQCSVETQLWTEAGTKQVQSEKMVEFQLNQLRMKPELTFPI
FFRSQSLPTYFNLWKVITSYRILKFLEKGDFTKAQKNFQDAIKRTESCLENLQSSYLTSQKSLYQGNPEIIMGVAMGLSQPATI
AVVNVVTQEVLTYRSLKQLLGKNYNLLNRQRQQKQKLSHQRHKAQKKDAFNQYGESELGQYVDRLIAKAIVQVAKEYQADSIAV
PKIRQMREIIQSEVQARAERKIQGYKEGQKKYAQQYRENVHQWSYGRLIESIHQASAKFGIRVEIASQSYQGSFQEQAQNLAIA
AYTNRLEAVG (SEQ ID NO: 8)
>WP_012596246.1
[Cyanothece sp. PCC 8801]
MTHITVVQCRLIAPESTLQHIWKMMAQQQTPLINQLLHDINTHPDINTWLTANQLPSKLVETLAQPLKTQSPYQGLPGRFITSA
IILVKEMYASWFAIQTQKRLSLEGKKRFLTILKSDKQLIQDSQTDFLTLCYKAQQLLKRTQNKLKLDEPQHSEKAHWSIINALY
PAYNNAKTPISRAAFALLIKNNGQVPDTPENPDYYQQRRKRKEIQIRRLEEQLKASLPKGRILDSKHWENTLKLAQTPITTIEE
ITSLQTQLLQKYSHLPFPVFYGTNTDLTWFKNPQGRICVKFNGLNQYPFQIACNKRQYPWFQRFFTDYQSYKSHKQQVPTGLMV
LRSARLLWQPTNGQGEPWNTHHLSLHCAIDNDLWTISGIQQVKQQKILQTEQKIANFHSKALEKELTPNQQQRLKASQTSLNLL
KTFDINEFFPSKCSLYQGSPDIILGVSIGLENPATIAIINISTQEILTYRTTKQLLSRTRKVRNKKPNSNNSNQSLSSAYKQIS
NYELFLQYQQQKHHNQHQRHNAQINDANNNYGEANLGLYLNRLLAKAILELAQQYQVSLIILPSLKNKRELIESEIRAKAELKY
PGCKEKQDSYAKDYRTNVHQWSYQQLIKCIESKAAQIGIDTATGKQMNLETSQDQARNLVLNFCQKFSPTQV
(SEQ ID NO: 9)
>WP_066116114.1
[Geminocystis sp. NIES-3709]
MAHVTIQCRLIASRDTRQFLWQLMAQKNTPLINEILLRIKQHPDFPHWRTKKRLPKDFLARQIAELKNNYPFEEQPSRFYASVN
KVIDYIYKSWFEVQKALDWKLQGNLRWVEMLLPDTELIKHFDNSLESLQQQATLILDSIDSTVSHDRISTILFEKCGKTKKPEI
KSAIIYLLKNGCTIPKKPETTEKYQDLKRKVEIKITKLHRQIESRIPLGRDLEDKKWLDTLITASTTAPIDQTEANTWFSILKQ
NQSSIPYPILYETNEDLKWSLNEKNRLSIRFSGLGEHSFQLCCDHRQLPYFQRFYEDQELKKASKDQLSSALFTLRSAMILWKE
DEGKGELWDRHKLYLHCTFETRCLTAEGTSTIVEEKQKEVTKIIDLMKAKEELSDSQQAFIRRKNSTLAKLNNTFPRPSKPVYQ
GKPNVHLGIAMGLEQPVTIAIVDIETDKVITYRNTKQLLREDYRLLRRRRIEKQKLSHQNHKARKRFNFQQKGESNLGEYLDRL
IAKAILTVAQEYQVSTILIPRLRDMRSITEAEIQLRAEKKIPEYKEGQKKYAQDYRVQVHQWSYGRLIENVKLICEKVGIVVVE
AKQPKQGTLTEKALQLVLSATEKNLKKK (SEQ ID NO: 10)
>WP_017289534.1
[Leptolyngbya boryana dg5]
MSVITIQCKLVATEETRRALWHLMAEKHTPLINELLKHIAQDSRFEEWSLTGKLPRLVVSEACNQLKQDPQFSGQPGRFYSSAI
STVHRIFLSWLALQTRLRNQISGQTRWLAMLQSDNELTIASQTDINTLRLKASELLTHLNEPISESDQPEVKKTRSKKKNQTSN
QAGANVSRTLFKLYDETEDPLTRCAIAYLLKNGCKLPDQNENPEKFIKRRRKTEIRLERLMNTFQTTRIPRGRHLSWHSWIEAL
ETATSHIPENEEEAAGWQARLLTKPAILPFPVNYETNEDLRWSLNSQGRICVSFNGLSEHFFEVYCDQRDLHWFNRFLEDQETK
KASKNQHSSSLFSLRSGQIAWQEGKGDAEHWVVHRLVLSCSIETDTWTQEGTEEIRQKKASDCAKVIASTKAKENRSQNQDAFI
RRRERMLELLENQFPRPSYPLYQGQPSILAGVSYGLDKPATLAIVNIQTGKAITYRSIRQILGKNYKLLNRYRLNQQRNAHKRH
NNQRKGGSSQLRESNQGQYLDRLIAHEIVAIAQEYQVSSLALPDLGDIREIVQSEVQARAEQKILGSIEQQRKYARQYRASVHR
WRYAQLTQFIQSQAAQVGISIEITKQPLSGTPQEKARNLAIAAYQSRK (SEQ ID NO: 11)
>WP_017289534.1
[Leptolyngbya boryana IAM M-101]
MSVITIQCKLVATEETRRALWHLMAEKHTPLINELLKHIAQDSRFEEWSLTGKLPRLVVSEACNQLKQDPQFSGQPGRFYSSAI
STVHRIFLSWLALQTRLRNQISGQTRWLAMLQSDNELTIASQTDINTLRLKASELLTHLNEPISESDQPEVKKTRSKKKNQTSN
QAGANVSRTLFKLYDETEDPLTRCAIAYLLKNGCKLPDQNENPEKFIKRRRKTEIRLERLMNTFQTTRIPRGRHLSWHSWIEAL
ETATSHIPENEEEAAGWQARLLTKPAILPFPVNYETNEDLRWSLNSQGRICVSFNGLSEHFFEVYCDQRDLHWFNRFLEDQETK
KASKNQHSSSLFSLRSGQIAWQEGKGDAEHWVVHRLVLSCSIETDTWTQEGTEEIRQKKASDCAKVIASTKAKENRSQNQDAFI
RRRERMLELLENQFPRPSYPLYQGQPSILAGVSYGLDKPATLAIVNIQTGKAITYRSIRQILGKNYKLLNRYRLNQQRNAHKRH
NNQRKGGSSQLRESNQGQYLDRLIAHEIVAIAQEYQVSSLALPDLGDIREIVQSEVQARAEQKILGSIEQQRKYARQYRASVHR
WRYAQLTQFIQSQAAQVGISIEITKQPLSGTPQEKARNLAIAAYQSRK (SEQ ID NO: 11)
>WP_017289534.1
[Leptolyngbya boryana PCC 6306]
MSVITIQCKLVATEETRRALWHLMAEKHTPLINELLKHIAQDSRFEEWSLTGKLPRLVVSEACNQLKQDPQFSGQPGRFYSSAI
STVHRIFLSWLALQTRLRNQISGQTRWLAMLQSDNELTIASQTDINTLRLKASELLTHLNEPISESDQPEVKKTRSKKKNQTSN
QAGANVSRTLFKLYDETEDPLTRCAIAYLLKNGCKLPDQNENPEKFIKRRRKTEIRLERLMNTFQTTRIPRGRHLSWHSWIEAL
ETATSHIPENEEEAAGWQARLLTKPAILPFPVNYETNEDLRWSLNSQGRICVSFNGLSEHFFEVYCDQRDLHWFNRFLEDQETK
KASKNQHSSSLFSLRSGQIAWQEGKGDAEHWVVHRLVLSCSIETDTWTQEGTEEIRQKKASDCAKVIASTKAKENRSQNQDAFI
RRRERMLELLENQFPRPSYPLYQGQPSILAGVSYGLDKPATLAIVNIQTGKAITYRSIRQILGKNYKLLNRYRLNQQRNAHKRH
NNQRKGGSSQLRESNQGQYLDRLIAHEIVAIAQEYQVSSLALPDLGDIREIVQSEVQARAEQKILGSIEQQRKYARQYRASVHR
WRYAQLTQFIQSQAAQVGISIEITKQPLSGTPQEKARNLAIAAYQSRK (SEQ ID NO: 11)
>WP_039730330.1
[Lyngbya confervoides BDU141951]
MSKITIQCRLVASEATRQYLWHLMADIYTPFVNEILRQIREDDNFEQWRQSGKIPASVFEDYRKTLKTESRFQGMPGRWYYAGR
EEVKRIYKSWLALRRRLRNQLAGQNRWLEVLQSDETLMEVSGLDLSALQAEASQLLNILGSKNKTSKNRSKKAKGKPKGKSAKD
PTLYQALWELYRETEDIAKKCVIAYLLKHKCQVPDKPEDPKKFRHRRREAEIRAERLNEQLIKTRLPKGRDLTNEQWLQVLEIA
TRQVPKDEDEAAIWQSRLLTDAAKFPFPVAYETNEDLKWFLNGKGRLCVSFNGLSEHTFEVYCGQRQLYWFNRFLEDQQIKKEN
QGERSAGLFTLRSGRLVWKPYSSDASRSDPWMANQLTLQCSVDTRLWTAEGTEQVRQEKATSIAKVIAGTKAKGNLNQKQQDFI
TKREKTLELLHNPFPRPSKPLYQGKPSIIAAVSFGLEKPATLAIVDIVTDKAITYRSIRQLLGQNYKLFTKHRLKQQQCAHQRH
QNQVESAENRISEGGLGEHLDSLIAKAILETAAEYGASSIVLPELGNIREIIHAEIQAKAERKIPGLKEKQDEYAAKFRASVHR
WSYGRLAQKVTTKASLHGLETESTRQSLQGTPQEKARNLAISAYESRKVAQRA (SEQ ID NO: 6)
>WP_036484397.1
[Myxosarcina sp. GI1]
MSQNAIQCRLIAPETTRRQQWQLMAEKNTPLINELLKQLAEHPELETWKRKGKIPPGTVKNLCQPLRTCPQYINQPGRFYSSVI
SLAEYIYRSWLKLQRRLIFRLNGQQRWLQMLKSDEELVAESGRSLKEIEAKASEALDRLNREENPSISNRLFDLYDETEDILIR
SAIVYLLKNGCKIRQKPEDPKKFARRRRKTEIRVKRLQEKLNGKAPQGRDLTGEKWLNTLFTATSQVPQDEAQAKSWQDILLTK
SKLVPYPIVYESNEDLTWSKNERGRLCVKFNGLSDHTFQIYCDRRQLKIFNRFYEDQQIKKASKNSHSSALFTLRSATIAWQEG
KGKGEPWNVNRLILYCTFDNLLLTTEGTEVVRQEKAEAIANTLTKIKEKGDLNQKQQAFIRRKETSLSRINNPFPRPSRPLYKG
KSNILLGVAIRLDKPATVAIVDGATDKAIAYLSTKQLLGKNYHLLNRKRQQQHILSHQRNVAQRHHANNKFGESELGQYIDRLL
AKAIIQLAKDYRVGSIVVPYMEDTREIIQAEVQARAEAKIPGCIEKQKEYAKKYRTNIHKWSYGRLIDLIKAQAAKAGIVIEES
KQSIRGDPKKQAKEIAVCAYRDRIVPF (SEQ ID NO: 12)
>WP_012411901.1
[Nostoc punctiforme PCC 73102]
MSQITIQCRLIASESTRQKLWKLMATLNTPLINELIEQLGKHPDFENWRQQGKLPTTVVSQLCQPLKTDPRFVGQPSRLYMSAI
HIVDYIYKSWLAIQKRLQQQLDGKMRWLEMLNSDVELVETSGSSMGAIRTKASEILAKAMPTSDSDSSQPKTKKGKEAKKSSSS
SSDRSLSNKLFEAYQETEDILSRSAISYLLKNGCKLSDKEEDSEKFAKRRRQVEIQIQRLTEKLISRMPKGRDLTNRKWLETLF
TATTTFPEDNAEAKRWQDILLTRPSSLPFPLVFETNEDMVWSKNQKGRLCVHFNGLSDLSFEVYCDNRQLHWFQRFLEDQQTKR
QSKSQYSSGLFTLRNGHLVWQEGEGKSEPWNLNRLNLYCCVDNRLWTADGTEQVRQEKAEEISKLITKMKEKSDLKDTQKAFIQ
RKESTLNRMNNSFERPSQPLYQGQSHILVGVSLGLEKPATVAVVDAIAGKVLAYRSIRQLLGDNYELLNRQRRQQRSSSHERHK
AQKSFSPNQFGTSELGQYVDRLLAKEIIAIAQTYKAGNIVLPKLGDMREIVQSEIQAIAEAKCPGSVEVQQKYAKQYRVNVHKW
SYGRLIQSIQSKGSQAGIVIEEGKQPVRGSPHEQAKELALSAYHDRLARRS (SEQ ID NO: 13)
>BAB75312.1
[Nostoc sp. PCC 7120]
MCFVWYFIFMSQKTIQCRLIASESTRQKLWKLMAESNTPLINELLQQLSKHPDFEKWRRNGKLPSTVVSQLCQPLKTDPSFTGQ
PSRFYISAIHIVDYIYKSWLTIQKRLQQQLDGKLRWIEMFNSDVELVEISGFSLEAIRTKASEILAITTPESDPKTLLTKRGKT
KQSKKSSASNPDRSLSRKLFDAYQETDDILSRSAISYLLKNGCKLNDKEENPEKFAKRRRKVEIQIQRLTDKLTSRIPKGRDLT
YSKWLETLFTATTTVPENNAEAKRWQDILLTRSSSIPFPVVFETNEDLVWSTNEKGRLCVHFNGLSDLIFEVYCDSRQLYWFKR
FLEDQQTKRKSKNQHSSGLFTLRNGRLAWQQGEGKGEPWNIGHLALYCCVDNRLWTAEGTEQVRQEKAEEITKFITKMKDKSDL
SETQLAFIKRKESTLTRINNSFDRPSKPLYQGQSHILVGVSLGLEKPATIAVVDAIAGKVLTYRSLRQLLGDNYELLNRQRRQQ
RSLSHERHKAQKSFSPNQFGASELGQYVDRLLAKEIVAIAQTYKAGSIVLPKLGDIREIVQSEIQAIAEAKCPSSSEIQQKYAK
QYRVNVHQWSYGRLIQSIQSKAAQIGIVIEEGKQPIRGSPQDKAKELALYAYSLRLARRS (SEQ ID NO: 14)
>WP_044521835.1
[Nostoc sp. PCC 7120]
MSQKTIQCRLIASESTRQKLWKLMAESNTPLINELLQQLSKHPDFEKWRRNGKLPSTVVSQLCQPLKTDPSFTGQPSRFYISAI
HIVDYIYKSWLTIQKRLQQQLDGKLRWIEMFNSDVELVEISGFSLEAIRTKASEILAITTPESDPKTLLTKRGKTKQSKKSSAS
NPDRSLSRKLFDAYQETDDILSRSAISYLLKNGCKLNDKEENPEKFAKRRRKVEIQIQRLTDKLTSRIPKGRDLTYSKWLETLF
TATTTVPENNAEAKRWQDILLTRSSSIPFPVVFETNEDLVWSTNEKGRLCVHFNGLSDLIFEVYCDSRQLYWFKRFLEDQQTKR
KSKNQHSSGLFTLRNGRLAWQQGEGKGEPWNIGHLALYCCVDNRLWTAEGTEQVRQEKAEEITKFITKMKDKSDLSETQLAFIK
RKESTLTRINNSFDRPSKPLYQGQSHILVGVSLGLEKPATIAVVDAIAGKVLTYRSLRQLLGDNYELLNRQRRQQRSLSHERHK
AQKSFSPNQFGASELGQYVDRLLAKEIVAIAQTYKAGSIVLPKLGDIREIVQSEIQAIAEAKCPSSSEIQQKYAKQYRVNVHQW
SYGRLIQSIQSKAAQIGIVIEEGKQPIRGSPQDKAKELALYAYSLRLARRS (SEQ ID NO: 15)
>WP_094343940.1
[Nostoc sp. ‘Peltigera membranacea cyanobiont’ 232]
MSVITIQCRLVASEDTRRHLWQLMAEKNTPLINELLKQVRIHPDMEQWLKKGKLPDGVIKPLCDSLSTQECFVNQPKRFYKSAI
EVVEYIYKSWLALQKERQQKIDKKEHWLNMLKSDIELEQESNFSLNIIRAKAIKVLAHYIAKVEENHKQDITNKNVNKRKKSKS
KNNNYTLFDILFKAYDQAKVSLNRCAIAYLLKNNCQVSEEEEDPNRYALRRSKKLKEIERLKEQLKSRIPSGRDLTGQEWLQSL
LVATTNVPESENQFKIWQKHLLQNSSSIPFPVQFTSNEDLIWSKNEKGRICVSFSGEEFNNHIFEIYCDKKQIYWFQRFLEDQS
IKRNNKKQYSSSLFTLRSGRLAWQDNKGNDLPWKIHRLTLYCSVDTRLWTIEGTQEFRREKVDEITEKLADMEKKENLNKNQQI
YVKRLNSTLTKIDTAYPRPNQNLYQGKTSILIGVSLGLEKPATIAIVDCPTNKVLAYRSVKQLLGDNYNLLNRQRQQQQRNSHE
RHKAQKSNTQIKLSELELGKHIDNLLAQAIITLAKSYQAGSIVLPTMKNVRESIQSEIEARAVKRCPNYKEGQQQYAKQYRQSI
HRWSYNRLIGCIKSQAAKANISIEQGPQPIRDSPQEKARDLAIAAYHFRQNKS (SEQ ID NO: 16)
>WP_029636312.1
[Scytonema hofmanni UTEX 2349]
MSQITIQARLISFESNRQQLWKLMADLNTPLINELLCQLGQHPDFEKWQQKGKLPSTVVSQLCQPLKTDPRFAGQPSRLYMSAI
HIVDYIYKSWLAIQKRLQQQLDGKTRWLEMLNSDAELVELSGDTLEAIRVKAAEILAIAMPASESDSASPKGKKGKKEKKPSSS
SPKRSLSKTLFDAYQETEDIKSRSAISYLLKNGCKLTDKEEDSEKFAKRRRQVEIQIQRLTEKLISRMPKGRDLTNAKWLETLL
TATTTVAEDNAQAKRWQDILLTRSSSLPFPLVFETNEDMVWSKNQKGRLCVHFNGLSDLIFEVYCGNRQLHWFQRFLEDQQTKR
KSKNQHSSGLFTLRNGHLVWLEGEGKGEPWNLHHLTLYCCVDNRLWTEEGTEIVRQEKADEITKFITNMKKKSDLSDTQQALIQ
RKQSTLTRINNSFERPSQPLYQGQSHILVGVSLGLEKPATVAVVDAIANKVLAYRSIKQLLGDNYELLNRQRRQQQYLSHERHK
AQKNFSPNQFGASELGQHIDRLLAKAIVALARTYKAGSIVLPKLGDMREVVQSEIQAIAEQKFPGYIEGQQKYAKQYRVNVHRW
SYGRLIQSIQSKAAQTGIVIEEGKQPIRGSPHDKAKELALSAYNLRLTRRS (SEQ ID NO: 17)
>WP_073629970.1
[Scytonema sp. HK-05]
MSVITIQCRLVAEENTLRQLWELMAEKNTPLINELLEQVGQHPNFEKWLKKGEVPEEAIDTIKKSLITQEPFAGQPGRFYTSAV
TLVKEIYKSWFALQQERQRKIEGKERWLKMLKSDIELQQESQCNLDIIRNKANEILTSFVANFTENRNQQFKKKGNKTKKNKKE
EEESTLFNALFKIYDKTKDCLSQCALAYLLKNNCQVSEIDEDPEEYVKRRRRKEIEIERLRKQLKSRKPKGRDLTGEKWLTALK
EATNQVPVDQLEAKSWQASLLKVTSDIPYPVDYESNTDLDWLIHSNDDDIKKKVILVWQIYFLKQLIKSGSYSFIKYLYFQRGC
LPKRDVNWLNLKNKAGRIFVKFNGLRKNIINPEFYICCDSRQRHYFQRLCQDWQVWHDNEETYSSSLFFLRSARLLWQKRKGTG
APWKVNRLILQCSIETRLWTEEETELVRIEKINQAETEIRESEQKGKPKQKVLSHRQKLNNLFPNRPSKPIYKGKPNIIVGVSF
GLDKPATVAVVDVANKKVLAYRSTKQLLGKNYNLLNRQRQQQQRLSHERHKAQKRNAPNSFGESELGQYVDRLLADAIIAIAKT
YQAGSIVIPKLRDMREQITSEIQSRAEKKCPGYKRAQQKYAKEYRLSVHRWSYGRLIESIKSQAAKVGISTEIGTQPIRGSPEE
KARDLAVFAYQERQAALV (SEQ ID NO: 18)
>WP_072619878.1
[Spirulina major PCC 6313]
MSQITIQCRLVASEATRQVLWTLMAERNTPLINELLAQMAQHPDLEEWRQKGKPTPGVVKKLCDPLRQDPRFMGQPGRFYSSAI
ALVEYIYKSWLKLQQRLQRKLEGQQRWLGMLKSDPELCEENHCTLDTLRDKAAEILASLESPQPKQGKVKTKKAKAQSSPRQSL
FEMHDGAEDGFVKSAIAYLLKNGGKLPTHEEDPKKFAKRRRKAEVKVERLIHQITASLPKGRDLTGERWLETLLTASYTAPKDA
QQTKVWQSILLTKTKAVPYPINYETNEDLTWSKNEKGRLCVRFNGLSEHTFQIYCDQRQLKWFQRFYEDQEVKRTSKNQHSTSL
FTLRSGRIVWQESDRNDKPWTANHITLCCTLDTRLWSAEGTEEVRTEKAIDIAKTLTNMNEKGDLNDKQQAFIKRKTATLDRIN
NPYPRPSKPLYHGQSHILVGVALGLDKPATVAVVDGTTGKAITYRNLKQLLGENYKLVNRQRQQKQAQSHQRHKAQKRSGTDQF
GDSELGQHIDRLLAKAIVAFAHSQSAGSIVVPKLEDIREIVQSEIQARAEEKVPGYIEGQKQYAKRYRVQVHQWSYGRLIDSIK
SKATQQQVVIEEGKQPVRGSPEAQATELAISTYHLRASS (SEQ ID NO: 19)
>BAQ60380.1
[Geminocystis sp. NIES-3708]
MAVITIHCRLISSKSNRHQLWNLMVQKNTPLINELLLELSQHEDLEQWCELGKLPSGLISKLCDQLKQRAEFEGQPSRFYASAI
NLVDYIYKSYLRTQRRLRFRLQGQQRWFEMFKSDTEFKNETNFSLTDIRVKARELLDKDLKDSSPDDYFKTYESTSDLLTRSAI
SYLLKNGRKLPEKPEDYQKFQKRRRKLQIKIEKLQKKIDSSPPMGRNLTNDSWLGMLNLVSNTIPQTDEEAKQWQDQLLRQSKS
VPYPVMFNTNEDLRWSKNKKGRLCVTFNGLGKLVFEIYCDQQQLKWFERFYEDQEVKRKGKNQHSSALFTLRSGMLLWQEHEGK
QEAWQNNHLTLYCSLDTCFETAEGTELVRQKKVKEVVNLIDAMNNKSERTKTQDAFIKRKQSTLARLDNSFPRPSKPLYQGNQN
IVVAVSMSLEYPATIAMFNMSSQEVLTYRSTKQLLDNNYHLLNRQRNQKQRLSHQRHKTQRQNSSDFFTQQESELGQYLDRLLA
QSIVSIAKQYQASTILLPNLKNIRDSIQAEIEAKAEAKIPNCKEAQKKYLKNYRINIHHWSYGRLIDSIQLQASKLDILIQEVK
QPIRGSPQEKAKQMAILTLE (SEQ ID NO: 20)
>BAQ63841.1
[Geminocystis sp. NIES-3709]
MAHVTIQCRLIASRDTRQFLWQLMAQKNTPLINEILLRIKQHPDFPHWRTKKRLPKDFLARQIAELKNNYPFEEQPSRFYASVN
KVIDYIYKSWFEVQKALDWKLQGNLRWVEMLLPDTELIKHFDNSLESLQQQATLILDSIDSTVSHDRISTILFEKCGKTKKPEI
KSAIIYLLKNGCTIPKKPETTEKYQDLKRKVEIKITKLHRQIESRIPLGRDLEDKKWLDTLITASTTAPIDQTEANTWFSILKQ
NQSSIPYPILYETNEDLKWSLNEKNRLSIRFSGLGEHSFQLCCDHRQLPYFQRFYEDQELKKASKDQLSSALFTLRSAMILWKE
DEGKGELWDRHKLYLHCTFETRCLTAEGTSTIVEEKQKEVTKIIDLMKAKEELSDSQQAFIRRKNSTLAKLNNTFPRPSKPVYQ
GKPNVHLGIAMGLEQPVTIAIVDIETDKVITYRNTKQLLREDYRLLRRRRIEKQKLSHQNHKARKRFNFQQKGESNLGEYLDRL
IAKAILTVAQEYQVSTILIPRLRDMRSITEAEIQLRAEKKIPEYKEGQKKYAQDYRVQVHQWSYGRLIENVKLICEKVGIVVVE
AKQPKQGTLTEKALQLVLSATEKNLKKK (SEQ ID NO: 10)
>BAT55382.1
[Nostoc sp. NIES-3756]
MSVITIQCRLVAEEDTLRQVWELMTDKNTPLVNELLAQVGKHPEFETWLEKGKIPTEFLKTLVNSLKNQERFSDQPGRFYTSAI
ALVDYVYKSWFALQKRRKRQIEGKERWLIILKSDLQLEQESQCSLNVIRTEANEILAKFTPQSDQNKNQRKSKRTRKSAKLQTP
SLFQNLLNTYEQTQETLTRCAIAYLLKNNCQISERDEDPEEFNRNRRKKEIEIERLKDQLQSRIPKGRDLTGEEWLKTLEIATT
NVPQNENEAKAWQAALLRKPADVPFPVAYESNEDMTWLQNDKGRLFVRFNGLGKLTFEIYCDKRHLHYFKRFLEDQELKRNSKN
QHSSSLFTLRSGRIAWSLGEEKGEPWKVNKLHLYCTLDTRMWTIEGTQQVVSEKTTKITETLNQAKRKDVLNDKQQAFVTRQQS
TLDRINNPFPRPSKPNYQGQPSILVGVSFGLEKPVTLAVVDVIKNEVLAYRTVKQLLGKNYNLLNRQRQQQQRLSHERHKVQKR
NAPNSFGESELGQYVDRLLADAIIAIAKTYQAGSIVIPKLRDMREQISSEIQSRAEKKCPGYKEVQQKYAKEYRMSVHRWGYGR
LIESIKSQAAKAGIFTEIGTQPIRGSPQEKARDLAVFAYQERQAALI (SEQ ID NO: 21)
>ACV01094.1
[Cyanothece sp. PCC 8802]
MSTITIQCRLVAPEATRQALWQLMAQKNTPLVSELLRQVAQHPDFETWRQQGKLEAGIIKKLCEPLKKDPRFNEQPARFYTSAI
ALVDYIYKSWLKLQQRLQRKLEGQNRWLAMLKSDDELIQISQTNIEIIQAKATEILSTLQPQDREQSSKKKAKKCKKSTNKNSL
FSQLDKLYNEINNNLTHCAIRYLLKNGGKIPQRPEDTEKFAQRRRKVEIKIERIIEQIESSIPQGRDLTGDSWLETLIIAANTA
TVEAEDVKSWQDKLLSQSKSIPYPVAYETNEDLTWSINEKGRLCVRFNGLGKHTFQIYCDQRQLKWFQRFYEDQQIKKDGKDHH
SSALFSLRSGRIVWQEGLGKGKPWNIHRLTLHCSLDTRFWTEEGTQQVQQEKSKKFQTNRLRMKPELTFSIFFRSQTLETYLQV
WLVITAYRLQSFLDKGNVAKAHQEFQKAIKRNESSLQKITSSYNRPHKTLYQGKSHIFVGVAMGLEKPATVAVVDGTTGKAIAY
RSLKQLLGNNYHLFNRQGKQKQNTSHQRHKSQKHFADNQFGESQLGQYIDCLLAKAIISVAQTYCAGSIVVPKLKDMRELIQSE
IQAKAEAKIPGYVEGQAKYAKSYRVQVHQWSHGRLIDNITSQASKFNITVEEGEQPHQGNPQDKAKLLAIAAYHSRLCA
(SEQ ID NO: 22)
>AFZ00435.1
[Calothrix sp. PCC 6303]
MSVITIQCRLVADEETLRHLWTLMAEKNTPFANEILEQLAQHAEFESWVKNSRVPATVIKELCDSLKNQELFAGQPGRFYTSAT
TLVTYIYKSWLAVNKRLQRKIEGKKQWLDMLRSDTELEQESNSNLEKIRAKATEILDSFATRQINQVNSKSKTSKNNKNKQEKE
VKSLSIQSNILFETYRQTEDNLTKCAIVYLLKNNCEVNDVEEDIEEYEKNKRKKEIQIKRLEDQLKSRVPKGRDLTGEKWVEVL
EKAVNSVPESENEAKSWQASLLRKSSQIPFPVVYETNEDIKWSINEKGRIFVSFNGLGKLKFEIFCDKRHLHYFQRFLEDQDIK
RQGKNQHSSSLFTLRSGRISWLEQPGKGKPWNINRLLLFCSIDTRMLTAEGTQQVIEEKIADTQNKIAKAQEKCEGELNPNQQA
HINRKKSTLARINTPFPRPSKPLYQGKSHIVVGVSLGLKATATIAVFDAMNNQVLAYRSTKQLLGDNYKLLNRQQQQKQRLSQQ
RHKSQKQFASNSFGESELGQYVDRLLAKEIVAVAKNFGAGSIVLPKLGDMREIIQSEVQAKAEKKIPGFIELQKNYAKEYRKSA
HNWSYGRLIENIQSQATKEGIEIETGKQPTRGIPQEQARDLALFAYQCRIA (SEQ ID NO: 23)
>AFZ58287.1
[Anabaena cylindrica PCC 7122]
MSQITIQCRLVASETTRQQLWQLMAEKNTPLINELLSQIGKHPEFETWRQKGKHPTGIVKELCEPLKTDPRFIGQPARFYTSAT
ASVNYIYESWFALMKRYQSQLDGKLRWLEMFNSDAELVEHSGVSLDTLRATSAEILAQFAPQDTNRDTSNKGKKSKMGKKSQKS
DSEGNLSKKLFDAYSSAEDNLTRCAISHLLKNGCKVSNKEENSEKFTQRRRKLEIQIQRLTEKLAARIPKGRDLTDTQWLETLF
TATYNVPEDETEAKLWQNSLLRKFSSLPFPVAYETNEDLVWSKNRFGRICLTFPTLREHIFQIYCDSRQLHWFQRFLEDQEIKK
NSKNQHSSALFTLRSGRIAWQEGEGKGEPWDIHHLTLYCCVDTRLWTEEGTNLVKEEKAEEIAKTITQTKAKGDLNDKQQAHLK
RKNSSLARINNPFPRPSQPLYKGQSHILLGVSLGLEKPATVAVVDGTTGKVLTYRNIKQLLGDNYKLLNRQRQQKHLLSHQRHI
AQRIAAPNNFGDSELGEYIDRLLAKEIIAIAQTYQAGSIVLPNLGDMREQIQSEIKAKAEQKSDLVEVQKKYAKQYPNSVHQWS
YGRLITNIQSQSKKAGIVIEEGKQQIRASPLEKAKELAINAYQSRKA (SEQ ID NO: 24)
>AFZ56196.1
[Anabaena cylindrica PCC 7122]
MSVITIQCRLVAEEDSLRQLWELMSEKNTPFINEILLQIGKHPEFETWLEKGRIPAELLKTLGNSLKTQEPFTGQPGRFYTSAI
TLVDYLYKSWFALQKRRKQQIEGKQRWLKMLKSDQELEQESQSSLEVIRNKATELFSKFTPQSDSEALRRNQNDKQKKVKKTKK
STKPKTSSIFKIFLSTYEEAEEPLTRCALAYLLKNNCQISELDENPEEFTRNKRRKEIEIERLKDQLQSRIPKGRDLTGEEWLE
TLEIATFNVPQNENEAKAWQAALLRKTANVPFPVAYESNEDMTWLKNDKNRLFVRFNGLGKLTFEIYCDKRHLHYFQRFLEDQE
ILRNSKRQHSSSLFTLRSGRIAWLPGEEKGEHWKVNQLNFYCSLDTRMLTTEGTQQVVEEKVTAITEILNKTKQKDDLNDKQQA
FITRQQSTLARINNPFPRPSKPNYQGKSSILIGVSFGLEKPVTVAVVDVVKNKVIAYRSVKQLLGENYNLLNRQRQQQQRLSHE
RHKAQKQNAPNSFGESELGQYVDRLLADAIIAIAKKYQAGSIVLPKLRDMREQISSEIQSRAENQCPGYKEGQQKYAKEYRINV
HRWSYGRLIESIKSQAAQAGIAIETGKQSIRGSPQEKARDLAVFTYQERQAALI (SEQ ID NO: 25)
>ODH02164.1
[Nostoc sp. KVJ20]
MSSDRKKKSTIPVHRTIRCHLDASEDILRKVWEEMTQKNTPLILKLLKSVSEQPEFEANKEKGEITKKEIVKLRKNVTKNPELE
EQSGRLRSSAESFVKEVYSSWLTLYQKRKRQKEGKEYFLKNILKSDVELIDESNCDLETIRSKAQEVLSQPEEFIKQLTINDED
VKPTKSARKRVNKNINNKSTDAEQRKDSSSTNNVDKNKLETLTNILYEIHKQTQDILTRCTVAYLIKNHNKISNLEEDIQKLKK
RRNEKIVQIKRLENQIQDNRLPSGRDITGERYSEAFGNLINQVPKNNQEWEDWIANLSKKISHLPYPIDYLYGDLSWYKNDVGN
IFVYFNGWSEYHFKICCNKRQRHFFERFLEDYKAFKVSQKGEEKLSGSLITLRSAQLLWQQGEGKGEPWKVHKLALHCTYDSRL
WTAEGTEEVRKEKTDKAQKRVSKAEENEKLDDIQQTQLNKDKSSLSRLKNSFNRPGKLIYQSQSNIIVGISFHPIELATVAIVD
INTKKVLACNTVKQLLGNAFHLLSRRRRQQVHLSKERKKAQKKDSPCNIGESKLGEYIDKLLAKRIVEIAKFYQAGCIILPRLK
DMKEIRTSAIQAKARAKIPGDVNAQKLYVKEYNRQIHNWSYNRLQESIKSKAAEFKISIEFGIQPHYGTLEEQAKDLAFYAYQS
RNHTLGR (SEQ ID NO: 26)
>WP_066425713.1
[Anabaena sp. 4-3]
MSVITIQCRLVAEEDTLRTLWELMADKNTPLINEILAQVGKHPEFETWLEKGKIPTELLKTLVNSLKTQERFASQPGRFYTSAI
ALVDYVYKSWFALQKRRKRQIEGKERWLTILKSDLELEQESQCSLNIIRTKATEIITEFTPQSDQNNSQKKRKKTTKSTKPSLF
QILLNNYEETQDILTRCALAYLLKNNCQISERDENPEEFTRNRRKKEIEIERLKDQLQSRIPKGRDLTGEEWLKTLEVVRANVT
QNENEAKAWQAAILRKSADVPFPVAYESNEDMTWLQNDKGRLFVRFNGLGKLTFEIYCDKRHLHYFKRFLEDQELKRNSKNQYS
SSLFTLRSGRLAWSPGEEKGEPWKVNQLHLYCTLDTRMWTIEGTQQVVDEKSTKITETLTKAKQKDDLNDKQQAFVTRQQSTLN
RINNLFPRPSKSRYQGQPSILVGVSFGLENPVTLAVVDVVKNEVLAYRSVKQLLGKNYNLLNRQRQQQQRLSHKRHKAQKRNAP
NSFGESELGQYVDRLLADAIIAIAKTYQAGSIVIPKLRDMREQISSEIQSRAEKKFPGYKRAQQKYAKEYRMSVHRWSYGRLIE
SIKSQAAKAGISTEIGTQPIRGSPQEKARDLAVFAYQERQAALI (SEQ ID NO: 27)

TABLE 3
Amino acid sequences of Representative CLUST.004377 Effector_B Proteins

>OBQ23832.1
[Anabaena sp. WA113]
MEGKFYTSTEAAEITNCSRRQLQYWREKGVIVPTVNASGKGRNVYYSKADLLALSVMEHLLSIGLNFEMCHVALETLRKREPWL
FEESVTEDKMKRLMFLPSKSPEQPLQLAEFDQQAALEALCEGQTVIPFWGDRIHELLHQNLKRFTE (SEQ ID NO: 28)
>WP_011318007.1
[Anabaena variabilis ATCC 29413]
MQETFFTSKEASKITGCTLRQLQYWREKGVVVPVISDTGTGRSIYYSKTNLVELAAMVYWLSVGLSFDIACETLKTLKDQEPEL
FSSGTGRRFMLLSEAQERSPLGAPKISLELMEFDRTKAIASLDEGKPVIPVWLDEIYQQLQQKLKA (SEQ ID NO: 29)
>WP_066116116.1
[Geminocystis sp. NIES-3709]
MKERFFSSKQTSLITGCSLRQLQYWRDKEVIVPFIQGTGTGKTIYYSPAELVEISIMVYLLSVGLSFEIGQIILSDLKEKERNF
RNPDYKGRFMIIPRDGKMSLLSYEREKAIELLDQGTSIVPLWLDRIHEQLKETLG (SEQ ID NO: 30)
>WP_052055737.1
[Myxosarcina sp. GI1]
MGEPFYTSTEASLITGCSRRQLQYWRKQGVVVPTVNPGGKGRNVYYTESDLLILSVMKYLLSLGLNFDVSLKVLETLREKEILC
FLDWSLSKNTKKRFMLVLDTEEKNTIHITNFDTELAVQKLQEGFAIAPFGRDRIYQKLQDNLQAFYRNRKSLKGDRRTKN
(SEQ ID NO: 31)
>WP_012411902.1
[Nostoc punctiforme PCC 73102]
MEEKFYTSTEAAEITNCSRRQLQYWRDKGVVIPTVNTTGKGRNVYYSVADLLTLTVMHYLLSVGLSFEVCREALAMLQDKEPWL
FEEFVSKKKMRRLMLISNASQQKYLTLAEFDKEAALEALCQGQTVIPFWCDRIHEVLHQNLKRFSE (SEQ ID NO: 32)
>WP_010997761.1
[Nostoc sp. PCC 7120]
MLYKALMCFRRCFYRMFLMEGKFYTSTEASEITHCSRRQLQYWREKGVIVPTVNSSGKGRNVYYSKADLLALTVMEQLLSTGLN
FDLCYAALQTLRKQEPWLFDESVPEEKMKRLMLLPTRSPEQPLQLAEFDKQAALEALCHGQTVIPFWSDRIHQQLRENLKSFSS
(SEQ ID NO: 33)
>WP_029636314.1
[Scytonema hofmanni UTEX 2349]
MEGKFYTSTEAAQITNCSRRQLQYWRDKGVVVPTVNTTGKGRNVYYSISDLLVLTVMHYLLSVGLSFEVSRQTLVILRQKEPWL
FEEFVPKEKMKRLMLLTTCSLEQPLTLAEFDKEA.ALEALCQGQTVIPFWCDRIHQQLRDNLKSFSS (SEQ ID NO: 34)
>WP_072619877.1
[Spirulina major PCC 6313]
MEQPFFSSREAADITGCTLRQLQYWREKEVVIPTINATGTGRSIYYSQANLVELSVMAYWLSLGLTFEVAHESLMKLRAIEPRF
VEPPIARRFMLKWEEMRGKLELVEFDREGAIASLDAGQPVIPVWLEQIYSKLSNKISPN (SEQ ID NO: 35)
>ACV01093.1
[Cyanothece sp. PCC 8802]
MEEMFFTSTEASKLTGCSRRQLQYWREKGVVVPTVNASGKGRNVYYSVSDLLKLMVMEYLLSVGLSFETAQESLEMLKKAHPEM
FKADVSSEDLKRLMILPSPKRLELKDFDPEEAFKRINTYAMPLVPFSGEMIHERLKESLQGFSGNNHLKSSENS
(SEQ ID NO: 36)
>AFZ58288.1
[Anabaena cylindrica PCC 7122]
MQETFFTSKEAAQLTGCTLRQIQYWREKGIVVPIISDTGTGRSIYYSRSNLVELAALVYWLSTGISFDIACETLKRLKAQEPEL
FISGKGKRFMLLLSAQNESLSLVEFDRKSAIASLDEGKAVIPVWLDIIYQKLASRLAQ (SEQ ID NO: 37)

TABLE 4
Amino acid sequences of Representative CLUST.004377 Effector_C1 Proteins

>OUC12067.1
[Alkalinema sp. CACIAM 70d]
MIVTTLSPDEERKIKVLQPLIEPCDRATYGKRLRYAAEQLGISVRSVQREIQRWEKNGIAGITRNCRADKGKHRISDFWQNFII
KTYKEGNTGSKRMTPKQVAVRVQAKAHEMRDENPPHYRTVLRVLKPIIERQEKTKSIRSPGWKGSTLSVKTRSGEDLVIAHSNH
VWQCDHTPVDVMLVDQYGEKRKRPWLTIVIDSYSRCIMGINLGFDAPSSQVVALALRHAILPKHYGAEYKLNCEWGTYGKPVHF
YTDGGKDFRSNHVQQIGHDLGFTCHLRDRPSEGGIVERPFKTLNEQLFSTLPGYTDSNVQKRPKDVEKDARLTLRDLERLVVRF
ICDCYNQSIDARIGDQTRFQRWEAGLPGVPDVIGERLLDPCLMNVSRRTIQRGGYLQFKNLMYRGEYLAGYAGEIVSVRFNPDD
ITTLLVYRRENNREVFLTRAFAQGLETEQLSLSEAQASSSRRKEAGKSLSNTAIFQEVLDRDALVNSKKSRKQRHKEEQKIARS
ETETPKTGAVSVEQPETAPTELEVALDLETETFEPIDFEELQGGW (SEQ ID NO: 38)
>OBQ23776.1
[Anabaena sp. WA113]
MNRDENSDLNTSAIPVESISEGDNTPPETNVIATELSEESQLKLEVLQSLLEPCDRTTYGQKLKEAAEKLAVSVRTVQRLVKKW
EQDGLVGLTQTGRTDKGKHRIGEFWEDFIVKTHKEGNKGSKRMSPKQVAIRVQAKAHELSDLKPPNYRTVLRVLAPILEKQEKT
KSIRSPGWRGTTLSVKTREGKDLSVDYSNHVWQCDHTPADVLLVDQHGELLSRPWLTTVIDTYSRCIMGINLGFDAPSSEVVAL
ALRHAILPKRYSLEYKLHCEWGTYGKPEHFYTDGGKDFRSNHLSQIGAQLGFVCHLRDRPSEGGIVERPFKTLNDQLFSTLPGY
TGSNVQERPEDAEKDAKLTLRELEQLIVRYIVDRYNQSIDARMGDQTRFGRWEAGLPSVPVPIEERDLDICLMKQSRRRVQKGG
HLQFQNLMYRGEYLGGYDGETVNLRFNPRDITTVLVYRQENSQEVFLTRAHAQGLETEQLSLDEAEAASRRLRNLGKTISNQAL
LQEVLDRDALVANKKSRKDRQKLEQEILRSTAVNDSKNESLASPVMEAEDVEFTTPVQSSSPELEVWDYEQLREEYGF
(SEQ ID NO: 39)
>ABA20966.1
[Anabaena variabilis ATCC 29413]
MNYLDVDNQFELEDEDDFLLDDEDADILDFSIEVESLSNDGNSVAEDKLVEFLDQRYLEDSELRLSGEQRLKLEIIRNLREPCD
RLTYGKRLEEAAKKLGKSERTVRRLIKSWEEKGIAALAEVPRADKGQVRKSEYWYNLSLKIYKQGNKGDKGDKGDKGSDRMTRT
QVAEKVETKAYEFAKKELEAEISKLESQGFRGQDLDWELKKLIKIKEKAEGFKYWSKYGKPPSTRTVERWLKPVEEKRHKSRTS
RSPGWHGSEHTIKTRDGQEISIKYTNQVWQIDHTKADILLVDEDGEEIGRPQLTTVIDCYSRCIVGFRLGFAAPSSQVVALALR
HAIMPKRYSSEYELRCKWSAYGVPKYVYTDGGKDFRSKHLVEWIANELDFEPILRSKPSDGGIVERPFRTISGLLSEMPGYTGS
SVKDRPEGAEKKACISLPELEKLIVGYIVDSYNQKPDARSQANPLTPKQSRIERWEKGLQMPPTLLNDRELDICLMKAAERVVY
DNGYLNFSGLRYRGENLGAYAGDKVILRFDPRDITMVLVYGRKNNKEIFLARAYAIGLEAASLSVEEVKYARKKAENSGKGINN
ISILEEALRRRNFLQQKKNKTKAERRRSEEKRIEKIPQVLTEKKNEQVAVLISQPEDDEPIEKLDLKLLREELGL
(SEQ ID NO: 40)
>WP_011317997.1
[Anabaena variabilis ATCC 29413]
MLDDPDKGNQEPETHEIVTELSLDEQHLLEMIQKLIEPCDRITYGERQREVAGKLGKSVRTVRRLVKKWEQEGLTALQTATRTD
KGTHRIDSDWQDFIIKTYKENNKDGKRMSPKQVALRVQTKAEELGQQKYPSYRTVYRVLQPIIEQKEQKEGIRHRGWHGSRLSV
KTRDGKDLFVEHSNHVWQCDHTRVDLLLVDQHGELLSRPWLTIVVDTYSRCIMGINLGFDAPSSQVIALALRHAILPKRYGSEY
GLHEEWGTYGKPEHFYTDGGKDFRSNHLQQIGVQLGFVCHLRDRPSEGGIVERPFGTFNTDLFSTLPGYTGSNVQERPEQAEKE
ACITLRELERLLVRYIVDKYNQSIDARLGDQTRYQRWEAGLIVAPNLISEEELRICLMKQTRRSIYRGGYVQFENLTYRGENLA
GYAGENVVLRYDPKDITTLLVYRQKGNQEEFLARAYAQDLETEELSVDEAKAMSRRIRQAGKAISNRSILAEVRDRETFVNQKK
TKKERQKAEQTIVQKAKKPVPVEPEEEIEVASVDSEPEYQMPEVFDYEQMREDYGW (SEQ ID NO: 41)
>WP_044151373.1
[Aphanocapsa montana BDHKU210001]
MELVNPDDLNSVESRLKLEIIEKLSEPCDRKTYGERLRSAAQQLKCSVRTVQRLMKKWEEEGLAALIDSGRIDKGKPRIAEDWQ
QFIKKVYSNDKCTPAQVFTKVRNKARQEGLKDYPSHMTVYRILRLVKEAKEKEESIRNLGWKGSRLALKTRDGEVLEIDYSNQV
WQCDHTRADILLVDKYGHQMGRPWLTTVIDTYSRAIVGINLGYDAPSSSVVALALRNAIMPKQYGVEYKLYADWPTCGTPDHLF
TDGGKDFRSNHLRQIGLQLGFICHLRDRPSEGGIVERPFGTINTQFLSTLPGYTGSNVQDRPPEAEAEACLTLQELEKLLVAYI
VNTYNQRLDARMGDQTRIQRWEAGLLKQPRVIPEHELHICLMRQTRRTIYRGGYLQFENLAYRGEALAEHAGENIVLRYDPRNI
AQVLVYRHDPDREVYLGVAQALEFEGEVLALDDAKAHSRRIREDGKAVSNDAMLDEMRDREAFVDQKNKSRKDRQKDEQADLRP
TTPPIIGPDSSDEPSVDVQPDESPEELDIPEFDIWDFDDDDA (SEQ ID NO: 42)
>WP_019497296.1
[Calothrix sp. PCC 7103]
MGETLNSDNINALQVFDDSGELEEISESDDTESKNTIVTELSAEAKLRMEVLQSLLEPCDRKTYGIKLHDAAEKLGKTIRTVQR
LVKKYQEEGLSAITEAERSDKGDYRIDKEWQEFIFKTYKEGNKNGRKMTPAQVAIRVQVKAEQLALSNYPSHMTVYRVLNPIIE
RKEQKQKVRNIGWRGSRVSHKTRDGQTLEPRYSNHTWQCDHTKLDIMLVDQYGETLARPWLTKITDSYSRCIMGINLGFDAPSS
LVVALAMRHAILPKSYGSEYKLHCKWETYGVPENLFTDGGKDFRSEHLKQIGFHLGFECHLRDRPPEGGIEERGFGTINTDFLA
GFYGYLGSNLQQRPEEAESEACITLRELHLLLVRYIVDNYNQRIDARSGNQTRFQRWEAGLPALAPLIDERQLDICLMKKTHRS
IYKGGYVSFENITYRGEYLAAYAGESVILRFDPRDEWHLLKGKRIGMIGFRALVMAINTVGAYINTNTYQLHIHVN
(SEQ ID NO: 43)
>WP_012596233.1
[Cyanothece sp. PCC 8801]
MSLSLSIPDDSPHSHRLPSDEMITDEVKAKIDIIQSLIEPCDRITYRQRKEQAAKQLGVTIRSVERLLKKYREQGLIALMKTRS
DKGKTRIDDEWKEFILNTYKEGNKGSKRITRHQVFLKVKGRAKQLGLNKEEFPSHQTVYRILDKFIEENERKKKARSPGYSGSR
LTHITRHGRELEVEGSNDVWQCDHTCLDIRLVDEFGVLSRPWLTIIIDSYSRCVMGFFLGFYAPSSHIDALALRHAILPKSYSS
DYQLKNEWNTYGIPTYYYTDGGKDFRSIHVTEQVAVSLGFNCFLRSRPSDGGIVERFFKTLNNSVLCELPGYTGSNVQQRPKNV
DKDACLSLKDLEKILVKYIVDEYNQKPDARMKNQSRITRWEAGLLTEPYLYDERELDIALMKEAKRTLQKSGTLQFENLTYRSP
LLKGREGERVAIRYDPDDVTTILVYEYLDDGTEAFLDYAHAQNLETEKLSYRELKAINKQLNQEEEAINNDKILDAMMERMEMT
EELVKKNRQQRQQNAHEAVNSRPSVTEKLSISQPDEEDWEDDSEDEPLPTYKVQYMDDLFEDD (SEQ ID NO: 44)
>WP_066116124.1
[Geminocystis sp. NIES-3709]
MTIENQISESQELISQLSPEEQAIADVIEDLVQPCDRKTYGAKLKKAAETLNKSVRTVQRYIKEWEEKGLLAIKKGNRSDQGTY
RIEKRLQDFIVKTYREGNKGSKRISPKQVYLRTMAQAKEWSIDPPSHMTVYRILNPIIEEKENKKRVRNPGWRGTKLAVSTRSG
NEINVEYSNHAWQCDHTRADILLVDQFGQLLGRPWLTTVVDTYSRCIMGINLGFDAPSSQVVSLALRHAMLLKSYSSDYGLHEE
WGTYGKPEYFYTDGGKDFRSNHLQQIGLQLGFTCHLRSRPSEGGVVERPFKTLNTEVFSTLPGYTGGNVQERSEDAEKDASLTL
RQLERIIVRYIVDNYNQRMDARMGEQTRFQRWDSGLLSIPDLLSERDLDICLMKQSRRRVQKGGYLQFENLMYQGEYLAGYEGE
TVILRYDPRDITAILVYRNEGNKEVFLTRAYGLDLETESMSWEDAKASAKKVRESGKNLSNRSILAEVKERHTFSDKKTKKERQ
RQEQEQVKPYIPSPVLKEVKEQTDKATDTDMSEEPIVEVFDYSQLQDDYGF (SEQ ID NO: 45)
>WP_017289545.1
[Leptolyngbya boryana dg5]
MQLPIEFPESEQVSRELVEQNQIVTELSDEAKLKIEVIQSLLEPCDRATYGNRLRDAATRLGKSVRTVQRMVKSWQEEGIAGLS
NGERTDKGEHRIEQEWQDFIIKTYQEGNKNGKRMTPAQVAIRVKVKAQQEGITKYPSHMTVYRVLNPLIQRKTEKQNVRSIGWQ
GSRLSLKTRDGNSLSVEYSNQVWQCDHTRADILLVDQHGELIGRPWLTTVVDTYSRCIVGVNLGFDAPSSDVVALALRHAILPK
TYPDRYQLNCDWGTYGKPEHFFTDGGKDFRSNHLQQIAVQIGFTCHLRNRPSEGGVVERPFGTLNTEFFSILPGYTGSNVQKRP
EEAEESASLTLRELEQFLVRYITDRYNQGIDARMGDQTRFQRWEAGLLANPSVLTERELDICLMKQTRRTVYREGYLRFENLIY
RGENLAGYAGETVTLRYEPRDITTVFVYHQEQGKEVFLTRAHAQDLETETISLYEAKASSRRIRDVGKTISNRSILEEVRDRDL
FVQKKTRKERQKAEQAEVKIDQVPSPPQVLHLDEASQFETEIVETQCVEISEIEDYEKLRDDFGW (SEQ ID NO: 46)
>WP_036484426.1
[Myxosarcina sp. GI1]
MNSESTSETNSKLAISDLEDDREIVITSQLEGKAKERLEVIQSLLEPCDRATYGERLRAGAKKLDISVRSVQRLFKKYQEQGLT
ALVSTNRVDKGNRRISSFWQDFILQTYIQGNKGSKRMSPKQVAIRVQAKASEIKDNKPPSYKTVLRLLKPIQKKKERTIRSPGW
QGTTLSVKTRDGQDIQINHSNQVWQCDHTLVDVLLVDRHGELIGRPWLTTVVDSYSRCVMGINLGFDAPSSLVVALALRHSILP
KNYSQDFQLYCDWGVFGLPECLFTDGGKDFRSNHLEEIATQLGFIRKLRDRPSEGGIVERPFKTLNQSLFSTLPGYTGSNVQER
PKDAEKDARLTLRDLEMLIVRFIVDKYNQSTIAGKDEQTRYQRWEAGLIKDPKIISERELDICLMKSKRRTVQRGGHLQFENII
YRGEYLAGYEGDIVNVRYNPINITTILVYRREQGKEVFLTRAHALGWETEIHSLSEARASVKRLRQAKKKISNESIHQEILLRD
SAVDKKKSRKQRQKEEQSYKLITSPKVVAQDIESQEIERDISAEIADVEVWDFDELEDE (SEQ ID NO: 47)
>WP_012411919.1
[Nostoc punctiforme PCC 73102]
MNSQQNPDLAVHPSAIPIEGLLGESDITLEKNVIATQLSEEAQLKLEVIQSLLEPCDRTTYGQKLREAAEKLGVSLRTVQRLVK
NWEHDGLVGLTQTGRADKGKHRIGEFWEKFITKTYNEGNKGSKRMTPKQVALRVEAKARELKDSKPPNYKTVLRVLAPILEKQE
KAKSIRSPGWRGTTLSVKTREGKDLSVDYSNHVWQCDHTRVDVLLVDQHGELLSRPWLTTVIDTYSRCIMGINLGFDAPSSGVV
ALALRHAILPKQYGFEYKLHCEWGTYGKPEHFYTDGGKDFRSNHLSQIGAQLGFVCHLRDRPSEGGVVERPFKTLNDQLFSTLP
GYTGSNVQERPKDAEKDARLTLRELEQLLIRYIVDRYNQSIDARMGDQTRFERWEAGLPTVPVPIPERDLDICLMKQSRRTVQR
GGCLQFQNLMYRGEYLAGYAGETVNLRFDPRDITTILVYRQENNQEVFLTRAHAQGLETEQLALDEAEAASRRLRNAGKTISNQ
SLLQEVVDRDALVATKKSRKERQKLEQAVLRSAGVDESKTESLSSQVVEPDEVESTETVHSQYEDMEVWDYEQLREEYGF
(SEQ ID NO: 48)
>BAB75329.1
[Nostoc sp. PCC 7120]
MIQTLLEPCDRTTYGQKLKEAADTLGVTVRTVQRLVKKWEEDGLVGFIQTGRADKGKHRIGEFWENFIIKTYKEGNKGSKRMTP
KQVALRVQAKARELGDSKPPNYRTVLRVLAPILEQKEKTKSIRSPGWRGTTLSVKTREGQDLSVDYSNHVWQCDHTRVDVLLVD
QHGELLSRPWLTTVIDTYSRCIMGINLGFDAPSSVVVALALRHAILPKKYGAEYKLHCEWGTYGKPEHFYTDGGKDFRSNHLSQ
IGAQLGFVCHLRDRPSEGGIVERPFKTLNDQLFSTLPGYTGSNVQERLEDAEKDAKLTLRELEQLLVRYIVDRYNQSIDARMGD
QTRFGRWEAGLPSVPVPIEERDLDICLMKQSRRTVQRGGCLQFQNVMYRGEYLAGYAGETVNLRYDPRDITTVLVYRQEKSQEV
FLTRTHAQGLETEQLSLDEARAASRRLRNAGKTVSNQALLQEVLERDAMVANKKSRKERQKLEQAILRSAAVNESKTESLASSV
MEAEEVESTTEVQSSSSELEVWDYEQLREEYGF (SEQ ID NO: 49)
>WP_044521841.1
[Nostoc sp. PCC 7120]
MTRQQHTSSPPETNLIVTELSEEAQLKLEVIQTLLEPCDRTTYGQKLKEAADTLGVTVRTVQRLVKKWEEDGLVGFIQTGRADK
GKHRIGEFWENFIIKTYKEGNKGSKRMTPKQVALRVQAKARELGDSKPPNYRTVLRVLAPILEQKEKTKSIRSPGWRGTTLSVK
TREGQDLSVDYSNHVWQCDHTRVDVLLVDQHGELLSRPWLTTVIDTYSRCIMGINLGFDAPSSVVVALALRHAILPKKYGAEYK
LHCEWGTYGKPEHFYTDGGKDFRSNHLSQIGAQLGFVCHLRDRPSEGGIVERPFKTLNDQLFSTLPGYTGSNVQERLEDAEKDA
KLTLRELEQLLVRYIVDRYNQSIDARMGDQTRFGRWEAGLPSVPVPIEERDLDICLMKQSRRTVQRGGCLQFQNVMYRGEYLAG
YAGETVNLRYDPRDITTVLVYRQEKSQEVFLTRTHAQGLETEQLSLDEAEAASRRLRNAGKTVSNQALLQEVLERDAMVANKKS
RKERQKLEQAILRSAAVNESKTESLASSVMEAEEVESTTEVQSSSSELEVWDYEQLREEYGF (SEQ ID NO: 50)
>WP_094343929.1
[Nostoc sp. ‘Peltigera membranacea cyanobiont’ 232]
MYQKLKNSCLSSEDDAVELQQHQNSTKPHKLPSEELITEQVKLRMEVIQSLTEPCDRKTYRAKKIEAAQKLGVSIRQVERLLQK
WREQGLVGLTTTRSDKGKYRLEQEWIDFIINTYKEGNKGSKRMTRNQVFLKVKGRAKQLSLNKSEHPSHQSVYRILDEHIEQKE
RKEKARSPGYLGERLTHMTRDGRELEVEGSNDVWQCDHTRLDVRLVDEYGVLDRPWLTIVIDSYSRCLVGFYVGFDHPSSQIDT
LALGRAILPKSYGSEYQLRNEWQAYGKPNYFYTDGGKDFTSIHTTEQVAVQIGFNCALRRRPSDGGIVERFFKTLNLQVLNTLP
GYTGSNVQERPENVDKDACLTLKDLELLLVRFIVNEYNPHTDARMKGQSRIGRWEAGLMADPYLYDQLDLAICLMKQERRKVQK
YGCTQFENLTYRAEHLRGRDGETVAFRYDPADITTLLVYKMNADGTEEFMDYAHAQALETECLSLRELKAINKRLNEASQEINN
DSILEAMIDRQAFVEETVKRNRKQRRQAANEQVNPVEQVAKKFATPEQKEVEPESEADIELPKYEVRYMDEFFEED
(SEQ ID NO: 51)
>WP_084763316.1
[Scytonema hofmanni UTEX 2349]
MNSQQNPDLAVHPLAIPMEGLLGESATTLEKNVIATQLSEEAQVKLEVIQSLLEPCDRTTYGQKLREAAEKLNVSLRTVQRLVK
NWEQDGLVGLTQTSRADKGKHRIGEFWENFITKTYKEGNKGSKRMTPKQVALRVEAKARELKDSKPPNYKTVLRVLAPILEKQQ
KAKSIRSPGWRGTTLSVKTREGKDLSVDYSNHVWQCDHTRVDVLLVDQHGEILSRPWLTTVIDTYSRCIMGINLGFDAPSSGVV
ALALRHAILPKRYGSEYKLHCEWGTYGKPEHFYTDGGKDFRSNHLSQIGAQLGFVCHLRDRPSEGGVVERPFKTLNDQLFSTLP
GYTGSNVQERPEDAEKDARLTLRELEQLLVRYIVDRYNQSIDARMGDQTRFERWEAGLPTVPVPIPERDLDICLMKQSRRTVQR
GGCLQFQNLMYRGEYLAGYAGETVNLRFDPRDITTILVYRQENNQEVFLTRAHAQGLETEQLALDEAEAASRRLRTAGKTISNQ
SLLQEVVDRDALVATKKSRKERQKLEQTVLRSAAVDESNRESLPSQIVEPDEVESTETVHSQYEDIEVWDYEQLREEYGF
(SEQ ID NO: 52)
>WP_073629994.1
[Scytonema sp. HK-05]
MGETLNSNEVDESLVLYDGSDEVDEISESEDTKQSNVIVTELSEEAKLRMQVLQSLIEPCDRKTYGIKLKQAAEKLGKTVRTVQ
RLVKKYQEQGLSGVTEVERSDKGGYRIDDDWQDFIVKTYKEGNKGGRKMTPAQVAIRVQVRAGQLGLEKYPCHMTVYRVLNPII
ERKEQKQKVRNIGWRGSRVSHQTRDGQTLDVHHSNHVWQCDHTKLDVMLVDQYGETLARPWLTKITDSYSRCIMGIHLGFDAPS
SLVVALAMRHAMLRKQYSSEYKLHCEWGTYGVPENLFTDGGKDFRSEHLKQIGFQLGFECHLRDRPPEGGIEERGFGTINTSFL
SGFYGYLDSNVQKRPEGAEEEACITLRELHLLIVRYIVDNYNQRIDARSGNQTRFQRWEAGLPALPNLVNERELDICLMKKTRR
SIYKGGYVSFENIMYRGDYLSAYAGESVLLRYDPRDISTVFVYRQDSGKEVLLSQAHAIDLETEQISLEETKAASRKIRNAGKQ
LSNKSILAEVQDRDTFIKQKKKSHKQRKKEEQAQVHSVKSFQTKEPVETVEEIPQPQKRRPRVFDYEQLRKDYDD
(SEQ ID NO: 53)
>WP_072619869.1
[Spirulina major PCC 6313]
MNTGNQEAHAVITDFSEEERLKLEVIQSLMEPCDHATYGQKLKDAAQKLGKSKRTVQRLVQQWEEMGLAAVTSKARADKGKHRI
SQEWQDFIVKTYRLGNKGSKRMSRKQVALRVQARAAELGEKMYPNERTVYRVLQPIIEAQEQKKSVRSAGWRGDRLSVKTRTGN
DLVVEYTNQVWQCDHTWVDVLVVDVEGNIIGRPWLTTVIDTYSRCILGIRLGFDAPSSQVVALALRHAMLPKQYPPTFGLQCEW
GTYGKPEYFYTDGGKDFRSEHLRQIGIQLGFTCELRDRPSEGGAVERPFGTLNTELFSTLPGYTGSNIQERPEDAEKDARMTLR
DLEQLIVRYLVDNYNQRLDKRMGDQTRYQRWESGLLATPALLSERELDICLMKQTNRSIYREGYIRFENLMYKGEYLAGYVGER
VVLRYDPRDITTVLVYRREKSQEVFLARAYAQDLETEQLTLEDAKAINKKIREKGKTISNRSILDEVRDRDLFVSKKKTKKERQ
KEEQTRLFTPVTTPNSKQETEEEEIEPVEKIDELPQVEILDYDELNDDYGW (SEQ ID NO: 54)
>BAQ60391.1
[Geminocystis sp. NIES-3708]
MLEEKHNIEDHVARSRLEDSSNNTIVSELPVEAQQKLKIIQSLLEPCDRATYGDRLREGAKELGISVRSVQRLFKRYQEEGLNA
LTINDQRDKGKHRIKDFWESFILKTYKEGNKGSKRMNVKQVAIRVQAKALELNETNPPSYRTVLRVLNPIINQEKKTIRSPGWE
GSTLSVKTRDGVDLNISYSNQVWQCDHTKVDVLLVDKNEVLLDRPWLTTVIDSYSRCIVGVNIGFDAPSSNVVGLALRHAILPK
HYPEEYRLNCEWGTYGLPQYLFTDGGKDFRSNHLEEIATQLGFVLKLRDRPSEGGIVERPFKTLNQSLFSTLPGYTGSNVQERP
EDAEKDAQLTLQELERLIVRFIVDKYNQSIDARMGDQTRFQRWEAGLRAIPEVMSERDLDICLMKQARRQVQRGGYLLFENIHY
KGEYLEGFAGKTVSLRYDPRDITTIWVYQHSRGQEEFLTRAYAQGLETESLSLADAKASAKRLREKEKNISNEAILQEVIDREV
TIKNKSRKQRQKEEQSYKKTPSLPVIEKEQESFEQVEEILLDTDDDFEVWDLDEMKDDYGW (SEQ ID NO: 55)
>BAT55397.1
[Nostoc sp. NIES-3756]
MPDKEFGLTGELTQITEAIFLSESNFVVDPLHIILESSDSQKLKFNLIQWLAESPNRQIKSQRKQAVADTLGVSTRQVERLLKE
YNGDRLNETAGVQRCDKGKHRVSEYWQQYIKTIYENSLKEKHPMSPASVVREVKRHAIVDLGLEHGDYPHPATVYRILNPLIEQ
QKRKKKIRNPGSGSWLTVETRDGKQLKAEFSNQIIQCDHTELDIRIVDSNGVLLPERPWLTTVVDTFSSCVLGFHLWIKQPGSA
EVALALRHSILPKQYPHDYELSKPWGYGPPFQYFFTDGGKDFRSKHLKAIGKKLRFQCELRDRPNQGGIVERIFKTINTQVLKD
LPGYTGSNVQERPENAEKEACLGIQDIDKILASFFCDIYNHEPYPKDPRETRFERWFKGMGGKLPEPLDERELDICLMKETQRV
VQAHGSIQFENLIYRGESLRAYKGEYVTLRYDPDHILTLYVYSCDANDDIGDFLGYVHAVNMDTQELSIEELKSLNKERSKARR
EHSNYDALLALGKRKELVKERKQEKKERRQAEQKRLRSGSKKNSNVVELRKSRAKNYVKNDDPIEVLPERVSREEIQVPKTEVQ
IEVSEQADNLKQERHQLVISRRKQNLKNIW (SEQ ID NO: 56)
>ACV01082.1
[Cyanothece sp. PCC 8802]
MNTFPNEQSNAIVLKNTIVSDLPETARTKMEVIQTLLEPCDRITYGERLREGAKKLGVSVRTVQRLFKQYQEQGLAALVSTERS
DKGKHRINEFWQDFIVKTYQQGNKGSKRMTPKQVALKVQAKALEIGDEQPPTYRTVLRVLKPIQEKQEKTKSIRSPGWRGSTLS
VKTRDGDDLEINYSNQVWQCDHTRADVLLVDRHGELIGRPWLTTVIDSYSRCIMGINLGFDAPSSQVVALALRHAILPKRYGDE
YKLHCEWETSGTPEYFYTDGGKDFRSNHLAQIGSQLGFVHKLRDRPSEGGIVERPFKTLNQSLFSTLPGYTGSNVQERPKEAEK
EASLTLRELEQLIVRFIVDKYNQSIDARMGDQTRYQRWEAGLRRQPEPISERELDICLMKAARRTVQRGGYLQFENVMYRGEYL
EGYAGDTVILRYEPRDITTIWVYRQKKHQEVFLTRAHAQDLETEQLSVDEAKASAKKLRDAGKTISNQSILQEVIEREALVEKK
SRKQRQKAEQAYKQEKQPSIIETVEPIEPEPLTQTEVDDIEVWDYDQLRDDYGW (SEQ ID NO: 57)
>AFZ00420.1
[Calothrix sp. PCC 6303]
MSVFALMADKKFELTEKFTQLPEAVFLGENNFVIDPSQIILETSDKHKLTFNLMQWLAESPNRTIKSQRKQAIASTLGVSTRQV
ERLLKQYDEDRLSETSGLQRSDKGKYRVSDYWQEFIKTTYEKSLKDKHPISPASIVREVKRHAIVDLKLEQGNYPHPATVYRIL
NPLIEQQERKKKVRNPGSGSWMTVETRDGKQLKVDFSNQIIQCDHTKLDIRIVDSDGILLTERPWLTTVVDTFSSCVNGFHLWI
KQPGSAEVAIALRHAILPKQYPDDYELGKPWKIYGHPFQYFFTDGGKDFRSKHLKAIGKKLGFQCELRDRPIQGGIVERIFNTI
NTQVLKDLPGYTGPNVQERPENAEKEACLSIHDLDKILASFFCDIYNHEPYPKDTRITRFERWFKGMGEKLPEPLNERELDICL
MKEAQRVVQAHGSIQFENLVYRGESLNAHKGEYVTLRYDPDHILTLYVYSYDVNDELENFLGYVHAINMDTQDLSLEELKSLNK
ERSKARREHSNYDALLALSKRKELVEERKQGKKEKRQAEQKRLRAASKKNSNVVELRQNRASSSSNKDEKDEKIELLPERVSRE
ELKVEKIEPQLEILDKAETPPQERHKLVISSRKQHLKKIW (SEQ ID NO: 58)
>AFZ58294.1
[Anabaena cylindrica PCC 7122]
MKESQVAIQTLDIDHLGIVAGIIDEMELVEEVNKIVGIKTKETLTPGQVVKAMILNGLGFLSAPLYLFGEFFVGKATEHLIGEG
VLPEHLNDDKLGRELDKYHQIGTTKIFTAVAIKAAHKFQVEMDSIHLDGTSMSVEGEYKKEIKEIDEIKQETEENKLEIEPEMK
AIEIVHGYSRDKRPDLKQFIIDMIVTGDGDIPLYLKVDSGNVDDKSVFVERLKEFKKQWTFEGISVADSALYTAENLAAMRELK
WITRVPLSIKEAKNKIVDIKEAEWKDSQISGYKIAAKESEYAGIKQRWIIVESEIRKKSSIQQVEKQVKKQEAKAKAALSKLSR
QEFACQPDAKIVIEKLSKSWKYHQIKEIEYIEKLEYKTAGRPSKLTEPSQIKYQIKGQIETREEVIETEKINAGRFILATNVLD
RNELSDEKVLEEYKAQQSNERGFRFLKDPLFFTSSVFVKTPERVEAIAMIMGLCLLVYNLAQRKLRQELAKFDDGIRNQVKKIT
NKPTMRWVFQMFQAVHLVIINGQKQMSNLTEEREKIVRYLGKSCSKYYLIT (SEQ ID NO: 59)
>AFZ56182.1
[Anabaena cylindrica PCC 7122]
MADEEFEFTEGTTQVPDAILLDKSNFVVDPSQIILATSDRHKLTFNLIQWLAESPNRTIKSQRKQAVANTLDVSTRQVERLLKQ
YDEDKLRETAGIERADKGKYRVSEYWQNFITTIYEKSLKEKHPISPASIVREVKRHAIVDLELKLGEYPHQATVYRILDPLIEQ
QKRKTRVRNPGSGSWMTVVTRDGELLRADFSNQIIQCDHTKLDVRIVDNHGNLLSDRPWLTTIVDTFSSCVVGFRLWIKQPGST
EVALALRHAILPKNYPEDYQLNKSWDVCGHPYQYFFTDGGKDFRSKHLKAIGKKLGFQCELRDRPPEGGIVERIFKTINTQVLK
ELPGYTGANVQERPENAEKEACLTIQDLDKILASFFCDIYNHEPYPKEPRDTRFERWFKGMGGKLPEPLDERELDICLMKEAQR
VVQAHGSIQFENLIYRGEFLKAHKGEYVTLRYDPDHILSLYIYSGETDDNAGEFLGYAHAVNMDTHDLSIEELKALNKERSNAR
KEHFNYDALLALGKRKELVEERKEDKKAKRNSEQKRLRSASKKNSNVIELRKSRTSKSLKKQENQEVLPERISREEIKLEKIEQ
QPQENLSASPNTQEEERHKLVFSNRQKNLNKIW (SEQ ID NO: 60)
>WP_066425682.1
[Anabaena sp. 4-3]
MADREIEFTEESTQDSDAILLDNSNFVVDPSQIILATSDKHKLTFNLIQWLAQSPTRTVKSERKQAIANTLSVSTRQVERLLKQ
YNEDKLRETAGTERADKGKHRVSEYWQEFIKTTYEKSLKDKHPISPASIVREVKRHAIVDLGLKPGDYPHQATVYRILEPLIAQ
HKRKTRVRNPGSGSWMTVVTRDGQLLRADFSNQIIQCDHTKLDIRIVDIHGDLLSERPWLTTVVDTYSSCVLGFRLWIKQPGST
EVALALRHAILPKQYPDDYQLNKSWNVYGNPFQYFFTDGGKDFRSKHLKAIGKKLGFQCELRDRPPEGGIVERIFKTINTQVLK
DLPGYTGANVQERPENAEKEACLTIQDLDKILASFFCDIYNHEPYPKEPRDTRFERWFKGMGEKLPERLDERELDICLMKETQR
VVQAHGSIQFENLIYRGESLKAHKGEYVTLRYDPDHILTLFVYSCETDDNLEEFLGYAHAVNMDTHDLSLEELKTLNKERSKAR
KEHFNYDALLALGKRKELVDERKADKKEKRHSEQKRLRSASKKDSNIIELRKSRVSKSLRKQETQEILPERVSREEIKFEKIEL
EPQETLSASPKPNPQEEQRHKLVLSKRQKNLKNIW (SEQ ID NO: 61)

TABLE 5
Amino acid sequences of Representative CLUST.004377 Effector_C2 Proteins

>OUC12055.1
[Alkalinema sp. CACIAM 70d]
MTIDSGIQPWLFQVEPYEGESLSHFLGRFRRPNHLTPSGLGQLAGIGAVVARWERFHWNPPPSRQELEALAKVVRVSVEQLIAM
LPPPGVGMQCSPIRLCSACYGETPCHRMEWQYKHIWKCNRHSLKLLAKCPVCSALFKAPAQWQDGMCRRCLTPFGQMQQQ
(SEQ ID NO: 62)
>OBQ23833.1
[Anabaena sp. WA113]
MIQPYEGESLSHFLGRFRRANHLSAAGLGNLAGIGAVIARWERFHFNPRPSQKELEAIASVVEVDAQRLAEMLPPAGVSMQHEP
IRLCGACYAETPCHQIKWQFKETGGCDRHYLRLLSKCPNCDARFKIPALWELGVCQRCLMTFAEMAGYQKSINGT
(SEQ ID NO: 63)
>WP_011318169.1
[Anabaena variabilis ATCC 29413]
MPEEIYNFEAWINVVEPLPGESISHFLGRFERANLLTGYQVGREVGVGAIVTRWKKLYLNPFPTLQELEALARFVEVEVEKLKE
MLPSQGMTMKPRPIKICAACYAEVPCHRIEWQYKDNMKCDRHNLRLLTKCTNCETPFPIPADWLQGECPRCFLPFAKMAKRQKL
NS (SEQ ID NO: 64)
>WP_011318975.1
[Anabaena variabilis ATCC 29413]
MRESINENKQFWLIRVEPLEGESISHFLGRFRREKGNKFSAPSGLGDVAGLGAVLARWEKFYFNPFPTHQELEALASVVQVDVD
RLRQMLPPLGVSMKHSPIRLCGACYAESPCHKIEWQFKKTVGCDRHQLRLLSKCPVCEKPFPVPALWVDGICNRCFTPFAEMAQ
YQKHY (SEQ ID NO: 65)
>WP_011317999.1
[Anabaena variabilis ATCC 29413]
MEVPQIQPWLFQIEPLEGESLSHFLGRFRRANDLTPTGLGKAAGLGGAIARWEKFRFNPPPSRQQLEALANVVGVDADRLAQML
PSAGVGMKMEPIRLCAACYAESPCHKIEWQFKVTRGCARHKITLLSECPNCKARFKVPALWVDGWCNRCFLRFEEMAKYQKGL
(SEQ ID NO: 66)
>WP_039728662.1
[Aphanocapsa montana BDHKU210001]
MVIPQIPAWVFPVEPSPGESLSHFLGRFCRENHTTLNQLGEKTGLGAVLGRWEKFRFIPQPSDAQLAALAKLVRLEVDQIKQML
PQETMQNRVIRLCAACYAEEPYHRIEWQYKLANRCDRHHLLLLLECPNCKAKLPMPSKWANGTCKRCLTPFEQMADLQKGI
(SEQ ID NO: 67)
>WP_019497300.1
[Calothrix sp. PCC 7103]
MNEPRFFEIEPFECESLSHFLGRFRRENYLTATQLGKLTGLGAVISRWENCYFNPFPTQQELEALASVVGAEVEKLREMLPPIG
VTMKPRPIRLCAACYAESPYHHIEWQFKDVMKCSYHQMRLLTKCTNCGAVFPIPADWVTGECPHCCLPFVTMVRKQQKG
(SEQ ID NO: 68)
>WP_012596235.1
[Cyanothece sp. PCC 8801]
MDKEYWLTKVEPYEGESISHFLGRFRRTKGNRFSAASGLGQVAGLGAILARWEKLYFNPFPNEQELEALGKVVMLDVEDLRKML
PQKGMVTQPKPIMLCAVCYGEKPYHRMVWQDKHQRGCEVHGLELLSRCINCKRLFPIPSKWEEGKCLLCGLAFAKIAKHQKPI
(SEQ ID NO: 69)
>WP_066116120.1
[Geminocystis sp. NIES-3709]
MDLQIQNWLFILVPYEGESISHFLGRFRRANSLSCGGLGQATGLYSAIARWEKFRFNPPPSLKQLEKLSEIVQVEMATLQTMFP
SAPMKMTPIRICSACYGENPYHQMSWQYKEIYRCDRHNLNILSECPNCAARFKFPNLWFEGFCHRCFTPFEQMPQS
(SEQ ID NO: 70)
>BA555900.1
[Leptolyngbya boryana dg5]
MIEHSEIQPWFFHVEALEGESISHFLGRFRQANELTPSGVGKISGLGGAIARWEKFRFNPYPTQQQFEKLSTATGISVEQLWKM
MPPEGVGMQLEPIRLCASCYAELPCHQIQWQFKDTQGCEVHGLRLLSECPNCKARFKPPATWSDSKCHRCFMLFSEMRNRQKRH
SFSR (SEQ ID NO: 71)
>WP_026148796.1
[Leptolyngbya boryana IAM M-101]
MEHSEIQPWFFHVEALEGESISHFLGRFRQANELTPSGVGKISGLGGAIARWEKFRFNPYPTQQQFEKLSTATGISVEQLWKMM
PPEGVGMQLEPIRLCASCYAELPCHQIQWQFKDTQGCEVHGLRLLSECPNCKARFKPPATWSDSKCHRCFMLFSEMRNRQKRHS
FSR (SEQ ID NO: 72)
>WP_036484423.1
[Myxosarcina sp. GI1]
MNNTKEAQLWLFPVEPSNGESLSHFLGRFRRSNHLSPSALGDLAGIGGVVARWERFHLNPFPTDEQFQALAEVVDVDSSTLREM
LPPKGTGMKCDRHNLKLISKCPNCRAKFKMPALWEYGCCHRCRLPFAAIAQYQQSV
(SEQ ID NO: 73)
>WP_012411917.1
[Nostoc punctiforme PCC 73102]
MTTPDVKPWLFIIEPYPGESLSHFLGRFRRANHLSPAGLGGLAGIGAVVARWERFHFNPRPSQKELEAIASVVEVDAQRLAQML
PPAGVGMQHEPIRLCGACYAEAPCHRIEWQYKSVWKCDRHQLKILAKCPNCQAPFKMPALWEDGCCHRCRTLFAEMAKQQKS
(SEQ ID NO: 74)
>BAB75327.1
[Nostoc sp. PCC 7120]
MMAAPDVKPWLFIIQPYEGESLSHFLGRFRRANHLSASGLGKLAGIGAVVARWERFHFNPRPSQKELEAIASLVEVDADRLAQM
LPPLGVGMQHEPIRLCGACYAEAPCHRIEWQYKSVWKCDRHELKILAKCPNCEAPFKIPALWEDKCCHRCRTPFAEMTKYQKIT
(SEQ ID NO: 75)
>WP_044521839.1
[Nostoc sp. PCC 7120]
MAAPDVKPWLFIIQPYEGESLSHFLGRFRRANHLSASGLGKLAGIGAVVARWERFHFNPRPSQKELEAIASLVEVDADRLAQML
PPLGVGMQHEPIRLCGACYAEAPCHRIEWQYKSVWKCDRHELKILAKCPNCEAPFKIPALWEDKCCHRCRTPFAEMTKYQKIT
(SEQ ID NO: 76)
>WP_094343931.1
[Nostoc sp. ‘Peltigera membranacea cyanobiont’ 232]
MEENIDEKRQIWLTRVEPFDGESISHFLGRFRREKGNKFSAPSGLGDVAGLGVVLARWEKFYFNPFPTHQELDALATVVELDAE
RLRQMLPPEGVGMKHSPIRLCGACYAESVCHKIEWQFKKRVGCDRHLLRLLSKCPVCEKPFPVPALWMDGQCQRCFTSFAEMAE
HQKHY (SEQ ID NO: 77)
>WP_029636334.1
[Scytonema hofmanni UTEX 2349]
MIEAPDVKPWLFLIKPYEGESLSHFLGRFRRANHLSASGLGTLAGIGAIVARWERFHFNPRPSQQELEAIASVVEVDAQRLAQM
LPPAGVGMQHEPIRLCGACYAESPCHRIEWQYKSVWKCDRHQLKILAKCPNCQAPFKMPALWEDGCCHRCRMPFAEMAKLQKV
(SEQ ID NO: 78)
>WP_073629990.1
[Scytonema sp. HK-05]
MEIPAEQPRFFQVEPLEGESLSHFLGRFRRENYLTATQLGKLTGIGAVISRWEKFYLNPFPTPQELEALAAVVEVKVDRLIEML
PPKGVTMKPRPIRLCGACYQESPCHRVEWQFKDKLKCVSEAHPKDARHQLGLLTKCTNCETPFPIPADWVQGECPHCFLPFAKM
ARRQKRY (SEQ ID NO: 79)
>WP_072619870.1
[Spirulina major PCC 6313]
MNDWEIQPWLFVVEPYEGESLSHFLGRFRRENDLTPAGLGREAEIGAVVSRWEKFRLIPFPSQRELEKLAQVVQVDAARLRVML
PPDGVGMKMTPIRLCGACYREVRCHRMEWQYKTSDRCDKHPLRLLSECPNCGARFPIPSLWQDGWCTRCFTTFGEMAESQKPL
(SEQ ID NO: 80)
>BAQ60389.1
[Geminocystis sp. NIES-3708]
MLDTDINTWLLPIEPLEGESLSHFLGRVRRRNYLSASALGELAGIGGAITRWEKFRHYPFPSDEELTALGNLLGLELFQLKAML
PSEPMKLEPIRLCGACYGEIPSSSY (SEQ ID NO: 81)
>BAT55395.1
[Nostoc sp. NIES-3756]
MEKDTFPPKTEIRIHDNHEALPRLGYVEPYEGESISHYLGRLRRFKANSLPSGYSLGKIAGIGAVTTRWEKLYFNPFPSSEELE
ALGKLIGVPANRIYEMLPPKGVTMKPRPIRLCAACYAEVPCHRIEWQYKDKLKCNHHNLGLLTKCTNCETPFPIPADWVQGECP
HCFLPFAKMAKRQKPR (SEQ ID NO: 82)
>AFZ00422.1
[Calothrix sp. PCC 6303]
MAEDIYLPKREIISNKEINKGDEILPRLGFVEPYECESISHYLGRVRRFKANSLPSGYSLGKIAGIGAVTTRWEKLYLNPFPSE
TELEALAKVIEVEVERLRQMLPPKGMTMKPRPIRLCAACYAESPHHRIEWQFKDVMVCDRHQLPLSTKCKNCGTPFPIPADWVR
GECPHCCLSFTKMAKRQKSG (SEQ ID NO: 83)
>AFZ58293.1
[Anabaena cylindrica PCC 7122]
MEVGEINPWLFQVEPYLGESLSHFLGRFRRANDLTTTGLGKAAGVGGAISRWEKFRFNPPPSRQQLEALAKVVGVDADRLEQML
PPAGVGMNLEPIRLCAACYVESPCHRIEWQFKVTQGCQHHHLSLLSECPNCGARFKVPALWVDGWCQRCFLPFGEMIEHQKRI
(SEQ ID NO: 84)
>AFZ56184.1
[Anabaena cylindrica PCC 7122]
MAQNIFLSKTEIGIDEDDEIRPKLGYVEPYEEESISHYLGRLRRFKANSLPSGYSLGKIAGLGAMISRWEKLYFNPFPTLQELE
ALSSVVGVNADRLIEMLPSQGMTMKPRPIRLCGACYAESPCHRIEWQCKDRMKCDRHNLRLLIKCTNCETPFPIPADWVKGQCP
HCSLPFAKMAKRQRRD (SEQ ID NO: 85)
>WP_066425687.1
[Anabaena sp. 4-3]
MNEDDEIRPKLGYVEPYEGESISHYLGRLRRFKANSLPSGYSLGKIAGLGAVTTRWEKLYFNPFPNRQELEALASVVGVSAERF
IEMLPPKGVTMKPRPIRLCAACYAEVPCHRIDWQFKDKMKCDRHNLRLLTKCTNCETPFPIPADWAQGECPHCSLSFAKMVKRQ
KLR (SEQ ID NO: 86)

TABLE 6
Amino acid sequences of Representative CLUST.004377 Effector_C3 Proteins

>OUC12056.1
[Alkalinema sp. CACIAM 70d]
MTQAKEWAEVLGEFQPDDDWLKAEIARLRKKTVMPLAQVSQLHDWLDGKRKSRQSCRIVGESRTGKSVACEAYFYRNKPQQEAG
KKPIVPVVYVQPPQKCGPKELFKEIIEFLKHRATRGTVSDLRGRAIEVLQACGVEMLIIDEADRLKPETFADVRDIYDKLGIAV
VLVGTDRLDAVIKRDEQVYNRFRACHRFGKLSGEEFKKTIALWEQKILNLPVASNLISKDMLRILTTGTEGYIGRMDEILREAA
IRSLSFGYKKVEKKCLEEVAKEYK (SEQ ID NO: 87)
>OBQ23775.1
[Anabaena sp. WA113]
MTDDAQAIAKQLGGVKPDEEWLQAEITHLTSKSIVPLQQVITLHDWLDGKRKARQSCRVVGESRTGKTVACDAYRYRHKPRQEM
GKTPIVPVVYIQPPSKCGAKDLFQEIIEYLKFKATRGTISDFRGRTMEVLKGCRVEMIIIDEADRIKPDTFADVRDIYDKLGIA
IVLVGTDRLEAVIKRDEQVYNRFRACHRFGKLAGKDFQDTVQAWEDKILKLPLPSNLISKDMLRILTSATEGYIGGLDEILREA
AIRSLSRGLKKIDKAVLQEVVQEFKL (SEQ ID NO: 88)
>WP_011318170.1
[Anabaena variabilis ATCC 29413]
MTNEEIQQEIERLRQPDILNLEQVKRFSSWLDERRKLRKPGRAVGESGLGKTTASLFYTYQNRAAKIPNQNPVAPVLYVELIGS
SCSPSLLFKTIIETLKFKAKGGTENQLRERAWYLIKQCKVEMLIIDEAHRLQFKTLTDVTDLSDKVKIIPILVGTSSRLDALIS
KNEQVGGRFAAYFSFEQLSGANFIKTVKIWEQQILKLPEPSNLAENQEIITILQAKTAGQIRLLDQILRDAAVKALESGVNKID
KSLLNSIEGDYSLVAS (SEQ ID NO: 89)
>WP_011318974.1
[Anabaena variabilis ATCC 29413]
MTDAKPLDFIQEPTREIQAHIERLSRAPYLELNQVKSCHTWMYELVISRMTGLLVGESRSGKTVTCKAFRNNYNNLRQGQEQRI
KPVVYIQISKNCGSRELFVKILKALNKPSNGTIADLRERTLDSLEIHQVEMLIIDEANHLKIETFSDVRHIYDEDSLKISVLLV
GTTSRLLAVVKRDEQVVNRFLEKFEIDKLEENQFKQMIQVWERDVLRLPEESKLASGESFKLLKQSTNKLIGRLDMILRKAAIR
SLLRGYKKVDQGVLKEIITATKF (SEQ ID NO: 90)
>WP_011317998.1
[Anabaena variabilis ATCC 29413]
MTSKQAQAVAQQLGDIPVNDEKIQAEIQRLNRKSFIPLEQVQMLHDWLDGKRQSRQSGRVVGESRTGKTMGCDAYRLRHKPKQE
PGRPPTVPVAYIQIPQECGAKELFGVLLEHLKYQMTKGTVAEIRDRTLRVLKGCGVEMLIIDEADRLKPKTFAEVPDIFDKLEI
AVILVGTDRLDAVIKRDEQVYNRFRACHRFGKFSGDEFKKIVDIWEKKVLQLPVASNLSSKTMLKTLGETTGGYIGLLDMILRE
SAIRALKKGLRKVDLATLKEVTEEYK (SEQ ID NO: 91)
>WP_044151374.1
[Aphanocapsa montana BDHKU210001]
MIALQDQEVQAHIERLRRDKTVALDSVKQAHTWLKRKRNARQCGRLTGDSRTGKTKTCESFLKLYGEPDLSGRVPIIPISYVHP
KQECTSRELFREILEQYGDDLPRGTVGDARSRTLKVLRACKTEMLMIDEADRLKPKTFADVRDIFDKLEISVILIGTKQRLDPA
VKKDEQVFNRFRSSYRIGTIPSNQLKTIVGLWERDILKLPVPSNLTSEAMLKELRKATGVSRKGYYIGLIDMVLREAAIRALEK
GQSKIELETLKEVAKEYS (SEQ ID NO: 92)
>WP_019497299.1
[Calothrix sp. PCC 7103]
MADEYLRKWVQNLWGDDPIPEELMPQIERLITPSIVELDHIQKIHDWLDSLRLSKQCGRIVAPPRAGKSVTCDVYKLLNKPQKR
TGKRDIVPVLYMQVPGDCSAGELLTLILESLKYEATSGKLTDLRRRVQRLLKESKVEMLIIDEANFLKLNTFSEIARIYDLLKV
SIVLVGTDGLDNLIKKEPYIHDRFIECYRLQLVSEKKFSELVQIWEDDVLCLPVPSNLVKRETLIPLYQKTGGKIGLVDRVLRR
AAILSLRKGLSIIDKITLDEVLEWFE (SEQ ID NO: 93)
>WP_012596234.1
[Cyanothece sp. PCC 8801]
MTDSSVIDKLAAEFGGFATPSPEIQAEIQRLSRQPYLEYEQVKNCHGWLYELVLSRMTGLLVGESRSGKTVTCKSFEKRYNKIK
TGNKKRIKPIVYIQSPQNCGAREFFTKILKALNKPTNGNVSDLRERTLDGLQIHECEMLIIDEANHLKQETFAEVRHVYDEEEL
NMAVLLVGTRHRLEAVVKRDEQVLNRFMEEYELDRLDDKEFKQLIKIWEQSVLCLPELSNLDTGDNLKLLKKTTRKLIGRLDMI
LRKSAIRALREGKQSISPELLKQVISSIKWSEGRKGNG (SEQ ID NO: 94)
>WP_066116122.1
[Geminocystis sp. NIES-3709]
MEAKAIAQELGNIEIPEEKLQMEIERLNSKTLVSLEQVAKLHEYFEGKRQSKQSCRVVGESRTGKTMACDSYRLRHKPIQKVGH
PPQVPVVYIQIPQDCGTKELFQGIIEYLKYQMTKGTIAEIRQRAIKVLQGCGVEMIIIDEADRFKPKTFAEVRDIFDRLNIPIV
LVGTDRLDTVIKRDEQVYNRFRSCYRFGKLSGADFQNTVNIWEKQVLKLPVASNLIQTKMLKLIAEATGGYIGLMDTILRESAI
RSLKRGLNKITFEILKEVTQEFK (SEQ ID NO: 95)
>WP_026148797.1
[Leptolyngbya boryana dg5]
MMANEAQSIAQTLGSLPLTSELLQAEIHRLTKKSVVSLSQVQSLHNWLEGKRQARQSCRVVGESRTGKTLACDAYRLRHKPTQQ
AGKPPIVPVVYIQVPQECGSKELFQIIIEHLKYQMVKGTVAEIRERTMRVLKGCGVEMLIIDEADRLKPKTFADVRDIFDKLEI
SVVLVGTDRLDAVIKRDEQVYNRFRACHRFGKLAGEEFRRTIEIWEKQILKLPVASNLTSKAALKILGETTAGYIGLLDMVLRE
AAIRALKQGKTKIDLEILKEVSTEYR (SEQ ID NO: 96)
>WP_036484424.1
[Myxosarcina sp. GI1]
MTEAKAIADKLGKIELDEEWVQKEIARLNRKSTVALEHIKELHDWLDGKRKSRRSCRIVGKSRTGKTVACEAYVMRNKLNKPPQ
ERQTKNQIPIEPVIMIMPPQKCGAKELFREIIECLKFRAVKGTISEFRSRAMDVLQKCQVEMLIIDEADRLKPETFSEVRDIYD
KLEIAVVLVGTERLDTAVKRDEQVENRFRANRRFGTLEGINFKKTVEIWEEKILKLPVASNLTNKTTWKILLIATEGFIGRLDE
ILREAAIASLSQGHKKVDPKILKEIAREYS (SEQ ID NO: 97)
>WP_012411918.1
[Nostoc punctiforme PCC 73102]
MTEAQAIAKQLGGVKPDDEWLQAEIARLKGKSIVPLQQVKTLHDWLDGKRKARQSCRVVGESRTGKTVACDAYRYRQKPQQEVG
RPPIVPVVYIQPPQKCGSKDLFKEMIEYLKFRATKGTVSDFRGRAMEVLKGCGVEMLIIDEADRLKPETFAEVRDIYDKLGIAV
VLVGTDRLEAVIKRDEQVYNRFRACHRFGKLSGKDFQDTVQAWEDRVLKLPVSSNLTSKDMLRILTSATEGYIGRLDEILRETA
IRSLSKGFKKIDKAVLQEVAKEYK (SEQ ID NO: 98)
>WP_010997774.1
[Nostoc sp. PCC 7120]
MTDAKAIAQQLGGVKPDEEWLQAEIARLKGKSIVPLQQVRSLHDWLDGKRKARQFCRVVGESRTGKTVACDAYRYRQKVQAEVG
RPPIVPVVYIQPPQKCGAKDLFQEIIEYLKFKATKGTVSDFRGRTMEVLKGCGVEMIIVDEADRLKPETFAEVRDIYDKLGIAV
VLVGTDRLEAVIKRDEQVYNRFRACHRFGKLSGKDFQDTVQAWEDKILKLPLPSNLISKDMLRILTSATEGYIGRLDEILREAA
IRSLSRGLKKIDKPVLQEVAQEYK (SEQ ID NO: 99)
>WP_094343930.1
[Nostoc sp. ‘Peltigera membranacea cyanobiont’ 232]
MTNAMSLAKQFGVIQELTPEIQAEIERLSRQPYLELDQVKRCHAWMYELVISRMTGLLLGESRCGKTVTCKAFANLYNKLRQTK
GQRMKPVVYIQVGKNCGSRDFFMKILKALNKPSNGTISDLRERTLDSLAIHQVEMLIIDEANHLKFETFSDVRHIYDDDELKIS
VLLVGTTSRLLAIVKRDEQVVNRFLEQFELDRLEDTQFKQMIQIWERDVLRLPEESKLASGDNLKILKQATKKLIGRLDMILRK
AAIRSLLRGQKQVDKDILKEVIAASKL (SEQ ID NO: 100)
>WP_029636336.1
[Scytonema hofmanni UTEX 2349]
MTEAQAIAKQLGGVKPDDEWLQAEIARLKGKSIVPLQQVKTLHDWLDGKRKARKSCRVVGESRTGKTVACDAYRYRHKPQQEAG
RPPTVPVVYIRPHQKCGPKDLFKKITEYLKYRVTKGTVSDFRDRTIEVLKGCGVEMLIIDEADRLKPETFADVRDIAEDLGIAV
VLVGTDRLDAVIKRDEQVLERFRAHLRFGKLSGEDFKNTVEMWEQMVLKLPVSSNLKSKEMLRILTSATEGYIGRLDEILREAA
IRSLSRGLKKIDKAVLQEVAKEYK (SEQ ID NO: 101)
>WP_073629992.1
[Scytonema sp. HK-05]
MAEDYLRKWVQNLWGDDPIPEELLPIIERLITPSVVELEHIQKIHDWLDSLRLSKQCGRIVAPPRAGKSVTCDVYKLLNKPQKR
TGKRDIVPVLYMQAPGDCSAGELLTLILESLKYDATSGKLTDLRRRVLRLLKESRVEMLVIDEANFLKLNTFSEIARIYDLLKI
SIVLVGTDGLDNLIKKERYIHDRFIECYKLPLVSENKFPEFVQIWEDEVLCLPVPSNLIKSETLKPLYQKTSGKIGLVDRVLRR
AAILSLRKGLKNIDKATLDEVLEWFE (SEQ ID NO: 102)
>WP_072622994.1
[Spirulina major PCC 6313]
MASAESKAKAEAVAQQLGNFEKTEEDLAKEIQRLRRRNVVQLEQVKQLHNWLEGKRRSRQCCRVVGESRTGKTIGCNAYRLRHK
PIQETGKPPIVPVVYIEPPQDCGSIDLFRAIIEYLKYKVQSREKVRELRSRAMKVLERCQVETLIIDEADRLKPKTFADVRDIF
DKRNISVVLVGTDRLDNVIKRDEQVHNRFRACYRFGKLTGTEFEQVVKIWERDILRLPIPSNLHAKNMLKILGQATGGYIGLLD
MILRETAVRALEKGLGKINLETLKEVAEEYS (SEQ ID NO: 103)
>BAQ60390.1
[Geminocystis sp. NIES-3708]
MTKAQSIAKELGDLGQDEQWLHQEICRLNRSSIVPLEHLKDLHNWLDEKRKARQSCRIVGESRTGKTIACESYKLRNKPSQKGQ
QTPSVPVVYIMPPAKCSAKDFFREIIEALRYRAVKGTVSDFRSRAMDVLKACDVEMLIVDEADRLKPDTFPEVRDISDKLEMLV
VLVGTDRLDAVIKRDEQVYNRFRSHRRFGKLTGEDFKETVSIWEKEVLNLPVASNLTKLDMFKIITKATGGYIGRLDELLREAA
IKSLSRGKKALKRIFYRR (SEQ ID NO: 104)
>BAT55396.1
[Nostoc sp. NIES-3756]
MAQSQLVIQPNVETLAPQLELNNQLAKVVEIEEIFSNCFIPTDRACEYFRWLDELRILKQCGRVVGPRDVGKSRASVHYREEDR
KKVSYVRAWSASSSKRLFSQILKDINHAAPTGKREDLRPRLAGSLELFGIEQVIVDNADNLQREALLDLKQLFDESNVSVVLVG
GQELDKILHDCDLLTSFPTLYEFDTLEDDDFKKTLSTIEFDVLALPQASNLCEGITFEILVQSTGGRIGLLVKILTKAVLHSLK
NGFGRVDQNILEKIANRYGKRYIPPENRNKNS (SEQ ID NO: 105)
>ACV01083.1
[Cyanothece sp. PCC 8802]
MTQAQEIAQKLGDLNPDEQWLQMEIARLNRQSIVPLEHIRDLHEWLDGKRKARQSCRLVGESRTGKTVACEAYTLRNKPIQQGR
QTPIVPVVYIMPPTKCGSKDLFKEIIEYLRYKAVKGTVSEFRSRAMEVLKGCEVEMIIIDEADRLKPDTFPDVRDINDKLEISV
VLVGNDRLDAVIKRDEQVYNRFRAHRRFGKLAGVEFKKTVAIWEEKVLKLPVASNLTSSALIKILVKATEGYIGRLDEILREAA
IKSLMKGHKRIEKEVLQEVAKEYS (SEQ ID NO: 106)
>AFZ00421.1
[Calothrix sp. PCC 6303]
MARSQLAIQSSVEVLVPQLDLNAQLAKVVEVEEIFSNYFIPTDRSSEYLRWLDELRILRQCGRVIGPRDVGKSRASLHYQGQDQ
KRISYVRAWSASSSKRLFSQILKDIKHAAPMGKRDDLRPRLAGSLEVFGFEQVIIDNAENLQKEALLDLKQLFDECHVPIVLIG
GQELDTILDEFDLLTCFPTLYEFDGLDENDFKKTLNTIEFDILALPEASNLSEGIIFELLAESTGARIGLLVKILTKAVLHSLK
NGFSKIDQNILEKIANRYGRRYIPPEKRNNK (SEQ ID NO: 107)
>AFZ56183.1
[Anabaena cylindrica PCC 7122]
MAQPQLATQSIVEVLAPRLDIKAQIAKTIDIEEIFRACFITTDRASECFRWLDELRILKQCGRIIGPRNVGKSRAALHYRDEDK
KRVSYVKAWSASSSKRLFSQILKDINHAAPTGKRQDLRPRLAGSLELFGLELVIIDNAENLQKEALLDLKQLFEECNVPIVLAG
GKELDDLLHDCDLLTNFPTLYEFERLEYDDFKKTLTTIELDVLSLPEASNLAEGNIFEILAVSTEARMGILIKILTKAVLHSLK
NGFHRVDESILEKIASRYGTKYIPLKNRNRD (SEQ ID NO: 108)
>WP_066425684.1
[Anabaena sp. 4-3]
MARSQLANQPIVEVLAPQLDLNAQIAKAIDIEEIFRNCFITTDRVSECFRWLDELRILKQCGRIIGPRNVGKSRAALHYRDEDK
KRISYVKAWSASSSKRIFSQILKDINHAAPTGKRQDLRPRLAGSLELFGLELVIIDNADNLQKEALIDLKQLFEECHVPIVLIG
GKELDNILQDCDLLTNFPTLYEFERLEYDDFRKTLSTIELDILSLPESSHLAEGNIFEILAVSTSGRMGILVKILTKAVLHSLK
NGFGRVDESILEKIASRYGTKYVPLENRNRNE (SEQ ID NO: 109)

TABLE 7
Nucleotide sequences of Representative CLUST.004377 Direct Repeats

| CLUST.004377 Effector_A Protein | |
|---|---|
| Accession | Direct Repeat Nucleotide Sequence |
| OUC12050.1 (SEQ ID NO: 1) | ATCGCAACCAACTGTCCTGAAGAAGGGCGATTGAAAG |
| | (SEQ ID NO: 110) |
| OBQ23770.1 (SEQ ID NO: 2) | GTAACAACAACCCTCCTAGTACAGGGTGGGTTGAAAG |
| | (SEQ ID NO: 111) |
| WP_011318157.1 (SEQ ID NO: 3) | GTTTCAACGACCATCCCGGCTAGGGGCGGGTTGAAA |
| | (SEQ ID NO: 112) |
| WP_011318983.1 (SEQ ID NO: 4) | GTGACAATAGCCCTTCCCGTGTTGAGCGGGTTGAAAG |
| | (SEQ ID NO: 113) |
| WP_011318008.1 (SEQ ID NO: 5) | GTTTCAACACCCCTCCCGAAGTGGGGCGGGTTGAAAG |
| | (SEQ ID NO: 114) |
| WP_039730330.1 (SEQ ID NO: 6) | GGTCGCCAAAAGCATTTCAGGGCAGGGC |
| | (SEQ ID NO: 115) |
| WP_019497312.1 (SEQ ID NO: 7) | GTGCTGTAACCTAAGATGTCGCAAGGCGTTGAGCAG |
| | (SEQ ID NO: 116) |
| WP_012596241.1 (SEQ ID NO: 8) | GAGTTTCATCAACCCTCCTGATGTGGGATGGGTTGAAAG |
| | (SEQ ID NO: 117) |
| WP_012596246.1 (SEQ ID NO: 9) | GTTTCATACAGGTTTTTGACCTCCCATTGATTGAAAGA |
| | (SEQ ID NO: 118) |
| WP_066116114.1 (SEQ ID NO: 10) | GTTGAAATAAGAAAATACCTTCTCTAGGGATTGAAAG |
| | (SEQ ID NO: 119) |
| WP_017289534.1 (SEQ ID NO: 11) | GTTTCATCCAGGTTTGCGGCAAGGGGGCGATTGAAAG |
| | (SEQ ID NO: 120) |
| WP_017289534.1 (SEQ ID NO: 11) | GTTTCATCCAGGTTTGCGGCAAGGGGGCGATTGAAAG |
| | (SEQ ID NO: 120) |
| WP_017289534.1 (SEQ ID NO: 11) | GTTTCATCCAGGTTTGCGGCAAGGGGGCGATTGAAAG |
| | (SEQ ID NO: 120) |
| WP_039730330.1 (SEQ ID NO: 6) | GGTCGCCAAAAGCATTTCAGGGCAGGGC |
| | (SEQ ID NO: 115) |
| WP_036484397.1 (SEQ ID NO: 12) | GTTTCAACGACCACTTTAAGATGGGTATGGTTGAAAG |
| | (SEQ ID NO: 121) |
| WP_012411901.1 (SEQ ID NO: 13) | GTGGCAACAACCCTCCAGGTACTGGGTGGGTTGAAAG |
| | (SEQ ID NO: 122) |
| BAB75312.1 (SEQ ID NO: 14) | CGTTGCAACCCTCCTTCCAGTAATGGGAGGGTTGAAAG |
| | (SEQ ID NO: 123) |
| WP_044521835.1 (SEQ ID NO: 15) | CGTTGCAACCCTCCTTCCAGTAATGGGAGGGTTGAAAG |
| | (SEQ ID NO: 123) |
| WP_094343940.1 (SEQ ID NO: 16) | GTTGCAATGACCCTTCCCGTGTTGAGCGGGTTGAAAG |
| | (SEQ ID NO: 124) |
| WP_029636312.1 (SEQ ID NO: 17) | GTGGCAACAACCTTCCAGGTACTAGGTGGGTTGAAAG |
| | (SEQ ID NO: 125) |
| WP_073629970.1 (SEQ ID NO: 18) | GTTTCAACAACCATCCCGGCTAGGGGTGGGTTGAAAG |
| | (SEQ ID NO: 126) |
| WP_072619878.1 (SEQ ID NO: 19) | GTTTCAATGACCATCCCACGTTGGGATGGATTGAAAGAG |
| | (SEQ ID NO: 127) |
| BAQ60380.1 (SEQ ID NO: 20) | GATTCTTCGTAAACTCAGAATGACA |
| | (SEQ ID NO: 128) |
| BAQ63841.1 (SEQ ID NO: 10) | GTTGCAGATGAATTTACTTCTCTGTGCGATCGAAAG |
| | (SEQ ID NO: 129) |
| BAT55382.1 (SEQ ID NO: 21) | GTTTCAACGACCATCCCGGCTAGGGGTGGGTTGAAAG |
| | (SEQ ID NO: 130) |
| ACV01094.1 (SEQ ID NO: 22) | GTTTCAACGACCATTCCCAACAGGGATGGGTTGAAAG |
| | (SEQ ID NO: 131) |
| AFZ00435.1 (SEQ ID NO: 23) | GTTTCAACTACCATCCCGACTAGGGGTGGGTTGAAAG |
| | (SEQ ID NO: 132) |
| AFZ58287.1 (SEQ ID NO: 24) | GTTTCAACACCCCTCCCGGAGTGGGGCGGGTTGAAAG |
| | (SEQ ID NO: 133) |
| AFZ56196.1 (SEQ ID NO: 25) | GTTTCAACTACCATCCCGACTAGGGGTGGGTTGAAAG |
| | (SEQ ID NO: 132) |
| ODH02164.1 (SEQ ID NO: 26) | GTTTCCAAAGCCCTCTCGTTAGGTGGTGGGTTGAAAGT |
| | (SEQ ID NO: 134) |
| WP_066425713.1 (SEQ ID NO: 27) | GTTTCAACGACCATCCCGGCTAGGGGTGGGTTGAAAG |
| | (SEQ ID NO: 130) |

TABLE 8
Nucleotide sequences of Representative CLUST.004377 Transposon End
Internal Repeat Motifs

| CLUST.004377 Effector_A Protein | | |
|---|---|---|
| Accession | Left Tnp End Motif | Right Tnp End Motif |
| WP_017289534.1 (SEQ ID NO: 11) | TCTAACAGATTGACTGTCG | AATAACACTTTATTTGTCG |
| | (SEQ ID NO: 135) | (SEQ ID NO: 136) |
| ODH02164.1 (SEQ ID NO: 26) | ATTCGCAAATTCAATGTCG | TTTGGACAATAATTCTTTAAAACTGGTTT |
| | (SEQ ID NO: 137) | (SEQ ID NO: 138) |
| ACV01094.1 (SEQ ID NO: 22) | AATGATAGAATAGCTGTAG | GGCAGCAATACGGGATGCA |
| | (SEQ ID NO: 139) | (SEQ ID NO: 140) |
| WP_072619878.1 (SEQ ID NO: 19) | ATTGACTCATTAACTGACA | CTTGACAAATTATTTGTCA |
| | (SEQ ID NO: 141) | (SEQ ID NO: 142) |
| WP_066425713.1 (SEQ ID NO: 27) | ATTCGCAAATTAAATGTCG | GGTGCATCCGACTTGTAAGCG |
| | (SEQ ID NO: 143) | (SEQ ID NO: 144) |
| OBQ23770.1 (SEQ ID NO: 2) | AGTGACTAATTATATGTCA | GAGAGTACATCGTTGACATA |
| | (SEQ ID NO: 145) | (SEQ ID NO: 146) |
| WP_012411901.1 (SEQ ID NO: 13) | AGTGACTAATTATATGTCG | GAGAGCGCTACGTTGACATT |
| | (SEQ ID NO: 147) | (SEQ ID NO: 148) |
| WP_011318983.1 (SEQ ID NO: 4) | TTCGACAAATTAGCTGTCA | GTAGTAAATGCTTGTTAT |
| | (SEQ ID NO: 149) | (SEQ ID NO: 150) |
| WP_011318157.1 (SEQ ID NO: 3) | TTCGACAAATTAGCTGTCA | GTAGTAAATGCTTGTTAT |
| | (SEQ ID NO: 149) | (SEQ ID NO: 150) |
| BAQ63841.1 (SEQ ID NO: 10) | AGTAACAGATTATTTGTCG | AGTAACAAATTATTTGTCA |
| | (SEQ ID NO: 151) | (SEQ ID NO: 152) |
| AFZ58287.1 (SEQ ID NO: 24) | GTTCGCAAATTAAATGTCG | ATTCGCAAATTAAATGTCG |
| | (SEQ ID NO: 153) | (SEQ ID NO: 143) |
| BAQ60380.1 (SEQ ID NO: 20) | TAATCAACAATAAAACAT | AGTGCCACATTAATTGTCA |
| | (SEQ ID NO: 154) | (SEQ ID NO: 155) |
| WP_011318008.1 (SEQ ID NO: 5) | AGCGACAAATAATTTGTCG | GGCGCACTTCGTTCGGGAT |
| | (SEQ ID NO: 156) | (SEQ ID NO: 157) |
| AFZ00435.1 (SEQ ID NO: 23) | ATTCGCAAATTGAATGTCG | GGAGCATCAGACTCTTAATTT |
| | (SEQ ID NO: 158) | (SEQ ID NO: 159) |
| WP_029636312.1 (SEQ ID NO: 17) | AGTGACTAATTATATGTCG | GCAAGTCCTTTTATCCGCT |
| | (SEQ ID NO: 147) | (SEQ ID NO: 160) |
| AFZ56196.1 (SEQ ID NO: 25) | TTTAACAAATTAAGTGTCA | GATCAGTAGGTCGTGGGTTTAA |
| | (SEQ ID NO: 161) | (SEQ ID NO: 162) |
| BAB75312.1 (SEQ ID NO: 14) | AGTGACTAATTATTTGTCA | AATGACAATAATTTGTCACAAA |
| | (SEQ ID NO: 163) | (SEQ ID NO: 164) |
| WP_073629970.1 (SEQ ID NO: 18) | AATAACTAATTAAATGTCG | AATGACAAATTGTTTGTCG |
| | (SEQ ID NO: 165) | (SEQ ID NO: 166) |

TABLE 9
Non-coding Transposon End Sequences of Representative CLUST.004377 Systems

>Left Transposon End for OBQ23770.1
TGATATTTATTTGTTCTATCACATCTTCATAAATATCTGCCATTGTCAAAGTTAAGTTAACTGAATCTAAATGTAATTTATCAT
CTTTTCCTAATACTTCCGTTGACCAATTTCCTTGATCATCTTGACGACAAACCTCCACTTTTATTTGATTTTGCTAAACTAGAA
CATATTCCTTTAGACTGTCTATAGTTTGATAATTAATGCGTTTTTCACGTTTGTCTATTGTTGCTGTGGAATTAGATAAAACTT
CAGAAATGTTGAATGTCGTTACAAAACTTTACATATTTAAGTATAGTAGTATTATCAAGGGTAAAAATGTCAACATTTCATAAT
ATCTTCCATCCAATGAACATAACTGATTTGTTGACACATACAAGATTGAAGATAGCGGAATTTATCTAACATCTTTTCACCCCA
TTCTTTCGGAACAGATAAATGTACAGTGACTAATTATATGTCATCGTGACAAATTAATGTCATCTTATAAATCCTTGCTGTAGA
AGGATTTTAGCAATTTAACGATATTATACTTCAATCCAGTTAGTGACAAAATAAATGTCGTTTCCCATGATTGTGACAAATTAA
CTGTCGCGTTACCGATTAAAAAAAGTTTTTGTATATTTTCATAATGACAAATTGACTGTCGCTTTCCAGTAAGCTAGAATAACA
TCATGTTTTTATAAAATCTGTTTGATTT (SEQ ID NO: 167)
>Right Transposon End for OBQ23770.1
AGGTAAAATGGTGTGCGATCGCCCAACACAGAATTAGGTAACAACAACCCACCCTATGGTGGGTTGAAAGGTTATCATATTCTT
ATAAAAAGAGGGTTGAAAGAGAGTACATCGTTGACATATCGCTCTGATATATCAATAAATGACATTAATTTGTCACTACCATTA
AAACGACAACAATTTGTCCTAACGACACTAAATTGTCACCGACGACATATAATTAGTCACTGTACAAAAAGTATGGACGGTATT
GGACTCGAACCAACGACCCCATCGATGTCAACGATGTACTCTAACCACCTGAGCTAACCGTCCTTAATCCCATAGACTAATAAC
GTATCACATAAAATATAATTTGTCAATCCCTAAAATCCAAATTTTATTCTACATATATCTAGTCAACTGTACCCGATAGCGATC
CTTTTTTGTCACCGCAATTTCCCCAACTTCCAACCGACCTTTTCCCCGAATAGCGATTAAATCCCCTGTTTTCACTTGAG
(SEQ ID NO: 168)
>Left Transposon End for BAT55382.1
AAAAATGATGCAGTTATGAATTTTTCGCTTCTTTGTCTATAGCTTTACACAATTTCTTGGCTTGGGTGAATTTTTGAGCAACAA
CTTAGTTTTTCTGTGGTATCGGGGTGCAAATAAGTAATTCAAGCTCTAGTTAACTAACAAAACTCAACTACGCGAAGACGGAAA
AAACACAGAATAAGTAAGGATAAGTCTACAAGGTTATCTGTTCTTGCTTCGCCATCACCAAAAAAGACCGCCGCACCCCTTCAA
TTTCGCGTGGAGCCAACTGTAGTGCATATCGCCTAGTTGTTTCTGAAGTTTTGGACAATAATTCTTTAAAACTGGATTGATGGC
TGAAAGCTAGGAAATACGTAAATTATGCGTTTAGCACTGTCAAATGGACAATAATTCTTTAAAACTGACAATAATTATTTAAAG
TACATATTGTACATTCGCACATTATATGTCGCAATTTGCAAACAACGACATTAGACGACATTAGCATCTTGAGCATCTAAAACG
CTTATCGTATAAAGCTTTCAGGAAATTGTTAATTAAAAACGTTAAATTCCTGACTTCGCACATTGTATGTCGCTAACATTAAAG
CTCGCAAATTAATGTCGTTAATCTAAATTTTGTCACATTGCAAATTCAATGTCGCATTTTCTTCAGTTAATGGTACATTAATAC
TACCAATAACTACATTCTCCTCGCTTAA (SEQ ID NO: 169)
>Right Transposon End for BAT55382.1
AGTAAGAAGAATAGAAGTAATTTCCTGAAATCAACTATATTTGGACATATTTTAGGAATATAACTAAATTATGGGAGGGTTGAA
AGGAGCGCTGCGATCAAACAAATGTTAAGGTAAAATAATTCGTCTGTAGCAAATACCACAGTTAATGAGTAAGCCATACGACGT
TAATTTGCGAAAAACTAATATGATTGAATTAACGACGCGAATTAGCGAAAGTAATGTTAATTACCTAAAAACGACATCAATTTG
CGAAAAGCGACAAATAATGTGCGAATGTACACATATGGAGAATAGGGAACTCGAATCCCTGACCTCTGCGGTGCGATCGCAGCG
CTCTACCAACTGAGCTAATTCCCCTGACTTTGTTGAGTGTTCAGTTAATCACACTCGTCCATCATAGAGTATTTTAACATTCAG
TTAGGGATGGCTGAGATTTGTTGCCAGAAAAAACTTCTTGTACTCGCTCTAGTTCTAATTCGCTCAGATAATCCACAGTC
(SEQ ID NO: 170)
>Left Transposon End for WP_029636312.1
GTGTTAATTTACTTACCTGAGATTAACAAACATTCTTTGAGAATAATCACAAAAAATGTAAAGACTAAATAAAAGCAGTCACCT
ATAAGTAAATGACCTATGGGCGTAACCCAATAATGTCTTCAAGCACAAGTTCCAACTGGTAACTTGCAGTATTAATACTTAAAT
ATTTTTTTGTATTCAAAAAAATATAAACTAAACTTAATAAAAATTAACTTTACTTATTGTTAACAATAATGAAGGAATTCTTGA
TAGATGTTTTTATTGTTTTTTGAGATTTAAAATGCGATCGCTCGTAATTATATTTGATATAAAGTATAATTTGACACAGTAACA
AAAATCTACCTACCCTCGTAGAGACGTTCCGCCGGAACGTCTTTACATTTATTATGTGTTGCCCAAAACCATTGTAGAGAGGTA
GCATTGCTACGTCTCTACGTGTACAGTGACTAATTATATGTCGTTGTGACAAATTATTGTCATCAGTAAAATCCTTATACAGTA
TAGATTATAGCGCTTTGGCAGTTTTAGCATAACCTCTTTGCAGTGACAAAATAGATGTCGTTGTCCGTGATTGTGACAAATTAG
CTGTCGCTTTGCAAGATAGGAAAAAGCTTTTGTGTATTTTCATAATGACAAATTGACTGTCGCTTTGTATATAGATAGAATAAC
AATATGTTTTTATAAAAAAGTTTTATAT (SEQ ID NO: 171)
>Right Transposon End for WP_029636312.1
AATCTTTTAAACCGAGATTAGTTTTGGGTTTCAGTAACTATTAAACTTAGGGGTGGGTTGAAAGCAAGTCCTTTTATCCGCTTG
TTTTAATTGCTTTGTATAATAATTGCAGAGCATATTATATTGATGACATTTAATTTGTCATCAATTAATTAAGCAACGCTGATG
GGTCACGACGACAATTAAATAGTCACAATGACATTAATCTGTCACCGACGACAGATAATTTGTCACTGTACACTACGCCTTTTG
TGGAGATGTCTAATAAACTAAAAATTTTTGATAGTGCGGATAAAAGGACTTGAACCTTCACTCCTTTCGGAACTAGAACCTAAA
TCTAGCGCGTCTGCCAATTTCGCCATATCCGCATCAGTTTCCTATATTACCATAACGAATTATCTGTGGCAATAGGGATTCGGG
ACTGGGACAAGGAGGAATTCCCAATGCCCCATGCCCCATGCCCAATGCCCCATGCCCAATGCCCCATGCCCCATGCCCAA
(SEQ ID NO: 172)
>Left Transposon End for WP_073629970.1
ATAAAATACCCAAAATCTTATTGATCTCCTTAATATAATAATTATTCAAATCAATAAACACATCACAGCCTTCTAACTTCAAAA
ACTTCCAAATATCCCCCGTTGTTACTGCACCATAAATAGTTGTCGAATAACTAATTAAATGTCGTCTTAACAAATTAATGTCGC
TCAAAACGTAAAGGCTATAATCGTTACTAAGCAAGGATTATAGCCTTTTTGATTTCTATTTAGCATTCCTAAATATTTAACTAA
TTAAACGTCGTAATTTGTAAATATAACAAAATAAGTGTCGTTTTTTCAAAAAATCTCTTTCCAAAGTTTTTAACGGCTCATAAC
AAAATAAGTGTCGTCTTTTGGAAGTGAGTAAAAAATCTAAAATTACGTGTCGCTTTTTGGAATAAAGTAGTAGTATATTTACTA
GGTAATAGTAATTATGTACGAATAAGGTGTTACGCATTTAATTTTTTTGCCCAAAAGCCTTGCAGCAAGGACTTTAGGCTGCAA
AATATTACATTTAGATGCGCGTCAGCTTACAAACATAGTTGTACTCAAGTCTTCTTATTTCTCTCTACTTGCAAAGTCATTCCT
CATATTGCGTTAAAGGTTGATGTGAGTTGAGGCTGTATACTTTGCTTTTTCGCTCTTAGACTTGCTGTACCGTATTGGCTAACC
AACTAGTTTCAAGCGATGAAGTTTGTTT (SEQ ID NO: 173)
>Right Transposon End for WP_073629970.1
GTCAAGTTTGCTTGTGAGGGTTGAAAGGAATGTCACCTTCCCAATACTAGAAGAGTGTCAAAAGCTATGCTGGCTAATGGAAGG
TTGAAATTAATCGCTATTTTTTTGCAAAGAAAAGATTCTACCAGTTATAAAAAAGCAAGAGTGACAAACAATTAGTCGTTCAAA
CAAATGACAAATTGTTTGTCGCTCTAAAGTATCCTAGATTACTTGACTATACGAGTTTATTTTTTAGGGATTAAGCTCGGGGAA
AAGTTTGTGATCTACTGACATTCAAGTAACACTGACAAATAATTTGTCACTGTACAATACTCAATAAGAGTCGGCTACATTAAA
TGACTATAAGACAAATAATTTGTCGCTTTACCACTTTTGACAAATAATTTGTCGCCACGGTCAAATAATTTGTCGCTCTACATT
TGAAAGCGGGCGATGGGACTCGAACCCACGACGTTCACCTTGGGAAGGTGACATTCTACCACTGAATTACACCCGCAAAT
(SEQ ID NO: 174)
>Left Transposon End for WP_011318157.1
GTGTTATGCAGCAATATGAGTAGCCGTCATGAACTGCGTACACCAAAACCTATGAAAAAGGCATCAAAGGATTTTCAAATCAGA
ACAACTGGAAAAAAACAACTATGTTAAATATTGTAAAAACAATAAGATTGAGTTCGTAGTAAGGACTTTAATCCTTGTAAAGTT
CTCACTACAAGTCTTTAATTATCCACCCTGTTCTACTCAGTTCATTTAAGGCTAACAACTTAGACGCGGTGGTAAGTTGAAGCC
ATCTCTCCACTGAACCATATTGATATATAATGTAGAGCGACACATTATTTGACCACTTCGACAAATTAGCTGTCACAGCATTAT
CTTGAACGAAAATCCATATATATCAATACTTTCTAGCAATGAAAGCTAAATCAAGCTCATTCAGTCTTGGACGACAAATTACAT
GACTTTTTTAAAAGTCCGCGACTTATTAGATGACAATTATTAATTTTGCGAAAAATTACGTGTCAATTATTCGCAGTTTCATAG
TAGTATAAATGTACCCAATAATCAAAGACAAATCACATCTATGAATTATTTAGATGTAGATAACCAGTTTGAACTGGAAGATGA
AGATGACTTTCTTTTGGATGATGAGGATGCAGACATACTCGATTTTTCAATTGAAGTTGAGTCATTGAGTAATGATGGAAACTC
TGTAGCAGAAGACAAATTAGTAGAGTTT (SEQ ID NO: 175)
>Right Transposon End for WP_011318157.1
AAGTTACAGTTATTAGGTGGGTTGAAAGTAGTAAATGCTTGTTATTTTAGGTTTAGACCTCAAAAAAATTACAGATATAACTTC
GAGAGCTAATGTAACCAAAAATAATACTTAGCCCAGTGGAGTGCTACTATGTAACTGCATAGAGGAAAAGACGATGACAAATAA
TTTGTCGCTATGATATTTATGACAAATATTTTGTCGCTACAGACAAATAATATGTCGCTCTACATATAACCAACAAAAAAGGCT
TGGTCGGGCCAAGCCTTTATGTATACCAAGCATTTACTACACCAAGCTTAATTTAAACCCTCTAGCACTTTAGTTCATCTGTCT
TTAGACTGTATGCAAATTCTTTCAAACCTGGTATGAACTGGCATATTTACATACAAAAAAGGCTTGGTTCAACACCAAACCTCG
ATAATTGCTGTGCTTAACAACGTACTAAATTAATTTAAACTGGATTGTTGTCCTTATCATCTTCCTTTAGTATGTGTTGC
(SEQ ID NO: 176)
>Left Transposon End for WP_072619878.1
TTCGGGAGTTTCTGCAAATCCTGGAGTGGGTGGCGACGAAGAAGGGGAAGCGGGTGGTCTATGTTGAGCGCTGGTTCCCTTCGA
GCAAGACCTGTTCAAGTTGTGGTCATATTTTGGAGCATCTGGATTTAGAGACTCGCCATTGGCGGTGTCCCAGTTGCTCGACAG
AGAATGACCGGGATGAGAATGCGGCGATGAATATTAAAGTGGCTGGGGCTTCAGCCATTGGGTTAGGTGATGTCAGACAGGCGT
TGCCTGCTATTGCTGTTTGATCCCAGAATCCCCCGTTTTCTAAACGGGGGAGTAAGTCAAAGGGTTCTTAACGTTGTACAGTCC
CTCGTTGGCGTGGTCGATCAGTTCTGAACTGTCTGCCCCCACACTGCCCCCAAAATCTTGATTTTAATGACATTTTTTTTCCAA
TTTTAGGGTGTGGGTGTCGATTGACTCATTAACTGACATCTTGACAAATTATTTGTCATTGCTATAGACAAGCCTGCAACCCTT
ACTATACATAAGGTTGTGGGCTTTTTGCATTGGAAATTTACTCAAAAGCAAAATTGACAAATTAGGTGTCGTATATTAGGAGTT
TGCCAAAATATGTGTCGCTTTTCATTTAGTCCAGATTTTCTGGCTTTTTGACACATTCTGTGTCGCTCAGGGTAAAATAACAAC
ATGTTTGTATTAAACACATTTGTTGTAA (SEQ ID NO: 177)
>Right Transposon End for WP_072619878.1
GTTTGCCTCGGAGATGTTTGCCCTAGTGAGATTGGTTGCTGAAACCATCCCAAGGTGGGATTAGGTTGAAAGAGTAATACAAGA
ATTGGGTGGATTGAAAGCAAGTCCCACCGTCCCCTACTTTAGTGCAATGCAAGATTACACAACAAACTTGCTCTGAATGTATGG
AAGGTTCGTAAAAGTGGTGACAAATAATTTGTCAAGATGACATCAATTTGACAACGATGACAAATAATTAGTCAATCGACATGT
GGGCAACACCGTCAGAACAACTCAGAAAACTCTGAAACGATGGGGGTGGTGGGACTTGAACCCACACGTCTTTTTACGGACAAC
GGATTTTAAGTCCGCAGCGTCTACCATTCCGCCACACCCCCAAGAGCAGTGACAGGGTTCTAGTTTAGCAGCAAATGGCGGCCA
CTGCATGGAACAGAACCCTCAGAAAATCTAATCAATCTGCCTCTTGCGCTCCGATGGGTTGAATTGTTTATGATGGGAGG
(SEQ ID NO: 178)
>Left Transposon End for WP_011318983.1
AACAGATTAAAAATTTAATAATTACCGTAATCTTTAGCTAACTATAATTATTTACAACTATGCAGACACAATAACCGCAATAAA
GGTTCCTTTTCCTCAAAAAAGAGCTTAGTAAAAATTGCCTTATATAGCAGATGTTATTTAAAATACCTGTGTGTGTTATGCAGC
AATATGAGTAGCCGTCATGAACTGCGTACACCAAAACCTATGAAAAAGGCATCAAAGGATTTTCAAATCAGAACAACTGGAAAA
AAACAACTATGTTAAATATTGTAAAAACAATAAGATTGAGTTCGTAGTAAGGACTTTAATCCTTGTAAAGTTCTCACTACAAGT
CTTTAATTATCCACCCTGTTCTACTCAGTTCATTTAAGGCTAACAACTTAGACGCGGTGGTAAGTTGAAGCCATCTCTCCACTG
AACCATATTGATATATAATGTAGAGCGACACATTATTTGACCACTTCGACAAATTAGCTGTCACAGCATTATCTTGAACGAAAA
TCCATATATATCAATACTTTCTAGCAATGAAAGCTAAATCAAGCTCATTCAGTCTTGGACGACAAATTACATGACTTTTTTAAA
AGTCCGCGACTTATTAGATGACAATTATTAATTTTGCGAAAAATTACGTGTCAATTATTCGCAGTTTCATAGTAGTATAAATGT
ACCCAATAATCAAAGACAAATCACATCT (SEQ ID NO: 179)
>Right Transposon End for WP_011318983.1
AAGTTACAGTTATTAGGTGGGTTGAAAGTAGTAAATGCTTGTTATTTTAGGTTTAGACCTCAAAAAAATTACAGATATAACTTC
GAGAGCTAATGTAACCAAAAATAATACTTAGCCCAGTGGAGTGCTACTATGTAACTGCATAGAGGAAAAGACGATGACAAATAA
TTTGTCGCTATGATATTTATGACAAATATTTTGTCGCTACAGACAAATAATATGTCGCTCTACATATAACCAACAAAAAAGGCT
TGGTCGGGCCAAGCCTTTATGTATACCAAGCATTTACTACACCAAGCTTAATTTAAACCCTCTAGCACTTTAGTTCATCTGTCT
TTAGACTGTATGCAAATTCTTTCAAACCTGGTATGAACTGGCATATTTACATACAAAAAAGGCTTGGTTCAACACCAAACCTCG
ATAATTGCTGTGCTTAACAACGTACTAAATTAATTTAAACTGGATTGTTGTCCTTATCATCTTCCTTTAGTATGTGTTGC
(SEQ ID NO: 176)
>Left Transposon End for AFZ58287.1
ACTGCGCCAGCAATTCACCAAACTGCCGCAATTTAATCAAACAGGTGTTGGGATCATCTGCAAAATATCGCTCGGCTAGAGTCG
CTAATCGAACCAGTTGTATGTCGTGGACTGCAAGGAATGAGAAGTTTAATGACTCTGGCATACTGGCTTTTGAGAGAATCTGTC
CAGTTAGTTTCAAGTTGTCTCAAATAACCTAATCAAGACTCCTCTTGTTGTAGGTTGCCCAAATTAACTTAGTCATAGTCTAGC
AGTAATTCAAGTCTCATAATATACATCTAACTAAAAATTTGGAAAATATTATTAATCTGTGAATATATAGCAATTCCCAAGCTC
ATGAAATACACCCCACCCGTGCTGTCGCGCACCGTAGCCTTACCAAGGGGAGGATTGGGGAGGGGTAATTTTGTATCTAACTAG
AGTGAGAAAGGCTATTGTACGTTCGCAAATTAAATGTCGCTTTTCGCAAATTAGTGTCGCAACTGCTTTTAAGGACTAAAACCT
TTATTCTATAAGCATCATAAGCTATTTACCCCTACAAAAGACTAAGTTACCTAATTCGCATATTGTATGTCGTAAAACCTGGTT
TCGCAAATTAAATGTCGTTTGTTAAGAATTTGGTATTTTGCAAATTAGATGTCGCATTTTTGAGTAATTCATGGTACATTAGTA
CCTTAATTATTCATGAGTGGGTTTCACT (SEQ ID NO: 180)
>Right Transposon End for AFZ58287.1
ATAACTAGAATTTACTTGCGTTAACCATTAAAATAAAATGGTTGCAAAATAGAAATGATCATGGGAGGGTTGAAAGGAGTGCTG
CGATCGAACACATATCAATAATCCAATAATGCCCTTGCAGCAATAATTTATTGTATAGTAAATGTTAAGTTCATGCGACATTAA
TTTGCGAAAACTTAGAATAATTAAATTGACTCTGAAAAACAACCACCACGACATTAATTTGCGAATAACGACACTAAATTGCGA
AAAGCGACATTTAATTTGCGAATGTACATATATAATGGAGAATAGGAGACTCGAACCCCTGACCTCTGCGGTGCGATCGCAGCA
CTCTACCAACTGAGCTAATTCCCCTTGCTAGTCTCAAGTTACTAACTCAGACAGCCGTACTATATGTTAACACTCAGGCAGGGT
AGTTTTCACCTCTTTTTGCAAAAAAACTTCCTGTACCCGGTCTAATTCCAAATCAGTCAGATAATCTACTGTCCAGTTAG
(SEQ ID NO: 181)
>Left Transposon End for WP_066116114.1
TCTCATCCAAAAACCTTAAAGATAGTTCAAGAAAAAATAGAAAATGAGTGGGAGATAAAAATAAGCAAAGATACGATAAAAAGA
TTAGCAAAAAAAAAGGGCATGGGGTGGTATCGGTTCAAGAAAAGGGTAAAAGGGGAAGTATGCCCTGAATTATACCAACAAAAA
AAGAGACAATTAGAGCAACTAAAAAGAGCAGAAAATGAAGGAAAAATAGATATATATTATGGTGATGAAAGTGGATTTAGTTTA
GTACCTTGTTTACCTTATGGATGGCAAGAAAAAGGGAGAAAAATAGAAAGAGAAAGTAGCCTAAGTAAAAGATTAAATGTGTTA
GGATTTATGAAAAAAAATAATGAGTTAGAAAGCTATGTATTTGAGTCATCAATCAATAGTGATGTCGTGATAGCTTGTATAGAT
AACTTGAGTAAAAAAATAAAGAAGGAAACAGTATTAGTAATGGATAATGCCTCGATTCACCAAAATAAAAAATTCTGGAATAAA
GAAGAAGAATGGGAAAAAAAAGGATTAAAAATATTCTTTCTACCACCCTATTCACCACAATTAAACAAAATAGAAATACTGTGG
AGATTTATGAAATATAAATGGTTAGAGAACTCCTGTTATAAAAGTTATTTAGATTTAGTAAAAGGAGTAGAAAATATTCTTATA
AACTTTGGTTCAAAATATACAATTAATT (SEQ ID NO: 182)
>Right Transposon End for WP_066116114.1
GATCATTATAAGACTGCAAAATACAAAAATAGCTATAATAGATATATATGTGGATTGAAAGAGGTCACGAGTTCAATCATAAAA
TGACATTTAATTTGTTAACGATGATTCAGCGACAGGAATTTGTTAAGACGACAATTAATTCGTTAAGATGACAGATAAAATGTT
ATTCGACAATACCTGTAATTTACCATTGCAAGGACATTCACACGAAAGCCTTTGACGAGGATTGAACTCGTGACCTCACCCTTA
CCAAGGGTGTGCTCTACCACTGAGCTACAAAGGCAGTGGGCCGAGCTGGATTTGAACCAGCGTAGGCATAGCCAGCGGATTTAC
AGTCCGCCCCCATTAACCACTCGGGCATCGACCCATTTGTTTTCACATTTACTAATTATAGCTCATCTTTTTTTTTAATGCCAA
CTTTTTTGTTTAATTTTTATCCAGTTTGCCCAATGCGAGGTTCTGTTCCTGCTATAATTCGATCGATATTAGTACGATGA
(SEQ ID NO: 183)
>Left Transposon End for WP_036484397.1
ACTATTTGAAACAGCCAATTACTTTTATCTTCATAAGCTACGGTTGAATCTATAACTTCTAAAAACTCCGTGTCGTAGAAATAG
AGAAAATCATCAATTAACTTTTCTTGCTTTACTCTCCCCGTAGTGTTGGTATAACCTCTTTCAATAAAAAATTTAATTGCCGAG
CGAAGCTATTATTGCCAACATAGAAATTACAGCAGCAATACCAATGACAATCAAAGCGAAAATCGCCAAGCCATATTGCAGCGT
GTATAGCTTGCGTAATTGGCTCATTGCGTTCATTAGGTTATCAATATCGTTACCTGAAGTTTTGACAATGCGCTGAAAGCCGCC
AGCCGCTAGTACATTCCAGCGTCCTATCAACAGAAGTAGAATTCCCTGAATGATGCCACTAATTCCACTATTAATAGGAACCTC
TTCTTTTTGAATAAATATGTACGGTGACTAATTATTTGACATAATGACAAATTGTTGTCATGCAATTCAAAGCTTTATATGCTA
GGCATCATAGCATTTTTATTATAATATTTGATTGACTTATATAATGACAAACAAAATGTCGTTTGTTTGCAAAATGACTAATTA
GCTGTCGTTTATTGAAAATTCAGTTTTAAAGAAAAGTGACAAGATCTGTGTCACTTTTTTATTTAAATATAGAATTACATTATG
TGTTTTTTAAAAAAACTAATTTACTAAA (SEQ ID NO: 184)
>Right Transposon End for WP_036484397.1
CGTAATTCTTAGGAGTGGGTGCAAAATTTAACCCCAGGTTTCAATGACCATTTTGTGATGGCTTGGGTTGAAAGATAAAGGCTT
AGTCATTGTTATTGATGAAGCGGAAGTGCCAACGACCACACGCAAATTTTAATTCTCAATAACTATCGTCTAATTCTCGCTAAG
AAACTGAAAGTAATTTTAAAGTCTTGTTCTTTGGTTACTCCAAGTAATTTATTACTTTTAGGCGATCGCCTTGCTTGCCGAGTT
TAATTTGTATCAGCAATTGCTGTATACAGTCTAAAACCAAGTAAATTCGATTAATTATTTTTTGTGCGACGCAGTAAGTCGCTT
GATTTAAGTGAAAATTTCATGACTGAGGTTATGGATTTCTAGTAGATTTCATCCTACTATTCCCACGTAAAGGAGTCTCGTACT
CTGGTCACATTGAAAGTAAATAATGAACTCAATGGAATGCAGACCATGAATGTATCCGAAGGTGGATAACCTCTATCCAG
(SEQ ID NO: 185)
>Left Transposon End for WP_066425713.1
CGAGGACAATACCGGTATATTTCAGCAAGTCTGCACTCAAAATACTGGGGTTGGGCTGTCGAAGCGCGATCGCTGTCAAAAGTT
CTGGAATTTCACAATTCTGCTTCCTTTTATTTTCTTGTATTTATAAGCACATAAGTAAAAATAAGTATTCTATGTATACCTAGT
GATATAAGAATACACATATTTAACAAGATATATGAATAAAATTTTGATAAATTTTCTGTAAAAACAGTGAATATACTTAATTTT
TAATTAAATTTTCCAAATTCAGGACTTTCAAGATATTGACATTTCTGTGGTTAGTATTATACCGTCCCAATATTCAGATTCCAA
AACATTAGTTTCTCTTCCATTCGCAAATTGAGGAAGCACAGACGGCACAAACCGATAATTATCAGAGTCAAAAATCGACAAAAG
GATGATTGAAAAGTGTACATTCGCAAATTAAATGTCGCTTTTCGCAAACAATGTCGCAACTGTATTGAATGGCTGAAAGCTACA
TCGTGTAAATATTATAGGTCATTTATCTCTAAGACAGGCTAATTTGCTTGTTTCGCATATTATATGTCGCATAATCACGTTTTT
GCAAATTAAATGTCGTTTGTTAAAGTTGGTGACTTTTGCAAATTAAATGTCGTATTCTTTATGAATATATGGTACATTAGTACC
TTAATTACTCATGAGTGGGTTTCACTCT (SEQ ID NO: 186)
>Right Transposon End for WP_066425713.1
ACTTAGATAATCAATCCTAGAAAAAACGCTCTTAGTTTATCCGTCTTTGCGCTACAAAAATCTTGTCAAATGGGGTAGGGTTGA
AAGGTGCATCCGACTTGTAAGCGTATTAAACCTTAAAAACATAAAACAAAAAATTAAAACTTATTTAATTCTGATTTTTTATAT
GTAAGAATTAGGTTCTGCACAACCATCAATTCACTTGTTGAAGTAATATTATTGTGTTAACACTTGCTTGTAAGCAACACTGTA
ACAATCTAGAAGGAAAAAACGACATTAATGTGCGAAAACCTAGCATAACTAAATTGATTATAAAAAATAGCCGAAGCGACATCA
ATTTGAGAAAACCGACACTAAATTGCGAAAAGCGACATTTAATTTGCGAATGTACAGATTGAAAAGCCAGCAGTCGGATTTGAA
CCGACGACCTTCCGATTACAAGTCGGATGCACTACCACTGTGCTATGCTGGCAAATTAGGTGATGCGTTTGAATCATTCA
(SEQ ID NO: 187)
>Left Transposon End for ACV01094.1
TTTCTAGTAAGGTTTCTAGCGCGATAGAACAGCCGTGGATATCACTGATGGCTAGTGTTCTCATAGCTTTGCTTTTGTTTTTCT
ATGATGCCTTTGATCCTATCTTGACTAGGTTAAGGTTTATTTAAGACAGAGAAACTATAGTAAAATTTTTAAGGCTAAGGTAAA
TCTTTGAACGACTGCTTCACCGCTTAAAATGGCATCCTCTCGATATTGAGTCATCGAACCATCTTGGCGATAAACAAACGCTTG
ACGATTAATGCGATCGATTAACCATCCTAATTTAACCCCATTGTTTCTATATTCTTGCATTTTTTGTTTAAGTTTACTTAAACT
ATCAGATTTAGAACGAATTTCAATAACAAAATCTGGCGCAAGATTGAAAAATTCATCCTCTTCCTCATCCCATCCTTGAGGTAA
TCTTTCCTTAGCAATTGTACAGTGACACATTATTTGACATGATGACAAATTAGTGTCATTCTTTAAACCCTTTACTCAATAAGG
CCTACAGCTATTCTATCATTATTTATAATCGTTCAGCTAAGTGACAAATTAAGCGTCGCTTTTTGCTTTTATGACAAATTAATT
GTCGTTTTTTCATAAAGCTTCAAAAATAATTGTGACAAATTCAATGTCACTTTTTCTATAAATGTCTGCTAAAATAACATTATG
GTTTTTAAAATAGTGTTCTAATTATGAG (SEQ ID NO: 188)
>Right Transposon End for ACV01094.1
ATGATTATAAAAATATTAGTATGGATTGAAAGGCAGCAATACGGGATGCAAAATGATGACATTTAATTTGTCACTAAAGCTTAA
ACGACAACAATTGGTCACAACGACAATTAAATTGTCTCCATGACACTAATCTGTCATCGATGACAAATAATCTGTCACTGTACA
ATAGTTCGTCAAAATGTAAAGGCCCGGAGTCTAAGCCTATCACAAGCATCCCGTATTGCTGCTACCTTCCAGTCCTGACAAGAT
TTGGGCGTTGTAATCGCATAGGTCCGAGCCAATTACTAGGATAACACAGAACTGAGGCACTCACTCTTGATCGTTGTTCATAAT
TTGAGGTACGCGGAAAAAGTCGCCCTCTTGTTCTGGGGCACTTTTAAGCAATGATTCGCGATCGCCATAGGTTTCTGTGCGATC
GCTGCGGGTAATATTGCTCAATTCGATCGCGCGTGTCATTGGGGGGACATTTTCGGTGTCAAGTTCGCTTAACTGATCGAAATA
TTGCAAAATGCTATTTAATTGGGGGGCAAACGCTTCTTCTTCGGCTGGTGTAATCTCCAGTCGGGCTAAATGGGCGATTTTTTC
AACTTGTTGGCGATCAATCATCGATGGTTTCTCACTCTGATTTAAAAGAAGACATCCATTTCAGAACGTCCAGTCACCTTGAGC
CAATTTTGGGCTTCAATATAGTTATTGG (SEQ ID NO: 189)
>Left Transposon End for ODH02164.1
GAACCAGCTTCCGTAGGCAAAGATTATTCAGACGAAGCAGTATTAAGGGATTTTTTCTTTCATGATTGTCAACCGGATGTAGCA
CAGTGGGCAATCTCGAAAAGTCGTCCGCAACAATCTATGGCATATATATTTGAAACGAATCCTCTAAAAGCTTTGCCGCATGTT
GAGCGTAAATATATTGTTTGTAGCAACGATCGCATAATATCTCCTACATGGTCGCGCTACGCTGCACGCAAGCGCTTAGGAGTC
GATGCCATTGAGCTACCTTCTGGACATTGCCCACATCTGTCTCGTCCTGACCTTCTTGCCTCAATATTAACTAGTAATTAATTT
TTTGATCCATATTTTAGGAAATTAACCGAAGCGTATTGAGTGTGAACAAGGGAATATCTTTGAAATTTTAATTCGGCATAGACG
AACGATATTATCTAATTAATAACGACATGAATTTGCAAATTGCGACATTGAATTTGCGAATGTACACATGGTGTCCATTAAATA
ATTATTGTCACTTTTAAAGAATTATTGTCCAGTTTTACGATATCTGTATAACAGGCTCAAAGGCTTACCAAACAAGTCTTTGAG
CTATATTTATACTTTTATTATTCTCTAGCCAGTTTTAAAGTAAATGATGTCATTTTTCTGATAAATTTTTAAAGTAAATGATGT
CATTTTTCTTGGAAGACGGGTCAAGTGT (SEQ ID NO: 190)
>Right Transposon End for ODH02164.1
TATGATCCCCGAAGTAGGCGCTTTGGTTATATAATTTTGTATTAGGGTGGTTTGAAAGGAGTGCTGCGATCGCACGTAAAACGC
AGCTATGTAGCAGTGGAGCCTCTGCTTGGGTGAAAACAGCAAAACAAGAGGTAGTATATTTTTGTAATTAGACTGCAAATAAAG
GGCTTTAAAGCTACTCTTGTAATTCAATAACCTTGAGCCTGCTGTAATAAATTAAATTAGTAACCATATATTTAGCCGAGTTTT
ACAGGCTGATTTTGGACAATAATTCTTTAAAACTGGTTTCAACTCTGGACAATAATTCTTTAAAACTGATTTAATTGCTGAAAG
CCGCGAAAATACGTAACTTATGCCCCTGAACTTGGAAAAAATGGACATTAATTCTTTAAAAGTGACAATAATTATTTAATGGAC
ACATGGAGAATAACGGATTTGAACCGATGGCCTCTGCGGTGCGATCGCAGCGCTCTACCAACTGAGCTAATTCCCCTTGA
(SEQ ID NO: 191)
>Left Transposon End for BAQ63841.1
GTTTTCTCATCCTAATCAACGGGGCTAAATATACTATATTATAATATCTATAACTAAAGTGTGGGTACAATAAATAAGCTAAAG
TCCATTTAAGTTTAATACTAACTGACGATAAGCCGTCTTGAATTTTTGTAACTCATTCATACCTTCTTGAAGAGTTTTAATATA
TGAGAGTCTATGATTAATGTTAAATAAGAATGCCACTTAGCCACTTAGTTTTTGATCTAATTTCAATTAAAGGTAATTTGAGAG
AAAAACACACACTATCAGAAAGTGGACGAGAAGCTCTATTTTGCTATCCTTGTTTTAATTTTGGCAGTAATTGTTTGACTACTT
ATTAAAATAAAATTGATGGAAAAGTTAAAAATCAGTCTGAAAATGCTTTTGAATAACTTTGATAAAAAATCTTGTACAGTAACA
GATTATTTGTCGTGATAACAAATTCGTGTCATCTAAAAATAAGACTCTTAAAATCCTTATATATCAACAATTATAACTTATCCT
TACCCTAAAATCACACTCGATTGTATCTTAAAATAGATTAACAAATTAAGTGTCATTTTCCCTAAAAATAACAAATTAGATGTC
GCCTTCGGAAAAGGCGATTTTTTTTGTTTTTGAGTGGCAATTCACAGATTAAATGTCATAGAATAATAAGTACACATCAACTTT
GATTTTATTAAACATAAAAATATCAGCT (SEQ ID NO: 192)
>Right Transposon End for BAQ63841.1
GCTTTTATTTTGAACTAAAACTCTACTAATTAGAATCTGTCATCGAGACGAAACAAAACGATCATATTTTCTTTTTAAAGTTAT
AATTTCAATAGTGCCGTAGATCAAGTTTTAACAACCTCTGTTCTATGAAAAATGAGGAGTAGTTTACTTTTTACAAGAAGTTTG
CTTTCGGCTCCTGCTAACTACTTGCCCTGATGCTGTCTATCTTAGGATAGAGAAACTAGGCGCACTCCCAGCAATAAGGGTGCA
GGTGTACTGCTATAGCGGTTAGCAAATCACTTTCGATCGAGGAAGAATTCTCTTTGAGAATTGAAAGCGAGTCCTGTCGCACCC
AATCATTTAGACGACATTAATCTGTTATCACGTCATCTAATTTGTTAAGACGACACTAATCTGTTACCGATGACAAATAATTTG
TTACTGTACATCAATTTAAAATACGAGCCAACGGCTAAGTAATACACGGGTGCGACAGGACTCGAACCTGTGACCGACTG
(SEQ ID NO: 193)
>Left Transposon End for WP_012596241.1
TCATATTCCTGTTGATAAGACAGGCGTTCTCTTGCTGCTGACTCCCCTTGAGATTGCTTTAAATCGTCAATTAACGCCTTTAAA
ACCTTTTTAATCACCGTAGCCGTCACATTTTCATCCTCTTCCGGCTCATAACTCGCCACCTCTTGCGCTGACTCCACCGCTTCA
CTTAACTCCCCTTGCGCTGCACTAATTTTACTCTCTAACTCTTCAATTTGCGCCTCCTCAGCTTGAAAAAAGGCAGTTAACAGA
TATTCATCAGGAATAAGGGTGTGATGCCAACCTGTATTAATAATCGTCTTCAAATCATAGCGAATCTGTTGCCACCAGTTAACA
AACACCCCCGCAGACTTAAACTCATCCAACACCCCCAAAGGAATAAGCTGCTGTTTCAGACTGTCTAGTAACTCTTGCCGGTGT
ACATTCACTAATTAAGTGTCGCAATTTGACAGATTAATGTCGCAAAATAAATAAGCTGTAAACCTTACACGACAAGCATTCTAG
CATTTTTCAGGGACTGTATACAAAAATGCTTGATTTAACGGATAATCTGTCGTAAATTAGATTCTTAATGTTTTATCTGTCGTT
TCTTGGATAGATAGTCAAATTGCTGGTCAACATTTTTTCATACATTAATTGTCGTTTCTTGGAAAAGAGTCTATACTATGGTTA
GTTCGTTTGTTGGATTCTATGCTAACTC (SEQ ID NO: 194)
>Right Transposon End for WP_012596241.1
TTGGGATGGGTTGGATTCATGATCATGTTCAAACATCGAGAAGGATTGAAAGGCGCACTTCGTTCGGGATCGTAGACTTATAAA
AATATTCGATCCCAAAATAGCGACACTTAATGTCTGAACAATTGTCCATTAAATAATTATTGTCACAATTAAAAAATTAATGTC
ACTTTTTTGAGAGAGTTTTAAGATACCGCAAGGTTCTGTTAAATAAAGCTTTCAGCGATTGAAAAGAGTTATATCCTGTCAATT
GTCATTTTAAACTAAAATGTGTCACTTATTAAAAAGCCTTGCTAGAATCTTTAAAGTAAAATATGTCA
(SEQ ID NO: 195)
>Left Transposon End for BAQ60380.1
TTATCAGCTAATTCTAAACGAATACCATCAGGAGTTTTAATTTTTTTGCCTTGAGAAGGTTGCCATATCTCCTGCAACCAATCA
CAAATAAGTTGAGAATTAGGGTTAGGAATTTCTAAATATAAAATTTCTTTTAGTTTCATTTATATAAGTACTTGATAAAAATAT
TTAGTTATTAATGAGATGATTTGATTTATTATCTAATAATAGGAGTCTCTTCGCCAAGAGAACAATATCTGAAGATAAACAGCC
AGGATTAAGAAATAGCTGATCCAGAAATGATCCAGTTTTCTAATTTTGAATGAAAACAAAAATATGAGAATAGCAAATATTAAT
AAATAATAAATGATCTAGCAATTATCCAGTTTTTTAATCAACAATAAAACATCAAAGAAAATAATCAACAATAAAACCTTAAAG
TGTAGAGATGGATGATGTACAGTGCCAAATTAAATGACATGAAGCCAAATTAGTGTCGTTGGGTTTAATCCTTATTATCTCTAC
ATTATAGTCTTTTTCTCTAATTTACGAAGCTAATTTTAAATGACAAATTCAATGTCGTCTTTATATTTAGTGACAGATTAAATG
GCATCTTTTCAAATATTCTTTTTTTTTGACGGTGACAAATTCATCGTCATTTTTTATTTACTGTGTTAAGATAACATTATGTTT
TTATTTAACAATTATACTATTTAGAGTG (SEQ ID NO: 196)
>Right Transposon End for BAQ60380.1
TATAATAAATTACAGGAAGGACGTATAGCTCAGTTGGTTAGAGTACATCGTTGACATCGATGGGGTCTCGTGTTCGAGTCACGA
TACGTCCATTTATTTTTGTACAGTGCCACATTAATTGTCATCGGTGACAAATTAATGTCGTTATGCCAAATTCTTGTCTTTTAA
TAAGCAGTGACAAATTAAATGTCGTCTAATAAATTTGCCTTGTGGTTTGTTTCGATGGTGGGGAAATTTAATTTTTATTTGTGA
CCCCATTGATGTCCTTTCAATCTATCCTAAGTCGATTATACTGTATTGATTTATTTCGGCTCGATCGATCATTAATTTTTTGTC
TTACCCTAATTTGATCGTTGCTCAGATATTTTCAATCAACCCCAAAGGGAAAACTTGATTGAAACCATCAATGGAGGCAATATT
CCCCGATTAGATAATTTTTCAATCAACCCCAAAGGGAAAACTTGATTGAAACCCCGTGCTTCGGAAGCCTTATACAGTAA
(SEQ ID NO: 197)
>Left Transposon End for WP_017289534.1
TCCGAATTAAGGATGGAGCAATTGTCGCGGCAAGAGTAGATACACAAACGACCCTGAAGTATTTCTACCGACAAGGAAGCGAAG
TCATTTTGAAACCTGCAAATGCTGCTTACGAACCCACCGTAGTTGAAGCGAACCAAGTAGATATCCAAGGGATGTACGTAGGAC
TGATACGGCGACTTTGGGAGAACGGTGAGCCATGACTTATAGTGCTTGCAATATCGATATTGATCTGCCTGATGAAGAGGCGGC
AGTCGATTTCGAAGACTGTGGCGATGCAGAATACGAGGATTGGCTCGCTGATTATTACTCGGATTGGGCAGATTTCTATCAACA
GGTGTCCGATGCAGAAGATGCCTTTCGACACAATTTAATAGTGAAGAATTATGAGTTTCAGGCACGCGGTGGCTTTTTACCGGG
GGACACTGTAATGGCGGAAGTCGAACCAGGGTATTGGCATCACTGCACCGTCCTCTTAGTAGGGATATCGACGTTAGTGGTTAG
TGCCGAGCGATCTACAACCGTTCAAGTCATAACTCAACTCAACACGTATTGGCTCGACTAGATCTGATTTTGGAATTCGCTCGT
CTTGCGATCGCTTCAAATAGTTAATTTGTATTAATTTTCCTGTCTAACAGATTGACTGTCGCTATTTCTTCCTCATAGGATTAG
TGCAGTTCCTGATAGGACTTATGTACTT (SEQ ID NO: 198)
>Right Transposon End for WP_017289534.1
AGTAACTTAGTCTTTTGACAACTTTAATAGGCACAATCTTTCGGTTAACAGTGGGTGGATTGAAAGGACTGCCTGACTCGGGGT
TTTAATTTAGAAATATTTGCCCCTCGCTATAACGAGCAGAGTTAGCATTCGTTTTAACTGGGTAGAGTCTGTACTGTAAAATGT
TGAAGTGGATAAGAATTTGAATGAGCATTCAACTGCTTGAAGCTCTGATGACAAGAATTTGTTAACGAATGATTTCTTACCCCT
CGACGACAAGAAGCTGTTAAAACGACACTAAATTGTTAACGACGACATCAATCCGTTAACGACGACAAATAAAGTGTTATTCGA
CAACAATCACACGAATTTAAACAAAAAATTGCCTGACTCTTAAAAGCCCCCAGAGTCAGGCAGTCTACCTGTAAGGCACGCCTT
TAACCGGATACACCAAACAAACTTAGTTGCCCCCTAGTTGCTTAGCTTTTCCGAGTGCGTTCCTCAGATTTGAATCTATT
(SEQ ID NO: 199)
>Left Transposon End for WP_012411901.1
TTTTTATCTCCATGGGAAATCCAGCAACTGGGGGAAGATTCTGCGGCGAAGCGGAGTGGCTGGTGCGGGCTGCTACCATCTTAA
CCAAGAGCCACAAGTCTTTTTGCCCGTACCAGCCACCGCAGAATCTAGCGCAGCGTTCCACAGTTGCGTCAATGTTAAGAGTAC
CGGTTTTTTGTTAACGATCGCTTGACTTTTAAGACAGTCGCTACAGAACTTAAGGTTTTTATGGCTAACTAAACCGTATTGGTT
TTTAGTTGAAATCGTGTCCGCTTAATCAACCTGAGTAAAAATGTCCTACTTTTAGCATGAACTTTCTGAATTCCTTGATATCCT
TTATCACCAAAGTATTCAATATTTTCTCTTAACCGAACTTTACTGTTTTTAAGTATGCAAAAATTATTGTACATTAACTAATTA
TTTGTCATTGTACAAAAATGTACAGTGACTAATTATATGTCGTCGAGACAAATTAATGTCATCCATTAAAATCTTGCTCGGTAT
AGGTTACAGCGTTTTAGTAGTATTAGGATAACATCTTGACAGTGACAGATTAGCTGTCATTTTGGGTAATAGTGACAAATTAGC
TGTCGCTTCATCAGAGATAAAAAAGCTTTTGTGTATTTTCATAATGACAAATTGACTGTCGCTTTCTGTTTAAGTAGAATAACA
ATATGTTTTTATAAAAAAGCTTCGCATT (SEQ ID NO: 200)
>Right Transposon End for WP_012411901.1
GCAAATCTGAAATCATTACGCACGCATACAAAAATGTTTTAATAACCTGCCAGGTACTAGGTGGGTTATCATAATTTTTATTAG
GGAGGGTTGAAAGAGAGCGCTACGTTGACATTATCGCTATAGTCTCTGTAAATAAATGACATTAATCTGTCACTGGCACCTAAA
CGACATCAATTCGTCCCGACGACAGTTAATTAGTCCCAACGACATTAATCTGTCACCGACGACAAATAATTAGTCACTGTACAA
AAAAAATGGACGTAACTGGATTCGAACCAGTGACCTCTACGATGTCAACGTAGCGCTCTAACCAACTGAGCTATACGTCCTTAA
CCACACGAATATTGATAGTAGCATATACTTTTGCGATCGCACAAGATAAAGACCCAAGCTTTTTAAGGGGGTTTTAGATTTTGA
GTGCGTAAATCCTAATTTCAAATAAAATCCTTATTATTTCGTTACAATCAGCTTGTGCGTTAAGCATTCAAGAGTTATCA
(SEQ ID NO: 201)
>Left Transposon End for WP_011318008.1
CAATGGTGGCTTGCGACAAATAGTCATTAATCTGCTACACAACAGCCTCAAGTTTACAGCCAATGGTGGTCGAGTGTGGGTGAG
AGCTAGAGTCCAAGGCGAATACGTCATACTAGAAATTCGTGACACAGGTATCGGTATTGCTGAAAGCGAAATTCCCAAAATATT
CGACTGCTTTTATCGTGTGCGATCGGGACTAATTGATGAGACGAATGGCGCAGGTTTAGGACTCACAATTGTCCAGAGATTGTT
ATGGCATTGTGGTGGTTCTGTTAATGTGAGAAGCAAGGTTGATGAAGGTACTATGGTAATAGTGCAAATGAAGATAGGAAGCAC
CTCGCCAACTTAGATCCGGAAAACGGGGTGAAGCGATCGCCTGTTGGTTGACAACTGCACTTGTAGAGCGACAAATAATTTGTC
GTTCTACAATTGTCGATTGCCAAATTATATGACCACTTGACAAGTTAATGTCATTCAAAATATTATCCTCAAAACCCTTGCCAA
GCAAGGGTTTTGTTATTTTAGGCTCAAAATACCCAGAAACTTATTGCCAAAATAGCTGTCCTCTTTTGAAAAGTTGACGAAATA
TCTGTCCTTGCTTGAAAAGGGACTTGAGGGAAGATTTTTACAGAAATTGACAAAATAAATGTCCTCCAAGTAGTACAATACAAC
TACGTTTTTATTGAACATTTATGTAAAT (SEQ ID NO: 202)
>Right Transposon End for WP_011318008.1
ACCTTCATGCAAGGATATAAAAAATTTTAGGTGGGTTGAAAGGCGCACTTCGTTCGGGATTTTCCCTGACCGAAACAGTACCAA
TAAATCAAAGCTATTATAATAACAGCTTCTAATGCCAATACACCTTGATGACTAAGTAATTTGGCAACGCGGACAACAACTTGG
CAATACGGACAACAATTTGTCAACGCAGACAAAGAATTTGGCAATCGACAACAATAATCGGGATGACTGGATTCGAACCAGCGG
CCCCTTCGTCCCGAACGAAGTGCGCTACCAAGCTGCGCTACATCCCGTTAAAAAATTTATACGCCTTTTTTAGCATAACATAAA
TAATAGTCAATACAGAAAAGACATCTACATCTATATATATAGATGAATCGGAACGGGATTTGCTTGCTAGGATTAGATTTGTAT
AACATTCAGTGGTAAAAGCGGATTGTGACTGTAAAACCAGACTGGTTGCGGGTAAAAGCGCCTCAATGGGAGCGCGTTGG
(SEQ ID NO: 203)
>Left Transposon End for AFZ00435.1
AAAGCAGTAGGACTATTATCTGCCCATATCCACAGTAATCCTTGTAATACTTGAGTCGGATATGTGGTAATTTGCGATCGCTCG
CTTTTACAAGCAGTTTCTAAAGCCTTTTCATCGCTCAACATGGGAATATTTGTACATGTTCCTGTCCCATCAAAACACCAACCA
TGATGGCGACACATGATGTTGCCATTTTTGTCAATACTTCCTAAAGATAACTGCGCTAATTTATGGGGACAAACATCATCCATT
GCCACCCATTTTTGCTGCTTATCTCTCCAAATTACCAGATTTTTACCCAGCAAAGTAATTGCAGTGGGATGAGAGGGTTCTAGA
TAGCTGATAGGAGTTACAGGATACCACTGTTTAGTCCAGGAAAAGTTAGTCATCATCAAATTAACTAATTTATTCGACTCAAAT
TTGAAAAATGAGTAAAAAGCGGCATTTAATGTGCGAATGTTGTACATTCGCACATTATATGTCGCTTTTCGCAAGTTAGGTCGC
AACCGCATTTAACTGCTATAAACCCTATTTTACAAAGGTTTGATGCTCTTAGCACATCAAGCCCACGAATTTACTTAATTCGCA
TATTCCATGTCGCAAACTAAATATTCGCAAATTGAATGTCGTTTATTAAAATTTGTCACTTCGCAAATTGTTTGTCGTATTATT
GAGCGATTCATGGTACATTGGTACTCTA (SEQ ID NO: 204)
>Right Transposon End for AFZ00435.1
TTGAAAATCTAAGAGCAACTACACTATAGATTTATTTAGAGGGTTGAAAGGAGCATCAGACTCTTAATTTAAGTGATAGAGATA
ATATTTAAGCACAGATATATTTCTTACTCAGCAATAGCATTTAGTATTTGCATGGAAATAAATAGCTTTGAGACAACATTAATT
TGTTAACGATGTCTTTGAGGATTTAGGGGACATCAATTTGTTAAAAAAGACATTAATTTTGTTAACGACGACAAATTATTTGTA
ATCGACTTTAGGACAAATAATTTGTCGCTTTATGTACTTTGACAAATAATGTGTCGCTCTACATCAGTTAATCGACAATAACCA
AGCGACGTTAATTTGCGAAAACCTATAATCATCAATATAGTACACAAATCTGTCGAAAGCGACACTAATTTGCTAATAACGACA
CTAATGTGCGAAAAGCGACATTTAATGTGCGAAAGTACAAATGTACAAAATGGGCGACCTGGGGCTCGAACCCAGAACCAGCAG
ATTAAGAGTCTGATGCTCTACCATTGAGCTAGTCGCCCTCACCATTTACTACTATAGCAAATTATTTGAACAATAGTTAACCTT
TATCGGAAAATCTTCATATAACTAGTTGACATTAGCAAACACATACCCTCTGATGCGGATAAAACTTGCTCATACAAAATCAAA
ACTAGGTAATAGCACCACTAATTATACT (SEQ ID NO: 205)
>Left Transposon End for AFZ56196.1
AGTCAGGAGTCAGGAGTCAGAAAGAAGAATTAGAAGGAGATCAGAATATTCTCAAATCTGGGTAAATGAACTATTTTTGATGCT
TTCATCGCTTATTTCTTGCACCTTCTCCAACTTTTTCAGCCTGAGACTGTTGATATATCTATGTTTTTGGTTTATTAAGCAAAC
CCTAAGTACTATCTGCAAGGGTTTCAGGCATGATAGACCCCTTAAATTGTCATTACGAGCGGAGCGAAGTAATCACATAATGCG
ATCGCACCACCTCAAGCTAGATGATGAAACCAAGTACTAGTAGGAATTTTAAGACTTGTGTGTACACCGTAGATTATTGAGTAG
GGGCATCTAACGATAACGAATAATGATAAATACCGTAGTGCAATATTATGCTACAGGAAACTTAATCAGTGTTTACATAATTTG
TGTACATTAACTAATTATTTGACAATTTAACAAAATATTGTCAAAAATCATAAAAATACCTTAAAACCTTCTACAGCAAAGATT
GTAGGAGGTTTTTATTTATCTAATTTGTGAACATCCTCCAGACATATATTTAACAAATTAAGTGTCAAAAGCCAGATAATTATC
AATTTATTTGTCAATTTGCCAAATACAGGTAAATTACTATATTTCTGAAAATTTCACACATTAAATGTCACTTTTACTCTATAC
TATACAAATATGTTGTAATTAAACATTA (SEQ ID NO: 206)
>Right Transposon End for AFZ56196.1
ACATTAATGCAAGTAGAATAAAAAATATCTAGGTGGGTTGAAAGATCAGTAGGTCGTGGGTTTAACTCTGAAAACACTTGAAAA
CACTATATAAATACAGTGTTGCTTGTGACATTTAGGGACAATTAATTTGTTAACAGTGACACGAATTAGTTAAAAATGACATTA
ATTTGTTAACAGTGACAAATAAATTGTTAATGTACAGACATATAGTGTGCGAATGTACACCAAAGCCGATGATGGGATTTGAAC
CCACGACCTACTGATTACGAATCAGTTGCTCTACCCCTGAGCCACATCGGCATACACAGTTTGATATCATAGCATTATTTACCT
ATGATAGACCCAAATTCCCAAAATCGTCTTAACTCTAAACAATATGCCCACCTGAAAGCTGAAATAGCAGCCCCTTATCGTGGT
TTGCGGCAATTTATCTATATAGGTGTTGGTGCTTCTGGTTTTATCGGCGCATTCACCTTCTTTTTTCAACTGCTTGCTGG
(SEQ ID NO: 207)
>Left Transposon End for BAB75312.1
CATTGGAAAACAGCGAACAGTGCTTTAGCCAGAATCAGAACAGCATTTGATCGGCACAGTCTTAAGCCATATACTTTTTTTGTG
GGATCTGCTATAGAAAAAAGCATGGCAGAAGAAATTTGGCATCAACTAAATTCTGGCATTCTTACTAATGCAGCAAACCTCACT
CAGCCTGACCAAGTTGCCTCTTTGTGCGCTTGGTTTATTCAACTTTGATGTACAGTGACTAATTATTTGTCACTGTACACAAGA
TGTACAGTGACTAATTATTTGTCACTGTACACAAGATGTACAGTGACTAATTGTTTGTCGTCGTGACAAATTAATGTCGTCGAA
TGAATCCTTGCAATACAAAGGTTTTAGCTATTTAAGAGTTATTACATTATTTCTCATACGTGACTAAATAAATGTCGTTTTCCG
CAAAAATGACAAATTAACTGTCGCTTCAGTAATTACAAAAAAGGTTTTGTATATTTTCATAATGACAAATTGACTGTCGTTTTC
TCCACGTTTAGAATAACATTATGTATTTATAAATTACCTTTGTTTCATGAACAAAAAATCTTATGTCTGATTTTACTGTTCATA
CCGCAGTGGATACTGCGGAAGCTTTATTACAAGACAACAACACACCTCCTCACCTCCTGAGACGAATCTGATTGTTACAGAACT
CTCAGAGGAAGCCCAACTCAAGCTAGAG (SEQ ID NO: 208)
>Right Transposon End for BAB75312.1
ACATGGTGAAAACAAATTGGATATGAAACTGCCTTACGTTGTCTGATGGTGTAATTAATTTCCAAGAAGTAGAGGGTTGAAAGC
AAATCCCGTCATCGGCTTGTAACTAAAGTTATTCAGGATAAGCTACTCATAGAAGTGATTAAAAGAGCTTTTTGAATGCAAACG
CTTATAATAGGGGCTAAATATATAGAGAAACTCCATATATAAATTGTTGCTTTTCCGAAAAATGACAATAATTTGTCACAAATA
TATATGGAAGCGAGTTACTAAGTTGGATGACAATAATTTGTCACAACGACATCAATTTGTCACCGACGACAAATAAGAGACCAT
TTATAAAGTAAATCTTTAGACGACTAGACGACGTAGCATAATACGAGTCATAACGGCATATATGGCAGCCTCACTCATTTCTGG
GAGACGCTCATAATCCTTACTGAGACGACGGTACTGGTTTAACCAGCCAAATGTTCTTTCTACTACCCACCGTTTGGGCAAAAC
CTGAAATTCTTGATTAGTACGCCGGATTACCTCAACATGAGCTTGAATCATCAGCCAAACAGAGAGCGCAAATTTATCACCGTC
ATAGCCGGAATCAACCCAGATGACTTCAACTTTTTCCAGTAATTCTGGACGCTCTTCTAACAGTTCCATCAAAGTATAGGCGGC
AAGTAATCTTTCTCCAGCATTTGCTTCACTTACAACCACTTTTAACAAAAGTCCCAGACTATCAACCAAAGTTTGCCGCTTTCG
TCCTTTTACCTTCTTGCCACCATCAAAACCGTACACATCCCCCTTTTTTCAGTCGTTTTTACCGACTGGCTGTCTGCCGCGATC
GCCGTGGGTTGAGTTGACTTCCCCATTTTTTGACGAACTTGATCGCGCAAAGTATGATTCATTTGCTCCCAGATACCTCGTTTT
TGCCATTTACGATAGTAGCTGTAAACAGTTGAACTAGGAGGAAAATCCCCTGGAAGCATATCCCACTGACAACCTGTTTTCAGA
TGGTAGTAGATAGCGTTGCATACTTCTCGCATATCAGTTGTTCGGGGATGCCCACCGCATTTAGCGGGTGGAATCAAAGGAGCT
AAAATTGCCCATTCTGAGTCATTAAGGTCTGTAGAATAAGACTTTCGTCTCATTGTTTCCTATGTAAATACACTCTACAAACAG
TATCTTATCGCTGCCTTTTTATCTGATAGCTCTCCTTTAGATTTACTTTATAAATAGCCTCTAATTTGTCACTGTACACAAGAA
TTAGTCAAGACAAAAATATGATTTTATGAAAACCAAGCCG (SEQ ID NO: 209)

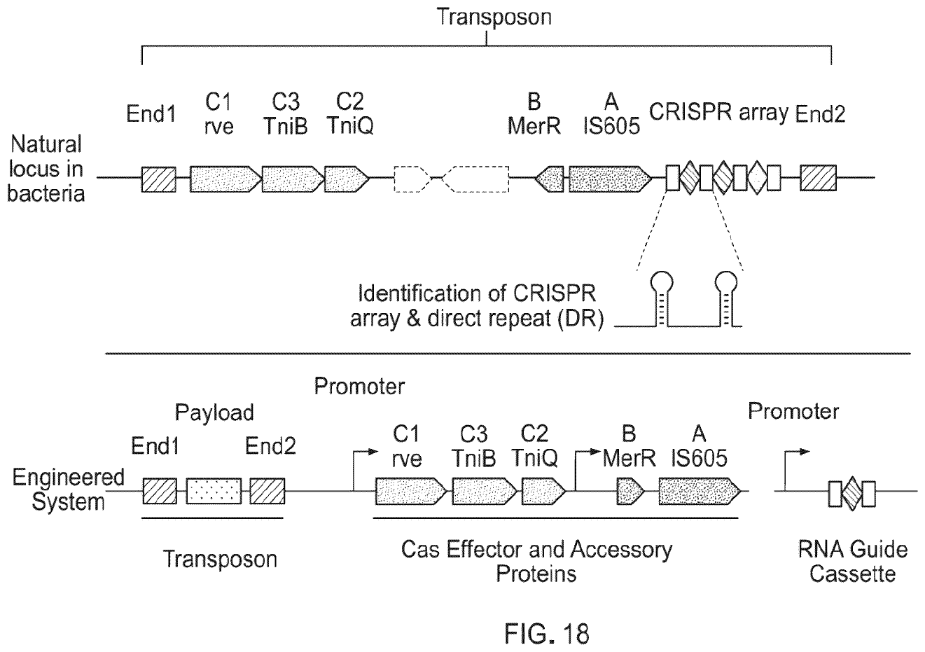

### Example 2—Design of Engineered System for a CLUST.004377 CRISPR System (FIG. 18)

Having identified the minimal CLUST.004377 CRISPR-Cas system components, we composed an engineered system for transposon excision, mobilization, and programmable insertion (FIG. 18). Minimally, the natural locus consists of a left Top end flanked on the right side by effectors C1-C3, and a right Tnp end flanked on the left side by effectors A-B and the CRISPR array. Transposon cargoes ranging from approximately 2.5 kb to over 25 kb in length are located between effectors C1-C3 and effectors A-B. These cargoes are composed of CDS and non-coding sequences. In some embodiments, the Cas Effector Cassette of an engineered CLUST.004377 system is composed of effectors C1-C3 under the control of a single artificial promoter, followed by effectors A-B under the control of a second artificial promoter. In some embodiments, the Transposon Payload Cassette of an engineered CLUST.004377 system is composed of Tnp ends flanking nucleic acid cargoes ranging from approximately less than 100 nt to greater than 25 kb (FIG. 18). In some embodiments, the RNA Guide Cassette of an engineered CLUST.004377 system is expressed under the control of a single artificial promoter. In some embodiments, the RNA guide may consist of a minimal CRISPR array containing two or more direct repeats and one or more spacers. In some embodiments, the RNA guide may consist of one or more crRNAs containing processed direct repeat and spacer components, and optionally a fused tracrRNA component. In some embodiments, the RNA guide cassette contains a tracrRNA expressed under a second artificial promoter.

We selected the KV757663 locus for functional validation of the engineered CLUST.004377 system.

### DNA Synthesis & Effector Library Cloning

To test the activity of KV757663 CLUST.004377 CRISPR-Cas system, we designed and synthesized a minimal engineered system as described above into the pET28a(+) vector. The synthesized system consisting of an Effector Cassette and acceptor site for an RNA Guide Cassette were included as the cargo region of a Transposon Payload Cassette. The synthesized combined IPTG inducible T7 and lac promoters were used to drive expression of the Effector Cassette, and each CDS sequence was codon optimized for E. coli expression and preceded by an E. coli ribosome binding sequence. A J23119 (Registry of Standard Biological Parts: http://parts.igem.org/Part:BBa_J23119) promoter was used for expression of the RNA Guide Cassette.

In tandem with the effector gene synthesis, we first computationally designed an oligonucleotide library synthesis (OLS) pool containing “repeat-spacer-repeat” sequences, where “repeat” represents the consensus direct repeat sequence found in the CRISPR array associated with the effector, and “spacer” represents sequences tiling the pACYC184 plasmid and E. coli essential genes. The spacer length was determined by the mode of the spacer lengths found in the endogenous CRISPR array. The repeat-spacer-repeat sequence was appended with restriction sites enabling the bi-directional cloning of the fragment into the acceptor site for the RNA Guide Cassette, as well as unique PCR priming sites to enable specific amplification of a specific repeat-spacer-repeat library from a larger pool. The library synthesis was performed by Agilent Genomics.

We next cloned the CRISPR array library into the plasmid containing the minimal engineered CLUST.004377 system using the Golden Gate assembly method. In brief, we first amplified each repeat-spacer-repeat from the CRISPR array library using unique PCR primers, and pre-linearized the plasmid backbone using Bsal to reduce potential background. Both DNA fragments were purified with Ampure XP (Beckman Coulter) prior to addition to Golden Gate Assembly Master Mix (New England Biolabs) and incubated as per manufacturer's instructions. We further purified and concentrated the Golden Gate reaction to enable maximum transformation efficiency in the subsequent steps of the bacterial screen.

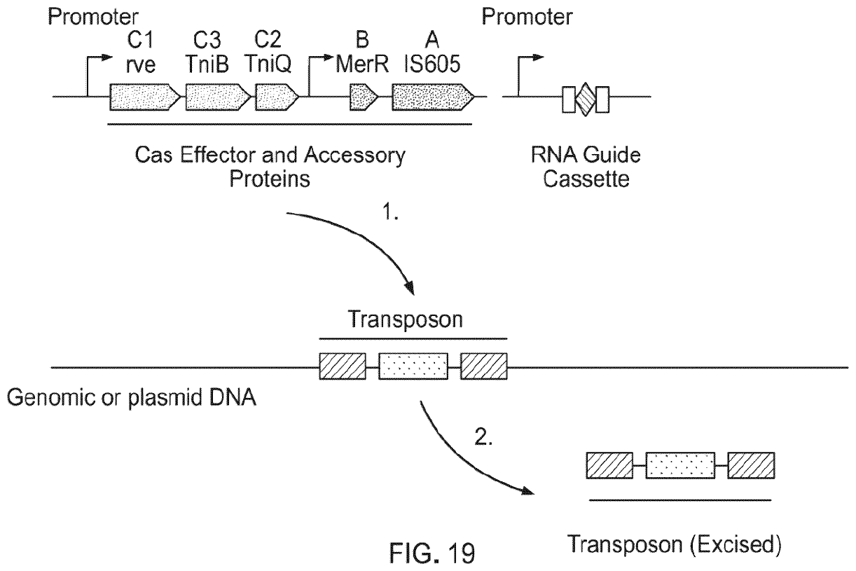

### Example 3—Functional Screening of CLUST.004377 CRISPR System (FIGS. 19-25) Functional screening for CLUST.004377

To accelerate functional screening of CLUST.004377 systems, we developed a strategy to derive the following functional information in a single screen: 1) crRNA expression direction and processing, 2) nucleic acid substrate type, and 3) targeting requirements such as protospacer adjacent motif (PAM), protospacer flanking sequence (PFS), or target secondary structure. We designed minimal CRISPR array libraries consisting of two consensus direct repeats, each flanking a unique natural-length spacer sequence targeting either the pACYC184 vector, E. coli essential genes, or an absent GFP sequence as a negative control. We also designed a bidirectional array library cloning strategy to test both possible CRISPR array expression directions in parallel.

The CRISPR array library for the was cloned into the KV757663 CLUST.004377 CRISPR-Cas system expression plasmid RNA Guide Cassette acceptor site such that each element in the resulting plasmid library contained a single library element and orientation. The resulting plasmid libraries were transformed with pACYC184 into E. coli using electroporation, yielding a maximum of one plasmid library element per cell. Transformed E. coli cells were plated on bioassay plates containing Kanamycin (selecting for the library plasmid), Chloramphenicol (CAM; selecting for intact pACYC184 CAM expression), and Tetracycline (TET; selecting for intact pACYC184 TET expression).

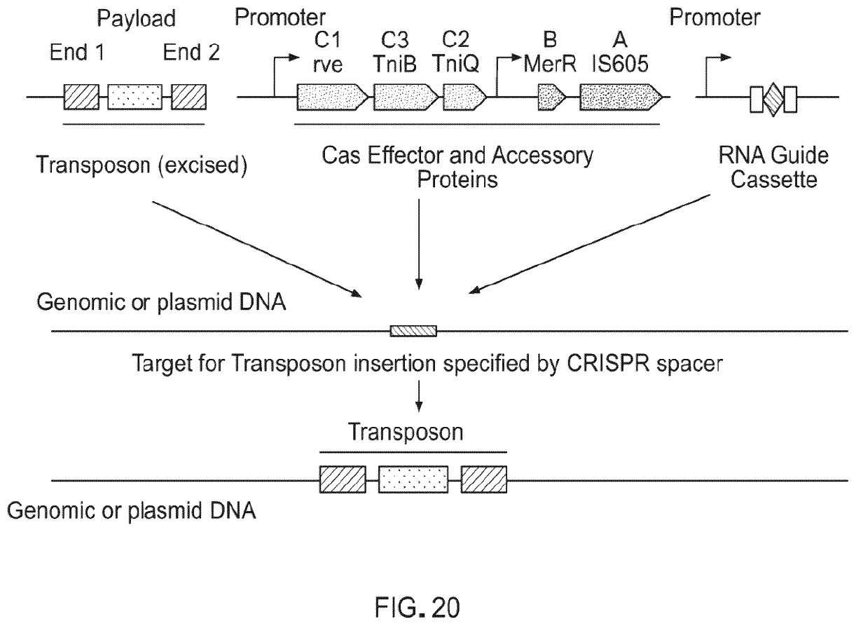

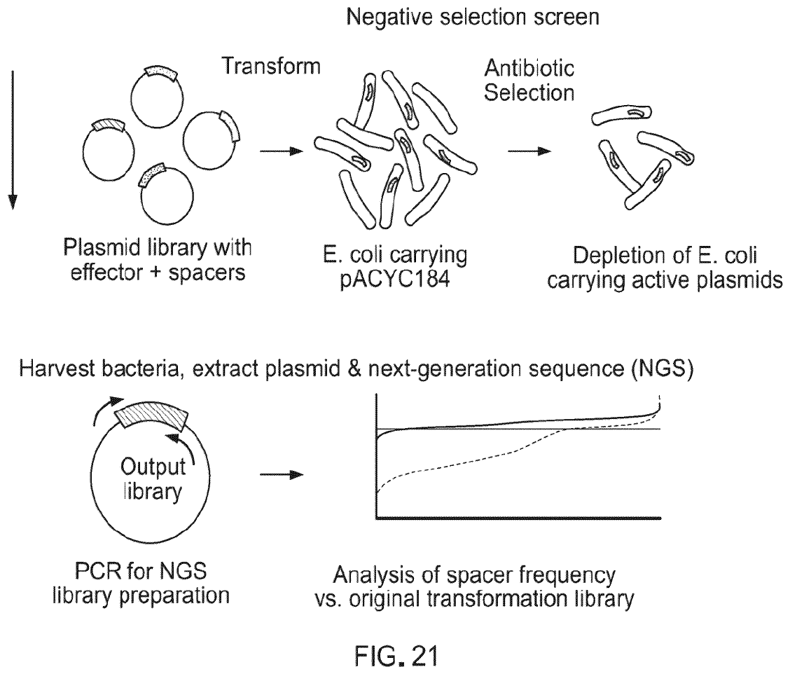

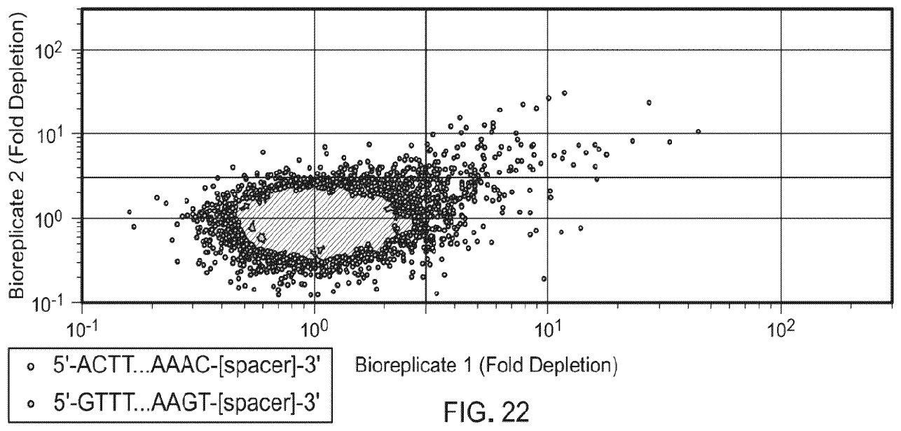

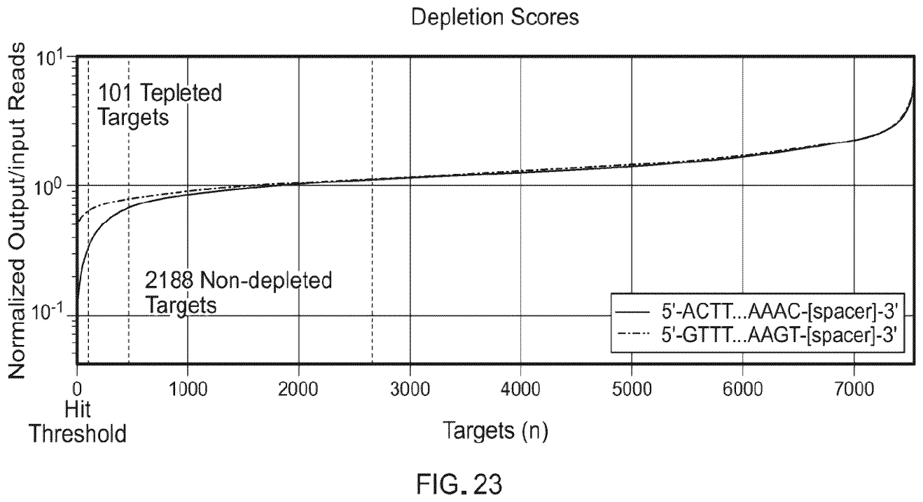

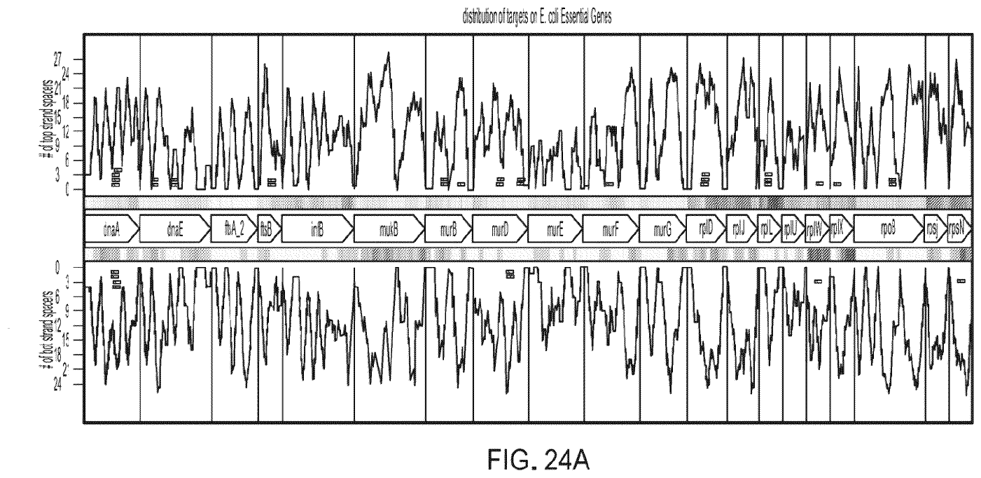

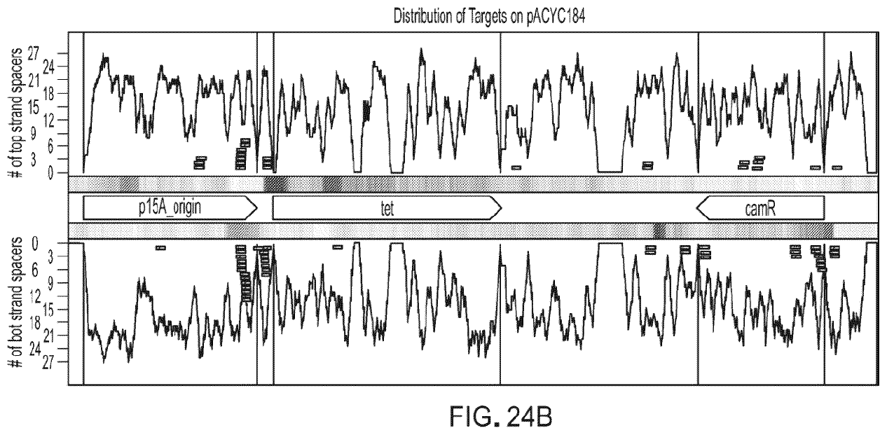

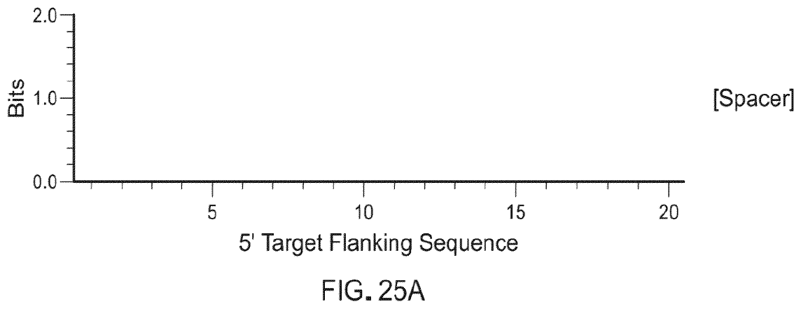

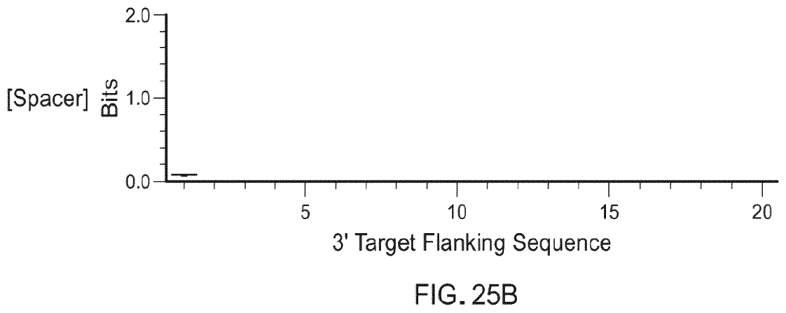

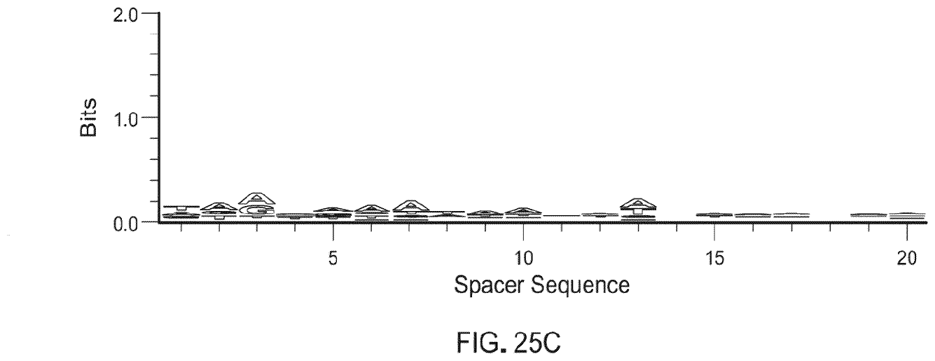

Programmable mobilization and insertion of the KV757663 CLUST.004377 Transposon Payload Cassette was assessed as follows. Following transformation, the CLUST.004377 system mobilizes the Transposon Payload Cassette by excising it from the donor plasmid followed by programmable insertion of the mobilized cassette at a target site specified by the spacer sequence of the expressed RNA guide (FIGS. 19-20). Interruption of pACYC184 antibiotic resistance genes or E. coli essential genes by targeted insertion of the Transposon Payload Cassette by the CLUST.004377 CRISPR-Cas system results in bacterial cell death and depletion of the targeting RNA guide (FIG. 21). To investigate negative selection of RNA guides resulting from programmable transposon insertion, bacteria were harvested 12 h after plating, and plasmid DNA was extracted. We PCR amplified the CRISPR array region of the input plasmid library prior to transformation and the output plasmid library after bacterial selection on antibiotic plates and compared the frequency of RNA guide elements in these samples to identify depleted elements (FIG. 21).

### Bacterial Screen Transformation

The plasmid library containing the distinct repeat-spacer-repeat elements and Cas proteins was electroporated into Endura or E. cloni electrocompetent E. coli (Lucigen) using a Gene Pulser Xcell (Bio-rad) following the protocol recommended by Lucigen. The library was either co-transformed with purified pACYC184 plasmid, or directly transformed into pACYC184—containing electrocompetent E. coli (Lucigen), plated onto agar containing Chloramphenicol (Fisher), Tetracycline (Alfa Aesar), and Kanamycin (Alfa Aesar) in BioAssay dishes (Thermo Fisher), and incubated for 10-12 h. After estimation of approximate colony count to ensure sufficient library representation on the bacterial plate, the bacteria were harvested and DNA plasmid extracted using a QIAprep Spin Miniprep Kit (Qiagen) to create the ‘output library’. By performing a PCR using custom primers containing barcodes and sites compatible with Illumina sequencing chemistry, we generated a barcoded next generation sequencing library from both the pre-transformation ‘input library’ and the post-harvest ‘output library’, which were then pooled and loaded onto a Nextseq 550 (Illumina) to evaluate the effectors. At least two independent biological replicates were performed for each screen to ensure consistency.

### Bacterial Screen Sequencing Analysis

Next generation sequencing data for screen input and output libraries were demultiplexed using Illumina bcl2fastq. Reads in resulting fastq files for each sample contained the CRISPR array elements for the screening plasmid library. The direct repeat sequence of the CRISPR array was used to determine the array orientation, and the spacer sequence was mapped to the source plasmid pACYC184 or negative control sequence (GFP) to determine the corresponding target. For each sample, the total number of reads for each unique array element (rₐ) in a given plasmid library was counted and normalized as follows: (rₐ+1)/total reads for all library array elements. The depletion score was calculated by dividing normalized output reads for a given array element by normalized input reads.

To identify specific parameters resulting in enzymatic activity and bacterial cell death, we used next generation sequencing (NGS) to quantify and compare the representation of individual CRISPR arrays (i.e., repeat-spacer-repeat) in the PCR of the input and output plasmid libraries. We define the array depletion ratio as the normalized output read count divided by the normalized input read count. An array was considered to be strongly depleted if the fold depletion was at least than 3. When calculating the array depletion ratio across biological replicates, we took the maximum depletion ratio value for a given CRISPR array across all experiments (i.e. a strongly depleted array must be strongly depleted in all biological replicates). We generated a matrix including array depletion ratios and the following features for each spacer target: target strand, transcript targeting, ORI targeting, target sequence motifs, flanking sequence motifs, and target secondary structure. We investigated the degree to which different features in this matrix explained target depletion for CLUST.004377 systems, thereby yielding a broad survey of functional parameters within a single screen.

### Bacterial Screening Indicates Programmable DNA Interference by the CLUST.004377 System

Comparison of two bioreplicate screens for the KV757663 CLUST.004377 CRISPR-Cas system showed replication of depleted RNA guide elements for a single direct repeat expression direction (5′ ACTT . . . AAAC-[spacer] . . . 3′). This indicates a specific interaction of the KV757663 CLUST.004377 effector proteins with a specific direct repeat orientation, and accompanying modification of targeted pACYC and E. coli essential gene target sequences (FIGS. 22-23). Targeting of both strands of pACYC and E. coli essential genes indicates DNA targeting is not confined to a specific strand orientation (FIGS. 24A-B). Additionally, localization of depleted targets to sequences either within or in close proximity to gene coding sequences is indicative of coding DNA interruption associated interference consistent with the interference arising from mobilization and programmable insertion of the Transposon Payload Cassette at or in close proximity to target sites (FIGS. 24A-B). In an attempt to understand targeting requirements for the CLUST.004377 system, we compiled all target and target-flanking sequences associated with depleted RNA guide elements (FIGS. 25A-C). Interestingly, while flanking sequences showed no evidence of PAM or PFS elements required for targeting, the KV757663 CLUST.004377 system shows a targeting preference for A and T-rich sequences.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An engineered, non-naturally occurring Clustered Interspaced Short Palindromic Repeat (CRISPR)—Cas system of CLUST.004377 comprising:
a Guide consisting of a direct repeat sequence and a spacer sequence capable of hybridizing to a target nucleic acid, wherein the Guide is CRISPR RNA (crRNA) or DNA, and any one of the following:
a. a CRISPR-associated protein containing charged N-terminal residues that are capable of binding to the Guide, either as a monomer or multimer, and of targeting the target nucleic acid sequence complementary to the Guide spacer;
b. a CRISPR-associated protein containing charged N-terminal residues and an HTH domain-containing protein that together are capable of binding to the Guide and of targeting the target nucleic acid sequence complementary to the spacer sequence;
c. a CRISPR-associated protein containing charged N-terminal residues and a transposase module that together are capable of binding to the Guide and of targeting the target nucleic acid sequence complementary to the spacer sequence;
d. a CRISPR-associated protein containing charged N-terminal residues, an HTH domain-containing protein, and a transposase module that together are capable of binding to the Guide and of targeting the target nucleic acid sequence complementary to the spacer sequence; and
e. a payload nucleic acid flanked by transposon end sequences.

2. The system of claim 1, wherein the target nucleic acid is a DNA or an RNA.

3. The system of claim 2, wherein the target nucleic acid is double-stranded DNA.

4. The system of claim 1, wherein targeting of the target nucleic acid by the protein and the Guide results in a modification in the target nucleic acid, which optionally is a double-stranded cleavage event or a single-stranded cleavage event.

5-6. (canceled)

7. The system of claim 1, further comprising a donor template nucleic acid, which optionally is a DNA.

8. (canceled)

9. The system of claim 1, wherein the target nucleic acid is a double-stranded DNA and the targeting of the double-stranded DNA results in scarless DNA insertion.

10. The system of claim 1, wherein the modification results in cell toxicity.

11. The system of claim 1, within a cell, which is a eukaryotic cell or a prokaryotic cell.

12-13. (canceled)

14. A method of targeting and editing a target nucleic acid, the method comprising contacting the target nucleic acid with a system of claim 1, wherein optionally the method results in an insertion or substitution of DNA to correct a native locus.

15. A method of targeting the insertion of a payload nucleic acid at a site of a target nucleic acid, the method comprising contacting the target nucleic acid with a system of claim 1, wherein optionally the method results in a targeted insertion or deletion of a DNA payload into a specific genomic target site.

16. A method of targeting the excision of a payload nucleic acid from a site at a target nucleic acid, the method comprising contacting the target nucleic acid with a system of claim 1, wherein optionally the method results in a targeted deletion of DNA to correct a native locus.

17. The system of claim 1, wherein the transposase module comprises Mu-transposase, TniQ, and/or TniB.

18. The system of claim 1, wherein the CRISPR-associated protein comprises an amino acid sequence having at least 85%, 90%, 95%, 96%, 97%, 98%, 99%, or 100% similarity to an amino acid sequence provided in any one of Tables 2-6.

19. The system of claim 1, wherein the crRNA or Guide comprises a nucleic acid sequence having at least 85%, 90%, 95%, 96%, 97%, 98%, 99%, or 100% similarity to a nucleic acid sequence provided in Table 7.

20. The system of claim 1, wherein the payload nucleic acid is flanked by transposon end sequences comprising a nucleic acid sequence having at least 85%, 90%, 95%, 96%, 97%, 98%, 99%, or 100% similarity to a nucleic acid sequence provided in Table 8 or Table 9.

21. (canceled)

22. The system of claim 1, wherein the CRISPR-associated protein comprises at least one nuclear localization signal or at least one nuclear export signal.

23. (canceled)

24. The system of claim 1, wherein at least one component of the system is encoded by a codon-optimized nucleic acid for expression in a cell, which optionally is present within at least one vector, which optionally comprises one or more regulatory elements operably-linked to a nucleic acid encoding the component of the system, wherein the one or more regulatory elements optionally comprises at least one promoter, which optionally comprises an inducible promoter or a constitutive promoter.

25-28. (canceled)

29. The system of claim 24, wherein the at least one vector comprises a plurality of vectors, and/or is a viral vector that is optionally selected from the group consisting of a retroviral vector, a lentiviral vector, an adenoviral vector, an adeno-associated vector, and a herpes simplex vector.

30-31. (canceled)

32. The system of claim 1, wherein the system is present in a delivery system, which optionally comprises a delivery vehicle selected from the group consisting of a liposome, an exosome, a microvesicle, and a gene-gun.

33. (canceled)

34. A cell comprising the system of claim 1, wherein optionally the cell is a eukaryotic cell, which optionally is a mammalian cell, such as a human cell, or a plant cell; or is a prokaryotic cell.

35-41. (canceled)


SEQUENCE LISTING (209 sequences)

SEQ ID NO: 1 - protein, 629 aa, unidentified
  source 1..629: note = Alkalinema sp.
MSQITIQCRLVAPPTARQHLWTLAAEKNTPLINTLIQEVANHEEFETWRLKGKHPTKIVSEICRRLRTEAPFSGQPARFYTSAEKTVNYIFKSWFTLQSRLQRQLSRKQIWLSILKSDPELVELCGHPLETIQNKATQLLTQIEKTLADSNPEDPQSPCTNDLIRAQLFKKYHGAKTPLIRCSIAYLIKNGGKLPKSPEIPHKFAQRRRKAEIQVQRLQDQLEGRLPKGRDLTGQAWLSTLITATSTVPKDNHEQKQWNDRLLAKPCTTPFPILFETNEDLNWSKNHTGRICVRFNGLSEHTFQVYCDQRQLPWFQRFLEDQQTKHTSKNQHSSALFTLRSAHLAWQETHSNGAGWDNHYITLYCTVDVRLWTAEGTEEVHQEKAVDIAKQLTYLDQKEDLSETQAAFAQRLTSTLDRLNKPFPRPHRHRAQQHSHIIVGLSVDWEAPLTLAIWNANTQEVLVYRSLRQLLGKDYPLFLQHRQEQQKQSHNRHKAQKRNKNCQFGTSHLGEHIDRLLAKAVITIAQQYNAGSIAVPNLDNIRETLQATIDAKAEQKAPGCIEAQKRYTKQYKITIHRWSYGRLIDQIISKAKQVGLGVEEAKQPLSGNVQEKAKLVAISAYNARLIVAF

SEQ ID NO: 2 - protein, 639 aa, Anabaena sp.
MSQITVQCRLIASESTRQQLWTLMAELNTPLINELLQQLSKHPDFEKWRKDGKFPSTVVSQLCQPLKTDPQFAGQPSRCYLSAIHVVDYIYKSWLTIQKRLQQQLDGKIRWLEMLNSDAELVETSGYSLEAIRTKAAEILAMTTPESDTNVPLTKKRNTKKSKKSSASNPEPSLSHKLFNAYQETDDILSRSAISYLLKNGCKLNDKEEDTEKFAKRRRKVEIQIQRLTDKLTSRIPKGRDLTNSKWLETLFTAITTVPEDNAEAKRWQDILSTRSSSLPFPLIFETNEDLKWSTNEKGRLCVHFNGLTDLTFEVYCDSRQLHWFKRFLEDQQTKRKSKNQHSSGLFTLRNGRLAWQEGEGKGETWQIHRLTLSCCVDNRLWTAEGTEQVRQEKAEDITKFITKMKEKSDLSDTQQAFIQRKQSTLTRINNSFDRPCKPLYQGQSHILVGVSMGLEKPATVAVVDASANKVLTYRSIKQILGENYELLNRQRRQQRSSSHERHKAQKSFSPNQFGTSELGQYIDRLLAKEIVAIAQTYKAGSIVLPKLGDMRENIQSEIQAIAEIKCPGSVEIQQKYAKQYRINVHKWSYGRLIQSIQSKAAQVGIVIEEGKQPVRDSPQDKAKELALSTYHLRLAKQS

SEQ ID NO: 3 - protein, 635 aa, Anabaena variabilis
MSVITIQCRLVAEEDILRQLWELMAEKNTPLINELLAQVGKHPEFETWLDKGRIPTELLKTLVNSFKTQERFADQPGRFYTSAIALVDYVYKSWFALQKRRKRQIEGKERWLTILKSDLQLEQESQCNLNVIRTKANEILTQFTPQSDQNKNPRKSKKAKKSAKLQKSSLFQILLNTHEQTQETLTRCAIAYLLKNNCQISERDEDPEEFNRNRRTKEIEIERLKDQLQSRIPKGRDLTGEEWLETLEIATVNVPQNEKEAKAWQAALLRKTADVPFPVAYESNEDMTWLQNDKDRLFVRFNGLGKMTFEVYCDKRHLHYFKRFLEDQEIKRNSKNQYSSSLFTLRSGRLAWLPGKQKGEPWKINQLHLYCTLDTRMWTAEGTQQVVNEKITKITETLTKAKQKDELNDKQQAFITRQQSTLDRINNPFPRPSQPNYQGRPSILVGVSFGLEKPVTLAVVDVVKNEVLAYRSVKQLLGKNYNLLNRQRQQQQRLSHERHKAQKQNAPNSFGESELGQYVDRLLADEIVAIAKTYQAGSIVIPKLRDMREQISSEIQSRAEKKCPGYKEVQQKYAKEYRMSVHRWSYGRLIESIKSQAAKAGIFTEIGTQPIRGSPQEKARDLAVFAYQERQAALI

SEQ ID NO: 4 - protein, 643 aa, Anabaena variabilis
MSVITIQCRLIASEATRSYLWQLMAQKNTPLINELIEQLGIHPEIEQWLKKGKLPDGVVKPLCDSLITQESFANQPKRFNKSAIEVVEYIYKSWLALQKERQQTIDRKEHWLKMLKSDVELEQESKCTLDAIRSQATKILPKYLAQSEQNNNQTQSQNKKKSKKSKTKNENSTLFDILFKAYDKAKNPLNRCTLAYLLKNNCQVSQKDEDPNQYALRRSKKEKEIERLKKQLQSRKPNGRDLTGREWQQTLIMATSSVPESNDEANIWQKRLLKKDISLPFPIRFRTNEDLIWSKNEEGRICVSFSGEGLNDHIFEIYCGNRQIHWFQRFLEDQNIKNDNNDQHSSALFTLRSAILAWQENKQHKENSLPWNTRRLTLYCTLDTRLWTTDGTEKVKQEKVDEFTQQLANMEQKENLNQNQQNYVKRLQSTLNKLNNAYPRHNHDLYQGKPSILVGVSLGLEKPATLAIVDSSTNIVLAYRSIKQLLGDNYKLLNRQRQQQQRNSHERHKAQKSNMPNKLSESDLGKYIDNLLAQAIIALAKNYQAGSIVLPTMKNVRESIQSEIEARAVKRCPNYKEGQQQYAKQYRQSIHRWSYNRLMQFIQSQAVKANISIEQGPQPIRGSSQEKARDLAIAAYYLRQNKS

SEQ ID NO: 5 - protein, 642 aa, Anabaena variabilis
MSQITIQCRLVASEPSRHQLWKLMVDLNTPLINELLVQVAQHPEFETWRQKGKHPAKIVKELCEPLRTDPRFIGQPGRFYTSAIATVNYIYKSWFALMKRSQSQLEGKMRWWEMLKSDAELVEVSGVTLESLRTKAAEILSQFAPQPDTVEAQPAKGKKRKKTKKSDGDCAERTLRERSISDYLFEAYRDTEEILTRCAINYLLKNGCKISNKEENAEKFAKRRRKLEIQIERLREKLEARIPKGRDLTDAKWLETLLLATLNVPENEAEAKSWQDSLLKKSITVPFPVAYETNEDMTWFKNERGRICVKFSGLSEHTFQVYCDSRQLQWFQRFLEDQQIKRNSKNQHSSSLFTLRSGRIAWQEGEGKSEPWKVNRLILYCSVDTRLWTAEGTNLVREEKAEEIAKAIAQTKAKGKLNDKQQAHIKRKNSSLARINNLFPRPSKPLYKGQSHILVGVSLGLEKPTTLAVVDGSIGKVLTYRNIKQLLGDNYRLLNRQRQQKHTLSHQRQVAQILASPNQLGESELGQYVDRLLAKEIVAITQTYKAGSIVLPKLGDMREQVQSEIQAKAEQKSDLIEVQQKYSKQYRVSVHQWSYGRLIASIRSSAAKVGIVIEESKQPIRGSPQEKARELAIAAYNSRRRT

SEQ ID NO: 6 - protein, 641 aa, Lyngbya confervoides
MSKITIQCRLVASEATRQYLWHLMADIYTPFVNEILRQIREDDNFEQWRQSGKIPASVFEDYRKTLKTESRFQGMPGRWYYAGREEVKRIYKSWLALRRRLRNQLAGQNRWLEVLQSDETLMEVSGLDLSALQAEASQLLNILGSKNKTSKNRSKKAKGKPKGKSAKDPTLYQALWELYRETEDIAKKCVIAYLLKHKCQVPDKPEDPKKFRHRRREAEIRAERLNEQLIKTRLPKGRDLTNEQWLQVLEIATRQVPKDEDEAAIWQSRLLTDAAKFPFPVAYETNEDLKWFLNGKGRLCVSFNGLSEHTFEVYCGQRQLYWFNRFLEDQQIKKENQGERSAGLFTLRSGRLVWKPYSSDASRSDPWMANQLTLQCSVDTRLWTAEGTEQVRQEKATSIAKVIAGTKAKGNLNQKQQDFITKREKTLELLHNPFPRPSKPLYQGKPSIIAAVSFGLEKPATLAIVDIVTDKAITYRSIRQLLGQNYKLFTKHRLKQQQCAHQRHQNQVESAENRISEGGLGEHLDSLIAKAILETAAEYGASSIVLPELGNIREIIHAEIQAKAERKIPGLKEKQDEYAAKFRASVHRWSYGRLAQKVTTKASLHGLETESTRQSLQGTPQEKARNLAISAYESRKVAQRA

SEQ ID NO: 7 - protein, 649 aa, Calothrix sp.
MSIITIQCRLVANDRTLQHLWELMAEKNTPLISELLEQLGKHPDFETWLKNGKVPKDTIKILCDSLKTQSRFAGQPGRFYTSAISQVKEIYKSWLTLQKRRQRQIEGKQRWLGMLKSDVELQEESNCSLEKIRAKGTEILAEFVSKFTKDTTKKSKTKIKSTKKSNKKTKKDTEESNSTLFQALCDIYDKTEDTLSKCAIIYLLKNNCQVIDTEENPDTFLKRKRAKEIEIKRLQDQIVGRIPKGRDLTDKKWLDTIKLASSQVPQDENEAKSWQNQLLKTSSSVPYSVDYETNTDIKWVKHNNGSIFVNFNGLGEHQFEVYCDSRQLPYFQRFCEDMQIWHNDEEKYSSALFMLRSARLVWLEKKGRGKPWNVNYLYLHCSLDTSLWTAEGTEQIRINKINETDEAIAKAKTKDKQELTENQLAYLQRQQSTRNKLNNSFPGRPSKPIYKGNSHILVGVSLGLEKPVTVAAVDVVSNKVLAYRSVKQLLGQNYKLLNRQRQQQKHLAQKRHESQKKQAPNQFGESELGLYVDRLLAKSIINFAKTYQASSIALPKLRDMREIIQSEIQAKAESKIPGYKEGQEKYAKEYRMSVHRWSYGRLIGNIQAQAAQAGILIETSSGQIRGSPQEQAKHLAISAYIERQTILNK

SEQ ID NO: 8 - protein, 682 aa, unidentified
  source 1..682: note = Cyanothece sp.
MTLKTLECRLYAPSDTLRYLWLLMAEKNTPLINEIINHLSEHPDFDQWFKAKQIPKSAISDICNDLKSQENYQNQPGRFYSSAISLTHYMFKSWFAVHKQLQRRIEGKRRWLNLLKSDQELEQNCGQSLEIIIQKAEEILKLMDSEKSQSSSKPKKPKKPKKKKKSSSEETITLFDRLFKAYNQGNDSLESYALAYLLKNNGQIPEDDEDLDKFALRKRKKEIEIERLQQQLENRIPLGRDLTGELWQEMLTIVNESIPQDENEASAWQAKLLKKSHNIPYPVAYETNTDLKWSKDSRGHLLVTFNGLVESLKKLNLNPEFEIRCDRRHLPWFQRFCKDQEIKANNDQHSSALFVLRSARLIWREGQGKEDPWKIHQLYLQCSVETQLWTEAGTKQVQSEKMVEFQLNQLRMKPELTFPIFFRSQSLPTYFNLWKVITSYRILKFLEKGDFTKAQKNFQDAIKRTESCLENLQSSYLTSQKSLYQGNPEIIMGVAMGLSQPATIAVVNVVTQEVLTYRSLKQLLGKNYNLLNRQRQQKQKLSHQRHKAQKKDAFNQYGESELGQYVDRLIAKAIVQVAKEYQADSIAVPKIRQMREIIQSEVQARAERKIQGYKEGQKKYAQQYRENVHQWSYGRLIESIHQASAKFGIRVEIASQSYQGSFQEQAQNLAIAAYTNRLEAVG

SEQ ID NO: 9 - protein, 660 aa, unidentified
  source 1..660: note = Cyanothece sp.
MTHITVVQCRLIAPESTLQHIWKMMAQQQTPLINQLLHDINTHPDINTWLTANQLPSKLVETLAQPLKTQSPYQGLPGRFITSAIILVKEMYASWFAIQTQKRLSLEGKKRFLTILKSDKQLIQDSQTDFLTLCYKAQQLLKRTQNKLKLDEPQHSEKAHWSIINALYPAYNNAKTPISRAAFALLIKNNGQVPDTPENPDYYQQRRKRKEIQIRRLEEQLKASLPKGRILDSKHWENTLKLAQTPITTIEEITSLQTQLLQKYSHLPFPVFYGTNTDLTWFKNPQGRICVKFNGLNQYPFQIACNKRQYPWFQRFFTDYQSYKSHKQQVPTGLMVLRSARLLWQPTNGQGEPWNTHHLSLHCAIDNDLWTISGIQQVKQQKILQTEQKIANFHSKALEKELTPNQQQRLKASQTSLNLLKTFDINEFFPSKCSLYQGSPDIILGVSIGLENPATIAIINISTQEILTYRTTKQLLSRTRKVRNKKPNSNNSNQSLSSAYKQISNYELFLQYQQQKHHNQHQRHNAQINDANNNYGEANLGLYLNRLLAKAILELAQQYQVSLIILPSLKNKRELIESEIRAKAELKYPGCKEKQDSYAKDYRTNVHQWSYQQLIKCIESKAAQIGIDTATGKQMNLETSQDQARNLVLNFCQKFSPTQV

SEQ ID NO: 10 - protein, 616 aa, unidentified
  source 1..616: note = Geminocystis sp.
MAHVTIQCRLIASRDTRQFLWQLMAQKNTPLINEILLRIKQHPDFPHWRTKKRLPKDFLARQIAELKNNYPFEEQPSRFYASVNKVIDYIYKSWFEVQKALDWKLQGNLRWVEMLLPDTELIKHFDNSLESLQQQATLILDSIDSTVSHDRISTILFEKCGKTKKPEIKSAIIYLLKNGCTIPKKPETTEKYQDLKRKVEIKITKLHRQIESRIPLGRDLEDKKWLDTLITASTTAPIDQTEANTWFSILKQNQSSIPYPILYETNEDLKWSLNEKNRLSIRFSGLGEHSFQLCCDHRQLPYFQRFYEDQELKKASKDQLSSALFTLRSAMILWKEDEGKGELWDRHKLYLHCTFETRCLTAEGTSTIVEEKQKEVTKIIDLMKAKEELSDSQQAFIRRKNSTLAKLNNTFPRPSKPVYQGKPNVHLGIAMGLEQPVTIAIVDIETDKVITYRNTKQLLREDYRLLRRRRIEKQKLSHQNHKARKRFNFQQKGESNLGEYLDRLIAKAILTVAQEYQVSTILIPRLRDMRSITEAEIQLRAEKKIPEYKEGQKKYAQDYRVQVHQWSYGRLIENVKLICEKVGIVVVEAKQPKQGTLTEKALQLVLSATEKNLKKK

SEQ ID NO: 11 - protein, 636 aa, unidentified
  source 1..636: note = Leptolyngbya boryana
MSVITIQCKLVATEETRRALWHLMAEKHTPLINELLKHIAQDSRFEEWSLTGKLPRLVVSEACNQLKQDPQFSGQPGRFYSSAISTVHRIFLSWLALQTRLRNQISGQTRWLAMLQSDNELTIASQTDINTLRLKASELLTHLNEPISESDQPEVKKTRSKKKNQTSNQAGANVSRTLFKLYDETEDPLTRCAIAYLLKNGCKLPDQNENPEKFIKRRRKTEIRLERLMNTFQTTRIPRGRHLSWHSWIEALETATSHIPENEEEAAGWQARLLTKPAILPFPVNYETNEDLRWSLNSQGRICVSFNGLSEHFFEVYCDQRDLHWFNRFLEDQETKKASKNQHSSSLFSLRSGQIAWQEGKGDAEHWVVHRLVLSCSIETDTWTQEGTEEIRQKKASDCAKVIASTKAKENRSQNQDAFIRRRERMLELLENQFPRPSYPLYQGQPSILAGVSYGLDKPATLAIVNIQTGKAITYRSIRQILGKNYKLLNRYRLNQQRNAHKRHNNQRKGGSSQLRESNQGQYLDRLIAHEIVAIAQEYQVSSLALPDLGDIREIVQSEVQARAEQKILGSIEQQRKYARQYRASVHRWRYAQLTQFIQSQAAQVGISIEITKQPLSGTPQEKARNLAIAAYQSRK

SEQ ID NO: 12 - protein, 615 aa, Myxosarcina sp.
MSQNAIQCRLIAPETTRRQQWQLMAEKNTPLINELLKQLAEHPELETWKRKGKIPPGTVKNLCQPLRTCPQYINQPGRFYSSVISLAEYIYRSWLKLQRRLIFRLNGQQRWLQMLKSDEELVAESGRSLKEIEAKASEALDRLNREENPSISNRLFDLYDETEDILIRSAIVYLLKNGCKIRQKPEDPKKFARRRRKTEIRVKRLQEKLNGKAPQGRDLTGEKWLNTLFTATSQVPQDEAQAKSWQDILLTKSKLVPYPIVYESNEDLTWSKNERGRLCVKFNGLSDHTFQIYCDRRQLKIFNRFYEDQQIKKASKNSHSSALFTLRSATIAWQEGKGKGEPWNVNRLILYCTFDNLLLTTEGTEVVRQEKAEAIANTLTKIKEKGDLNQKQQAFIRRKETSLSRINNPFPRPSRPLYKGKSNILLGVAIRLDKPATVAIVDGATDKAIAYLSTKQLLGKNYHLLNRKRQQQHILSHQRNVAQRHHANNKFGESELGQYIDRLLAKAIIQLAKDYRVGSIVVPYMEDTREIIQAEVQARAEAKIPGCIEKQKEYAKKYRTNIHKWSYGRLIDLIKAQAAKAGIVIEESKQSIRGDPKKQAKEIAVCAYRDRIVPF

SEQ ID NO: 13 - protein, 639 aa, unidentified
  source 1..639: note = Nostoc punctiforme
MSQITIQCRLIASESTRQKLWKLMATLNTPLINELIEQLGKHPDFENWRQQGKLPTTVVSQLCQPLKTDPRFVGQPSRLYMSAIHIVDYIYKSWLAIQKRLQQQLDGKMRWLEMLNSDVELVETSGSSMGAIRTKASEILAKAMPTSDSDSSQPKTKKGKEAKKSSSSSSDRSLSNKLFEAYQETEDILSRSAISYLLKNGCKLSDKEEDSEKFAKRRRQVEIQIQRLTEKLISRMPKGRDLTNRKWLETLFTATTTFPEDNAEAKRWQDILLTRPSSLPFPLVFETNEDMVWSKNQKGRLCVHFNGLSDLSFEVYCDNRQLHWFQRFLEDQQTKRQSKSQYSSGLFTLRNGHLVWQEGEGKSEPWNLNRLNLYCCVDNRLWTADGTEQVRQEKAEEISKLITKMKEKSDLKDTQKAFIQRKESTLNRMNNSFERPSQPLYQGQSHILVGVSLGLEKPATVAVVDAIAGKVLAYRSIRQLLGDNYELLNRQRRQQRSSSHERHKAQKSFSPNQFGTSELGQYVDRLLAKEIIAIAQTYKAGNIVLPKLGDMREIVQSEIQAIAEAKCPGSVEVQQKYAKQYRVNVHKWSYGRLIQSIQSKGSQAGIVIEEGKQPVRGSPHEQAKELALSAYHDRLARRS

SEQ ID NO: 14 - protein, 648 aa, Nostoc sp.
MCFVWYFIFMSQKTIQCRLIASESTRQKLWKLMAESNTPLINELLQQLSKHPDFEKWRRNGKLPSTVVSQLCQPLKTDPSFTGQPSRFYISAIHIVDYIYKSWLTIQKRLQQQLDGKLRWIEMFNSDVELVEISGFSLEAIRTKASEILAITTPESDPKTLLTKRGKTKQSKKSSASNPDRSLSRKLFDAYQETDDILSRSAISYLLKNGCKLNDKEENPEKFAKRRRKVEIQIQRLTDKLTSRIPKGRDLTYSKWLETLFTATTTVPENNAEAKRWQDILLTRSSSIPFPVVFETNEDLVWSTNEKGRLCVHFNGLSDLIFEVYCDSRQLYWFKRFLEDQQTKRKSKNQHSSGLFTLRNGRLAWQQGEGKGEPWNIGHLALYCCVDNRLWTAEGTEQVRQEKAEEITKFITKMKDKSDLSETQLAFIKRKESTLTRINNSFDRPSKPLYQGQSHILVGVSLGLEKPATIAVVDAIAGKVLTYRSLRQLLGDNYELLNRQRRQQRSLSHERHKAQKSFSPNQFGASELGQYVDRLLAKEIVAIAQTYKAGSIVLPKLGDIREIVQSEIQAIAEAKCPSSSEIQQKYAKQYRVNVHQWSYGRLIQSIQSKAAQIGIVIEEGKQPIRGSPQDKAKELALYAYSLRLARRS

SEQ ID NO: 15 - protein, 639 aa, Nostoc sp.
MSQKTIQCRLIASESTRQKLWKLMAESNTPLINELLQQLSKHPDFEKWRRNGKLPSTVVSQLCQPLKTDPSFTGQPSRFYISAIHIVDYIYKSWLTIQKRLQQQLDGKLRWIEMFNSDVELVEISGFSLEAIRTKASEILAITTPESDPKTLLTKRGKTKQSKKSSASNPDRSLSRKLFDAYQETDDILSRSAISYLLKNGCKLNDKEENPEKFAKRRRKVEIQIQRLTDKLTSRIPKGRDLTYSKWLETLFTATTTVPENNAEAKRWQDILLTRSSSIPFPVVFETNEDLVWSTNEKGRLCVHFNGLSDLIFEVYCDSRQLYWFKRFLEDQQTKRKSKNQHSSGLFTLRNGRLAWQQGEGKGEPWNIGHLALYCCVDNRLWTAEGTEQVRQEKAEEITKFITKMKDKSDLSETQLAFIKRKESTLTRINNSFDRPSKPLYQGQSHILVGVSLGLEKPATIAVVDAIAGKVLTYRSLRQLLGDNYELLNRQRRQQRSLSHERHKAQKSFSPNQFGASELGQYVDRLLAKEIVAIAQTYKAGSIVLPKLGDIREIVQSEIQAIAEAKCPSSSEIQQKYAKQYRVNVHQWSYGRLIQSIQSKAAQIGIVIEEGKQPIRGSPQDKAKELALYAYSLRLARRS

SEQ ID NO: 16 - protein, 641 aa, Nostoc sp.
MSVITIQCRLVASEDTRRHLWQLMAEKNTPLINELLKQVRIHPDMEQWLKKGKLPDGVIKPLCDSLSTQECFVNQPKRFYKSAIEVVEYIYKSWLALQKERQQKIDKKEHWLNMLKSDIELEQESNFSLNIIRAKAIKVLAHYIAKVEENHKQDITNKNVNKRKKSKSKNNNYTLFDILFKAYDQAKVSLNRCAIAYLLKNNCQVSEEEEDPNRYALRRSKKLKEIERLKEQLKSRIPSGRDLTGQEWLQSLLVATTNVPESENQFKIWQKHLLQNSSSIPFPVQFTSNEDLIWSKNEKGRICVSFSGEEFNNHIFEIYCDKKQIYWFQRFLEDQSIKRNNKKQYSSSLFTLRSGRLAWQDNKGNDLPWKIHRLTLYCSVDTRLWTIEGTQEFRREKVDEITEKLADMEKKENLNKNQQIYVKRLNSTLTKIDTAYPRPNQNLYQGKTSILIGVSLGLEKPATIAIVDCPTNKVLAYRSVKQLLGDNYNLLNRQRQQQQRNSHERHKAQKSNTQIKLSELELGKHIDNLLAQAIITLAKSYQAGSIVLPTMKNVRESIQSEIEARAVKRCPNYKEGQQQYAKQYRQSIHRWSYNRLIGCIKSQAAKANISIEQGPQPIRDSPQEKARDLAIAAYHFRQNKS

SEQ ID NO: 17 - protein, 639 aa, unidentified
  source 1..639: note = Scytonema hofmanni
MSQITIQARLISFESNRQQLWKLMADLNTPLINELLCQLGQHPDFEKWQQKGKLPSTVVSQLCQPLKTDPRFAGQPSRLYMSAIHIVDYIYKSWLAIQKRLQQQLDGKTRWLEMLNSDAELVELSGDTLEAIRVKAAEILAIAMPASESDSASPKGKKGKKEKKPSSSSPKRSLSKTLFDAYQETEDIKSRSAISYLLKNGCKLTDKEEDSEKFAKRRRQVEIQIQRLTEKLISRMPKGRDLTNAKWLETLLTATTTVAEDNAQAKRWQDILLTRSSSLPFPLVFETNEDMVWSKNQKGRLCVHFNGLSDLIFEVYCGNRQLHWFQRFLEDQQTKRKSKNQHSSGLFTLRNGHLVWLEGEGKGEPWNLHHLTLYCCVDNRLWTEEGTEIVRQEKADEITKFITNMKKKSDLSDTQQALIQRKQSTLTRINNSFERPSQPLYQGQSHILVGVSLGLEKPATVAVVDAIANKVLAYRSIKQLLGDNYELLNRQRRQQQYLSHERHKAQKNFSPNQFGASELGQHIDRLLAKAIVALARTYKAGSIVLPKLGDMREVVQSEIQAIAEQKFPGYIEGQQKYAKQYRVNVHRWSYGRLIQSIQSKAAQTGIVIEEGKQPIRGSPHDKAKELALSAYNLRLTRRS

SEQ ID NO: 18 - protein, 690 aa, Scytonema sp.
MSVITIQCRLVAEENTLRQLWELMAEKNTPLINELLEQVGQHPNFEKWLKKGEVPEEAIDTIKKSLITQEPFAGQPGRFYTSAVTLVKEIYKSWFALQQERQRKIEGKERWLKMLKSDIELQQESQCNLDIIRNKANEILTSFVANFTENRNQQFKKKGNKTKKNKKEEEESTLFNALFKIYDKTKDCLSQCALAYLLKNNCQVSEIDEDPEEYVKRRRRKEIEIERLRKQLKSRKPKGRDLTGEKWLTALKEATNQVPVDQLEAKSWQASLLKVTSDIPYPVDYESNTDLDWLIHSNDDDIKKKVILVWQIYFLKQLIKSGSYSFIKYLYFQRGCLPKRDVNWLNLKNKAGRIFVKFNGLRKNIINPEFYICCDSRQRHYFQRLCQDWQVWHDNEETYSSSLFFLRSARLLWQKRKGTGAPWKVNRLILQCSIETRLWTEEETELVRIEKINQAETEIRESEQKGKPKQKVLSHRQKLNNLFPNRPSKPIYKGKPNIIVGVSFGLDKPATVAVVDVANKKVLAYRSTKQLLGKNYNLLNRQRQQQQRLSHERHKAQKRNAPNSFGESELGQYVDRLLADAIIAIAKTYQAGSIVIPKLRDMREQITSEIQSRAEKKCPGYKEAQQKYAKEYRLSVHRWSYGRLIESIKSQAAKVGISTEIGTQPIRGSPEEKARDLAVFAYQERQAALV

SEQ ID NO: 19 - protein, 627 aa, Spirulina major
MSQITIQCRLVASEATRQVLWTLMAERNTPLINELLAQMAQHPDLEEWRQKGKPTPGVVKKLCDPLRQDPRFMGQPGRFYSSAIALVEYIYKSWLKLQQRLQRKLEGQQRWLGMLKSDPELCEENHCTLDTLRDKAAEILASLESPQPKQGKVKTKKAKAQSSPRQSLFEMHDGAEDGFVKSAIAYLLKNGGKLPTHEEDPKKFAKRRRKAEVKVERLIHQITASLPKGRDLTGERWLETLLTASYTAPKDAQQTKVWQSILLTKTKAVPYPINYETNEDLTWSKNEKGRLCVRFNGLSEHTFQIYCDQRQLKWFQRFYEDQEVKRTSKNQHSTSLFTLRSGRIVWQESDRNDKPWTANHITLCCTLDTRLWSAEGTEEVRTEKAIDIAKTLTNMNEKGDLNDKQQAFIKRKTATLDRINNPYPRPSKPLYHGQSHILVGVALGLDKPATVAVVDGTTGKAITYRNLKQLLGENYKLVNRQRQQKQAQSHQRHKAQKRSGTDQFGDSELGQHIDRLLAKAIVAFAHSQSAGSIVVPKLEDIREIVQSEIQARAEEKVPGYIEGQKQYAKRYRVQVHQWSYGRLIDSIKSKATQQQVVIEEGKQPVRGSPEAQATELAISTYHLRASS

SEQ ID NO: 20 - protein, 608 aa, unidentified
  source 1..608: note = Geminocystis sp.
MAVITIHCRLISSKSNRHQLWNLMVQKNTPLINELLLELSQHEDLEQWCELGKLPSGLISKLCDQLKQRAEFEGQPSRFYASAINLVDYIYKSYLRTQRRLRFRLQGQQRWFEMFKSDTEFKNETNFSLTDIRVKARELLDKDLKDSSPDDYFKTYESTSDLLTRSAISYLLKNGRKLPEKPEDYQKFQKRRRKLQIKIEKLQKKIDSSPPMGRNLTNDSWLGMLNLVSNTIPQTDEEAKQWQDQLLRQSKSVPYPVMFNTNEDLRWSKNKKGRLCVTFNGLGKLVFEIYCDQQQLKWFERFYEDQEVKRKGKNQHSSALFTLRSGMLLWQEHEGKQEAWQNNHLTLYCSLDTCFETAEGTELVRQKKVKEVVNLIDAMNNKSERTKTQDAFIKRKQSTLARLDNSFPRPSKPLYQGNQNIVVAVSMSLEYPATIAMFNMSSQEVLTYRSTKQLLDNNYHLLNRQRNQKQRLSHQRHKTQRQNSSDFFTQQESELGQYLDRLLAQSIVSIAKQYQASTILLPNLKNIRDSIQAEIEAKAEAKIPNCKEAQKKYLKNYRINIHHWSYGRLIDSIQLQASKLDILIQEVKQPIRGSPQEKAKQMAILTLE

SEQ ID NO: 21 - protein, 635 aa, Nostoc sp.
MSVITIQCRLVAEEDTLRQVWELMTDKNTPLVNELLAQVGKHPEFETWLEKGKIPTEFLKTLVNSLKNQERFSDQPGRFYTSAIALVDYVYKSWFALQKRRKRQIEGKERWLIILKSDLQLEQESQCSLNVIRTEANEILAKFTPQSDQNKNQRKSKRTRKSAKLQTPSLFQNLLNTYEQTQETLTRCAIAYLLKNNCQISERDEDPEEFNRNRRKKEIEIERLKDQLQSRIPKGRDLTGEEWLKTLEIATTNVPQNENEAKAWQAALLRKPADVPFPVAYESNEDMTWLQNDKGRLFVRFNGLGKLTFEIYCDKRHLHYFKRFLEDQELKRNSKNQHSSSLFTLRSGRIAWSLGEEKGEPWKVNKLHLYCTLDTRMWTIEGTQQVVSEKTTKITETLNQAKRKDVLNDKQQAFVTRQQSTLDRINNPFPRPSKPNYQGQPSILVGVSFGLEKPVTLAVVDVIKNEVLAYRTVKQLLGKNYNLLNRQRQQQQRLSHERHKVQKRNAPNSFGESELGQYVDRLLADAIIAIAKTYQAGSIVIPKLRDMREQISSEIQSRAEKKCPGYKEVQQKYAKEYRMSVHRWGYGRLIESIKSQAAKAGIFTEIGTQPIRGSPQEKARDLAVFAYQERQAALI

SEQ ID NO: 22 - protein, 667 aa, unidentified
  source 1..667: note = Cyanothece sp.
MSTITIQCRLVAPEATRQALWQLMAQKNTPLVSELLRQVAQHPDFETWRQQGKLEAGIIKKLCEPLKKDPRFNEQPARFYTSAIALVDYIYKSWLKLQQRLQRKLEGQNRWLAMLKSDDELIQISQTNIEIIQAKATEILSTLQPQDREQSSKKKAKKCKKSTNKNSLFSQLDKLYNEINNNLTHCAIRYLLKNGGKIPQRPEDTEKFAQRRRKVEIKIERIIEQIESSIPQGRDLTGDSWLETLIIAANTATVEAEDVKSWQDKLLSQSKSIPYPVAYETNEDLTWSINEKGRLCVRFNGLGKHTFQIYCDQRQLKWFQRFYEDQQIKKDGKDHHSSALFSLRSGRIVWQEGLGKGKPWNIHRLTLHCSLDTRFWTEEGTQQVQQEKSKKFQTNRLRMKPELTFSIFFRSQTLETYLQVWLVITAYRLQSFLDKGNVAKAHQEFQKAIKRNESSLQKITSSYNRPHKTLYQGKSHIFVGVAMGLEKPATVAVVDGTTGKAIAYRSLKQLLGNNYHLFNRQGKQKQNTSHQRHKSQKHFADNQFGESQLGQYIDCLLAKAIISVAQTYCAGSIVVPKLKDMRELIQSEIQAKAEAKIPGYVEGQAKYAKSYRVQVHQWSHGRLIDNITSQASKFNITVEEGEQPHQGNPQDKAKLLAIAAYHSRLCA

SEQ ID NO: 23 - protein, 639 aa, Calothrix sp.
MSVITIQCRLVADEETLRHLWTLMAEKNTPFANEILEQLAQHAEFESWVKNSRVPATVIKELCDSLKNQELFAGQPGRFYTSATTLVTYIYKSWLAVNKRLQRKIEGKKQWLDMLRSDTELEQESNSNLEKIRAKATEILDSFATRQINQVNSKSKTSKNNKNKQEKEVKSLSIQSNILFETYRQTEDNLTKCAIVYLLKNNCEVNDVEEDIEEYEKNKRKKEIQIKRLEDQLKSRVPKGRDLTGEKWVEVLEKAVNSVPESENEAKSWQASLLRKSSQIPFPVVYETNEDIKWSINEKGRIFVSFNGLGKLKFEIFCDKRHLHYFQRFLEDQDIKRQGKNQHSSSLFTLRSGRISWLEQPGKGKPWNINRLLLFCSIDTRMLTAEGTQQVIEEKIADTQNKIAKAQEKCEGELNPNQQAHINRKKSTLARINTPFPRPSKPLYQGKSHIVVGVSLGLKATATIAVFDAMNNQVLAYRSTKQLLGDNYKLLNRQQQQKQRLSQQRHKSQKQFASNSFGESELGQYVDRLLAKEIVAVAKNFGAGSIVLPKLGDMREIIQSEVQAKAEKKIPGFIELQKNYAKEYRKSAHNWSYGRLIENIQSQATKEGIEIETGKQPTRGIPQEQARDLALFAYQCRIA

SEQ ID NO: 24 - protein, 635 aa, Anabaena cylindrica
MSQITIQCRLVASETTRQQLWQLMAEKNTPLINELLSQIGKHPEFETWRQKGKHPTGIVKELCEPLKTDPRFIGQPARFYTSATASVNYIYESWFALMKRYQSQLDGKLRWLEMFNSDAELVEHSGVSLDTLRATSAEILAQFAPQDTNRDTSNKGKKSKMGKKSQKSDSEGNLSKKLFDAYSSAEDNLTRCAISHLLKNGCKVSNKEENSEKFTQRRRKLEIQIQRLTEKLAARIPKGRDLTDTQWLETLFTATYNVPEDETEAKLWQNSLLRKFSSLPFPVAYETNEDLVWSKNRFGRICLTFPTLREHIFQIYCDSRQLHWFQRFLEDQEIKKNSKNQHSSALFTLRSGRIAWQEGEGKGEPWDIHHLTLYCCVDTRLWTEEGTNLVKEEKAEEIAKTITQTKAKGDLNDKQQAHLKRKNSSLARINNPFPRPSQPLYKGQSHILLGVSLGLEKPATVAVVDGTTGKVLTYRNIKQLLGDNYKLLNRQRQQKHLLSHQRHIAQRIAAPNNFGDSELGEYIDRLLAKEIIAIAQTYQAGSIVLPNLGDMREQIQSEIKAKAEQKSDLVEVQKKYAKQYPNSVHQWSYGRLITNIQSQSKKAGIVIEEGKQQIRASPLEKAKELAINAYQSRKA

SEQ ID NO: 25 - protein, 642 aa, Anabaena cylindrica
MSVITIQCRLVAEEDSLRQLWELMSEKNTPFINEILLQIGKHPEFETWLEKGRIPAELLKTLGNSLKTQEPFTGQPGRFYTSAITLVDYLYKSWFALQKRRKQQIEGKQRWLKMLKSDQELEQESQSSLEVIRNKATELFSKFTPQSDSEALRRNQNDKQKKVKKTKKSTKPKTSSIFKIFLSTYEEAEEPLTRCALAYLLKNNCQISELDENPEEFTRNKRRKEIEIERLKDQLQSRIPKGRDLTGEEWLETLEIATFNVPQNENEAKAWQAALLRKTANVPFPVAYESNEDMTWLKNDKNRLFVRFNGLGKLTFEIYCDKRHLHYFQRFLEDQEILRNSKRQHSSSLFTLRSGRIAWLPGEEKGEHWKVNQLNFYCSLDTRMLTTEGTQQVVEEKVTAITEILNKTKQKDDLNDKQQAFITRQQSTLARINNPFPRPSKPNYQGKSSILIGVSFGLEKPVTVAVVDVVKNKVIAYRSVKQLLGENYNLLNRQRQQQQRLSHERHKAQKQNAPNSFGESELGQYVDRLLADAIIAIAKKYQAGSIVLPKLRDMREQISSEIQSRAENQCPGYKEGQQKYAKEYRINVHRWSYGRLIESIKSQAAQAGIAIETGKQSIRGSPQEKARDLAVFTYQERQAALI

SEQ ID NO: 26 - protein, 679 aa, Nostoc sp.
MSSDRKKKSTIPVHRTIRCHLDASEDILRKVWEEMTQKNTPLILKLLKSVSEQPEFEANKEKGEITKKEIVKLRKNVTKNPELEEQSGRLRSSAESFVKEVYSSWLTLYQKRKRQKEGKEYFLKNILKSDVELIDESNCDLETIRSKAQEVLSQPEEFIKQLTINDEDVKPTKSARKRVNKNINNKSTDAEQRKDSSSTNNVDKNKLETLTNILYEIHKQTQDILTRCTVAYLIKNHNKISNLEEDIQKLKKRRNEKIVQIKRLENQIQDNRLPSGRDITGERYSEAFGNLINQVPKNNQEWEDWIANLSKKISHLPYPIDYLYGDLSWYKNDVGNIFVYFNGWSEYHFKICCNKRQRHFFERFLEDYKAFKVSQKGEEKLSGSLITLRSAQLLWQQGEGKGEPWKVHKLALHCTYDSRLWTAEGTEEVRKEKTDKAQKRVSKAEENEKLDDIQQTQLNKDKSSLSRLKNSFNRPGKLIYQSQSNIIVGISFHPIELATVAIVDINTKKVLACNTVKQLLGNAFHLLSRRRRQQVHLSKERKKAQKKDSPCNIGESKLGEYIDKLLAKRIVEIAKFYQAGCIILPRLKDMKEIRTSAIQAKAEAKIPGDVNAQKLYVKEYNRQIHNWSYNRLQESIKSKAAEFKISIEFGIQPHYGTLEEQAKDLAFYAYQSRNHTLGR

SEQ ID NO: 27 - protein, 632 aa, Anabaena sp.
MSVITIQCRLVAEEDTLRTLWELMADKNTPLINEILAQVGKHPEFETWLEKGKIPTELLKTLVNSLKTQERFASQPGRFYTSAIALVDYVYKSWFALQKRRKRQIEGKERWLTILKSDLELEQESQCSLNIIRTKATEIITEFTPQSDQNNSQKKRKKTTKSTKPSLFQILLNNYEETQDILTRCALAYLLKNNCQISERDENPEEFTRNRRKKEIEIERLKDQLQSRIPKGRDLTGEEWLKTLEVVRANVTQNENEAKAWQAAILRKSADVPFPVAYESNEDMTWLQNDKGRLFVRFNGLGKLTFEIYCDKRHLHYFKRFLEDQELKRNSKNQYSSSLFTLRSGRLAWSPGEEKGEPWKVNQLHLYCTLDTRMWTIEGTQQVVDEKSTKITETLTKAKQKDDLNDKQQAFVTRQQSTLNRINNLFPRPSKSRYQGQPSILVGVSFGLENPVTLAVVDVVKNEVLAYRSVKQLLGKNYNLLNRQRQQQQRLSHKRHKAQKRNAPNSFGESELGQYVDRLLADAIIAIAKTYQAGSIVIPKLRDMREQISSEIQSRAEKKFPGYKEAQQKYAKEYRMSVHRWSYGRLIESIKSQAAKAGISTEIGTQPIRGSPQEKARDLAVFAYQERQAALI

SEQ ID NO: 28 - protein, 150 aa, Anabaena sp.
MEGKFYTSTEAAEITNCSRRQLQYWREKGVIVPTVNASGKGRNVYYSKADLLALSVMEHLLSIGLNFEMCHVALETLRKREPWLFEESVTEDKMKRLMFLPSKSPEQPLQLAEFDQQAALEALCEGQTVIPFWGDRIHELLHQNLKRFTE

SEQ ID NO: 29 - protein, 150 aa, Anabaena variabilis
MQETFFTSKEASKITGCTLRQLQYWREKGVVVPVISDTGTGRSIYYSKTNLVELAAMVYWLSVGLSFDIACETLKTLKDQEPELFSSGTGRRFMLLSEAQERSPLGAPKISLELMEFDRTKAIASLDEGKPVIPVWLDEIYQQLQQKLKA

SEQ ID NO: 30 - protein, 139 aa, unidentified
  source 1..139: note = Geminocystis sp.
MKERFFSSKQTSLITGCSLRQLQYWRDKEVIVPFIQGTGTGKTIYYSPAELVEISIMVYLLSVGLSFEIGQIILSDLKEKERNFRNPDYKGRFMIIPRDGKMSLLSYEREKAIELLDQGTSIVPLWLDRIHEQLKETLG

SEQ ID NO: 31 - protein, 164 aa, Myxosarcina sp.
MGEPFYTSTEASLITGCSRRQLQYWRKQGVVVPTVNPGGKGRNVYYTESDLLILSVMKYLLSLGLNFDVSLKVLETLREKEILCFLDWSLSKNTKKRFMLVLDTEEKNTIHITNFDTELAVQKLQEGFAIAPFGRDRIYQKLQDNLQAFYRNRKSLKGDRRTKN

SEQ ID NO: 32 - protein, 150 aa, unidentified
  source 1..150: note = Nostoc punctiforme
MEEKFYTSTEAAEITNCSRRQLQYWRDKGVVIPTVNTTGKGRNVYYSVADLLTLTVMHYLLSVGLSFEVCREALAMLQDKEPWLFEEFVSKKKMRRLMLISNASQQKYLTLAEFDKEAALEALCQGQTVIPFWCDRIHEVLHQNLKRFSE

SEQ ID NO: 33 - protein, 168 aa, Nostoc sp.
MLYKALMCFRRCFYRMFLMEGKFYTSTEASEITHCSRRQLQYWREKGVIVPTVNSSGKGRNVYYSKADLLALTVMEQLLSTGLNFDLCYAALQTLRKQEPWLFDESVPEEKMKRLMLLPTRSPEQPLQLAEFDKQAALEALCHGQTVIPFWSDRIHQQLRENLKSFSS

SEQ ID NO: 34 - protein, 150 aa, unidentified
  source 1..150: note = Scytonema hofmanni
MEGKFYTSTEAAQITNCSRRQLQYWRDKGVVVPTVNTTGKGRNVYYSISDLLVLTVMHYLLSVGLSFEVSRQTLVILRQKEPWLFEEFVPKEKMKRLMLLTTCSLEQPLTLAEFDKEAALEALCQGQTVIPFWCDRIHQQLRDNLKSFSS

SEQ ID NO: 35 - protein, 143 aa, Spirulina major
MEQPFFSSREAADITGCTLRQLQYWREKEVVIPTINATGTGRSIYYSQANLVELSVMAYWLSLGLTFEVAHESLMKLRAIEPRFVEPPIARRFMLKWEEMRGKLELVEFDREGAIASLDAGQPVIPVWLEQIYSKLSNKISPN

SEQ ID NO: 36 - protein, 158 aa, unidentified
  source 1..158: note = Cyanothece sp.
MEEMFFTSTEASKLTGCSRRQLQYWREKGVVVPTVNASGKGRNVYYSVSDLLKLMVMEYLLSVGLSFETAQESLEMLKKAHPEMFKADVSSEDLKRLMILPSPKRLELKDFDPEEAFKRINTYAMPLVPFSGEMIHERLKESLQGFSGNNHLKSSENS

SEQ ID NO: 37 - protein, 142 aa, Anabaena cylindrica
MQETFFTSKEAAQLTGCTLRQIQYWREKGIVVPIISDTGTGRSIYYSRSNLVELAALVYWLSTGISFDIACETLKRLKAQEPELFISGKGKRFMLLLSAQNESLSLVEFDRKSAIASLDEGKAVIPVWLDIIYQKLASRLAQ

SEQ ID NO: 38 - protein, 549 aa, unidentified
  source 1..549: note = Alkalinema sp.
MIVTTLSPDEERKIKVLQPLIEPCDRATYGKRLRYAAEQLGISVRSVQREIQRWEKNGIAGITRNCRADKGKHRISDFWQNFIIKTYKEGNTGSKRMTPKQVAVRVQAKAHEMRDENPPHYRTVLRVLKPIIERQEKTKSIRSPGWKGSTLSVKTRSGEDLVIAHSNHVWQCDHTPVDVMLVDQYGEKRKRPWLTIVIDSYSRCIMGINLGFDAPSSQVVALALRHAILPKHYGAEYKLNCEWGTYGKPVHFYTDGGKDFRSNHVQQIGHDLGFTCHLRDRPSEGGIVERPFKTLNEQLFSTLPGYTDSNVQKRPKDVEKDARLTLRDLERLVVRFICDCYNQSIDARIGDQTRFQRWEAGLPGVPDVIGERLLDPCLMNVSRRTIQRGGYLQFKNLMYRGEYLAGYAGEIVSVRFNPDDITTLLVYRRENNREVFLTRAFAQGLETEQLSLSEAQASSSRRKEAGKSLSNTAIFQEVLDRDALVNSKKSRKQRHKEEQKIARSETETPKTGAVSVEQPETAPTELEVALDLETETFEPIDFEELQGGW

SEQ ID NO: 39 - protein, 582 aa, Anabaena sp.
MNRDENSDLNTSAIPVESISEGDNTPPETNVIATELSEESQLKLEVLQSLLEPCDRTTYGQKLKEAAEKLAVSVRTVQRLVKKWEQDGLVGLTQTGRTDKGKHRIGEFWEDFIVKTHKEGNKGSKRMSPKQVAIRVQAKAHELSDLKPPNYRTVLRVLAPILEKQEKTKSIRSPGWRGTTLSVKTREGKDLSVDYSNHVWQCDHTPADVLLVDQHGELLSRPWLTTVIDTYSRCIMGINLGFDAPSSEVVALALRHAILPKRYSLEYKLHCEWGTYGKPEHFYTDGGKDFRSNHLSQIGAQLGFVCHLRDRPSEGGIVERPFKTLNDQLFSTLPGYTGSNVQERPEDAEKDAKLTLRELEQLIVRYIVDRYNQSIDARMGDQTRFGRWEAGLPSVPVPIEERDLDICLMKQSRRRVQKGGHLQFQNLMYRGEYLGGYDGETVNLRFNPRDITTVLVYRQENSQEVFLTRAHAQGLETEQLSLDEAEAASRRLRNLGKTISNQALLQEVLDRDALVANKKSRKDRQKLEQEILRSTAVNDSKNESLASPVMEAEDVEFTTPVQSSSPELEVWDYEQLREEYGF

SEQ ID NO: 40 - protein, 663 aa, Anabaena variabilis
MNYLDVDNQFELEDEDDFLLDDEDADILDFSIEVESLSNDGNSVAEDKLVEFLDQRYLEDSELRLSGEQRLKLEIIRNLREPCDRLTYGKRLEEAAKKLGKSERTVRRLIKSWEEKGIAALAEVPRADKGQVRKSEYWYNLSLKIYKQGNKGDKGDKGDKGSDRMTRTQVAEKVETKAYEFAKKELEAEISKLESQGFRGQDLDWELKKLIKIKEKAEGFKYWSKYGKPPSTRTVERWLKPVEEKRHKSRTSRSPGWHGSEHTIKTRDGQEISIKYTNQVWQIDHTKADILLVDEDGEEIGRPQLTTVIDCYSRCIVGFRLGFAAPSSQVVALALRHAIMPKRYSSEYELRCKWSAYGVPKYVYTDGGKDFRSKHLVEWIANELDFEPILRSKPSDGGIVERPFRTISGLLSEMPGYTGSSVKDRPEGAEKKACISLPELEKLIVGYIVDSYNQKPDARSQANPLTPKQSRIERWEKGLQMPPTLLNDRELDICLMKAAERVVYDNGYLNFSGLRYRGENLGAYAGDKVILRFDPRDITMVLVYGRKNNKEIFLARAYAIGLEAASLSVEEVKYARKKAENSGKGINNISILEEALRRRNFLQQKKNKTKAERRRSEEKRIEKIPQVLTEKKNEQVAVLISQPEDDEPIEKLDLKLLREELGL

SEQ ID NO: 41 - protein, 560 aa, Anabaena variabilis
MLDDPDKGNQEPETHEIVTELSLDEQHLLEMIQKLIEPCDRITYGERQREVAGKLGKSVRTVRRLVKKWEQEGLTALQTATRTDKGTHRIDSDWQDFIIKTYKENNKDGKRMSPKQVALRVQTKAEELGQQKYPSYRTVYRVLQPIIEQKEQKEGIRHRGWHGSRLSVKTRDGKDLFVEHSNHVWQCDHTRVDLLLVDQHGELLSRPWLTIVVDTYSRCIMGINLGFDAPSSQVIALALRHAILPKRYGSEYGLHEEWGTYGKPEHFYTDGGKDFRSNHLQQIGVQLGFVCHLRDRPSEGGIVERPFGTFNTDLFSTLPGYTGSNVQERPEQAEKEACITLRELERLLVRYIVDKYNQSIDARLGDQTRYQRWEAGLIVAPNLISEEELRICLMKQTRRSIYRGGYVQFENLTYRGENLAGYAGENVVLRYDPKDITTLLVYRQKGNQEEFLARAYAQDLETEELSVDEAKAMSRRIRQAGKAISNRSILAEVRDRETFVNQKKTKKERQKAEQTIVQKAKKPVPVEPEEEIEVASVDSEPEYQMPEVFDYEQMREDYGW

SEQ ID NO: 42 - protein, 546 aa, unidentified
  source 1..546: note = Aphanocapsa montana
MELVNPDDLNSVESRLKLEIIEKLSEPCDRKTYGERLRSAAQQLKCSVRTVQRLMKKWEEEGLAALIDSGRIDKGKPRIAEDWQQFIKKVYSNDKCTPAQVFTKVRNKARQEGLKDYPSHMTVYRILRLVKEAKEKEESIRNLGWKGSRLALKTRDGEVLEIDYSNQVWQCDHTRADILLVDKYGHQMGRPWLTTVIDTYSRAIVGINLGYDAPSSSVVALALRNAIMPKQYGVEYKLYADWPTCGTPDHLFTDGGKDFRSNHLRQIGLQLGFICHLRDRPSEGGIVERPFGTINTQFLSTLPGYTGSNVQDRPPEAEAEACLTLQELEKLLVAYIVNTYNQRLDARMGDQTRIQRWEAGLLKQPRVIPEHELHICLMRQTRRTIYRGGYLQFENLAYRGEALAEHAGENIVLRYDPRNIAQVLVYRHDPDREVYLGVAQALEFEGEVLALDDAKAHSRRIREDGKAVSNDAMLDEMRDREAFVDQKNKSRKDRQKDEQADLRPTTPPIIGPDSSDEPSVDVQPDESPEELDIPEFDIWDFDDDDA

SEQ ID NO: 43 - protein, 496 aa, Calothrix sp.
MGETLNSDNINALQVFDDSGELEEISESDDTESKNTIVTELSAEAKLRMEVLQSLLEPCDRKTYGIKLHDAAEKLGKTIRTVQRLVKKYQEEGLSAITEAERSDKGDYRIDKEWQEFIFKTYKEGNKNGRKMTPAQVAIRVQVKAEQLALSNYPSHMTVYRVLNPIIERKEQKQKVRNIGWRGSRVSHKTRDGQTLEPRYSNHTWQCDHTKLDIMLVDQYGETLARPWLTKITDSYSRCIMGINLGFDAPSSLVVALAMRHAILPKSYGSEYKLHCKWETYGVPENLFTDGGKDFRSEHLKQIGFHLGFECHLRDRPPEGGIEERGFGTINTDFLAGFYGYLGSNLQQRPEEAESEACITLRELHLLLVRYIVDNYNQRIDARSGNQTRFQRWEAGLPALAPLIDERQLDICLMKKTHRSIYKGGYVSFENITYRGEYLAAYAGESVILRFDPRDEWHLLKGKRIGMIGFHALVMAINTVGAYINTNTYQLHIHVN

SEQ ID NO: 44 - protein, 567 aa, unidentified
  source 1..567: note = Cyanothece sp.
MSLSLSIPDDSPHSHRLPSDEMITDEVKAKIDIIQSLIEPCDRITYRQRKEQAAKQLGVTIRSVERLLKKYREQGLIALMKTRSDKGKTRIDDEWKEFILNTYKEGNKGSKRITRHQVFLKVKGRAKQLGLNKEEFPSHQTVYRILDKFIEENERKKKARSPGYSGSRLTHITRHGRELEVEGSNDVWQCDHTCLDIRLVDEFGVLSRPWLTIIIDSYSRCVMGFFLGFYAPSSHIDALALRHAILPKSYSSDYQLKNEWNTYGIPTYYYTDGGKDFRSIHVTEQVAVSLGFNCFLRSRPSDGGIVERFFKTLNNSVLCELPGYTGSNVQQRPKNVDKDACLSLKDLEKILVKYIVDEYNQKPDARMKNQSRITRWEAGLLTEPYLYDERELDIALMKEAKRTLQKSGTLQFENLTYRSPLLKGREGERVAIRYDPDDVTTILVYEYLDDGTEAFLDYAHAQNLETEKLSYRELKAINKQLNQEEEAINNDKILDAMMERMEMTEELVKKNRQQRQQNAHEAVNSRPSVTEKLSISQPDEEDWEDDSEDEPLPTYKVQYMDDLFEDD

SEQ ID NO: 45 - protein, 555 aa, unidentified
  source 1..555: note = Geminocystis sp.
MTIENQISESQELISQLSPEEQAIADVIEDLVQPCDRKTYGAKLKKAAETLNKSVRTVQRYIKEWEEKGLLAIKKGNRSDQGTYRIEKRLQDFIVKTYREGNKGSKRISPKQVYLRTMAQAKEWSIDPPSHMTVYRILNPIIEEKENKKRVRNPGWRGTKLAVSTRSGNEINVEYSNHAWQCDHTRADILLVDQFGQLLGRPWLTTVVDTYSRCIMGINLGFDAPSSQVVSLALRHAMLLKSYSSDYGLHEEWGTYGKPEYFYTDGGKDFRSNHLQQIGLQLGFTCHLRSRPSEGGVVERPFKTLNTEVFSTLPGYTGGNVQERSEDAEKDASLTLRQLERIIVRYIVDNYNQRMDARMGEQTRFQRWDSGLLSIPDLLSERDLDICLMKQSRRRVQKGGYLQFENLMYQGEYLAGYEGETVILRYDPRDITAILVYRNEGNKEVFLTRAYGLDLETESMSWEDAKASAKKVRESGKNLSNRSILAEVKERHTFSDKKTKKERQRQEQEQVKPYIPSPVLKEVKEQTDKATDTDMSEEPIVEVFDYSQLQDDYGF

SEQ ID NO: 46 - protein, 569 aa, unidentified
  source 1..569: note = Leptolyngbya boryana
MQLPIEFPESEQVSRELVEQNQIVTELSDEAKLKIEVIQSLLEPCDRATYGNRLRDAATRLGKSVRTVQRMVKSWQEEGIAGLSNGERTDKGEHRIEQEWQDFIIKTYQEGNKNGKRMTPAQVAIRVKVKAQQEGITKYPSHMTVYRVLNPLIQRKTEKQNVRSIGWQGSRLSLKTRDGNSLSVEYSNQVWQCDHTRADILLVDQHGELIGRPWLTTVVDTYSRCIVGVNLGFDAPSSDVVALALRHAILPKTYPDRYQLNCDWGTYGKPEHFFTDGGKDFRSNHLQQIAVQIGFTCHLRNRPSEGGVVERPFGTLNTEFFSILPGYTGSNVQKRPEEAEESASLTLRELEQFLVRYITDRYNQGIDARMGDQTRFQRWEAGLLANPSVLTERELDICLMKQTRRTVYREGYLRFENLIYRGENLAGYAGETVTLRYEPRDITTVFVYHQEQGKEVFLTRAHAQDLETETISLYEAKASSRRIRDVGKTISNRSILEEVRDRDLFVQKKTRKERQKAEQAEVKIDQVPSPPQVLHLDEASQFETEIVETQCVEISEIEDYEKLRDDFGW

SEQ ID NO: 47 - protein, 563 aa, Myxosarcina sp.
MNSESTSETNSKLAISDLEDDREIVITSQLEGKAKERLEVIQSLLEPCDRATYGERLRAGAKKLDISVRSVQRLFKKYQEQGLTALVSTNRVDKGNRRISSFWQDFILQTYIQGNKGSKRMSPKQVAIRVQAKASEIKDNKPPSYKTVLRLLKPIQKKKERTIRSPGWQGTTLSVKTRDGQDIQINHSNQVWQCDHTLVDVLLVDRHGELIGRPWLTTVVDSYSRCVMGINLGFDAPSSLVVALALRHSILPKNYSQDFQLYCDWGVFGLPECLFTDGGKDFRSNHLEEIATQLGFIRKLRDRPSEGGIVERPFKTLNQSLFSTLPGYTGSNVQERPKDAEKDARLTLRDLEMLIVRFIVDKYNQSTIAGKDEQTRYQRWEAGLIKDPKIISERELDICLMKSKRRTVQRGGHLQFENIIYRGEYLAGYEGDIVNVRYNPINITTILVYRREQGKEVFLTRAHALGWETEIHSLSEARASVKRLRQAKKKISNESIHQEILLRDSAVDKKKSRKQRQKEEQSYKLITSPKVVAQDIESQEIERDISAEIADVEVWDFDELEDE

SEQ ID NO: 48 - protein, 584 aa, unidentified
  source 1..584: note = Nostoc punctiforme
MNSQQNPDLAVHPSAIPIEGLLGESDITLEKNVIATQLSEEAQLKLEVIQSLLEPCDRTTYGQKLREAAEKLGVSLRTVQRLVKNWEHDGLVGLTQTGRADKGKHRIGEFWEKFITKTYNEGNKGSKRMTPKQVALRVEAKARELKDSKPPNYKTVLRVLAPILEKQEKAKSIRSPGWRGTTLSVKTREGKDLSVDYSNHVWQCDHTRVDVLLVDQHGELLSRPWLTTVIDTYSRCIMGINLGFDAPSSGVVALALRHAILPKQYGFEYKLHCEWGTYGKPEHFYTDGGKDFRSNHLSQIGAQLGFVCHLRDRPSEGGVVERPFKTLNDQLFSTLPGYTGSNVQERPKDAEKDARLTLRELEQLLIRYIVDRYNQSIDARMGDQTRFERWEAGLPTVPVPIPERDLDICLMKQSRRTVQRGGCLQFQNLMYRGEYLAGYAGETVNLRFDPRDITTILVYRQENNQEVFLTRAHAQGLETEQLALDEAEAASRRLRNAGKTISNQSLLQEVVDRDALVATKKSRKERQKLEQAVLRSAGVDESKTESLSSQVVEPDEVESTETVHSQYEDMEVWDYEQLREEYGF

SEQ ID NO: 49 - protein, 537 aa, Nostoc sp.
MIQTLLEPCDRTTYGQKLKEAADTLGVTVRTVQRLVKKWEEDGLVGFIQTGRADKGKHRIGEFWENFIIKTYKEGNKGSKRMTPKQVALRVQAKARELGDSKPPNYRTVLRVLAPILEQKEKTKSIRSPGWRGTTLSVKTREGQDLSVDYSNHVWQCDHTRVDVLLVDQHGELLSRPWLTTVIDTYSRCIMGINLGFDAPSSVVVALALRHAILPKKYGAEYKLHCEWGTYGKPEHFYTDGGKDFRSNHLSQIGAQLGFVCHLRDRPSEGGIVERPFKTLNDQLFSTLPGYTGSNVQERLEDAEKDAKLTLRELEQLLVRYIVDRYNQSIDARMGDQTRFGRWEAGLPSVPVPIEERDLDICLMKQSRRTVQRGGCLQFQNVMYRGEYLAGYAGETVNLRYDPRDITTVLVYRQEKSQEVFLTRTHAQGLETEQLSLDEAEAASRRLRNAGKTVSNQALLQEVLERDAMVANKKSRKERQKLEQAILRSAAVNESKTESLASSVMEAEEVESTTEVQSSSSELEVWDYEQLREEYGF

SEQ ID NO: 50 - protein, 566 aa, Nostoc sp.
MTRQQHTSSPPETNLIVTELSEEAQLKLEVIQTLLEPCDRTTYGQKLKEAADTLGVTVRTVQRLVKKWEEDGLVGFIQTGRADKGKHRIGEFWENFIIKTYKEGNKGSKRMTPKQVALRVQAKARELGDSKPPNYRTVLRVLAPILEQKEKTKSIRSPGWRGTTLSVKTREGQDLSVDYSNHVWQCDHTRVDVLLVDQHGELLSRPWLTTVIDTYSRCIMGINLGFDAPSSVVVALALRHAILPKKYGAEYKLHCEWGTYGKPEHFYTDGGKDFRSNHLSQIGAQLGFVCHLRDRPSEGGIVERPFKTLNDQLFSTLPGYTGSNVQERLEDAEKDAKLTLRELEQLLVRYIVDRYNQSIDARMGDQTRFGRWEAGLPSVPVPIEERDLDICLMKQSRRTVQRGGCLQFQNVMYRGEYLAGYAGETVNLRYDPRDITTVLVYRQEKSQEVFLTRTHAQGLETEQLSLDEAEAASRRLRNAGKTVSNQALLQEVLERDAMVANKKSRKERQKLEQAILRSAAVNESKTESLASSVMEAEEVESTTEVQSSSSELEVWDYEQLREEYGF

SEQ ID NO: 51 - protein, 580 aa, Nostoc sp.
MYQKLKNSCLSSEDDAVELQQHQNSTKPHKLPSEELITEQVKLRMEVIQSLTEPCDRKTYRAKKIEAAQKLGVSIRQVERLLQKWREQGLVGLTTTRSDKGKYRLEQEWIDFIINTYKEGNKGSKRMTRNQVFLKVKGRAKQLSLNKSEHPSHQSVYRILDEHIEQKERKEKARSPGYLGERLTHMTRDGRELEVEGSNDVWQCDHTRLDVRLVDEYGVLDRPWLTIVIDSYSRCLVGFYVGFDHPSSQIDTLALGHAILPKSYGSEYQLRNEWQAYGKPNYFYTDGGKDFTSIHTTEQVAVQIGFNCALRRRPSDGGIVERFFKTLNLQVLNTLPGYTGSNVQERPENVDKDACLTLKDLELLLVRFIVNEYNPHTDARMKGQSRIGRWEAGLMADPYLYDQLDLAICLMKQERRKVQKYGCIQFENLTYRAEHLRGRDGETVAFRYDPADITTLLVYKMNADGTEEFMDYAHAQALETECLSLRELKAINKRLNEASQEINNDSILEAMIDRQAFVEETVKRNRKQRRQAANEQVNPVEQVAKKFATPEQKEVEPESEADIELPKYEVRYMDEFFEED

SEQ ID NO: 52 - protein, 584 aa, unidentified
  source 1..584: note = Scytonema hofmanni
MNSQQNPDLAVHPLAIPMEGLLGESATTLEKNVIATQLSEEAQVKLEVIQSLLEPCDRTTYGQKLREAAEKLNVSLRTVQRLVKNWEQDGLVGLTQTSRADKGKHRIGEFWENFITKTYKEGNKGSKRMTPKQVALRVEAKARELKDSKPPNYKTVLRVLAPILEKQQKAKSIRSPGWRGTTLSVKTREGKDLSVDYSNHVWQCDHTRVDVLLVDQHGEILSRPWLTTVIDTYSRCIMGINLGFDAPSSGVVALALRHAILPKRYGSEYKLHCEWGTYGKPEHFYTDGGKDFRSNHLSQIGAQLGFVCHLRDRPSEGGVVERPFKTLNDQLFSTLPGYTGSNVQERPEDAEKDARLTLRELEQLLVRYIVDRYNQSIDARMGDQTRFERWEAGLPTVPVPIPERDLDICLMKQSRRTVQRGGCLQFQNLMYRGEYLAGYAGETVNLRFDPRDITTILVYRQENNQEVFLTRAHAQGLETEQLALDEAEAASRRLRTAGKTISNQSLLQEVVDRDALVATKKSRKERQKLEQTVLRSAAVDESNRESLPSQIVEPDEVESTETVHSQYEDIEVWDYEQLREEYGF

SEQ ID NO: 53 - protein, 579 aa, Scytonema sp.
MGETLNSNEVDESLVLYDGSDEVDEISESEDTKQSNVIVTELSEEAKLRMQVLQSLIEPCDRKTYGIKLKQAAEKLGKTVRTVQRLVKKYQEQGLSGVTEVERSDKGGYRIDDDWQDFIVKTYKEGNKGGRKMTPAQVAIRVQVRAGQLGLEKYPCHMTVYRVLNPIIERKEQKQKVRNIGWRGSRVSHQTRDGQTLDVHHSNHVWQCDHTKLDVMLVDQYGETLARPWLTKITDSYSRCIMGIHLGFDAPSSLVVALAMRHAMLRKQYSSEYKLHCEWGTYGVPENLFTDGGKDFRSEHLKQIGFQLGFECHLRDRPPEGGIEERGFGTINTSFLSGFYGYLDSNVQKRPEGAEEEACITLRELHLLIVRYIVDNYNQRIDARSGNQTRFQRWEAGLPALPNLVNERELDICLMKKTRRSIYKGGYVSFENIMYRGDYLSAYAGESVLLRYDPRDISTVFVYRQDSGKEVLLSQAHAIDLETEQISLEETKAASRKIRNAGKQLSNKSILAEVQDRDTFIKQKKKSHKQRKKEEQAQVHSVKSFQTKEPVETVEEIPQPQKRRPRVFDYEQLRKDYDD

SEQ ID NO: 54 - protein, 555 aa, Spirulina major
MNTGNQEAHAVITDFSEEERLKLEVIQSLMEPCDHATYGQKLKDAAQKLGKSKRTVQRLVQQWEEMGLAAVTSKARADKGKHRISQEWQDFIVKTYRLGNKGSKRMSRKQVALRVQARAAELGEKMYPNERTVYRVLQPIIEAQEQKKSVRSAGWRGDRLSVKTRTGNDLVVEYTNQVWQCDHTWVDVLVVDVEGNIIGRPWLTTVIDTYSRCILGIRLGFDAPSSQVVALALRHAMLPKQYPPTFGLQCEWGTYGKPEYFYTDGGKDFRSEHLRQIGIQLGFTCELRDRPSEGGAVERPFGTLNTELFSTLPGYTGSNIQERPEDAEKDARMTLRDLEQLIVRYLVDNYNQRLDKRMGDQTRYQRWESGLLATPALLSERELDICLMKQTNRSIYREGYIRFENLMYKGEYLAGYVGERVVLRYDPRDITTVLVYRREKSQEVFLARAYAQDLETEQLTLEDAKAINKKIREKGKTISNRSILDEVRDRDLFVSKKKTKKERQKEEQTRLFTPVTTPNSKQETEEEEIEPVEKIDELPQVEILDYDELNDDYGW

SEQ ID NO: 55 - protein, 565 aa, unidentified
  source 1..565: note = Geminocystis sp.
MLEEKHNIEDHVARSRLEDSSNNTIVSELPVEAQQKLKIIQSLLEPCDRATYGDRLREGAKELGISVRSVQRLFKRYQEEGLNALTINDQRDKGKHRIKDFWESFILKTYKEGNKGSKRMNVKQVAIRVQAKALELNETNPPSYRTVLRVLNPIINQEKKTIRSPGWEGSTLSVKTRDGVDLNISYSNQVWQCDHTKVDVLLVDKNEVLLDRPWLTTVIDSYSRCIVGVNIGFDAPSSNVVGLALRHAILPKHYPEEYRLNCEWGTYGLPQYLFTDGGKDFRSNHLEEIATQLGFVLKLRDRPSEGGIVERPFKTLNQSLFSTLPGYTGSNVQERPEDAEKDAQLTLQELERLIVRFIVDKYNQSIDARMGDQTRFQRWEAGLRAIPEVMSERDLDICLMKQARRQVQRGGYLLFENIHYKGEYLEGFAGKTVSLRYDPRDITTIWVYQHSRGQEEFLTRAYAQGLETESLSLADAKASAKRLREKEKNISNEAILQEVIDREVTIKNKSRKQRQKEEQSYKKTPSLPVIEKEQESFEQVEEILLDTDDDFEVWDLDEMKDDYGW

SEQ ID NO: 56 - protein, 618 aa, Nostoc sp.
MPDKEFGLTGELTQITEAIFLSESNFVVDPLHIILESSDSQKLKFNLIQWLAESPNRQIKSQRKQAVADTLGVSTRQVERLLKEYNGDRLNETAGVQRCDKGKHRVSEYWQQYIKTIYENSLKEKHPMSPASVVREVKRHAIVDLGLEHGDYPHPATVYRILNPLIEQQKRKKKIRNPGSGSWLTVETRDGKQLKAEFSNQIIQCDHTELDIRIVDSNGVLLPERPWLTTVVDTFSSCVLGFHLWIKQPGSAEVALALRHSILPKQYPHDYELSKPWGYGPPFQYFFTDGGKDFRSKHLKAIGKKLRFQCELRDRPNQGGIVERIFKTINTQVLKDLPGYTGSNVQERPENAEKEACLGIQDIDKILASFFCDIYNHEPYPKDPRETRFERWFKGMGGKLPEPLDERELDICLMKETQRVVQAHGSIQFENLIYRGESLRAYKGEYVTLRYDPDHILTLYVYSCDANDDIGDFLGYVHAVNMDTQELSIEELKSLNKERSKARREHSNYDALLALGKRKELVKERKQEKKERRQAEQKRLRSGSKKNSNVVELRKSRAKNYVKNDDPIEVLPERVSREEIQVPKTEVQIEVSEQADNLKQERHQLVISRRKQNLKNIW

SEQ ID NO: 57 - protein, 558 aa, unidentified
  source 1..558: note = Cyanothece sp.
MNTFPNEQSNAIVLKNTIVSDLPETARTKMEVIQTLLEPCDRITYGERLREGAKKLGVSVRTVQRLFKQYQEQGLAALVSTERSDKGKHRINEFWQDFIVKTYQQGNKGSKRMTPKQVALKVQAKALEIGDEQPPTYRTVLRVLKPIQEKQEKTKSIRSPGWRGSTLSVKTRDGDDLEINYSNQVWQCDHTRADVLLVDRHGELIGRPWLTTVIDSYSRCIMGINLGFDAPSSQVVALALRHAILPKRYGDEYKLHCEWETSGTPEYFYTDGGKDFRSNHLAQIGSQLGFVHKLRDRPSEGGIVERPFKTLNQSLFSTLPGYTGSNVQERPKEAEKEASLTLRELEQLIVRFIVDKYNQSIDARMGDQTRYQRWEAGLRRQPEPISERELDICLMKAARRTVQRGGYLQFENVMYRGEYLEGYAGDTVILRYEPRDITTIWVYRQKKHQEVFLTRAHAQDLETEQLSVDEAKASAKKLRDAGKTISNQSILQEVIEREALVEKKSRKQRQKAEQAYKQEKQPSIIETVEPIEPEPLTQTEVDDIEVWDYDQLRDDYGW

SEQ ID NO: 58 - protein, 628 aa, Calothrix sp.
MSVFALMADKKFELTEKFTQLPEAVFLGENNFVIDPSQIILETSDKHKLTFNLMQWLAESPNRTIKSQRKQAIASTLGVSTRQVERLLKQYDEDRLSETSGLQRSDKGKYRVSDYWQEFIKTTYEKSLKDKHPISPASIVREVKRHAIVDLKLEQGNYPHPATVYRILNPLIEQQERKKKVRNPGSGSWMTVETRDGKQLKVDFSNQIIQCDHTKLDIRIVDSDGILLTERPWLTTVVDTFSSCVNGFHLWIKQPGSAEVAIALRHAILPKQYPDDYELGKPWKIYGHPFQYFFTDGGKDFRSKHLKAIGKKLGFQCELRDRPIQGGIVERIFNTINTQVLKDLPGYTGPNVQERPENAEKEACLSIHDLDKILASFFCDIYNHEPYPKDTRITRFERWFKGMGEKLPEPLNERELDICLMKEAQRVVQAHGSIQFENLVYRGESLNAHKGEYVTLRYDPDHILTLYVYSYDVNDELENFLGYVHAINMDTQDLSLEELKSLNKERSKARREHSNYDALLALSKRKELVEERKQGKKEKRQAEQKRLRAASKKNSNVVELRQNRASSSSNKDEKDEKIELLPERVSREELKVEKIEPQLEILDKAETPPQERHKLVISSRKQHLKKIW

SEQ ID NO: 59 - protein, 555 aa, Anabaena cylindrica
MKESQVAIQTLDIDHLGIVAGIIDEMELVEEVNKIVGIKTKETLTPGQVVKAMILNGLGFLSAPLYLFGEFFVGKATEHLIGEGVLPEHLNDDKLGRELDKYHQIGTTKIFTAVAIKAAHKFQVEMDSIHLDGTSMSVEGEYKKEIKEIDEIKQETEENKLEIEPEMKAIEIVHGYSRDKRPDLKQFIIDMIVTGDGDIPLYLKVDSGNVDDKSVFVERLKEFKKQWTFEGISVADSALYTAENLAAMRELKWITRVPLSIKEAKNKIVDIKEAEWKDSQISGYKIAAKESEYAGIKQRWIIVESEIRKKSSIQQVEKQVKKQEAKAKAALSKLSRQEFACQPDAKIVIEKLSKSWKYHQIKEIEYIEKLEYKTAGRPSKLTEPSQIKYQIKGQIETREEVIETEKINAGRFILATNVLDRNELSDEKVLEEYKAQQSNERGFRFLKDPLFFTSSVFVKTPERVEAIAMIMGLCLLVYNLAQRKLRQELAKFDDGIRNQVKKITNKPTMRWVFQMFQAVHLVIINGQKQMSNLTEEREKIVRYLGKSCSKYYLIT

SEQ ID NO: 60 - protein, 621 aa, Anabaena cylindrica
MADEEFEFTEGTTQVPDAILLDKSNFVVDPSQIILATSDRHKLTFNLIQWLAESPNRTIKSQRKQAVANTLDVSTRQVERLLKQYDEDKLRETAGIERADKGKYRVSEYWQNFITTIYEKSLKEKHPISPASIVREVKRHAIVDLELKLGEYPHQATVYRILDPLIEQQKRKTRVRNPGSGSWMTVVTRDGELLRADFSNQIIQCDHTKLDVRIVDNHGNLLSDRPWLTTIVDTFSSCVVGFRLWIKQPGSTEVALALRHAILPKNYPEDYQLNKSWDVCGHPYQYFFTDGGKDFRSKHLKAIGKKLGFQCELRDRPPEGGIVERIFKTINTQVLKELPGYTGANVQERPENAEKEACLTIQDLDKILASFFCDIYNHEPYPKEPRDTRFERWFKGMGGKLPEPLDERELDICLMKEAQRVVQAHGSIQFENLIYRGEFLKAHKGEYVTLRYDPDHILSLYIYSGETDDNAGEFLGYAHAVNMDTHDLSIEELKALNKERSNARKEHFNYDALLALGKRKELVEERKEDKKAKRNSEQKRLRSASKKNSNVIELRKSRTSKSLKKQENQEVLPERISREEIKLEKIEQQPQENLSASPNTQEEERHKLVFSNRQKNLNKIW

SEQ ID NO: 61 - protein, 623 aa, Anabaena sp.
MADREIEFTEESTQDSDAILLDNSNFVVDPSQIILATSDKHKLTFNLIQWLAQSPTRTVKSERKQAIANTLSVSTRQVERLLKQYNEDKLRETAGTERADKGKHRVSEYWQEFIKTTYEKSLKDKHPISPASIVREVKRHAIVDLGLKPGDYPHQATVYRILEPLIAQHKRKTRVRNPGSGSWMTVVTRDGQLLRADFSNQIIQCDHTKLDIRIVDIHGDLLSERPWLTTVVDTYSSCVLGFRLWIKQPGSTEVALALRHAILPKQYPDDYQLNKSWNVYGNPFQYFFTDGGKDFRSKHLKAIGKKLGFQCELRDRPPEGGIVERIFKTINTQVLKDLPGYTGANVQERPENAEKEACLTIQDLDKILASFFCDIYNHEPYPKEPRDTRFERWFKGMGEKLPERLDERELDICLMKETQRVVQAHGSIQFENLIYRGESLKAHKGEYVTLRYDPDHILTLFVYSCETDDNLEEFLGYAHAVNMDTHDLSLEELKTLNKERSKARKEHFNYDALLALGKRKELVDERKADKKEKRHSEQKRLRSASKKDSNIIELRKSRVSKSLRKQETQEILPERVSREEIKFEKIELEPQETLSASPKPNPQEEQRHKLVLSKRQKNLKNIW

SEQ ID NO: 62 - protein, 164 aa, unidentified
  source 1..164: note = Alkalinema sp.
MTIDSGIQPWLFQVEPYEGESLSHFLGRFRRPNHLTPSGLGQLAGIGAVVARWERFHWNPPPSRQELEALAKVVRVSVEQLIAMLPPPGVGMQCSPIRLCSACYGETPCHRMEWQYKHIWKCNRHSLKLLAKCPVCSALFKAPAQWQDGMCRRCLTPFGQMQQQ

SEQ ID NO: 63 - protein, 159 aa, Anabaena sp.
MIQPYEGESLSHFLGRFRRANHLSAAGLGNLAGIGAVIARWERFHFNPRPSQKELEAIASVVEVDAQRLAEMLPPAGVSMQHEPIRLCGACYAETPCHQIKWQFKETGGCDRHYLRLLSKCPNCDARFKIPALWELGVCQRCLMTFAEMAGYQKSINGT

SEQ ID NO: 64 - protein, 170 aa, Anabaena variabilis
MPEEIYNFEAWINVVEPLPGESISHFLGRFERANLLTGYQVGREVGVGAIVTRWKKLYLNPFPTLQELEALARFVEVEVEKLKEMLPSQGMTMKPRPIKICAACYAEVPCHRIEWQYKDNMKCDRHNLRLLTKCTNCETPFPIPADWLQGECPRCFLPFAKMAKRQKLNS

SEQ ID NO: 65 - protein, 173 aa, Anabaena variabilis
MRESINENKQFWLIRVEPLEGESISHFLGRFRREKGNKFSAPSGLGDVAGLGAVLARWEKFYFNPFPTHQELEALASVVQVDVDRLRQMLPPLGVSMKHSPIRLCGACYAESPCHKIEWQFKKTVGCDRHQLRLLSKCPVCEKPFPVPALWVDGICNRCFTPFAEMAQYQKHY

SEQ ID NO: 66 - protein, 167 aa, Anabaena variabilis
MEVPQIQPWLFQIEPLEGESLSHFLGRFRRANDLTPTGLGKAAGLGGAIARWEKFRFNPPPSRQQLEALANVVGVDADRLAQMLPSAGVGMKMEPIRLCAACYAESPCHKIEWQFKVTRGCARHKITLLSECPNCKARFKVPALWVDGWCNRCFLRFEEMAKYQKGL

SEQ ID NO: 67 - protein, 165 aa, unidentified
  source 1..165: note = Aphanocapsa montana
MVIPQIPAWVFPVEPSPGESLSHFLGRFCRENHTTLNQLGEKTGLGAVLGRWEKFRFIPQPSDAQLAALAKLVRLEVDQIKQMLPQETMQNRVIRLCAACYAEEPYHRIEWQYKLANRCDRHHLLLLLECPNCKAKLPMPSKWANGTCKRCLTPFEQMADLQKGI

SEQ ID NO: 68 - protein, 163 aa, Calothrix sp.
MNEPRFFEIEPFECESLSHFLGRFRRENYLTATQLGKLTGLGAVISRWENCYFNPFPTQQELEALASVVGAEVEKLREMLPPIGVTMKPRPIRLCAACYAESPYHHIEWQFKDVMKCSYHQMRLLTKCTNCGAVFPIPADWVTGECPHCCLPFVTMVRKQQKG

SEQ ID NO: 69 - protein, 167 aa, unidentified
  source 1..167: note = Cyanothece sp.
MDKEYWLTKVEPYEGESISHFLGRFRRTKGNRFSAASGLGQVAGLGAILARWEKLYFNPFPNEQELEALGKVVMLDVEDLRKMLPQKGMVTQPKPIMLCAVCYGEKPYHRMVWQDKHQRGCEVHGLELLSRCINCKRLFPIPSKWEEGKCLLCGLAFAKIAKHQKPI

SEQ ID NO: 70 - protein, 160 aa, unidentified
  source 1..160: note = Geminocystis sp.
MDLQIQNWLFILVPYEGESISHFLGRFRRANSLSCGGLGQATGLYSAIARWEKFRFNPPPSLKQLEKLSEIVQVEMATLQTMFPSAPMKMTPIRICSACYGENPYHQMSWQYKEIYRCDRHNLNILSECPNCAARFKFPNLWFEGFCHRCFTPFEQMPQS

SEQ ID NO: 71 - protein, 172 aa, unidentified
  source 1..172: note = Leptolyngbya boryana
MIEHSEIQPWFFHVEALEGESISHFLGRFRQANELTPSGVGKISGLGGAIARWEKFRFNPYPTQQQFEKLSTATGISVEQLWKMMPPEGVGMQLEPIRLCASCYAELPCHQIQWQFKDTQGCEVHGLRLLSECPNCKARFKPPATWSDSKCHRCFMLFSEMRNRQKRHSFSR

SEQ ID NO: 72 - protein, 171 aa, unidentified
  source 1..171: note = Leptolyngbya boryana
MEHSEIQPWFFHVEALEGESISHFLGRFRQANELTPSGVGKISGLGGAIARWEKFRFNPYPTQQQFEKLSTATGISVEQLWKMMPPEGVGMQLEPIRLCASCYAELPCHQIQWQFKDTQGCEVHGLRLLSECPNCKARFKPPATWSDSKCHRCFMLFSEMRNRQKRHSFSR

SEQ ID NO: 73 - protein, 140 aa, Myxosarcina sp.
MNNTKEAQLWLFPVEPSNGESLSHFLGRFRRSNHLSPSALGDLAGIGGVVARWERFHLNPFPTDEQFQALAEVVDVDSSTLREMLPPKGTGMKCDRHNLKLISKCPNCRAKFKMPALWEYGCCHRCRLPFAAIAQYQQSV

SEQ ID NO: 74 - protein, 166 aa, unidentified
  source 1..166: note = Nostoc punctiforme
MTTPDVKPWLFIIEPYPGESLSHFLGRFRRANHLSPAGLGGLAGIGAVVARWERFHFNPRPSQKELEAIASVVEVDAQRLAQMLPPAGVGMQHEPIRLCGACYAEAPCHRIEWQYKSVWKCDRHQLKILAKCPNCQAPFKMPALWEDGCCHRCRTLFAEMAKQQKS

SEQ ID NO: 75 - protein, 168 aa, Nostoc sp.
MMAAPDVKPWLFIIQPYEGESLSHFLGRFRRANHLSASGLGKLAGIGAVVARWERFHFNPRPSQKELEAIASLVEVDADRLAQMLPPLGVGMQHEPIRLCGACYAEAPCHRIEWQYKSVWKCDRHELKILAKCPNCEAPFKIPALWEDKCCHRCRTPFAEMTKYQKIT

SEQ ID NO: 76 - protein, 167 aa, Nostoc sp.
MAAPDVKPWLFIIQPYEGESLSHFLGRFRRANHLSASGLGKLAGIGAVVARWERFHFNPRPSQKELEAIASLVEVDADRLAQMLPPLGVGMQHEPIRLCGACYAEAPCHRIEWQYKSVWKCDRHELKILAKCPNCEAPFKIPALWEDKCCHRCRTPFAEMTKYQKIT

SEQ ID NO: 77 - protein, 173 aa, Nostoc sp.
MEENIDEKRQIWLTRVEPFDGESISHFLGRFRREKGNKFSAPSGLGDVAGLGVVLARWEKFYFNPFPTHQELDALATVVELDAERLRQMLPPEGVGMKHSPIRLCGACYAESVCHKIEWQFKKRVGCDRHLLRLLSKCPVCEKPFPVPALWMDGQCQRCFTSFAEMAEHQKHY

SEQ ID NO: 78 - protein, 167 aa, unidentified
  source 1..167: note = Scytonema hofmanni
MIEAPDVKPWLFLIKPYEGESLSHFLGRFRRANHLSASGLGTLAGIGAIVARWERFHFNPRPSQQELEAIASVVEVDAQRLAQMLPPAGVGMQHEPIRLCGACYAESPCHRIEWQYKSVWKCDRHQLKILAKCPNCQAPFKMPALWEDGCCHRCRMPFAEMAKLQKV

SEQ ID NO: 79 - protein, 175 aa, Scytonema sp.
MEIPAEQPRFFQVEPLEGESLSHFLGRFRRENYLTATQLGKLTGIGAVISRWEKFYLNPFPTPQELEALAAVVEVKVDRLIEMLPPKGVTMKPRPIRLCGACYQESPCHRVEWQFKDKLKCVSEAHPKDARHQLGLLTKCTNCETPFPIPADWVQGECPHCFLPFAKMARRQKRY

SEQ ID NO: 80 - protein, 167 aa, Spirulina major
MNDWEIQPWLFVVEPYEGESLSHFLGRFRRENDLTPAGLGREAEIGAVVSRWEKFRLIPFPSQRELEKLAQVVQVDAARLRVMLPPDGVGMKMTPIRLCGACYREVRCHRMEWQYKTSDRCDKHPLRLLSECPNCGARFPIPSLWQDGWCTRCFTTFGEMAESQKPL

SEQ ID NO: 81 - protein, 109 aa, unidentified
  source 1..109: note = Geminocystis sp.
MLDTDINTWLLPIEPLEGESLSHFLGRVRRRNYLSASALGELAGIGGAITRWEKFRHYPFPSDEELTALGNLLGLELFQLKAMLPSEPMKLEPIRLCGACYGEIPSSSY

SEQ ID NO: 82 - protein, 184 aa, Nostoc sp.
MEKDTFPPKTEIRIHDNHEALPRLGYVEPYEGESISHYLGRLRRFKANSLPSGYSLGKIAGIGAVTTRWEKLYFNPFPSSEELEALGKLIGVPANRIYEMLPPKGVTMKPRPIRLCAACYAEVPCHRIEWQYKDKLKCNHHNLGLLTKCTNCETPFPIPADWVQGECPHCFLPFAKMAKRQKPR

SEQ ID NO: 83 - protein, 188 aa, Calothrix sp.
MAEDIYLPKREIISNKEINKGDEILPRLGFVEPYECESISHYLGRVRRFKANSLPSGYSLGKIAGIGAVTTRWEKLYLNPFPSETELEALAKVIEVEVERLRQMLPPKGMTMKPRPIRLCAACYAESPHHRIEWQFKDVMVCDRHQLPLSTKCKNCGTPFPIPADWVRGECPHCCLSFTKMAKRQKSG

SEQ ID NO: 84 - protein, 167 aa, Anabaena cylindrica
MEVGEINPWLFQVEPYLGESLSHFLGRFRRANDLTTTGLGKAAGVGGAISRWEKFRFNPPPSRQQLEALAKVVGVDADRLEQMLPPAGVGMNLEPIRLCAACYVESPCHRIEWQFKVTQGCQHHHLSLLSECPNCGARFKVPALWVDGWCQRCFLPFGEMIEHQKRI

SEQ ID NO: 85 - protein, 184 aa, Anabaena cylindrica
MAQNIFLSKTEIGIDEDDEIRPKLGYVEPYEEESISHYLGRLRRFKANSLPSGYSLGKIAGLGAMISRWEKLYFNPFPTLQELEALSSVVGVNADRLIEMLPSQGMTMKPRPIRLCGACYAESPCHRIEWQCKDRMKCDRHNLRLLIKCTNCETPFPIPADWVKGQCPHCSLPFAKMAKRQRRD

SEQ ID NO: 86 - protein, 171 aa, Anabaena sp.
MNEDDEIRPKLGYVEPYEGESISHYLGRLRRFKANSLPSGYSLGKIAGLGAVTTRWEKLYFNPFPNRQELEALASVVGVSAERFIEMLPPKGVTMKPRPIRLCAACYAEVPCHRIDWQFKDKMKCDRHNLRLLTKCTNCETPFPIPADWAQGECPHCSLSFAKMVKRQKLR

SEQ ID NO: 87 - protein, 276 aa, unidentified
  source 1..276: note = Alkalinema sp.
MTQAKEWAEVLGEFQPDDDWLKAEIARLRKKTVMPLAQVSQLHDWLDGKRKSRQSCRIVGESRTGKSVACEAYFYRNKPQQEAGKKPIVPVVYVQPPQKCGPKELFKEIIEFLKHRATRGTVSDLRGRAIEVLQACGVEMLIIDEADRLKPETFADVRDIYDKLGIAVVLVGTDRLDAVIKRDEQVYNRFRACHRFGKLSGEEFKKTIALWEQKILNLPVASNLISKDMLRILTTGTEGYIGRMDEILREAAIRSLSFGYKKVEKKCLEEVAKEYK

SEQ ID NO: 88 - protein, 278 aa, Anabaena sp.
MTDDAQAIAKQLGGVKPDEEWLQAEITHLTSKSIVPLQQVITLHDWLDGKRKARQSCRVVGESRTGKTVACDAYRYRHKPRQEMGKTPIVPVVYIQPPSKCGAKDLFQEIIEYLKFKATRGTISDFRGRTMEVLKGCRVEMIIIDEADRIKPDTFADVRDIYDKLGIAIVLVGTDRLEAVIKRDEQVYNRFRACHRFGKLAGKDFQDTVQAWEDKILKLPLPSNLISKDMLRILTSATEGYIGGLDEILREAAIRSLSRGLKKIDKAVLQEVVQEFKL

SEQ ID NO: 89 - protein, 268 aa, Anabaena variabilis
MTNEEIQQEIERLRQPDILNLEQVKRFSSWLDERRKLRKPGRAVGESGLGKTTASLFYTYQNRAAKIPNQNPVAPVLYVELIGSSCSPSLLFKTIIETLKFKAKGGTENQLRERAWYLIKQCKVEMLIIDEAHRLQFKTLTDVTDLSDKVKIIPILVGTSSRLDALISKNEQVGGRFAAYFSFEQLSGANFIKTVKIWEQQILKLPEPSNLAENQEIITILQAKTAGQIRLLDQILRDAAVKALESGVNKIDKSLLNSIEGDYSLVAS

SEQ ID NO: 90 - protein, 275 aa, Anabaena variabilis
MTDAKPLDFIQEPTREIQAHIERLSRAPYLELNQVKSCHTWMYELVISRMTGLLVGESRSGKTVTCKAFRNNYNNLRQGQEQRIKPVVYIQISKNCGSRELFVKILKALNKPSNGTIADLRERTLDSLEIHQVEMLIIDEANHLKIETFSDVRHIYDEDSLKISVLLVGTTSRLLAVVKRDEQVVNRFLEKFEIDKLEENQFKQMIQVWERDVLRLPEESKLASGESFKLLKQSTNKLIGRLDMILRKAAIRSLLRGYKKVDQGVLKEIITATKF

SEQ ID NO: 91 - protein, 278 aa, Anabaena variabilis
MTSKQAQAVAQQLGDIPVNDEKIQAEIQRLNRKSFIPLEQVQMLHDWLDGKRQSRQSGRVVGESRTGKTMGCDAYRLRHKPKQEPGRPPTVPVAYIQIPQECGAKELFGVLLEHLKYQMTKGTVAEIRDRTLRVLKGCGVEMLIIDEADRLKPKTFAEVPDIFDKLEIAVILVGTDRLDAVIKRDEQVYNRFRACHRFGKFSGDEFKKIVDIWEKKVLQLPVASNLSSKTMLKTLGETTGGYIGLLDMILRESAIRALKKGLRKVDLATLKEVTEEYK

SEQ ID NO: 92 - protein, 270 aa, unidentified
  source 1..270: note = Aphanocapsa montana
MIALQDQEVQAHIERLRRDKTVALDSVKQAHTWLKRKRNARQCGRLTGDSRTGKTKTCESFLKLYGEPDLSGRVPIIPISYVHPKQECTSRELFREILEQYGDDLPRGTVGDARSRTLKVLRACKTEMLMIDEADRLKPKTFADVRDIFDKLEISVILIGTKQRLDPAVKKDEQVFNRFRSSYRIGTIPSNQLKTIVGLWERDILKLPVPSNLTSEAMLKELRKATGVSRKGYYIGLIDMVLREAAIRALEKGQSKIELETLKEVAKEYS

SEQ ID NO: 93 - protein, 278 aa, Calothrix sp.
MADEYLRKWVQNLWGDDPIPEELMPQIERLITPSIVELDHIQKIHDWLDSLRLSKQCGRIVAPPRAGKSVTCDVYKLLNKPQKRTGKRDIVPVLYMQVPGDCSAGELLTLILESLKYEATSGKLTDLRRRVQRLLKESKVEMLIIDEANFLKLNTFSEIARIYDLLKVSIVLVGTDGLDNLIKKEPYIHDRFIECYRLQLVSEKKFSELVQIWEDDVLCLPVPSNLVKRETLIPLYQKTGGKIGLVDRVLRRAAILSLRKGLSIIDKITLDEVLEWFE

SEQ ID NO: 94 - protein, 290 aa, unidentified
  source 1..290: note = Cyanothece sp.
MTDSSVIDKLAAEFGGFATPSPEIQAEIQRLSRQPYLEYEQVKNCHGWLYELVLSRMTGLLVGESRSGKTVTCKSFEKRYNKIKTGNKKRIKPIVYIQSPQNCGAREFFTKILKALNKPTNGNVSDLRERTLDGLQIHECEMLIIDEANHLKQETFAEVRHVYDEEELNMAVLLVGTRHRLEAVVKRDEQVLNRFMEEYELDRLDDKEFKQLIKIWEQSVLCLPELSNLDTGDNLKLLKKTTRKLIGRLDMILRKSAIRALREGKQSISPELLKQVISSIKWSEGRKGNG

SEQ ID NO: 95 - protein, 275 aa, unidentified
  source 1..275: note = Geminocystis sp.
MEAKAIAQELGNIEIPEEKLQMEIERLNSKTLVSLEQVAKLHEYFEGKRQSKQSCRVVGESRTGKTMACDSYRLRHKPIQKVGHPPQVPVVYIQIPQDCGTKELFQGIIEYLKYQMTKGTIAEIRQRAIKVLQGCGVEMIIIDEADRFKPKTFAEVRDIFDRLNIPIVLVGTDRLDTVIKRDEQVYNRFRSCYRFGKLSGADFQNTVNIWEKQVLKLPVASNLIQTKMLKLIAEATGGYIGLMDTILRESAIRSLKRGLNKITFEILKEVTQEFK

SEQ ID NO: 96 - protein, 278 aa, unidentified
  source 1..278: note = Leptolyngbya boryana
MMANEAQSIAQTLGSLPLTSELLQAEIHRLTKKSVVSLSQVQSLHNWLEGKRQARQSCRVVGESRTGKTLACDAYRLRHKPTQQAGKPPIVPVVYIQVPQECGSKELFQIIIEHLKYQMVKGTVAEIRERTMRVLKGCGVEMLIIDEADRLKPKTFADVRDIFDKLEISVVLVGTDRLDAVIKRDEQVYNRFRACHRFGKLAGEEFRRTIEIWEKQILKLPVASNLTSKAALKILGETTAGYIGLLDMVLREAAIRALKQGKTKIDLEILKEVSTEYR

SEQ ID NO: 97 - protein, 282 aa, Myxosarcina sp.
MTEAKAIADKLGKIELDEEWVQKEIARLNRKSTVALEHIKELHDWLDGKRKSRRSCRIVGKSRTGKTVACEAYVMRNKLNKPPQERQTKNQIPIEPVIMIMPPQKCGAKELFREIIECLKFRAVKGTISEFRSRAMDVLQKCQVEMLIIDEADRLKPETFSEVRDIYDKLEIAVVLVGTERLDTAVKRDEQVENRFRANRRFGTLEGINFKKTVEIWEEKILKLPVASNLTNKTTWKILLIATEGFIGRLDEILREAAIASLSQGHKKVDPKILKEIAREYS

SEQ ID NO: 98 - protein, 276 aa, unidentified
  source 1..276: note = Nostoc punctiforme
MTEAQAIAKQLGGVKPDDEWLQAEIARLKGKSIVPLQQVKTLHDWLDGKRKARQSCRVVGESRTGKTVACDAYRYRQKPQQEVGRPPIVPVVYIQPPQKCGSKDLFKEMIEYLKFRATKGTVSDFRGRAMEVLKGCGVEMLIIDEADRLKPETFAEVRDIYDKLGIAVVLVGTDRLEAVIKRDEQVYNRFRACHRFGKLSGKDFQDTVQAWEDRVLKLPVSSNLTSKDMLRILTSATEGYIGRLDEILRETAIRSLSKGFKKIDKAVLQEVAKEYK

SEQ ID NO: 99 - protein, 276 aa, Nostoc sp.
MTDAKAIAQQLGGVKPDEEWLQAEIARLKGKSIVPLQQVRSLHDWLDGKRKARQFCRVVGESRTGKTVACDAYRYRQKVQAEVGRPPIVPVVYIQPPQKCGAKDLFQEIIEYLKFKATKGTVSDFRGRTMEVLKGCGVEMIIVDEADRLKPETFAEVRDIYDKLGIAVVLVGTDRLEAVIKRDEQVYNRFRACHRFGKLSGKDFQDTVQAWEDKILKLPLPSNLISKDMLRILTSATEGYIGRLDEILREAAIRSLSRGLKKIDKPVLQEVAQEYK

SEQ ID NO: 100 - protein, 279 aa, Nostoc sp.
MTNAMSLAKQFGVIQELTPEIQAEIERLSRQPYLELDQVKRCHAWMYELVISRMTGLLLGESRCGKTVTCKAFANLYNKLRQTKGQRMKPVVYIQVGKNCGSRDFFMKILKALNKPSNGTISDLRERTLDSLAIHQVEMLIIDEANHLKFETFSDVRHIYDDDELKISVLLVGTTSRLLAIVKRDEQVVNRFLEQFELDRLEDTQFKQMIQIWERDVLRLPEESKLASGDNLKILKQATKKLIGRLDMILRKAAIRSLLRGQKQVDKDILKEVIAASKL

SEQ ID NO: 101 - protein, 276 aa, unidentified
  source 1..276: note = Scytonema hofmanni
MTEAQAIAKQLGGVKPDDEWLQAEIARLKGKSIVPLQQVKTLHDWLDGKRKARKSCRVVGESRTGKTVACDAYRYRHKPQQEAGRPPTVPVVYIRPHQKCGPKDLFKKITEYLKYRVTKGTVSDFRDRTIEVLKGCGVEMLIIDEADRLKPETFADVRDIAEDLGIAVVLVGTDRLDAVIKRDEQVLERFRAHLRFGKLSGEDFKNTVEMWEQMVLKLPVSSNLKSKEMLRILTSATEGYIGRLDEILREAAIRSLSRGLKKIDKAVLQEVAKEYK

SEQ ID NO: 102 - protein, 278 aa, Scytonema sp.
MAEDYLRKWVQNLWGDDPIPEELLPIIERLITPSVVELEHIQKIHDWLDSLRLSKQCGRIVAPPRAGKSVTCDVYKLLNKPQKRTGKRDIVPVLYMQAPGDCSAGELLTLILESLKYDATSGKLTDLRRRVLRLLKESRVEMLVIDEANFLKLNTFSEIARIYDLLKISIVLVGTDGLDNLIKKERYIHDRFIECYKLPLVSENKFPEFVQIWEDEVLCLPVPSNLIKSETLKPLYQKTSGKIGLVDRVLRRAAILSLRKGLKNIDKATLDEVLEWFE

SEQ ID NO: 103 - protein, 283 aa, Spirulina major
MASAESKAKAEAVAQQLGNFEKTEEDLAKEIQRLRRRNVVQLEQVKQLHNWLEGKRRSRQCCRVVGESRTGKTIGCNAYRLRHKPIQETGKPPIVPVVYIEPPQDCGSIDLFRAIIEYLKYKVQSREKVRELRSRAMKVLERCQVETLIIDEADRLKPKTFADVRDIFDKRNISVVLVGTDRLDNVIKRDEQVHNRFRACYRFGKLTGTEFEQVVKIWERDILRLPIPSNLHAKNMLKILGQATGGYIGLLDMILRETAVRALEKGLGKINLETLKEVAEEYS

SEQ ID NO: 104 - protein, 270 aa, unidentified
  source 1..270: note = Geminocystis sp.
MTKAQSIAKELGDLGQDEQWLHQEICRLNRSSIVPLEHLKDLHNWLDEKRKARQSCRIVGESRTGKTIACESYKLRNKPSQKGQQTPSVPVVYIMPPAKCSAKDFFREIIEALRYRAVKGTVSDFRSRAMDVLKACDVEMLIVDEADRLKPDTFPEVRDISDKLEMLVVLVGTDRLDAVIKRDEQVYNRFRSHRRFGKLTGEDFKETVSIWEKEVLNLPVASNLTKLDMFKIITKATGGYIGRLDELLREAAIKSLSRGKKALKRIFYRR

SEQ ID NO: 105 - protein, 284 aa, Nostoc sp.
MAQSQLVIQPNVETLAPQLELNNQLAKVVEIEEIFSNCFIPTDRACEYFRWLDELRILKQCGRVVGPRDVGKSRASVHYREEDRKKVSYVRAWSASSSKRLFSQILKDINHAAPTGKREDLRPRLAGSLELFGIEQVIVDNADNLQREALLDLKQLFDESNVSVVLVGGQELDKILHDCDLLTSFPTLYEFDTLEDDDFKKTLSTIEFDVLALPQASNLCEGITFEILVQSTGGRIGLLVKILTKAVLHSLKNGFGRVDQNILEKIANRYGKRYIPPENRNKNS

SEQ ID NO: 106 - protein, 276 aa, unidentified
  source 1..276: note = Cyanothece sp.
MTQAQEIAQKLGDLNPDEQWLQMEIARLNRQSIVPLEHIRDLHEWLDGKRKARQSCRLVGESRTGKTVACEAYTLRNKPIQQGRQTPIVPVVYIMPPTKCGSKDLFKEIIEYLRYKAVKGTVSEFRSRAMEVLKGCEVEMIIIDEADRLKPDTFPDVRDINDKLEISVVLVGNDRLDAVIKRDEQVYNRFRAHRRFGKLAGVEFKKTVAIWEEKVLKLPVASNLTSSALIKILVKATEGYIGRLDEILREAAIKSLMKGHKRIEKEVLQEVAKEYS

SEQ ID NO: 107 - protein, 283 aa, Calothrix sp.
MARSQLAIQSSVEVLVPQLDLNAQLAKVVEVEEIFSNYFIPTDRSSEYLRWLDELRILRQCGRVIGPRDVGKSRASLHYQGQDQKRISYVRAWSASSSKRLFSQILKDIKHAAPMGKRDDLRPRLAGSLEVFGFEQVIIDNAENLQKEALLDLKQLFDECHVPIVLIGGQELDTILDEFDLLTCFPTLYEFDGLDENDFKKTLNTIEFDILALPEASNLSEGIIFELLAESTGARIGLLVKILTKAVLHSLKNGFSKIDQNILEKIANRYGRRYIPPEKRNNK

SEQ ID NO: 108 - protein, 283 aa, Anabaena cylindrica
MAQPQLATQSIVEVLAPRLDIKAQIAKTIDIEEIFRACFITTDRASECFRWLDELRILKQCGRIIGPRNVGKSRAALHYRDEDKKRVSYVKAWSASSSKRLFSQILKDINHAAPTGKRQDLRPRLAGSLELFGLELVIIDNAENLQKEALLDLKQLFEECNVPIVLAGGKELDDLLHDCDLLTNFPTLYEFERLEYDDFKKTLTTIELDVLSLPEASNLAEGNIFEILAVSTEARMGILIKILTKAVLHSLKNGFHRVDESILEKIASRYGTKYIPLKNRNRD

SEQ ID NO: 109 - protein, 284 aa, Anabaena sp.
MARSQLANQPIVEVLAPQLDLNAQIAKAIDIEEIFRNCFITTDRVSECFRWLDELRILKQCGRIIGPRNVGKSRAALHYRDEDKKRISYVKAWSASSSKRIFSQILKDINHAAPTGKRQDLRPRLAGSLELFGLELVIIDNADNLQKEALIDLKQLFEECHVPIVLIGGKELDNILQDCDLLTNFPTLYEFERLEYDDFRKTLSTIELDILSLPESSHLAEGNIFEILAVSTSGRMGILVKILTKAVLHSLKNGFGRVDESILEKIASRYGTKYVPLENRNRNE

SEQ ID NO: 110 - DNA, 37 bp, unidentified
  misc_feature 1..37: note = source = /note="Description of Unknown: Direct repeat nucleotide sequence"
  source 1..37: mol_type = unassigned DNA
atcgcaaccaactgtcctgaagaagggcgattgaaag

SEQ ID NO: 111 - DNA, 37 bp, unidentified
  misc_feature 1..37: note = source = /note="Description of Unknown: Direct repeat nucleotide sequence"
  source 1..37: mol_type = unassigned DNA
gtaacaacaaccctcctagtacagggtgggttgaaag

SEQ ID NO: 112 - DNA, 36 bp, unidentified
  misc_feature 1..36: note = source = /note="Description of Unknown: Direct repeat nucleotide sequence"
  source 1..36: mol_type = unassigned DNA
gtttcaacgaccatcccggctaggggcgggttgaaa

SEQ ID NO: 113 - DNA, 37 bp, unidentified
  misc_feature 1..37: note = source = /note="Description of Unknown: Direct repeat nucleotide sequence"
  source 1..37: mol_type = unassigned DNA
gtgacaatagcccttcccgtgttgagcgggttgaaag

SEQ ID NO: 114 - DNA, 37 bp, unidentified
  misc_feature 1..37: note = source = /note="Description of Unknown: Direct repeat nucleotide sequence"
  source 1..37: mol_type = unassigned DNA
gtttcaacacccctcccgaagtggggcgggttgaaag

SEQ ID NO: 115 - DNA, 28 bp, unidentified
  misc_feature 1..28: note = source = /note="Description of Unknown: Direct repeat nucleotide sequence"
  source 1..28: mol_type = unassigned DNA
ggtcgccaaaagcatttcagggcagggc

SEQ ID NO: 116 - DNA, 36 bp, unidentified
  misc_feature 1..36: note = source = /note="Description of Unknown: Direct repeat nucleotide sequence"
  source 1..36: mol_type = unassigned DNA
gtgctgtaacctaagatgtcgcaaggcgttgagcag

SEQ ID NO: 117 - DNA, 39 bp, unidentified
  misc_feature 1..39: note = source = /note="Description of Unknown: Direct repeat nucleotide sequence"
  source 1..39: mol_type = unassigned DNA
gagtttcatcaaccctcctgatgtgggatgggttgaaag

SEQ ID NO: 118 - DNA, 38 bp, unidentified
  misc_feature 1..38: note = source = /note="Description of Unknown: Direct repeat nucleotide sequence"
  source 1..38: mol_type = unassigned DNA
gtttcatacaggtttttgacctcccattgattgaaaga

SEQ ID NO: 119 - DNA, 37 bp, unidentified
  misc_feature 1..37: note = source = /note="Description of Unknown: Direct repeat nucleotide sequence"
  source 1..37: mol_type = unassigned DNA
gttgaaataagaaaataccttctctagggattgaaag

SEQ ID NO: 120 - DNA, 37 bp, unidentified
  misc_feature 1..37: note = source = /note="Description of Unknown: Direct repeat nucleotide sequence"
  source 1..37: mol_type = unassigned DNA
gtttcatccaggtttgcggcaagggggcgattgaaag

SEQ ID NO: 121 - DNA, 37 bp, unidentified
  misc_feature 1..37: note = source = /note="Description of Unknown: Direct repeat nucleotide sequence"
  source 1..37: mol_type = unassigned DNA
gtttcaacgaccactttaagatgggtatggttgaaag

SEQ ID NO: 122 - DNA, 37 bp, unidentified
  misc_feature 1..37: note = source = /note="Description of Unknown: Direct repeat nucleotide sequence"
  source 1..37: mol_type = unassigned DNA
gtggcaacaaccctccaggtactgggtgggttgaaag

SEQ ID NO: 123 - DNA, 38 bp, unidentified
  misc_feature 1..38: note = source = /note="Description of Unknown: Direct repeat nucleotide sequence"
  source 1..38: mol_type = unassigned DNA
cgttgcaaccctccttccagtaatgggagggttgaaag

SEQ ID NO: 124 - DNA, 37 bp, unidentified
  misc_feature 1..37: note = source = /note="Description of Unknown: Direct repeat nucleotide sequence"
  source 1..37: mol_type = unassigned DNA
gttgcaatgacccttcccgtgttgagcgggttgaaag

SEQ ID NO: 125 - DNA, 37 bp, unidentified
  misc_feature 1..37: note = source = /note="Description of Unknown: Direct repeat nucleotide sequence"
  source 1..37: mol_type = unassigned DNA
gtggcaacaaccttccaggtactaggtgggttgaaag

SEQ ID NO: 126 - DNA, 37 bp, unidentified
  misc_feature 1..37: note = source = /note="Description of Unknown: Direct repeat nucleotide sequence"
  source 1..37: mol_type = unassigned DNA
gtttcaacaaccatcccggctaggggtgggttgaaag

SEQ ID NO: 127 - DNA, 39 bp, unidentified
  misc_feature 1..39: note = source = /note="Description of Unknown: Direct repeat nucleotide sequence"
  source 1..39: mol_type = unassigned DNA
gtttcaatgaccatcccacgttgggatggattgaaagag

SEQ ID NO: 128 - DNA, 25 bp, unidentified
  misc_feature 1..25: note = source = /note="Description of Unknown: Direct repeat nucleotide sequence"
  source 1..25: mol_type = unassigned DNA
gattcttcgtaaactcagaatgaca

SEQ ID NO: 129 - DNA, 36 bp, unidentified
  misc_feature 1..36: note = source = /note="Description of Unknown: Direct repeat nucleotide sequence"
  source 1..36: mol_type = unassigned DNA
gttgcagatgaatttacttctctgtgcgatcgaaag

SEQ ID NO: 130 - DNA, 37 bp, unidentified
  misc_feature 1..37: note = source = /note="Description of Unknown: Direct repeat nucleotide sequence"
  source 1..37: mol_type = unassigned DNA
gtttcaacgaccatcccggctaggggtgggttgaaag

SEQ ID NO: 131 - DNA, 37 bp, unidentified
  misc_feature 1..37: note = source = /note="Description of Unknown: Direct repeat nucleotide sequence"
  source 1..37: mol_type = unassigned DNA
gtttcaacgaccattcccaacagggatgggttgaaag

SEQ ID NO: 132 - DNA, 37 bp, unidentified
  misc_feature 1..37: note = source = /note="Description of Unknown: Direct repeat nucleotide sequence"
  source 1..37: mol_type = unassigned DNA
gtttcaactaccatcccgactaggggtgggttgaaag

SEQ ID NO: 133 - DNA, 37 bp, unidentified
  misc_feature 1..37: note = source = /note="Description of Unknown: Direct repeat nucleotide sequence"
  source 1..37: mol_type = unassigned DNA
gtttcaacacccctcccggagtggggcgggttgaaag

SEQ ID NO: 134 - DNA, 38 bp, unidentified
  misc_feature 1..38: note = source = /note="Description of Unknown: Direct repeat nucleotide sequence"
  source 1..38: mol_type = unassigned DNA
gtttccaaagccctctcgttaggtggtgggttgaaagt

SEQ ID NO: 135 - DNA, 19 bp, unidentified
  misc_feature 1..19: note = source = /note="Description of Unknown: Transposon end motif"
  source 1..19: mol_type = unassigned DNA
tctaacagattgactgtcg

SEQ ID NO: 136 - DNA, 19 bp, unidentified
  misc_feature 1..19: note = source = /note="Description of Unknown: Transposon end motif"
  source 1..19: mol_type = unassigned DNA
aataacactttatttgtcg

SEQ ID NO: 137 - DNA, 19 bp, unidentified
  misc_feature 1..19: note = source = /note="Description of Unknown: Transposon end motif"
  source 1..19: mol_type = unassigned DNA
attcgcaaattcaatgtcg

SEQ ID NO: 138 - DNA, 29 bp, unidentified
  misc_feature 1..29: note = source = /note="Description of Unknown: Transposon end motif"
  source 1..29: mol_type = unassigned DNA
tttggacaataattctttaaaactggttt

SEQ ID NO: 139 - DNA, 19 bp, unidentified
  misc_feature 1..19: note = source = /note="Description of Unknown: Transposon end motif"
  source 1..19: mol_type = unassigned DNA
aatgatagaatagctgtag

SEQ ID NO: 140 - DNA, 19 bp, unidentified
  misc_feature 1..19: note = source = /note="Description of Unknown: Transposon end motif"
  source 1..19: mol_type = unassigned DNA
ggcagcaatacgggatgca

SEQ ID NO: 141 - DNA, 19 bp, unidentified
  misc_feature 1..19: note = source = /note="Description of Unknown: Transposon end motif"
  source 1..19: mol_type = unassigned DNA
attgactcattaactgaca

SEQ ID NO: 142 - DNA, 19 bp, unidentified
  misc_feature 1..19: note = source = /note="Description of Unknown: Transposon end motif"
  source 1..19: mol_type = unassigned DNA
cttgacaaattatttgtca

SEQ ID NO: 143 - DNA, 19 bp, unidentified
  misc_feature 1..19: note = source = /note="Description of Unknown: Transposon end motif"
  source 1..19: mol_type = unassigned DNA
attcgcaaattaaatgtcg

SEQ ID NO: 144 - DNA, 21 bp, unidentified
  misc_feature 1..21: note = source = /note="Description of Unknown: Transposon end motif"
  source 1..21: mol_type = unassigned DNA
ggtgcatccgacttgtaagcg

SEQ ID NO: 145 - DNA, 19 bp, unidentified
  misc_feature 1..19: note = source = /note="Description of Unknown: Transposon end motif"
  source 1..19: mol_type = unassigned DNA
agtgactaattatatgtca

SEQ ID NO: 146 - DNA, 20 bp, unidentified
  misc_feature 1..20: note = source = /note="Description of Unknown: Transposon end motif"
  source 1..20: mol_type = unassigned DNA
gagagtacatcgttgacata

SEQ ID NO: 147 - DNA, 19 bp, unidentified
  misc_feature 1..19: note = source = /note="Description of Unknown: Transposon end motif"
  source 1..19: mol_type = unassigned DNA
agtgactaattatatgtcg

SEQ ID NO: 148 - DNA, 20 bp, unidentified
  misc_feature 1..20: note = source = /note="Description of Unknown: Transposon end motif"
  source 1..20: mol_type = unassigned DNA
gagagcgctacgttgacatt

SEQ ID NO: 149 - DNA, 19 bp, unidentified
  misc_feature 1..19: note = source = /note="Description of Unknown: Transposon end motif"
  source 1..19: mol_type = unassigned DNA
ttcgacaaattagctgtca

SEQ ID NO: 150 - DNA, 18 bp, unidentified
  misc_feature 1..18: note = source = /note="Description of Unknown: Transposon end motif"
  source 1..18: mol_type = unassigned DNA
gtagtaaatgcttgttat

SEQ ID NO: 151 - DNA, 19 bp, unidentified
  misc_feature 1..19: note = source = /note="Description of Unknown: Transposon end motif"
  source 1..19: mol_type = unassigned DNA
agtaacagattatttgtcg

SEQ ID NO: 152 - DNA, 19 bp, unidentified
  misc_feature 1..19: note = source = /note="Description of Unknown: Transposon end motif"
  source 1..19: mol_type = unassigned DNA
agtaacaaattatttgtca

SEQ ID NO: 153 - DNA, 19 bp, unidentified
  misc_feature 1..19: note = source = /note="Description of Unknown: Transposon end motif"
  source 1..19: mol_type = unassigned DNA
gttcgcaaattaaatgtcg

SEQ ID NO: 154 - DNA, 18 bp, unidentified
  misc_feature 1..18: note = source = /note="Description of Unknown: Transposon end motif"
  source 1..18: mol_type = unassigned DNA
taatcaacaataaaacat

SEQ ID NO: 155 - DNA, 19 bp, unidentified
  misc_feature 1..19: note = source = /note="Description of Unknown: Transposon end motif"
  source 1..19: mol_type = unassigned DNA
agtgccacattaattgtca

SEQ ID NO: 156 - DNA, 19 bp, unidentified
  misc_feature 1..19: note = source = /note="Description of Unknown: Transposon end motif"
  source 1..19: mol_type = unassigned DNA
agcgacaaataatttgtcg

SEQ ID NO: 157 - DNA, 19 bp, unidentified
  misc_feature 1..19: note = source = /note="Description of Unknown: Transposon end motif"
  source 1..19: mol_type = unassigned DNA
ggcgcacttcgttcgggat

SEQ ID NO: 158 - DNA, 19 bp, unidentified
  misc_feature 1..19: note = source = /note="Description of Unknown: Transposon end motif"
  source 1..19: mol_type = unassigned DNA
attcgcaaattgaatgtcg

SEQ ID NO: 159 - DNA, 21 bp, unidentified
  misc_feature 1..21: note = source = /note="Description of Unknown: Transposon end motif"
  source 1..21: mol_type = unassigned DNA
ggagcatcagactcttaattt

SEQ ID NO: 160 - DNA, 19 bp, unidentified
  misc_feature 1..19: note = source = /note="Description of Unknown: Transposon end motif"
  source 1..19: mol_type = unassigned DNA
gcaagtccttttatccgct

SEQ ID NO: 161 - DNA, 19 bp, unidentified
  misc_feature 1..19: note = source = /note="Description of Unknown: Transposon end motif"
  source 1..19: mol_type = unassigned DNA
tttaacaaattaagtgtca

SEQ ID NO: 162 - DNA, 22 bp, unidentified
  misc_feature 1..22: note = source = /note="Description of Unknown: Transposon end motif"
  source 1..22: mol_type = unassigned DNA
gatcagtaggtcgtgggtttaa

SEQ ID NO: 163 - DNA, 19 bp, unidentified
  misc_feature 1..19: note = source = /note="Description of Unknown: Transposon end motif"
  source 1..19: mol_type = unassigned DNA
agtgactaattatttgtca

SEQ ID NO: 164 - DNA, 22 bp, unidentified
  misc_feature 1..22: note = source = /note="Description of Unknown: Transposon end motif"
  source 1..22: mol_type = unassigned DNA
aatgacaataatttgtcacaaa

SEQ ID NO: 165 - DNA, 19 bp, unidentified
  misc_feature 1..19: note = source = /note="Description of Unknown: Transposon end motif"
  source 1..19: mol_type = unassigned DNA
aataactaattaaatgtcg

SEQ ID NO: 166 - DNA, 19 bp, unidentified
  misc_feature 1..19: note = source = /note="Description of Unknown: Transposon end motif"
  source 1..19: mol_type = unassigned DNA
aatgacaaattgtttgtcg

SEQ ID NO: 167 - DNA, 700 bp, unidentified
  misc_feature 1..700: note = source = /note="Description of Unknown: Non-coding transposon end sequence"
  source 1..700: mol_type = unassigned DNA
tgatatttatttgttctatcacatcttcataaatatctgccattgtcaaagttaagttaactgaatctaaatgtaatttatcatcttttcctaatacttccgttgaccaatttccttgatcatcttgacgacaaacctccacttttatttgattttgctaaactagaacatattcctttagactgtctatagtttgataattaatgcgtttttcacgtttgtctattgttgctgtggaattagataaaacttcagaaatgttgaatgtcgttacaaaactttacatatttaagtatagtagtattatcaagggtaaaaatgtcaacatttcataatatcttccatccaatgaacataactgatttgttgacacatacaagattgaagatagcggaatttatctaacatcttttcaccccattctttcggaacagataaatgtacagtgactaattatatgtcatcgtgacaaattaatgtcatcttataaatccttgctgtagaaggattttagcaatttaacgatattatacttcaatccagttagtgacaaaataaatgtcgtttcccatgattgtgacaaattaactgtcgcgttaccgattaaaaaaagtttttgtatattttcataatgacaaattgactgtcgctttccagtaagctagaataacatcatgtttttataaaatctgtttgattt

SEQ ID NO: 168 - DNA, 500 bp, unidentified
  misc_feature 1..500: note = source = /note="Description of Unknown: Non-coding transposon end sequence"
  source 1..500: mol_type = unassigned DNA
aggtaaaatggtgtgcgatcgcccaacacagaattaggtaacaacaacccaccctatggtgggttgaaaggttatcatattcttataaaaagagggttgaaagagagtacatcgttgacatatcgctctgatatatcaataaatgacattaatttgtcactaccattaaaacgacaacaatttgtcctaacgacactaaattgtcaccgacgacatataattagtcactgtacaaaaagtatggacggtattggactcgaaccaacgaccccatcgatgtcaacgatgtactctaaccacctgagctaaccgtccttaatcccatagactaataacgtatcacataaaatataatttgtcaatccctaaaatccaaattttattctacatatatctagtcaactgtacccgatagcgatccttttttgtcaccgcaatttccccaacttccaaccgaccttttccccgaatagcgattaaatcccctgttttcacttgag

SEQ ID NO: 169 - DNA, 700 bp, unidentified
  misc_feature 1..700: note = source = /note="Description of Unknown: Non-coding transposon end sequence"
  source 1..700: mol_type = unassigned DNA
aaaaatgatgcagttatgaatttttcgcttctttgtctatagctttacacaatttcttggcttgggtgaatttttgagcaacaacttagtttttctgtggtatcggggtgcaaataagtaattcaagctctagttaactaacaaaactcaactacgcgaagacggaaaaaacacagaataagtaaggataagtctacaaggttatctgttcttgcttcgccatcaccaaaaaagaccgccgcaccccttcaatttcgcgtggagccaactgtagtgcatatcgcctagttgtttctgaagttttggacaataattctttaaaactggattgatggctgaaagctaggaaatacgtaaattatgcgtttagcactgtcaaatggacaataattctttaaaactgacaataattatttaaagtacatattgtacattcgcacattatatgtcgcaatttgcaaacaacgacattagacgacattagcatcttgagcatctaaaacgcttatcgtataaagctttcaggaaattgttaattaaaaacgttaaattcctgacttcgcacattgtatgtcgctaacattaaagctcgcaaattaatgtcgttaatctaaattttgtcacattgcaaattcaatgtcgcattttcttcagttaatggtacattaatactaccaataactacattctcctcgcttaa

SEQ ID NO: 170 - DNA, 500 bp, unidentified
  misc_feature 1..500: note = source = /note="Description of Unknown: Non-coding transposon end sequence"
  source 1..500: mol_type = unassigned DNA
agtaagaagaatagaagtaatttcctgaaatcaactatatttggacatattttaggaatataactaaattatgggagggttgaaaggagcgctgcgatcaaacaaatgttaaggtaaaataattcgtctgtagcaaataccacagttaatgagtaagccatacgacgttaatttgcgaaaaactaatatgattgaattaacgacgcgaattagcgaaagtaatgttaattacctaaaaacgacatcaatttgcgaaaagcgacaaataatgtgcgaatgtacacatatggagaatagggaactcgaatccctgacctctgcggtgcgatcgcagcgctctaccaactgagctaattcccctgactttgttgagtgttcagttaatcacactcgtccatcatagagtattttaacattcagttagggatggctgagatttgttgccagaaaaaacttcttgtactcgctctagttctaattcgctcagataatccacagtc

SEQ ID NO: 171 - DNA, 700 bp, unidentified
  misc_feature 1..700: note = source = /note="Description of Unknown: Non-coding transposon end sequence"
  source 1..700: mol_type = unassigned DNA
gtgttaatttacttacctgagattaacaaacattctttgagaataatcacaaaaaatgtaaagactaaataaaagcagtcacctataagtaaatgacctatgggcgtaacccaataatgtcttcaagcacaagttccaactggtaacttgcagtattaatacttaaatatttttttgtattcaaaaaaatataaactaaacttaataaaaattaactttacttattgttaacaataatgaaggaattcttgatagatgtttttattgttttttgagatttaaaatgcgatcgctcgtaattatatttgatataaagtataatttgacacagtaacaaaaatctacctaccctcgtagagacgttccgccggaacgtctttacatttattatgtgttgcccaaaaccattgtagagaggtagcattgctacgtctctacgtgtacagtgactaattatatgtcgttgtgacaaattattgtcatcagtaaaatccttatacagtatagattatagcgctttggcagttttagcataacctctttgcagtgacaaaatagatgtcgttgtccgtgattgtgacaaattagctgtcgctttgcaagataggaaaaagcttttgtgtattttcataatgacaaattgactgtcgctttgtatatagatagaataacaatatgtttttataaaaaagttttatat

SEQ ID NO: 172 - DNA, 500 bp, unidentified
  misc_feature 1..500: note = source = /note="Description of Unknown: Non-coding transposon end sequence"
  source 1..500: mol_type = unassigned DNA
aatcttttaaaccgagattagttttgggtttcagtaactattaaacttaggggtgggttgaaagcaagtccttttatccgcttgttttaattgctttgtataataattgcagagcatattatattgatgacatttaatttgtcatcaattaattaagcaacgctgatgggtcacgacgacaattaaatagtcacaatgacattaatctgtcaccgacgacagataatttgtcactgtacactacgccttttgtggagatgtctaataaactaaaaatttttgatagtgcggataaaaggacttgaaccttcactcctttcggaactagaacctaaatctagcgcgtctgccaatttcgccatatccgcatcagtttcctatattaccataacgaattatctgtggcaatagggattcgggactgggacaaggaggaattcccaatgccccatgccccatgcccaatgccccatgcccaatgccccatgccccatgcccaa

SEQ ID NO: 173 - DNA, 700 bp, unidentified
  misc_feature 1..700: note = source = /note="Description of Unknown: Non-coding transposon end sequence"
  source 1..700: mol_type = unassigned DNA
ataaaatacccaaaatcttattgatctccttaatataataattattcaaatcaataaacacatcacagccttctaacttcaaaaacttccaaatatcccccgttgttactgcaccataaatagttgtcgaataactaattaaatgtcgtcttaacaaattaatgtcgctcaaaacgtaaaggctataatcgttactaagcaaggattatagcctttttgatttctatttagcattcctaaatatttaactaattaaacgtcgtaatttgtaaatataacaaaataagtgtcgttttttcaaaaaatctctttccaaagtttttaacggctcataacaaaataagtgtcgtcttttggaagtgagtaaaaaatctaaaattacgtgtcgctttttggaataaagtagtagtatatttactaggtaatagtaattatgtacgaataaggtgttacgcatttaatttttttgcccaaaagccttgcagcaaggactttaggctgcaaaatattacatttagatgcgcgtcagcttacaaacatagttgtactcaagtcttcttatttctctctacttgcaaagtcattcctcatattgcgttaaaggttgatgtgagttgaggctgtatactttgctttttcgctcttagacttgctgtaccgtattggctaaccaactagtttcaagcgatgaagtttgttt

SEQ ID NO: 174 - DNA, 500 bp, unidentified
  misc_feature 1..500: note = source = /note="Description of Unknown: Non-coding transposon end sequence"
  source 1..500: mol_type = unassigned DNA
gtcaagtttgcttgtgagggttgaaaggaatgtcaccttcccaatactagaagagtgtcaaaagctatgctggctaatggaaggttgaaattaatcgctatttttttgcaaagaaaagattctaccagttataaaaaagcaagagtgacaaacaattagtcgttcaaacaaatgacaaattgtttgtcgctctaaagtatcctagattacttgactatacgagtttattttttagggattaagctcggggaaaagtttgtgatctactgacattcaagtaacactgacaaataatttgtcactgtacaatactcaataagagtcggctacattaaatgactataagacaaataatttgtcgctttaccacttttgacaaataatttgtcgccacggtcaaataatttgtcgctctacatttgaaagcgggcgatgggactcgaacccacgacgttcaccttgggaaggtgacattctaccactgaattacacccgcaaat

SEQ ID NO: 175 - DNA, 700 bp, unidentified
  misc_feature 1..700: note = source = /note="Description of Unknown: Non-coding transposon end sequence"
  source 1..700: mol_type = unassigned DNA
gtgttatgcagcaatatgagtagccgtcatgaactgcgtacaccaaaacctatgaaaaaggcatcaaaggattttcaaatcagaacaactggaaaaaaacaactatgttaaatattgtaaaaacaataagattgagttcgtagtaaggactttaatccttgtaaagttctcactacaagtctttaattatccaccctgttctactcagttcatttaaggctaacaacttagacgcggtggtaagttgaagccatctctccactgaaccatattgatatataatgtagagcgacacattatttgaccacttcgacaaattagctgtcacagcattatcttgaacgaaaatccatatatatcaatactttctagcaatgaaagctaaatcaagctcattcagtcttggacgacaaattacatgacttttttaaaagtccgcgacttattagatgacaattattaattttgcgaaaaattacgtgtcaattattcgcagtttcatagtagtataaatgtacccaataatcaaagacaaatcacatctatgaattatttagatgtagataaccagtttgaactggaagatgaagatgactttcttttggatgatgaggatgcagacatactcgatttttcaattgaagttgagtcattgagtaatgatggaaactctgtagcagaagacaaattagtagagttt

SEQ ID NO: 176 - DNA, 500 bp, unidentified
  misc_feature 1..500: note = source = /note="Description of Unknown: Non-coding transposon end sequence"
  source 1..500: mol_type = unassigned DNA
aagttacagttattaggtgggttgaaagtagtaaatgcttgttattttaggtttagacctcaaaaaaattacagatataacttcgagagctaatgtaaccaaaaataatacttagcccagtggagtgctactatgtaactgcatagaggaaaagacgatgacaaataatttgtcgctatgatatttatgacaaatattttgtcgctacagacaaataatatgtcgctctacatataaccaacaaaaaaggcttggtcgggccaagcctttatgtataccaagcatttactacaccaagcttaatttaaaccctctagcactttagttcatctgtctttagactgtatgcaaattctttcaaacctggtatgaactggcatatttacatacaaaaaaggcttggttcaacaccaaacctcgataattgctgtgcttaacaacgtactaaattaatttaaactggattgttgtccttatcatcttcctttagtatgtgttgc

SEQ ID NO: 177 - DNA, 700 bp, unidentified
  misc_feature 1..700: note = source = /note="Description of Unknown: Non-coding transposon end sequence"
  source 1..700: mol_type = unassigned DNA
ttcgggagtttctgcaaatcctggagtgggtggcgacgaagaaggggaagcgggtggtctatgttgagcgctggttcccttcgagcaagacctgttcaagttgtggtcatattttggagcatctggatttagagactcgccattggcggtgtcccagttgctcgacagagaatgaccgggatgagaatgcggcgatgaatattaaagtggctggggcttcagccattgggttaggtgatgtcagacaggcgttgcctgctattgctgtttgatcccagaatcccccgttttctaaacgggggagtaagtcaaagggttcttaacgttgtacagtccctcgttggcgtggtcgatcagttctgaactgtctgcccccacactgcccccaaaatcttgattttaatgacattttttttccaattttagggtgtgggtgtcgattgactcattaactgacatcttgacaaattatttgtcattgctatagacaagcctgcaacccttactatacataaggttgtgggctttttgcattggaaatttactcaaaagcaaaattgacaaattaggtgtcgtatattaggagtttgccaaaatatgtgtcgcttttcatttagtccagattttctggctttttgacacattctgtgtcgctcagggtaaaataacaacatgtttgtattaaacacatttgttgtaa

SEQ ID NO: 178 - DNA, 500 bp, unidentified
  misc_feature 1..500: note = source = /note="Description of Unknown: Non-coding transposon end sequence"
  source 1..500: mol_type = unassigned DNA
gtttgcctcggagatgtttgccctagtgagattggttgctgaaaccatcccaaggtgggattaggttgaaagagtaatacaagaattgggtggattgaaagcaagtcccaccgtcccctactttagtgcaatgcaagattacacaacaaacttgctctgaatgtatggaaggttcgtaaaagtggtgacaaataatttgtcaagatgacatcaatttgacaacgatgacaaataattagtcaatcgacatgtgggcaacaccgtcagaacaactcagaaaactctgaaacgatgggggtggtgggacttgaacccacacgtctttttacggacaacggattttaagtccgcagcgtctaccattccgccacacccccaagagcagtgacagggttctagtttagcagcaaatggcggccactgcatggaacagaaccctcagaaaatctaatcaatctgcctcttgcgctccgatgggttgaattgtttatgatgggagg

SEQ ID NO: 179 - DNA, 700 bp, unidentified
  misc_feature 1..700: note = source = /note="Description of Unknown: Non-coding transposon end sequence"
  source 1..700: mol_type = unassigned DNA
aacagattaaaaatttaataattaccgtaatctttagctaactataattatttacaactatgcagacacaataaccgcaataaaggttccttttcctcaaaaaagagcttagtaaaaattgccttatatagcagatgttatttaaaatacctgtgtgtgttatgcagcaatatgagtagccgtcatgaactgcgtacaccaaaacctatgaaaaaggcatcaaaggattttcaaatcagaacaactggaaaaaaacaactatgttaaatattgtaaaaacaataagattgagttcgtagtaaggactttaatccttgtaaagttctcactacaagtctttaattatccaccctgttctactcagttcatttaaggctaacaacttagacgcggtggtaagttgaagccatctctccactgaaccatattgatatataatgtagagcgacacattatttgaccacttcgacaaattagctgtcacagcattatcttgaacgaaaatccatatatatcaatactttctagcaatgaaagctaaatcaagctcattcagtcttggacgacaaattacatgacttttttaaaagtccgcgacttattagatgacaattattaattttgcgaaaaattacgtgtcaattattcgcagtttcatagtagtataaatgtacccaataatcaaagacaaatcacatct

SEQ ID NO: 180 - DNA, 700 bp, unidentified
  misc_feature 1..700: note = source = /note="Description of Unknown: Non-coding transposon end sequence"
  source 1..700: mol_type = unassigned DNA
actgcgccagcaattcaccaaactgccgcaatttaatcaaacaggtgttgggatcatctgcaaaatatcgctcggctagagtcgctaatcgaaccagttgtatgtcgtggactgcaaggaatgagaagtttaatgactctggcatactggcttttgagagaatctgtccagttagtttcaagttgtctcaaataacctaatcaagactcctcttgttgtaggttgcccaaattaacttagtcatagtctagcagtaattcaagtctcataatatacatctaactaaaaatttggaaaatattattaatctgtgaatatatagcaattcccaagctcatgaaatacaccccacccgtgctgtcgcgcaccgtagccttaccaaggggaggattggggaggggtaattttgtatctaactagagtgagaaaggctattgtacgttcgcaaattaaatgtcgcttttcgcaaattagtgtcgcaactgcttttaaggactaaaacctttattctataagcatcataagctatttacccctacaaaagactaagttacctaattcgcatattgtatgtcgtaaaacctggtttcgcaaattaaatgtcgtttgttaagaatttggtattttgcaaattagatgtcgcatttttgagtaattcatggtacattagtaccttaattattcatgagtgggtttcact

SEQ ID NO: 181 - DNA, 500 bp, unidentified
  misc_feature 1..500: note = source = /note="Description of Unknown: Non-coding transposon end sequence"
  source 1..500: mol_type = unassigned DNA
ataactagaatttacttgcgttaaccattaaaataaaatggttgcaaaatagaaatgatcatgggagggttgaaaggagtgctgcgatcgaacacatatcaataatccaataatgcccttgcagcaataatttattgtatagtaaatgttaagttcatgcgacattaatttgcgaaaacttagaataattaaattgactctgaaaaacaaccaccacgacattaatttgcgaataacgacactaaattgcgaaaagcgacatttaatttgcgaatgtacatatataatggagaataggagactcgaacccctgacctctgcggtgcgatcgcagcactctaccaactgagctaattccccttgctagtctcaagttactaactcagacagccgtactatatgttaacactcaggcagggtagttttcacctctttttgcaaaaaaacttcctgtacccggtctaattccaaatcagtcagataatctactgtccagttag

SEQ ID NO: 182 - DNA, 700 bp, unidentified
  misc_feature 1..700: note = source = /note="Description of Unknown: Non-coding transposon end sequence"
  source 1..700: mol_type = unassigned DNA
tctcatccaaaaaccttaaagatagttcaagaaaaaatagaaaatgagtgggagataaaaataagcaaagatacgataaaaagattagcaaaaaaaaagggcatggggtggtatcggttcaagaaaagggtaaaaggggaagtatgccctgaattataccaacaaaaaaagagacaattagagcaactaaaaagagcagaaaatgaaggaaaaatagatatatattatggtgatgaaagtggatttagtttagtaccttgtttaccttatggatggcaagaaaaagggagaaaaatagaaagagaaagtagcctaagtaaaagattaaatgtgttaggatttatgaaaaaaaataatgagttagaaagctatgtatttgagtcatcaatcaatagtgatgtcgtgatagcttgtatagataacttgagtaaaaaaataaagaaggaaacagtattagtaatggataatgcctcgattcaccaaaataaaaaattctggaataaagaagaagaatgggaaaaaaaaggattaaaaatattctttctaccaccctattcaccacaattaaacaaaatagaaatactgtggagatttatgaaatataaatggttagagaactcctgttataaaagttatttagatttagtaaaaggagtagaaaatattcttataaactttggttcaaaatatacaattaatt

SEQ ID NO: 183 - DNA, 500 bp, unidentified
  misc_feature 1..500: note = source = /note="Description of Unknown: Non-coding transposon end sequence"
  source 1..500: mol_type = unassigned DNA
gatcattataagactgcaaaatacaaaaatagctataatagatatatatgtggattgaaagaggtcacgagttcaatcataaaatgacatttaatttgttaacgatgattcagcgacaggaatttgttaagacgacaattaattcgttaagatgacagataaaatgttattcgacaatacctgtaatttaccattgcaaggacattcacacgaaagcctttgacgaggattgaactcgtgacctcacccttaccaagggtgtgctctaccactgagctacaaaggcagtgggccgagctggatttgaaccagcgtaggcatagccagcggatttacagtccgcccccattaaccactcgggcatcgacccatttgttttcacatttactaattatagctcatctttttttttaatgccaacttttttgtttaatttttatccagtttgcccaatgcgaggttctgttcctgctataattcgatcgatattagtacgatga

SEQ ID NO: 184 - DNA, 700 bp, unidentified
  misc_feature 1..700: note = source = /note="Description of Unknown: Non-coding transposon end sequence"
  source 1..700: mol_type = unassigned DNA
actatttgaaacagccaattacttttatcttcataagctacggttgaatctataacttctaaaaactccgtgtcgtagaaatagagaaaatcatcaattaacttttcttgctttactctccccgtagtgttggtataacctctttcaataaaaaatttaattgccgagcgaagctattattgccaacatagaaattacagcagcaataccaatgacaatcaaagcgaaaatcgccaagccatattgcagcgtgtatagcttgcgtaattggctcattgcgttcattaggttatcaatatcgttacctgaagttttgacaatgcgctgaaagccgccagccgctagtacattccagcgtcctatcaacagaagtagaattccctgaatgatgccactaattccactattaataggaacctcttctttttgaataaatatgtacggtgactaattatttgacataatgacaaattgttgtcatgcaattcaaagctttatatgctaggcatcatagcatttttattataatatttgattgacttatataatgacaaacaaaatgtcgtttgtttgcaaaatgactaattagctgtcgtttattgaaaattcagttttaaagaaaagtgacaagatctgtgtcacttttttatttaaatatagaattacattatgtgttttttaaaaaaactaatttactaaa

SEQ ID NO: 185 - DNA, 500 bp, unidentified
  misc_feature 1..500: note = source = /note="Description of Unknown: Non-coding transposon end sequence"
  source 1..500: mol_type = unassigned DNA
cgtaattcttaggagtgggtgcaaaatttaaccccaggtttcaatgaccattttgtgatggcttgggttgaaagataaaggcttagtcattgttattgatgaagcggaagtgccaacgaccacacgcaaattttaattctcaataactatcgtctaattctcgctaagaaactgaaagtaattttaaagtcttgttctttggttactccaagtaatttattacttttaggcgatcgccttgcttgccgagtttaatttgtatcagcaattgctgtatacagtctaaaaccaagtaaattcgattaattattttttgtgcgacgcagtaagtcgcttgatttaagtgaaaatttcatgactgaggttatggatttctagtagatttcatcctactattcccacgtaaaggagtctcgtactctggtcacattgaaagtaaataatgaactcaatggaatgcagaccatgaatgtatccgaaggtggataacctctatccag

SEQ ID NO: 186 - DNA, 700 bp, unidentified
  misc_feature 1..700: note = source = /note="Description of Unknown: Non-coding transposon end sequence"
  source 1..700: mol_type = unassigned DNA
cgaggacaataccggtatatttcagcaagtctgcactcaaaatactggggttgggctgtcgaagcgcgatcgctgtcaaaagttctggaatttcacaattctgcttccttttattttcttgtatttataagcacataagtaaaaataagtattctatgtatacctagtgatataagaatacacatatttaacaagatatatgaataaaattttgataaattttctgtaaaaacagtgaatatacttaatttttaattaaattttccaaattcaggactttcaagatattgacatttctgtggttagtattataccgtcccaatattcagattccaaaacattagtttctcttccattcgcaaattgaggaagcacagacggcacaaaccgataattatcagagtcaaaaatcgacaaaaggatgattgaaaagtgtacattcgcaaattaaatgtcgcttttcgcaaacaatgtcgcaactgtattgaatggctgaaagctacatcgtgtaaatattataggtcatttatctctaagacaggctaatttgcttgtttcgcatattatatgtcgcataatcacgtttttgcaaattaaatgtcgtttgttaaagttggtgacttttgcaaattaaatgtcgtattctttatgaatatatggtacattagtaccttaattactcatgagtgggtttcactct

SEQ ID NO: 187 - DNA, 500 bp, unidentified
  misc_feature 1..500: note = source = /note="Description of Unknown: Non-coding transposon end sequence"
  source 1..500: mol_type = unassigned DNA
acttagataatcaatcctagaaaaaacgctcttagtttatccgtctttgcgctacaaaaatcttgtcaaatggggtagggttgaaaggtgcatccgacttgtaagcgtattaaaccttaaaaacataaaacaaaaaattaaaacttatttaattctgattttttatatgtaagaattaggttctgcacaaccatcaattcacttgttgaagtaatattattgtgttaacacttgcttgtaagcaacactgtaacaatctagaaggaaaaaacgacattaatgtgcgaaaacctagcataactaaattgattataaaaaatagccgaagcgacatcaatttgagaaaaccgacactaaattgcgaaaagcgacatttaatttgcgaatgtacagattgaaaagccagcagtcggatttgaaccgacgaccttccgattacaagtcggatgcactaccactgtgctatgctggcaaattaggtgatgcgtttgaatcattca

SEQ ID NO: 188 - DNA, 700 bp, unidentified
  misc_feature 1..700: note = source = /note="Description of Unknown: Non-coding transposon end sequence"
  source 1..700: mol_type = unassigned DNA
tttctagtaaggtttctagcgcgatagaacagccgtggatatcactgatggctagtgttctcatagctttgcttttgtttttctatgatgcctttgatcctatcttgactaggttaaggtttatttaagacagagaaactatagtaaaatttttaaggctaaggtaaatctttgaacgactgcttcaccgcttaaaatggcatcctctcgatattgagtcatcgaaccatcttggcgataaacaaacgcttgacgattaatgcgatcgattaaccatcctaatttaaccccattgtttctatattcttgcattttttgtttaagtttacttaaactatcagatttagaacgaatttcaataacaaaatctggcgcaagattgaaaaattcatcctcttcctcatcccatccttgaggtaatctttccttagcaattgtacagtgacacattatttgacatgatgacaaattagtgtcattctttaaaccctttactcaataaggcctacagctattctatcattatttataatcgttcagctaagtgacaaattaagcgtcgctttttgcttttatgacaaattaattgtcgttttttcataaagcttcaaaaataattgtgacaaattcaatgtcactttttctataaatgtctgctaaaataacattatggtttttaaaatagtgttctaattatgag

SEQ ID NO: 189 - DNA, 700 bp, unidentified
  misc_feature 1..700: note = source = /note="Description of Unknown: Non-coding transposon end sequence"
  source 1..700: mol_type = unassigned DNA
atgattataaaaatattagtatggattgaaaggcagcaatacgggatgcaaaatgatgacatttaatttgtcactaaagcttaaacgacaacaattggtcacaacgacaattaaattgtctccatgacactaatctgtcatcgatgacaaataatctgtcactgtacaatagttcgtcaaaatgtaaaggcccggagtctaagcctatcacaagcatcccgtattgctgctaccttccagtcctgacaagatttgggcgttgtaatcgcataggtccgagccaattactaggataacacagaactgaggcactcactcttgatcgttgttcataatttgaggtacgcggaaaaagtcgccctcttgttctggggcacttttaagcaatgattcgcgatcgccataggtttctgtgcgatcgctgcgggtaatattgctcaattcgatcgcgcgtgtcattggggggacattttcggtgtcaagttcgcttaactgatcgaaatattgcaaaatgctatttaattggggggcaaacgcttcttcttcggctggtgtaatctccagtcgggctaaatgggcgattttttcaacttgttggcgatcaatcatcgatggtttctcactctgatttaaaagaagacatccatttcagaacgtccagtcaccttgagccaattttgggcttcaatatagttattgg

SEQ ID NO: 190 - DNA, 700 bp, unidentified
  misc_feature 1..700: note = source = /note="Description of Unknown: Non-coding transposon end sequence"
  source 1..700: mol_type = unassigned DNA
gaaccagcttccgtaggcaaagattattcagacgaagcagtattaagggattttttctttcatgattgtcaaccggatgtagcacagtgggcaatctcgaaaagtcgtccgcaacaatctatggcatatatatttgaaacgaatcctctaaaagctttgccgcatgttgagcgtaaatatattgtttgtagcaacgatcgcataatatctcctacatggtcgcgctacgctgcacgcaagcgcttaggagtcgatgccattgagctaccttctggacattgcccacatctgtctcgtcctgaccttcttgcctcaatattaactagtaattaattttttgatccatattttaggaaattaaccgaagcgtattgagtgtgaacaagggaatatctttgaaattttaattcggcatagacgaacgatattatctaattaataacgacatgaatttgcaaattgcgacattgaatttgcgaatgtacacatggtgtccattaaataattattgtcacttttaaagaattattgtccagttttacgatatctgtataacaggctcaaaggcttaccaaacaagtctttgagctatatttatacttttattattctctagccagttttaaagtaaatgatgtcatttttctgataaatttttaaagtaaatgatgtcatttttcttggaagacgggtcaagtgt

SEQ ID NO: 191 - DNA, 500 bp, unidentified
  misc_feature 1..500: note = source = /note="Description of Unknown: Non-coding transposon end sequence"
  source 1..500: mol_type = unassigned DNA
tatgatccccgaagtaggcgctttggttatataattttgtattagggtggtttgaaaggagtgctgcgatcgcacgtaaaacgcagctatgtagcagtggagcctctgcttgggtgaaaacagcaaaacaagaggtagtatatttttgtaattagactgcaaataaagggctttaaagctactcttgtaattcaataaccttgagcctgctgtaataaattaaattagtaaccatatatttagccgagttttacaggctgattttggacaataattctttaaaactggtttcaactctggacaataattctttaaaactgatttaattgctgaaagccgcgaaaatacgtaacttatgcccctgaacttggaaaaaatggacattaattctttaaaagtgacaataattatttaatggacacatggagaataacggatttgaaccgatggcctctgcggtgcgatcgcagcgctctaccaactgagctaattccccttga

SEQ ID NO: 192 - DNA, 700 bp, unidentified
  misc_feature 1..700: note = source = /note="Description of Unknown: Non-coding transposon end sequence"
  source 1..700: mol_type = unassigned DNA
gttttctcatcctaatcaacggggctaaatatactatattataatatctataactaaagtgtgggtacaataaataagctaaagtccatttaagtttaatactaactgacgataagccgtcttgaatttttgtaactcattcataccttcttgaagagttttaatatatgagagtctatgattaatgttaaataagaatgccacttagccacttagtttttgatctaatttcaattaaaggtaatttgagagaaaaacacacactatcagaaagtggacgagaagctctattttgctatccttgttttaattttggcagtaattgtttgactacttattaaaataaaattgatggaaaagttaaaaatcagtctgaaaatgcttttgaataactttgataaaaaatcttgtacagtaacagattatttgtcgtgataacaaattcgtgtcatctaaaaataagactcttaaaatccttatatatcaacaattataacttatccttaccctaaaatcacactcgattgtatcttaaaatagattaacaaattaagtgtcattttccctaaaaataacaaattagatgtcgccttcggaaaaggcgattttttttgtttttgagtggcaattcacagattaaatgtcatagaataataagtacacatcaactttgattttattaaacataaaaatatcagct

SEQ ID NO: 193 - DNA, 500 bp, unidentified
  misc_feature 1..500: note = source = /note="Description of Unknown: Non-coding transposon end sequence"
  source 1..500: mol_type = unassigned DNA
gcttttattttgaactaaaactctactaattagaatctgtcatcgagacgaaacaaaacgatcatattttctttttaaagttataatttcaatagtgccgtagatcaagttttaacaacctctgttctatgaaaaatgaggagtagtttactttttacaagaagtttgctttcggctcctgctaactacttgccctgatgctgtctatcttaggatagagaaactaggcgcactcccagcaataagggtgcaggtgtactgctatagcggttagcaaatcactttcgatcgaggaagaattctctttgagaattgaaagcgagtcctgtcgcacccaatcatttagacgacattaatctgttatcacgtcatctaatttgttaagacgacactaatctgttaccgatgacaaataatttgttactgtacatcaatttaaaatacgagccaacggctaagtaatacacgggtgcgacaggactcgaacctgtgaccgactg

SEQ ID NO: 194 - DNA, 700 bp, unidentified
  misc_feature 1..700: note = source = /note="Description of Unknown: Non-coding transposon end sequence"
  source 1..700: mol_type = unassigned DNA
tcatattcctgttgataagacaggcgttctcttgctgctgactccccttgagattgctttaaatcgtcaattaacgcctttaaaacctttttaatcaccgtagccgtcacattttcatcctcttccggctcataactcgccacctcttgcgctgactccaccgcttcacttaactccccttgcgctgcactaattttactctctaactcttcaatttgcgcctcctcagcttgaaaaaaggcagttaacagatattcatcaggaataagggtgtgatgccaacctgtattaataatcgtcttcaaatcatagcgaatctgttgccaccagttaacaaacacccccgcagacttaaactcatccaacacccccaaaggaataagctgctgtttcagactgtctagtaactcttgccggtgtacattcactaattaagtgtcgcaatttgacagattaatgtcgcaaaataaataagctgtaaaccttacacgacaagcattctagcatttttcagggactgtatacaaaaatgcttgatttaacggataatctgtcgtaaattagattcttaatgttttatctgtcgtttcttggatagatagtcaaattgctggtcaacattttttcatacattaattgtcgtttcttggaaaagagtctatactatggttagttcgtttgttggattctatgctaactc

SEQ ID NO: 195 - DNA, 320 bp, unidentified
  misc_feature 1..320: note = source = /note="Description of Unknown: Non-coding transposon end sequence"
  source 1..320: mol_type = unassigned DNA
ttgggatgggttggattcatgatcatgttcaaacatcgagaaggattgaaaggcgcacttcgttcgggatcgtagacttataaaaatattcgatcccaaaatagcgacacttaatgtctgaacaattgtccattaaataattattgtcacaattaaaaaattaatgtcacttttttgagagagttttaagataccgcaaggttctgttaaataaagctttcagcgattgaaaagagttatatcctgtcaattgtcattttaaactaaaatgtgtcacttattaaaaagccttgctagaatctttaaagtaaaatatgtca

SEQ ID NO: 196 - DNA, 700 bp, unidentified
  misc_feature 1..700: note = source = /note="Description of Unknown: Non-coding transposon end sequence"
  source 1..700: mol_type = unassigned DNA
ttatcagctaattctaaacgaataccatcaggagttttaatttttttgccttgagaaggttgccatatctcctgcaaccaatcacaaataagttgagaattagggttaggaatttctaaatataaaatttcttttagtttcatttatataagtacttgataaaaatatttagttattaatgagatgatttgatttattatctaataataggagtctcttcgccaagagaacaatatctgaagataaacagccaggattaagaaatagctgatccagaaatgatccagttttctaattttgaatgaaaacaaaaatatgagaatagcaaatattaataaataataaatgatctagcaattatccagttttttaatcaacaataaaacatcaaagaaaataatcaacaataaaaccttaaagtgtagagatggatgatgtacagtgccaaattaaatgacatgaagccaaattagtgtcgttgggtttaatccttattatctctacattatagtctttttctctaatttacgaagctaattttaaatgacaaattcaatgtcgtctttatatttagtgacagattaaatggcatcttttcaaatattctttttttttgacggtgacaaattcatcgtcattttttatttactgtgttaagataacattatgtttttatttaacaattatactatttagagtg

SEQ ID NO: 197 - DNA, 500 bp, unidentified
  misc_feature 1..500: note = source = /note="Description of Unknown: Non-coding transposon end sequence"
  source 1..500: mol_type = unassigned DNA
tataataaattacaggaaggacgtatagctcagttggttagagtacatcgttgacatcgatggggtctcgtgttcgagtcacgatacgtccatttatttttgtacagtgccacattaattgtcatcggtgacaaattaatgtcgttatgccaaattcttgtcttttaataagcagtgacaaattaaatgtcgtctaataaatttgccttgtggtttgtttcgatggtggggaaatttaatttttatttgtgaccccattgatgtcctttcaatctatcctaagtcgattatactgtattgatttatttcggctcgatcgatcattaattttttgtcttaccctaatttgatcgttgctcagatattttcaatcaaccccaaagggaaaacttgattgaaaccatcaatggaggcaatattccccgattagataatttttcaatcaaccccaaagggaaaacttgattgaaaccccgtgcttcggaagccttatacagtaa

SEQ ID NO: 198 - DNA, 700 bp, unidentified
  misc_feature 1..700: note = source = /note="Description of Unknown: Non-coding transposon end sequence"
  source 1..700: mol_type = unassigned DNA
tccgaattaaggatggagcaattgtcgcggcaagagtagatacacaaacgaccctgaagtatttctaccgacaaggaagcgaagtcattttgaaacctgcaaatgctgcttacgaacccaccgtagttgaagcgaaccaagtagatatccaagggatgtacgtaggactgatacggcgactttgggagaacggtgagccatgacttatagtgcttgcaatatcgatattgatctgcctgatgaagaggcggcagtcgatttcgaagactgtggcgatgcagaatacgaggattggctcgctgattattactcggattgggcagatttctatcaacaggtgtccgatgcagaagatgcctttcgacacaatttaatagtgaagaattatgagtttcaggcacgcggtggctttttaccgggggacactgtaatggcggaagtcgaaccagggtattggcatcactgcaccgtcctcttagtagggatatcgacgttagtggttagtgccgagcgatctacaaccgttcaagtcataactcaactcaacacgtattggctcgactagatctgattttggaattcgctcgtcttgcgatcgcttcaaatagttaatttgtattaattttcctgtctaacagattgactgtcgctatttcttcctcataggattagtgcagttcctgataggacttatgtactt

SEQ ID NO: 199 - DNA, 500 bp, unidentified
  misc_feature 1..500: note = source = /note="Description of Unknown: Non-coding transposon end sequence"
  source 1..500: mol_type = unassigned DNA
agtaacttagtcttttgacaactttaataggcacaatctttcggttaacagtgggtggattgaaaggactgcctgactcggggttttaatttagaaatatttgcccctcgctataacgagcagagttagcattcgttttaactgggtagagtctgtactgtaaaatgttgaagtggataagaatttgaatgagcattcaactgcttgaagctctgatgacaagaatttgttaacgaatgatttcttacccctcgacgacaagaagctgttaaaacgacactaaattgttaacgacgacatcaatccgttaacgacgacaaataaagtgttattcgacaacaatcacacgaatttaaacaaaaaattgcctgactcttaaaagcccccagagtcaggcagtctacctgtaaggcacgcctttaaccggatacaccaaacaaacttagttgccccctagttgcttagcttttccgagtgcgttcctcagatttgaatctatt

SEQ ID NO: 200 - DNA, 700 bp, unidentified
  misc_feature 1..700: note = source = /note="Description of Unknown: Non-coding transposon end sequence"
  source 1..700: mol_type = unassigned DNA
tttttatctccatgggaaatccagcaactgggggaagattctgcggcgaagcggagtggctggtgcgggctgctaccatcttaaccaagagccacaagtctttttgcccgtaccagccaccgcagaatctagcgcagcgttccacagttgcgtcaatgttaagagtaccggttttttgttaacgatcgcttgacttttaagacagtcgctacagaacttaaggtttttatggctaactaaaccgtattggtttttagttgaaatcgtgtccgcttaatcaacctgagtaaaaatgtcctacttttagcatgaactttctgaattccttgatatcctttatcaccaaagtattcaatattttctcttaaccgaactttactgtttttaagtatgcaaaaattattgtacattaactaattatttgtcattgtacaaaaatgtacagtgactaattatatgtcgtcgagacaaattaatgtcatccattaaaatcttgctcggtataggttacagcgttttagtagtattaggataacatcttgacagtgacagattagctgtcattttgggtaatagtgacaaattagctgtcgcttcatcagagataaaaaagcttttgtgtattttcataatgacaaattgactgtcgctttctgtttaagtagaataacaatatgtttttataaaaaagcttcgcatt

SEQ ID NO: 201 - DNA, 500 bp, unidentified
  misc_feature 1..500: note = source = /note="Description of Unknown: Non-coding transposon end sequence"
  source 1..500: mol_type = unassigned DNA
gcaaatctgaaatcattacgcacgcatacaaaaatgttttaataacctgccaggtactaggtgggttatcataatttttattagggagggttgaaagagagcgctacgttgacattatcgctatagtctctgtaaataaatgacattaatctgtcactggcacctaaacgacatcaattcgtcccgacgacagttaattagtcccaacgacattaatctgtcaccgacgacaaataattagtcactgtacaaaaaaaatggacgtaactggattcgaaccagtgacctctacgatgtcaacgtagcgctctaaccaactgagctatacgtccttaaccacacgaatattgatagtagcatatacttttgcgatcgcacaagataaagacccaagctttttaagggggttttagattttgagtgcgtaaatcctaatttcaaataaaatccttattatttcgttacaatcagcttgtgcgttaagcattcaagagttatca

SEQ ID NO: 202 - DNA, 700 bp, unidentified
  misc_feature 1..700: note = source = /note="Description of Unknown: Non-coding transposon end sequence"
  source 1..700: mol_type = unassigned DNA
caatggtggcttgcgacaaatagtcattaatctgctacacaacagcctcaagtttacagccaatggtggtcgagtgtgggtgagagctagagtccaaggcgaatacgtcatactagaaattcgtgacacaggtatcggtattgctgaaagcgaaattcccaaaatattcgactgcttttatcgtgtgcgatcgggactaattgatgagacgaatggcgcaggtttaggactcacaattgtccagagattgttatggcattgtggtggttctgttaatgtgagaagcaaggttgatgaaggtactatggtaatagtgcaaatgaagataggaagcacctcgccaacttagatccggaaaacggggtgaagcgatcgcctgttggttgacaactgcacttgtagagcgacaaataatttgtcgttctacaattgtcgattgccaaattatatgaccacttgacaagttaatgtcattcaaaatattatcctcaaaacccttgccaagcaagggttttgttattttaggctcaaaatacccagaaacttattgccaaaatagctgtcctcttttgaaaagttgacgaaatatctgtccttgcttgaaaagggacttgagggaagatttttacagaaattgacaaaataaatgtcctccaagtagtacaatacaactacgtttttattgaacatttatgtaaat

SEQ ID NO: 203 - DNA, 500 bp, unidentified
  misc_feature 1..500: note = source = /note="Description of Unknown: Non-coding transposon end sequence"
  source 1..500: mol_type = unassigned DNA
accttcatgcaaggatataaaaaattttaggtgggttgaaaggcgcacttcgttcgggattttccctgaccgaaacagtaccaataaatcaaagctattataataacagcttctaatgccaatacaccttgatgactaagtaatttggcaacgcggacaacaacttggcaatacggacaacaatttgtcaacgcagacaaagaatttggcaatcgacaacaataatcgggatgactggattcgaaccagcggccccttcgtcccgaacgaagtgcgctaccaagctgcgctacatcccgttaaaaaatttatacgccttttttagcataacataaataatagtcaatacagaaaagacatctacatctatatatatagatgaatcggaacgggatttgcttgctaggattagatttgtataacattcagtggtaaaagcggattgtgactgtaaaaccagactggttgcgggtaaaagcgcctcaatgggagcgcgttgg

SEQ ID NO: 204 - DNA, 700 bp, unidentified
  misc_feature 1..700: note = source = /note="Description of Unknown: Non-coding transposon end sequence"
  source 1..700: mol_type = unassigned DNA
aaagcagtaggactattatctgcccatatccacagtaatccttgtaatacttgagtcggatatgtggtaatttgcgatcgctcgcttttacaagcagtttctaaagccttttcatcgctcaacatgggaatatttgtacatgttcctgtcccatcaaaacaccaaccatgatggcgacacatgatgttgccatttttgtcaatacttcctaaagataactgcgctaatttatggggacaaacatcatccattgccacccatttttgctgcttatctctccaaattaccagatttttacccagcaaagtaattgcagtgggatgagagggttctagatagctgataggagttacaggataccactgtttagtccaggaaaagttagtcatcatcaaattaactaatttattcgactcaaatttgaaaaatgagtaaaaagcggcatttaatgtgcgaatgttgtacattcgcacattatatgtcgcttttcgcaagttaggtcgcaaccgcatttaactgctataaaccctattttacaaaggtttgatgctcttagcacatcaagcccacgaatttacttaattcgcatattccatgtcgcaaactaaatattcgcaaattgaatgtcgtttattaaaatttgtcacttcgcaaattgtttgtcgtattattgagcgattcatggtacattggtactcta

SEQ ID NO: 205 - DNA, 700 bp, unidentified
  misc_feature 1..700: note = source = /note="Description of Unknown: Non-coding transposon end sequence"
  source 1..700: mol_type = unassigned DNA
ttgaaaatctaagagcaactacactatagatttatttagagggttgaaaggagcatcagactcttaatttaagtgatagagataatatttaagcacagatatatttcttactcagcaatagcatttagtatttgcatggaaataaatagctttgagacaacattaatttgttaacgatgtctttgaggatttaggggacatcaatttgttaaaaaagacattaattttgttaacgacgacaaattatttgtaatcgactttaggacaaataatttgtcgctttatgtactttgacaaataatgtgtcgctctacatcagttaatcgacaataaccaagcgacgttaatttgcgaaaacctataatcatcaatatagtacacaaatctgtcgaaagcgacactaatttgctaataacgacactaatgtgcgaaaagcgacatttaatgtgcgaaagtacaaatgtacaaaatgggcgacctggggctcgaacccagaaccagcagattaagagtctgatgctctaccattgagctagtcgccctcaccatttactactatagcaaattatttgaacaatagttaacctttatcggaaaatcttcatataactagttgacattagcaaacacataccctctgatgcggataaaacttgctcatacaaaatcaaaactaggtaatagcaccactaattatact

SEQ ID NO: 206 - DNA, 700 bp, unidentified
  misc_feature 1..700: note = source = /note="Description of Unknown: Non-coding transposon end sequence"
  source 1..700: mol_type = unassigned DNA
agtcaggagtcaggagtcagaaagaagaattagaaggagatcagaatattctcaaatctgggtaaatgaactatttttgatgctttcatcgcttatttcttgcaccttctccaactttttcagcctgagactgttgatatatctatgtttttggtttattaagcaaaccctaagtactatctgcaagggtttcaggcatgatagaccccttaaattgtcattacgagcggagcgaagtaatcacataatgcgatcgcaccacctcaagctagatgatgaaaccaagtactagtaggaattttaagacttgtgtgtacaccgtagattattgagtaggggcatctaacgataacgaataatgataaataccgtagtgcaatattatgctacaggaaacttaatcagtgtttacataatttgtgtacattaactaattatttgacaatttaacaaaatattgtcaaaaatcataaaaataccttaaaaccttctacagcaaagattgtaggaggtttttatttatctaatttgtgaacatcctccagacatatatttaacaaattaagtgtcaaaagccagataattatcaatttatttgtcaatttgccaaatacaggtaaattactatatttctgaaaatttcacacattaaatgtcacttttactctatactatacaaatatgttgtaattaaacatta

SEQ ID NO: 207 - DNA, 500 bp, unidentified
  misc_feature 1..500: note = source = /note="Description of Unknown: Non-coding transposon end sequence"
  source 1..500: mol_type = unassigned DNA
acattaatgcaagtagaataaaaaatatctaggtgggttgaaagatcagtaggtcgtgggtttaactctgaaaacacttgaaaacactatataaatacagtgttgcttgtgacatttagggacaattaatttgttaacagtgacacgaattagttaaaaatgacattaatttgttaacagtgacaaataaattgttaatgtacagacatatagtgtgcgaatgtacaccaaagccgatgatgggatttgaacccacgacctactgattacgaatcagttgctctacccctgagccacatcggcatacacagtttgatatcatagcattatttacctatgatagacccaaattcccaaaatcgtcttaactctaaacaatatgcccacctgaaagctgaaatagcagccccttatcgtggtttgcggcaatttatctatataggtgttggtgcttctggttttatcggcgcattcaccttcttttttcaactgcttgctgg

SEQ ID NO: 208 - DNA, 700 bp, unidentified
  misc_feature 1..700: note = source = /note="Description of Unknown: Non-coding transposon end sequence"
  source 1..700: mol_type = unassigned DNA
cattggaaaacagcgaacagtgctttagccagaatcagaacagcatttgatcggcacagtcttaagccatatactttttttgtgggatctgctatagaaaaaagcatggcagaagaaatttggcatcaactaaattctggcattcttactaatgcagcaaacctcactcagcctgaccaagttgcctctttgtgcgcttggtttattcaactttgatgtacagtgactaattatttgtcactgtacacaagatgtacagtgactaattatttgtcactgtacacaagatgtacagtgactaattgtttgtcgtcgtgacaaattaatgtcgtcgaatgaatccttgcaatacaaaggttttagctatttaagagttattacattatttctcatacgtgactaaataaatgtcgttttccgcaaaaatgacaaattaactgtcgcttcagtaattacaaaaaaggttttgtatattttcataatgacaaattgactgtcgttttctccacgtttagaataacattatgtatttataaattacctttgtttcatgaacaaaaaatcttatgtctgattttactgttcataccgcagtggatactgcggaagctttattacaagacaacaacacacctcctcacctcctgagacgaatctgattgttacagaactctcagaggaagcccaactcaagctagag

SEQ ID NO: 209 - DNA, 1300 bp, unidentified
  misc_feature 1..1300: note = source = /note="Description of Unknown: Non-coding transposon end sequence"
  source 1..1300: mol_type = unassigned DNA
acatggtgaaaacaaattggatatgaaactgccttacgttgtctgatggtgtaattaatttccaagaagtagagggttgaaagcaaatcccgtcatcggcttgtaactaaagttattcaggataagctactcatagaagtgattaaaagagctttttgaatgcaaacgcttataataggggctaaatatatagagaaactccatatataaattgttgcttttccgaaaaatgacaataatttgtcacaaatatatatggaagcgagttactaagttggatgacaataatttgtcacaacgacatcaatttgtcaccgacgacaaataagagaccatttataaagtaaatctttagacgactagacgacgtagcataatacgagtcataacggcatatatggcagcctcactcatttctgggagacgctcataatccttactgagacgacggtactggtttaaccagccaaatgttctttctactacccaccgtttgggcaaaacctgaaattcttgattagtacgccggattacctcaacatgagcttgaatcatcagccaaacagagagcgcaaatttatcaccgtcatagccggaatcaacccagatgacttcaactttttccagtaattctggacgctcttctaacagttccatcaaagtataggcggcaagtaatctttctccagcatttgcttcacttacaaccacttttaacaaaagtcccagactatcaaccaaagtttgccgctttcgtccttttaccttcttgccaccatcaaaaccgtacacatcccccttttttcagtcgtttttaccgactggctgtctgccgcgatcgccgtgggttgagttgacttccccattttttgacgaacttgatcgcgcaaagtatgattcatttgctcccagatacctcgtttttgccatttacgatagtagctgtaaacagttgaactaggaggaaaatcccctggaagcatatcccactgacaacctgttttcagatggtagtagatagcgttgcatacttctcgcatatcagttgttcggggatgcccaccgcatttagcgggtggaatcaaaggagctaaaattgcccattctgagtcattaaggtctgtagaataagactttcgtctcattgtttcctatgtaaatacactctacaaacagtatcttatcgctgcctttttatctgatagctctcctttagatttactttataaatagcctctaatttgtcactgtacacaagaattagtcaagacaaaaatatgattttatgaaaaccaagccg